SEQUENCE LISTING (104 sequences)

SEQ ID NO: 1 - protein, 94 aa, Klebsiella pneumoniae
MKKLALLSAVMTLGMSSWAFAADNPPPPPEKGAQHQGKPPVKNGQHEGKQAQYNRKQPQRDGKQPQHDGKQPQHNGKQPPKGSEHSGKPLPPKA

SEQ ID NO: 2 - protein, 117 aa, Klebsiella pneumoniae
MKRYATALLFCTLSLTSLAARADIIDDAIGNIQQAINDAYNPGSSRSDDDDRYDDDGRYDDGRYQGSRQQSRDSQRQYDERQRQLDERRRQLDERQRQLDRDRRQLESDQRRLDDSY

SEQ ID NO: 3 - protein, 119 aa, Klebsiella pneumoniae
MFRSLILAAVLLAAGPLVANAGEITLLPSVKLQIGDRDNYGNYWDGGSWRDRDYWRRHYEWRDNRWHRHDNGWHKGWYKGRDKAWERGYRAGWNDRDDHRGGWGRGPGGRGHGHGHGHH

SEQ ID NO: 4 - protein, 120 aa, Klebsiella pneumoniae
MKEIGLPLLLLTALASPAFAADCQPNGIGGSFCINDDGTTTDTVPNEVNGMDTYSNNGGYTSSLPDRSGADEALEGSSLSTQQGVGSGQSDSALAGRDWHSPANLNDGAATSSMSLLDKP

SEQ ID NO: 5 - protein, 121 aa, Klebsiella pneumoniae
MNMKKLTTLLLTATLGLASGAALAADTGAQSNNGQANSSADAGQVAPDARENVAPNNVDNSQINSGSGGTTGSTMTQDNMSSNEVHKNSMCKDGRCPDTGKKLDNGGNTTQDNSKTDGTTQ

SEQ ID NO: 6 - protein, 122 aa, Klebsiella pneumoniae
MKHRIALLLVLTSLSASALAASPCQEKEQDIQREISYAEKHHNQSRIDGLNTALRQVRENCSDSKLKADHQQKIAKQREEIAERQRDLQEARKKGDADKINKRQHKLNEAQQELKTLESRDY

SEQ ID NO: 7 - protein, 141 aa, Klebsiella pneumoniae
MRLITRHVREDIMKKAMIALSAILVAAPVFAATTHATDDTVAAANANANTAKEKLHQAQHEGEEQQLKAKHAAEGKQDSVGSQVSEGAQKTWNKTKEGTEKGWDKTKEVSEKGWNATKSGAEKGWDKTKTGAEELKNKVTE

SEQ ID NO: 8 - protein, 151 aa, Klebsiella pneumoniae
MKKMISLAVILSCVLSVPAFADGPNDGHRPEQPTVWQNGPDHDGHAPQGGPDAHHQGDHDQRGPDRDGHDKRDLARHEQDHFAWRGNDFRKGHPAPAPFRGDEYRVRDWSDRGLPPPPEGHHWSYIDGNYVLIAAATGIITSILVSGALGH

SEQ ID NO: 9 - protein, 168 aa, Klebsiella pneumoniae
MKKPTSATRGKSGRKSREELNQEARDRKRQKKHRGHAAGSRANGGDAASAGKKQRQAQDPRVGSKKPIPLGVSESSVPAPKQHKPKSEKPMLSPQAELELLENDERLDALLERLEEGGTLNAEEQSWVDAKLDRIDELMQQLGLSYDDEDEEEEERQEDMMRLLKGGN

SEQ ID NO: 10 - protein, 229 aa, Klebsiella pneumoniae
MASKFQNRLVGTIVLVALGVIILPGLLDGQKKHYQDEFAAIPLVPKPGDRDEPDMLPAATQALPSQPPEGAAEEVRAGDAAAPSLDPSRIPVNSNSFDDVQEPVVAAKPQPKPQPKPQPQQQASTPTPPPAKPQQQQPPQQQAALPAPTGKAYVVQLGALKNADKVNEIVGKLRASGFKVYTSPSTPVQGKITRILVGPDASKDKLKGQLGDLQQISGLSGVVMGFTPN

SEQ ID NO: 11 - protein, 322 aa, Klebsiella pneumoniae
MAQRDYVRRSQPASSRRKKSTTRSSRNKQSSLPAISPAMVAIAAAVLVAFIGGLYFITHHKKEEAEAMQNRQAAGNGLPPKPEERWRYIKELESRQPGVRAPTEPTAGGEVMKPEQLTDEQRQLLAQMQADMRQQPTQLTEVPWNEQTPAQRQQTLQRQRLAQQQQQAQQQQWAQTQAQTVQQQPPRVQQPKPVQQQQPKQTASNQQPYQDLLQTPAHTNTTQPRTQAAAPVTRVEEAPKTTAEKKDDRSWMIQCGSFKGAEQAETVRAQLAFEGFASHITTNNGWNRVVIGPLKGKESANEMITRLKMAGHANCIRLAARG

SEQ ID NO: 12 - protein, 376 aa, Klebsiella pneumoniae
MSAGSTKFTVSRIAALSLVSLWLAGCTNTNNPPAPVSSAGGAASSSTNSGMLITPPPSGVKSAPQAQPIQPMQTQTIQPAPVAQEPVQTVNGRIVYNRKYGDIPKGSYTGGSTYTVKRGDTLFYIAWVTGNDFRDLAQRNNIPAPYALNVGQVLQVGNASGQPITGENAVSQASARASGGATTSTTSAQKSTAVVASQPTITYSESSGEQSATKMLPNNKPATTTTTVVAPVTAPTTVSTTQPTASSTSTSSPISAWRWPTDGKVIENFSGAEGGNKGIDIAGSKGQAIVATADGRVVYAGNALRGYGNLIIIKHNDDYLSAYAHNDTMLVREQQEVKAGQKIATMGSTGTSSTRLHFEIRYKGKSVNPLQYLPQR

SEQ ID NO: 13 - protein, 382 aa, Klebsiella pneumoniae
MRKQWLGICIAAGLLAACSSDDVQQKTVSTPQPAVCNGPTVEISGADPQYETPNATANQDYERDGKSYKIVQDPANFTQAGFAAIYDAEPNSNLTASGEAFDPTQLTAAHPTLPIPSYARITNLANGRMIVVRINDRGPYGNDRVISLSRASADRLNTSNNTKVRIDPIIVAPDGSLSGPGMACTTVAKQTYALPARPNLDGGDAAGMSQPAPTDVRPISNSTLTPADSVGAPVNSGGFLGAPTPLNNGVLESSEPAAAAATAPAAGATPTAPVTAPGSIQGNVVPAAATAAAAGAVAASSSATSSASGNFVVQVGAVSDQTRAQQYQQRLSQQFSVPGRVMQNGAVWRIQLGPFADKAQASAVQQRLQSEAQLQSFITRAN

SEQ ID NO: 14 - protein, 428 aa, Klebsiella pneumoniae
MDDFKPEDDMKADRNDRRAGRSRQSSERDADPQINFDDVDLDADEGRPTRAGKARREREEEEFEEELDAQDEEMLEEQPVERRPRKRKKAPAKPASRQYIMMGVGILVLLLLIVGIGSALKSPSSSSQQTASGEKSINLSDDQSASMPAAGQDQTAAANSTSQQDVTVPPIAANPTQGQAAVAPQGQQRIEVQGDLNNALTQQQGQLDGAVANSTLPTEPATVAPIRNGANGTAAPRQATERQTAATPRPAERKHTVIEAKPQSKPQAVAKTPVESKPVQPKHVESTATTAPAKTSVSESKPVATAQSKPTTTTAAPAATAAAAAPAAKTGKTAGDVSSMKTAPSGHYTLQLSSSSNYDNLNNWAKKEKLDKYVVYETSRNGQPWYVLVSGIYASKDEAKRAVTSLPADVQAKNPWAKPLHQVQADLK

SEQ ID NO: 15 - protein, 441 aa, Klebsiella pneumoniae
MSKATEQNDKLKRAIIISVALHIILIALLIWSSFDEHLDASAGGGGGSSIDAVMVDPGAVVNNYNRQQQQQASARRAAEQREKQAQQQAEELREKQAAEQERLKQLEQERLQAQEAAKEAKEQQKQAEEAAAKAAAAAKAKADAQAKEAQEAAAKAAAEAKAKADAQAKAAEQAAAKAAADAKKQAEAAAAKAAAEAKKQAEAEAAKAAAEAQKKAEAAAAKKAQQEAEKKAQQEAAKQAAAEKAAAEKAAEKAAAQKAAAEKAAAEKAAAAEKAAAAKAAAAEKAAADKAAKAAAAKAAAAKKAAAAKEADGVDNLLGDLSSGKNAPKTGGGAKGNNASAAGSGNTKNSASGADINNYAGQIKSAIESKFYDASSYAGKTCTLRIKLAPDGLLLNIQSEGGDPALCQAALAAARQAKFPKPPSQAVYEVFKNAPLDFKPQ

SEQ ID NO: 16 - protein, 484 aa, Klebsiella pneumoniae
MFFLSIFYMEMTKVKLSALFIALIPLLGSPVIHAETTAAPVLENRAAQGDITTPGGARRLTGDQTEALRASLINKPAKNVILLIGDGMGDSEITAARNYAEGAGGFFKGIDALPLTGQYTHYSLDKKTGKPDYVTDSAASATAWTTGVKTYNGALGVDIHENAHQTILELAKAAGLATGNVSTAELQDATPAALVAHVTSRKCYGPTVTSEKCPSNALEKGGKGSITEQLLNARPDVTLGGGAKTFTETATAGEWQGKTLREQAQARGYQIVTDAASLAAATEASQDKPLLGLFADGNMPVRWEGPKASYHGNIDKPPVTCTPNPKRDASVPTLAQMTEKAIDLLSRNEKGFFLQVEGASIDKQDHAANPCGQIGETVDLDEAVQKALEFARKDGNTLVIVTADHAHASQIIPADSKAPGLTQALNTHDGAVMVMSYGNSEEESMEHTGTQLRIAAYGPHAANVVGLTDQTDLFTTMKAALSLK

SEQ ID NO: 17 - protein, 535 aa, Klebsiella pneumoniae
MSLPFKPHIIALLCSAGLLAAAGTLYVQSRTPATIAEPPAQQAPAPAASTTQPVAATYTQAQIDQWVAPIALYPDSLLSQVLMASTYPDNVLQAVQWSQDNPAMKGDAAVQAVASQPWDPSVKSLVAFPALLAMMGENPPWVENLGNAFLAQPHDVMDSVQRLRAIAQQTGTLKSTPQQKVIVTPAAPVSASSSTAATATAHTAAPAPTQVIKIEPTNPQVVYVPSYNPSTVYGTWPNSAYPPVYLPPPPGEQFTDSFVKGFGYSLGVATTWALFSSIDWDDDDHHHHDDDYHHGDYSHNGDNININVNNFNHITGENLPGNHVNWQHNPAYRGHTPYPDNTVAQRFHQTNVSGGLSATQHAPVDREAQRQAAMTQLQHNVPTATAGNLAANNASRDAQRQAASAQLKQATQRSNYRGYDSTPTQQQRREAAKTQLKNPTPQQQQRREAARSHEQNRTPQQQQRRQQFQSATPAQRQQTLSHLRANALSGNESRAPSWQAQQERGLQSRQFSGVNRELRDGTRERLSEHHELRRR

SEQ ID NO: 18 - protein, 597 aa, Klebsiella pneumoniae
MFKFKASYVALAAVLTSSVVYADPTSYTHSSGATVIDIEKPNAAGVSHNLYRDFNVGANGTILNNSGDDVSHSTFGNIARNNNLTAGSASVILNEVTSKNASSLKGFIEVNGQKADVVIANPNGITCSGCSFVNTNKAILTTGKVNMTDDGAIGSYTVTGGTLTIGENGMNAANGYAVLLADAININGKVQANNALVSAGNFTMDNSSGSVTSAGKKATLIQMTVNPQYSIDVSSLGGIEANSISMVGNNIGFGVRNKGSIVANSSLQLTSNGNLLNKGTIKSNGLLSQVATASGITNDGSIAGAYYLMLSSGDYIVNTGSLSGGQLIATANGNITNGDSGTMTGTSGLSLTSGGKIRNEEKASLLSNNQIAATAIGDFLNEGKISAKHTSLTFVGDSFKNTGNINSTGQTTIQSLKQDGSANTGEIYNLGNITGENINLQTNGTLAQSSSGRIEATNAITAHSYWLNQNGYMNAADITTDHGVVNNYGNITAKNISITTYSDITNEGQISSTGDLTLNTKNKGAIYNYSTLSAGGNMTLTATKVVNGGKSCGILGLAKCGVGTLTADKLVLNSSQKYVSDMGGKQYFKSTEVNTVK

SEQ ID NO: 19 - protein, 624 aa, Klebsiella pneumoniae
MMDNLRTAANSVVLKIIFGIIIVSFILTGVSGYLIGGGKNYAAKVNGQEIGRGQFENAVASERNRMQQQLGDQFSELAANENYMKTMRQQVLNRLIDESLLDQYARELGLSISDEQVKQAIFQTQAFQTNGKFDNQRFSGIVAQMGMTTDQYAQALRNQLTTQQLINAIAGTDFMLPGESDQLAALVSQQRVVREATINVNALAAKQTASDEEINAFWQQNQARFMAPEQFRVSYIKMDAASMQESASDEEIQSWYDQHKDQFTQPQRNRYSVIQTKTEADAKAVLAELQKGADFATLAKEKSTDIISARNGGDMGWMEDASTVPELKDAGLKEKGQLSGVIKSSVGFLVARLDDVQPAQVKPLADVRNDIAAKVKQEKALDAYYALQQKVSDAASNDNESLASAAQVAGLKVVETGWFGRDNLPEELNFKPVADAIFNGGLVGENGAPGSNSDIITVDGDRAFVLRISEHKAEAVKPLAEVKAQVSDIVKHNKAEQQAKLEADKLLAALKDGKGDEAMKAAGLSFGAPQTLSRTGQDPLSQLAFTLPLPQQGKPVYGVGSNMQGDVVLVALDEVKAGSMPEEQKKAMVQGITQNNAQIAFEALMSNLRKAAKIKLGDSIDQQQ

SEQ ID NO: 20 - protein, 657 aa, Klebsiella pneumoniae
MFRLNPFIRAGLSASVVSLAFPALADVNEETLVVTASATEQNVKDAPASISVITQQDLQRKPVQNLKDVLRDVPGVQLTNEGDNRKGVSIRGLSSSYTLILVDGKRVNSRNAVFRHNDFDLNWIPVDAIERIEVVRGPMSSLYGSDALGGVVNIITKKIGQKWTGTLSADTTIQEHRDRGDTWNGQFFTSGPLIDGVLGMKAYGSLAKRAKDDPQSSSNATGETPRIEGFTSRDGNVEFAWTPNENHDFTAGYGFDRQDRDSDSLDRNRLERENYSLSHNGRWDIGNSELKFYGEKVDNKNPGQSGTITSESNAIDGKYVLPLGMINQLVTFGGEWRHDKLKDPVNLSSGGQSTSASQYALFIEDEWRIIEPLALTTGIRMDDHQTYGDHWSPRAYLVYNATDTVTVKGGWATAFKAPSLLQLNPDWTTNSCRGSCSIVGNPDLKPETSESFELGLYYRGEEGWLENVEGSITTFQNNVDDMIDVLRTSSASEAPGYPNFVGWKTVNGKRVPIFRYFNVNKARIKGVETEVKIPFGDEWKLTVNYTYNDGRDLSNGGDKPLQTLPFHTANGTLDWKPLDDWSFYVTANYTGQQRAVSATGKTPGGYTLFDVGAAWQVTKNVKLRSGVQNVGDKDLSRDDYSYTEEGRRYFMAVDYRF

SEQ ID NO: 21 - protein, 675 aa, Klebsiella pneumoniae
MNRAATLTLNAPLLMLVAALALSTPFTAGAAPAFLDYAQQQTQQSQAQEKNDAASAKQTQESRQSADNKKTGTSTSQLQKRITSQQAAIAQKDKLIQQLKKQLAATPQTDTAGANEQAALNKRINELQVALSAATAEKEALIKKAGVVQNNNLQQSQAAARQQIQQLTTQIQQAEAENKRLSASFTTLNKDKHALMTQLAATEKEKQAALEQVKALNADKQPLTTRLAAAEKEKQAVLEQVKALNADKQSLTIRLAAAEKAQQAAVDQAKALNADKQPLATRLAAAEKEKQAVLEQVKALSADKQSLTIRLAAAEKAQQAALDQAKALNADKQPLATRLAAAEKEKQAVLEQVKALNADKQSLTIRLAAAEKTQQAALDQVKALNADKQSLSTRLAAADKAPHGPANDAAAPKNEPPEMAAIVAAYRLQADKDNAQLRMKEDEIELLRTQLSVQSKTRSGESAAAKLSASGEQQAYAIGASMGSEALNVLTTRRTQGVTVDAGLVLQGIEDAFRGQLRLGEQERNKALFDVSQQVFQNLNKIEQKNISAGKKYQQAFARKKDVVFKEGVYSRVDYLGKGKISGNDLVTVVIKEMLTDGTVINDMEAKDQALTQKLDAYPPVFREPLKRLQNHGSVTLVVPPEKAYGSKGLPPKIPPGATMVYSVRIVDSQPEPAK

SEQ ID NO: 22 - protein, 701 aa, Klebsiella pneumoniae
MKILSVRHAALPALLLPLIAAAQAADEQTMVVTAAPTTVSELDTPAAVSVVNGDEMRQAAPRVNLSESLGAVPGLQVQNRQNYAQDLQLSIRGFGSRSTYGVRGLRIYVDGIPATMPDGQGQTSNIDIGSVDTIEVLRGPFSALYGNSSGGVINVTSQTGTQPPTVEASSYYGSFGTWHYGMKATGAVGDGSHAGDVDYTVSTNRFTTHGYRDHSGARKNLANARLGVRINDVSKLTLLLNSVDIKANDAGGLTADEWRDNPRQSPRGDQYNTRKNTRQTQAGLRYERQLSAQDDLSVMMYAGERETTQFQSIPRAPQLKPSHAGGVIDLTRHYQGIDTRLTHRGELLVPVTLTAGLDYENMSERRKGYENFVMVNGAPQYGEQGALRRNERNLMWNVDPYLQTQWQLTDKLSLDAGVRYSSVWFDSNDYYITPGNGDDSGDASYHKWLPAGSLKYALTDAWNVYLSAGRGFETPTINELSYRSDNQSGLNFGLKPSTNDTVEIGSKTRIGNGLFTAALFQTNTDNEIVVDSSSGGRTSYKNAGKTRRQGMELGLDQQFGESWRLKAAWTWLDATYRTNVCDDASCNGNRIPGIARNMGYASFGYQPEQGWYAGSDIRYMSDIMANDENTAKAPSWTVVGLTTGYKWSYGRMDMDLFGRIDNLFDREYVGSVIVNESNGRYYEPAPGRNYGIGLNLAWRFE

SEQ ID NO: 23 - protein, 752 aa, Klebsiella pneumoniae
MKYTSHFPLGIVIPLLACSVPLQAAENMTEQSTPDESAATAENHEETMVITAARQNLQAPGVSTITAEEIRKHPPARDVSELIRTQPGVNLTGNSTSGQRGNNRQIDIRGMGPENTLVLVDGKPVTSRNSVRYGWRGDRDSRGDTSWVPAEMIDHIDVIRGPAAARYGNGAMGGVVNIVTKPTTREWHGSWNTYMNAPQHRKEGATKRTNFSLNGPLSDSVSFNLWGNLSKTQADAQDINAGHEAERTGSYAGSYPAGREGVVNKDIHSKLRWEFAPMQALEFEAGYSRQGNLYAGDTQNTNTSTLVKSMYGKETNRLYRQTYGVTWTGGWDNGVTSNSYAQYEHTRNSRMDEGLAGGTEGIFSSSEFSDIDLADVLLHSEVNIPFTLGVDQNLTLGAEWNQQRMKDGVSTTQALSYGTIDGVSATGRSPYSSAEIFSLFTEDNMALTDSTMLTPALRFDHHSIVGNNWSPSLNLSQELTDDWTLKLGIARAYKAPNLYQLNPNYILYSNGQGCYASSSACYLMGNSDLKAETSVNKEIGLEYKHDGYQAGITWFRNDYHNKIESGYAAVGTASNGTTNIYQWENVPKALVEGLEGTLNLPVGEAVNWSNNLTWMLQSKNKTTGDRLSVIPQFTLNSTLSWQVREDLSLQSTFTWYGRQKPKRFNYKGEAVSGSELNEVSPYSIVGLSATWDVNKNLSFTSGIDNLFDIRHYRAGNAQTTGNATTGAYLYGAGAETYNESGRTFFMSVNTHF

SEQ ID NO: 24 - protein, 761 aa, Klebsiella pneumoniae
MEKNASLPFGSFNSLALFTGLCLGASPAAGIAAENSVKNSEETLVVEAAPPSLYSPGASADPKFNKPLVDTTRTITVIPEQVIKDQGVTNLTDALKNVPGVGAFYAGENGSSTTGDAIFMRGVDTSNSIYVDGIRDIGSVTRDTFNTQQVEVIKGPAGTDYGRSAPSGSINMISKQPRLDSGIDGSASIGSAWSRRGTLDLNQAFSDNAAFRLNLMGEKTHDAGRDRIENERYGIAPSLAFGLDTPTRLYLNYLHVRQNNTPDGGIPTVGLPGYSAPSPKYAALNSAGKVDTSNFYGTDSDYDKSTTDSGTLRFEHDLTENTTVRNTTRWSRVKQEYLLTAVMGGANNITAPDINDVNTWSWSRLVNTKDVSNRILTNQTNITSTFNTGSIGHDVSAGVEFTRENQTNYGVNARTAPAVNLYHPVSNLSIGGLDRNGANANGQTDTFGIYAFDTLTLTERIEINGGLRLDNYHTKYDSATACGGSGRGAIACPPGQSTGSPVTTVDTAKSGNLVNWKAGALYRLTEQGNVYVNYAISQQPPGGSSFALAASGSGNSANRTDFKPQKAKSSELGTKWQIFDNRLLLSAALFRTDIENEVAANDDGTWSQYGKKRVEGYELSATGNLTPDWTIIAGYTQQHATVTEGQNVAQDGSSALAYTPKHAFTLWTQYQATSDLSVGGGVRYVGSLRRGSDGAVGTPDHTEGYWVADAKLGYRVNSNLDLQLNMYNLFDTDYVASINKSGYRYHPGEPRTFMLTANVHF

SEQ ID NO: 25 - protein, 790 aa, Klebsiella pneumoniae
MATMYKSTPSAAWCKKRLLVTSLFAAIYQTSAIAADTSAVSGEAVDDTSEQMTVTAPAPVQKAGSEHSISARELENKGANDFGSIMRYEPLISATGASGGSGNGKSGFDRGGYTGYNIRGMESNRVGIDVDGIAQPNATGRGYVGRAGLNTFGIGRDYIDPYMYGSVDIQSGATSTETANSAIGGNVSFRPKSADDYLRPGKTSAFGYRSGYDSADRSWHNGVTVAGGDEFLRGILVYSRRDGQETENNSGTVDAYPANWHSDAFLASGIWQPNDEHKLTSTFDYYHKTNHTHYDTWDSSGNSTIGTANQTSQTRRWGLSLKDDWTPMNDYLDSVSTKIYYQHTEAHDWTYMPDSVTRKMQTVNSNYDTDTWGLQTALAKTLGRHDLSAGFNASTSKTQRPFSQSPIPSVYSEIMQPEADSRSYTLGGFVQDKINFDLDSHNFAVIPGVRVVHQSTKPENLSDLAANSSVLSESSVANLYGKNSDTQVLPSLTFQYDLTPRLMTYLQYQRGAQFPNASQLYGSWNLGSSYAGSQQYALIGNTDLKTETSDNLEWGLKGEVTEGITLRTALFYNSYKNFIAYTRYTRANNPGQFTNVPSNIYTIYQAENRDKAYIYGGEISTKFNFGTWFEQVDGLSATLALGYSEGKSKSSYSGDKYVDLDSVAPMKAIVGVAWDDPAKRYGTALTATFVKGKQATATNRESYSNSGSAITDASSDYMRVPGYGMLDWTAYWQVAKNVRLNGGVYNLTDRKYWDYLSSRNIETGTNQDANDKALAVMPGRTWQLGVNVDF

SEQ ID NO: 26 - protein, 809 aa, Klebsiella pneumoniae
MAMKKLLIASLLFSSATVYGAEGFVVKDIHFEGLQRVAVGAALLSMPVRPGDTVTDDDISNTIRALFATGNFEDVRVLRDGDTLLVQVKERPTIASITFSGNKSVKDDMLKQNLEASGVRVGESLDRTTIADIEKGLEDFYYSVGKYSASVKAVVTPLPRNRVDLKLVFQEGVSAKIQQINIVGNHAFSTDELISHFQLRDEVPWWNVVGDRKYQKQKLAGDLETLRSYYLDRGYARFNIDSTQVSLTPDKKGIYITVNITEGDQYKFSGVQVTGNLAGHSAEIEALTKVEPGELYNGAKVTKMENDIKKLLGRYGYAYPRVQSQPEINDSDKTVKLHVNVDAGNRYYVRKIRFEGNDTSKDAVLRREMRQMEGAWLGSDLVDQGKDRLNRLGFFETVDTDTQRVPGSPDQVDVVYKVKERNTGSFNFGIGYGTESGVSFQAGVQQDNWLGTGYAVGINGTKNDYQTYTELSVTNPYFTVDGVSLGGRVFYNDFDANDADLSDYTNKSYGTDITLGFPVNEYNTLRAGVGYVHNSLSNMQPQVAMWRYLNSMGQYPDNTNDRNSFSANDFTFNYGWTYNKLDRGFFPTEGSRVNLNGKVTIPGSDNEYYKATLDTATYVPIDNDHQWVVLGRTRFGYGDGIGGKEMPFYENFYAGGSSTVRGFQSNTIGPKAVYFPASSRHDDDDSYDNECKSTESAPCKSDDAVGGNAMAVASLELITPTPFISDKYANSVRTSVFWDMGTVWDTHWDSSAYAGYPDYSDPSNIRMSAGIAVQWMSPLGPLVFSYAQPFKKYDGDKAEQFQFNIGKTW

SEQ ID NO: 27 - protein, 896 aa, Klebsiella pneumoniae
MTDVTIKALASEIQTSVDRLIQQFADAGIRKSADDSVTSQEKQTLLTHLNREHGSAPDKLTLQRKTRSTLNIPGTGGKSKSVQIEVRKKRTFVKRDPQEAERLAAEEQAQREAEEQARREAEEAAKREAQLKAEREAAEQAKREVADKAKREAAEKDKVSNQHTDEMTKTAQAEKIRRENEAAELKRKSEEEARRKLEEEARRVAEEARRMAEENEKNWSETSDSPEDSSDYHVTTSQHARQAEDDNDREVEGGRGRSRSSKAARPAKKGNKHAESKADREEARAAVRGGKGGKHRKGSALQQGFQKPAQAVNRDVVIGETITVGELANKMAVKGSQVIKAMMKLGAMATINQVIDQETAQLVAEEMGHKVILRRENELEEAVMSDRDTGAAAEPRAPVVTIMGHVDHGKTSLLDYIRSTKVASGEAGGITQHIGAYHVETDNGMITFLDTPGHAAFTSMRARGAQATDIVVLVVAADDGVMPQTIEAIQHAKAAQVPVVVAVNKIDKPEADPDRVKNELSQYGILPEEWGGESQFVHVSAKAGTGIDDLLDAILLQAEVLELKAVRNGMASGAVIESFLDKGRGPVATVLVREGTLHKGDIVLCGFEYGRVRAMRDELGREVLEAGPSIPVEILGLSGVPAAGDEVTVVRDEKKAREVALYRQGKFREVKLARQQKSKLENMFANMTEGEVHEVNIVLKADVQGSVEAISDSLLKLSTDEVKVKIIGSGVGGITETDATLAAASNAILVGFNVRADASARKVIEAESLDLRYYSVIYNLIDEVKAAMSGMLSPELKQQIIGLAEVRDVFKSPKFGAIAGCMVTEGTIKRHNPIRVLRDNVVIYEGELESLRRFKDDVNEVRNGMECGIGVKNYNDVRVGDMIEVFEIIEIQRSID

SEQ ID NO: 28 - protein, 1077 aa, Klebsiella pneumoniae
MKRMLINATQQEELRVALVDGQRLYDLDIESPGHEQKKANIYKGKITRIEPSLEAAFVDYGAERHGFLPLKEIAREYFPANYNAHGRPNIKDVLREGQEVIVQIDKEERGNKGAALTTFISLAGSYLVLMPNNPRAGGISRRIEGDDRTELKEALASLELPDGMGLIVRTAGVGKSAEALQWDLSFRLKHWEAIQKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTKEVHAIVPVPIASYLLNEKRAAVSAIESRQGDVRVIIVPNDEMQTPHYSVLRVRKGEETSTLSYLLPKLHEEEMALPGDDEPAERKRPEQPALAAFVMPDAPPAPMLEEPAAAPVAAAAPVAAAAPAQPGLLSRFFSALKNIFSGAEEAKPAEVQVEKKAEEKPERQQERRKPRANNRRDRNDRRDNRDNRDNRDNRDNRDTRADNAEGREPRESREENRRNRREKPSQNVEARDVRQTSGDDAEKAKSRDEQQPRRERTRRRSDDKRQAQQEAKAQTREEPVVQETEQEERVQTLPRRKPRQLAQKVRVESAVVEPVAEIVPEAVVAEVIAPHSEPVKAELPAGVESVADQDENGESREANGMPRRSRRSPRHLRVSGQRRRRYRDERYPTQSPMPLTVACASPEMASGKVWIRYPVVRPQDQQPEEVQVQDASVAKTVEAVAAPVAVVETVTAAPVTVEPATMEPVTAEPVVVEPVAAAEPLVVDAAEVVAPAAVEPAPQEPVTEAPAVEAPQAIAPVTLDAEPVVVEPEAVETTPVVAAPVETIAPVAETVEQAPVTEAAPAEPVKAEPPVSKPVVVAGHRHATAPMTRAPAPDYVPEAPRHSTWVRPPFAFEGKGAAGGHSATHKATAEPTRPQPVE

SEQ ID NO: 29 - protein, 1159 aa, Klebsiella pneumoniae
MRKLSLSLLTLSLGVALLPLAQAATTPAQEHLLEQVRLGEASNREDLVRQSLYRLELIDPNNPELIAARMRYLLRQGDAAGAQKELERLTKQAPDSPELKASRNEMKSNTGEGRQALQQARLLGVAGKVDEAIAAYEKLYGGVPDDVDVAIEYWTLVARLPARHSEGVSQLKKLNASAPGNVSLLTSLAKQMFADNKPQEGFAYLAEMARSASGRGIAADMWFSEVKSMPVSKASVQALQQFLLQFPTGSVAANARVLLDQQQAQLQDPTFRARSEGLAAVKSGNTTQAVADLQKAVQADSRDSDAVGALGQAYSQRGDRARAVAQLSKAIAMDPDSPNRGKWDSLLQTNRYWLLIKQGDNALKAGQLSQAQNYYAQAQRVDRTDSYAVLGLGDVAAARKEAAAAERYYQQALRLDRGNNLAVRGLANLYRAESPEKASAWIAGLPPAQRRSIDDIERSLTNDRLEKQAQALESQGNWAQAAEVQRRRLALDPDSVWITYRLARDLVSAGERQEADALMRTMVNRQPQDAERVYASGLYLSGNDQDDLALAQIAALPRSAWTDNIRELEARLQSDRVLRQANQLRDSGNEAQAIALIRQQPASVRYDLTLADWAQQRGDSQTAIANYQRVLRQEADNGDARLGLAEVYLAEGDKPAARAQVMQLKGAETESMNMQRRVALARAGLGDTADAQRIFNQIVPQAKAQPPSMESALVLRDAARFATQSGAPQQALTHYREAMVASGITPAQPQDNDTFTRLTRNDSHDDWLKRGIRSDAADLYRQQDLNVTLEHDFWGSSGTGGYSDLKAHTTMLQMDAPLADGRMFFRTDLVNMDAGSFSTHSDGSYSPSWGTCGEIACTSGSKNQTDSGASVAVGWKNDTWSGDIGTTPMGFNVVDVVGGLSYSSDVGPVGYTVNVHRRPISSSLLSFGGQKDSSSHTGATWGGVRADGGGLSLSYDRGEAHGIWSSLGADSLTGKNVADNWRVRWMTGYYYKVINENNRRVTVGLNNMIWHYDKDLSGYTLGQGGYYSPQEYLSFAVPVTWRQRTENWSWELGGSVSWSHSRTQTQARYPLLNLIPSDYRQRASELTEEGSSSHGFGYTARALVERRVTSNWFVGAAVDIQQAKDYTPSHALLYVRYSAAGWQGDLDMPPQPLVPYADW

SEQ ID NO: 30 - protein, 1411 aa, Klebsiella pneumoniae
MSQEYTEDKEVKLTKLSSGRRLLEAMLILCSLFAIWLMAALLSFNPSDPSWSQTAWHEPIHNLGGAPGAWLADTLFFIFGVMAYTIPVIIIGGCWFAWRHQENDEYIDYFAVSLRLIGALALILTSCGLAAINADDIWYFASGGVIGSLLSTTLQPLLHSSGGTIALLCIWAAGLTLFTGWSWVSIAEKLGGGILSVLTFASNRTRRDDTWVDEGEYEDDEEEYDDEEAARPQESRRARILRSALARRKRLAEKFTNPMGRKTDAALFSGKRMDDGEEVVQYSASGAPVAADDVLFSGASAARPAEDDVLFSGASAVRPGDFDPYDPLLNGHSIAEPVSAAAAATAAPQAWAESPVGHHGAAPAYQPEASYPPQQAYQPEPAPFQQAYQPEPAPFQQAAYQPPAGQTAPQAYQPEPAPYQQPVYDPRAGQPAPQAYQPEPAPYQQPAYDPYAGQPAPQAYQPEPAPYQQPAYDPHAGQPAPQAYQPEPAPYQQPAYDPYAGQPAPQAYQPEPAPYQQPTYDPYAGQPAPQTYQQPAYDPNAGQPAPQPYQPEPAAYQPQSAPVPPPEPEPEVVQEEVKRPPLYYFEEVEEKRARERELLASWYQPIPEPESPIATKPLTPPTTASKPPVETTVVSAVAAGVHQATAASGGAAAATSSTAASAAATPLFSPASSGPRVQVKEGIGPKLPRPNRVRVPTRRELASYGIKLPSQREAEQRARQAERDPHYDDELLSDEEADAMEQDELARQFAATQQQRYGHRWEDDNATDDDEADAAAEAELARQFAATQQQRYATEQPPGANPFSPADYEFSPMKTLVNDGPSEPLFTPTPEVQPQQPAQRYQQPAAAPQQGYQPAQHQPIHHQPVPPQPQSYPTASQPVQPQQPVAPQGHQPAAPAPQESLIHPLLMRNGDSRPLQKPTTPLPSLDLLTPPPSEVEPVDTFALEQMARLVEARLADFRIKADVVNYSPGPVITRFELNLAPGVKAARISNLSRDLARSLSTVAVRVVEVIPGKPYVGLELPNKKRQTVYLREVLDNAKFRDNPSPLTVVLGKDIAGDPVVADLAKMPHLLVAGTTGSGKSVGVNAMILSMLYKAQPEDVRFIMIDPKMLELSVYEGIPHLLTEVVTDMKDAANALRWSVNEMERRYKLMSALGVRNLAGYNEKIAEAARMGRPIPDPYWKPGDSMDAVHPVLEKLPYIVVLVDEFADLMMTVGKKVEELIARLAQKARAAGIHLVLATQRPSVDVITGLIKANIPTRIAFTVSSKIDSRTILDQGGAESLLGMGDMLYSGPNSTTPVRVHGAFVRDQEVHAVVQDWKARGRPQYVDGITSDSESEGGGGGFDGGEELDPLFDQAVNFVTEKRKASISGVQRQFRIGYNRAARIIEQMEAQGIVSEQGHNGNREVLAPPPFE

SEQ ID NO: 31 - DNA, 285 bp, Klebsiella pneumoniae
  source 1..285: mol_type = genomic DNA
atgaagaagttagctttactctccgccgtaatgacgcttggaatgtcgtcatgggcttttgctgccgacaacccgccgccgccgccggaaaaaggcgcgcagcatcagggtaaaccgccggtgaaaaacggccaacacgaaggtaagcaagcgcaatacaacagaaaacagccacaacgagacggcaaacagccgcagcacgacggtaaacagccgcagcacaacggcaagcagccgccaaaagggagcgagcacagcgggaaaccgctgccgccgaaagcgtaa

SEQ ID NO: 32 - DNA, 354 bp, Klebsiella pneumoniae
  source 1..354: mol_type = genomic DNA
atgaaacgttacgcaaccgcactgctcttttgcactctgtcgctgaccagcctggccgctcgcgccgatattatcgatgacgcgatcggcaatattcagcaagccattaacgacgcctataaccccggcagcagtcgctccgatgacgacgacagatacgatgacgatggccggtatgatgacgggcgctatcaggggagccgtcagcagagccgtgacagtcagcgccagtatgacgagcggcaacgccagctggacgagcgccgccgccagctggatgaacgccagcgtcagctcgaccgcgatcgtcgtcagttagaaagcgaccagcgtcgtctggatgatagctactga

SEQ ID NO: 33 - DNA, 360 bp, Klebsiella pneumoniae
  source 1..360: mol_type = genomic DNA
atgttcaggtcactgattctggcagcagtactgctggccgcagggccactggtcgctaacgctggtgaaatcaccctgctgccatcggtaaaattacaaataggcgatcgtgacaattacggtaactactgggacggtggcagctggcgcgaccgtgattactggcgtcgtcactatgaatggcgtgataaccgttggcatcgtcatgacaacggctggcacaaaggctggtacaaaggcagagataaagcctgggagcgcggctatcgtgctggctggaacgaccgcgatgaccaccgcggcggctggggtcgcggcccgggcgggcgcggtcacggtcatggacatggccatcactaa

SEQ ID NO: 34 - DNA, 363 bp, Klebsiella pneumoniae
  source 1..363: mol_type = genomic DNA
atgaaggaaatcggcttaccgttattgctactgaccgcgctggccagtccggcttttgctgcagactgtcagccaaacggcattggcggctcgttttgcattaacgatgacggtacgactaccgacacggtgcctaacgaagtcaacggcatggatacgtactcgaataatggcggctataccagttccctgcccgatcggtcaggggcggatgaagcactggaaggttcatcgctgtcgacgcagcaaggcgtcggcagcggacagagcgacagtgcgctggcgggtcgcgactggcattcgcccgccaatctgaatgatggcgccgccacctccagtatgagcctgctggataaaccctga

SEQ ID NO: 35 - DNA, 366 bp, Klebsiella pneumoniae
  source 1..366: mol_type = genomic DNA
atgaatatgaaaaaactgacgacccttttgctcaccgccaccttaggtcttgccagcggcgcggccctggcggcagacaccggcgcccagtccaataatggccaggccaactcttccgcggatgccggtcaggtggcgccggatgcccgtgagaacgtggcgccgaacaacgtggacaatagtcagatcaactctggctctgggggcaccacgggctcgacgatgacccaggataatatgtcgagcaatgaggtacataaaaactcgatgtgtaaagacggccgctgtccggacaccggtaaaaaactggacaacggtggcaatacgacccaagacaacagcaaaaccgacggcaccacccagtaa

SEQ ID NO: 36 - DNA, 369 bp, Klebsiella pneumoniae
  source 1..369: mol_type = genomic DNA
atgaaacaccgcatcgctctgcttctggtcctgacttcacttagcgccagcgccctagccgcctctccctgccaggaaaaagagcaggatattcaacgagagatcagctacgccgaaaagcatcataatcaaagtcgcattgatgggctaaataccgcgctacgtcaggttcgggaaaactgtagcgacagtaaactcaaagccgatcatcagcaaaaaattgccaaacagcgggaagagatcgctgaacgtcagcgcgatctgcaggaagcccggaagaaaggcgatgcggacaaaattaacaaacgccagcataaactcaatgaagcgcaacaggagttaaaaacgctggagtctcgggattactaa

SEQ ID NO: 37 - DNA, 426 bp, Klebsiella pneumoniae
  source 1..426: mol_type = genomic DNA
atgcgactcataacacgacacgtgagagaggatattatgaaaaaagcaatgattgcgttatcggctattctggttgcggctccggtttttgctgcgacaacacatgcaacagatgataccgtcgcggcggcgaatgccaacgccaacaccgctaaagagaagctgcatcaggcccagcacgagggcgaagagcagcagctgaaggcgaaacacgccgccgaaggcaagcaggacagcgtcggcagccaggtgagcgaaggcgcgcagaaaacctggaacaagaccaaagaaggcaccgagaaggggtgggataagaccaaagaggtcagtgaaaaaggctggaacgccaccaaatccggtgcggaaaagggctgggataaaaccaaaaccggcgccgaagagttaaaaaataaagtgactgaataa

SEQ ID NO: 38 - DNA, 456 bp, Klebsiella pneumoniae
  source 1..456: mol_type = genomic DNA
atgaaaaagatgatttctctggcagtaattttatcctgtgtgctgagcgtcccggcctttgccgatggcccgaacgacggccatcgcccggagcagcccacggtgtggcagaacggtccggaccatgacgggcatgcaccgcagggcggacctgacgcgcatcatcagggcgaccatgaccagcgtggcccggatcgcgacggccatgacaaacgcgatctggcacgtcatgagcaggaccatttcgcctggcgcgggaacgatttccgcaaaggccacccggctccggcgccgttccgtggcgatgaatatcgcgtccgcgactggagcgaccgcggcctgccgcccccgccggaaggccatcactggtcctatatcgacggtaactatgtgctgatcgccgcggcgaccgggatcatcacctcgattctggtgagcggcgccctcggccactaa

SEQ ID NO: 39 - DNA, 507 bp, Klebsiella pneumoniae
  source 1..507: mol_type = genomic DNA
atgaaaaaaccgacatccgccacccgtggcaaatccggccgcaagtcgcgtgaagagttaaatcaggaagctcgcgatcgcaaacggcagaagaaacatcgtggccacgcggcaggcagtcgcgcgaacggcggcgatgcggcttcagcgggtaaaaaacagcgtcaggcgcaagatccgcgcgtgggtagcaaaaaaccgatcccgctgggcgtgagcgaaagcagcgttccagctcccaagcagcataaaccaaagagcgagaaacctatgctttcaccgcaggctgagctggagttgctggagaatgatgagcgcctggacgcgctgctggaacgtctggaagagggcggcaccctgaatgctgaagagcagagctgggtggacgccaaactggatcgcattgatgagctgatgcagcagctcggcctctcttacgatgatgaagatgaagaagaggaagagcgtcaggaagatatgatgcgtctgctgaagggtggaaactaa

SEQ ID NO: 40 - DNA, 690 bp, Klebsiella pneumoniae
  source 1..690: mol_type = genomic DNA
atggcgagtaagtttcagaaccgtttagtcgggacaatcgtgctggtggcgctgggggtgattatcctgccagggctgctggacgggcagaaaaagcattaccaggatgagtttgccgcgatcccgctggtaccgaaaccaggcgatcgcgatgaaccggatatgttgccggcggcaacccaggcgttgccttcgcaaccgccggaaggggcggcggaagaggtgcgggcgggcgatgccgccgcgccatcgttagatccatcgcgtattccggtgaacagcaacagcttcgatgacgttcaggagccggtggtggccgcgaaaccgcagcccaagccgcagccgaaaccgcagccgcaacagcaggcctcgacgccaacgccgccgccggctaagccacagcagcaacagccaccgcagcagcaggcggccctgccggcgccgaccggcaaagcctatgtggttcagctgggcgcgttgaagaacgccgataaggtgaatgagattgtcggtaaactgcgggcctcgggtttcaaagtctatacgtcgccttcgacgccggtacagggtaaaattacccgcatcctcgtcggcccggatgcgtcaaaagacaagctgaaaggccagctgggcgatctgcagcagatctccgggcttagcggggtggtgatgggcttcaccccgaactga

SEQ ID NO: 41 - DNA, 969 bp, Klebsiella pneumoniae
  source 1..969: mol_type = genomic DNA
atggcacaacgagattatgtacgccgcagccaaccggcttcttcgcggcgcaaaaagagcacgacccgaagctcaaggaataagcaaagcagccttccggcgatttcaccggcgatggtggcgatcgcggcggctgtgctggtggcctttatcggtggcctctatttcattacgcatcataagaaagaagaagcggaagcgatgcaaaatcgccaggccgccggcaacggcttgccgcccaaaccggaagagcgctggcgctatattaaagagctggaaagccgccagcctggcgtccgcgcgccgaccgaaccgaccgccggtggcgaagtcatgaaaccggaacagctgaccgacgagcagcgccagctgctcgcccagatgcaggccgatatgcgccagcagccgacccagctgaccgaagtgccgtggaacgaacaaacgccggcgcagcgccagcagacgcttcagcgtcagcgtttagcgcagcaacagcagcaggcgcagcagcaacagtgggcgcagactcaggcgcagaccgtccaacagcagccgccgcgcgttcagcagccgaagccggttcagcagcaacagccgaagcagaccgcgtcaaaccagcagccgtaccaggatctgctgcagacgccagcgcataccaataccacgcagccgcgtacccaggccgcggcgccggtaactcgggtggaagaagcgccgaaaaccaccgccgagaagaaagacgatcgtagctggatgatccagtgcggctcttttaaaggcgccgagcaggccgaaaccgtccgcgctcagctggctttcgaagggtttgcttcgcacattaccactaacaacggctggaaccgcgtggttattggcccgttgaaaggcaaagaaagcgccaacgagatgatcacccgcctgaagatggctggacacgcgaactgcattcgtctcgccgccaggggttga

SEQ ID NO: 42 - DNA, 1131 bp, Klebsiella pneumoniae
  source 1..1131: mol_type = genomic DNA
atgagcgcgggaagcaccaaatttaccgtcagccgtattgcggctctttcactggtttcactctggctggccgggtgtaccaacaccaataatccgcctgcgccggttagctctgccggcggcgccgcctcttccagcaccaactccggcatgctgattacgccgccaccctccggcgtcaagtccgctcctcaggcgcagccgattcagccgatgcagacccagaccattcagccggcgccggtggcgcaggagccggtacagacggtaaatggccggatcgtttacaaccgcaaatatggcgatattccgaaaggtagctataccggcggcagtacctatacggtaaaacgcggcgacacgctattctatatcgcctgggtcaccggcaacgatttccgcgacctggcgcaacgtaacaatatcccggccccgtacgcgctgaacgtggggcaggtactgcaggtcggtaacgcctcaggccagccgatcaccggcgaaaacgccgtttctcaggccagcgcaagagcgagcggcggtgcgacgaccagcacaacttctgcacaaaaatcgaccgcggtggttgcttcacaaccgactattacgtattctgaatcttcaggtgaacagagtgctaccaagatgttgcctaataataaaccagcgaccacaaccacaacggttgtcgcgccggtgacggcaccaacaacggtgagcacaacccagccgactgcaagcagtacgtcaaccagttcgccgatctcagcatggcgctggccgactgatggcaaggttatcgagaactttagcggcgcggaaggcggcaataaaggcatcgatattgcaggcagtaagggacaggctattgtcgcgaccgccgatgggcgcgtcgtctatgccggtaacgcactgcgcggctacggtaatcttattatcatcaaacacaacgatgattacctgagtgcctacgctcataacgataccatgctggttcgggagcaacaggaagtcaaagcggggcagaaaatcgctaccatgggtagcaccggaaccagctcaacaagattacattttgaaattcgttacaaggggaaatccgtcaacccgctgcagtacttaccgcagcgataa

SEQ ID NO: 43 - DNA, 1149 bp, Klebsiella pneumoniae
  source 1..1149: mol_type = genomic DNA
atgcgtaagcaatggctggggatctgcatagcagcggggctgctggcggcatgttcgagtgatgacgtgcaacaaaaaacggtcagtactccacagccggccgtctgtaatggcccgacggttgagatcagcggcgccgatccgcagtatgaaacgccgaacgccacggcgaatcaggattatgagcgcgacggtaaaagctacaaaatcgttcaggatccggccaactttactcaggccggtttcgcggcgatctatgacgcagaacccaacagcaacctgaccgccagcggcgaagccttcgatccgactcagttgaccgcagcgcacccgacgctgccgatcccgagctacgcgcggatcactaaccttgccaacggacggatgatcgtcgtgcggattaacgatcgcggtccctatggcaacgatcgggtcatctcgctttcccgcgcatccgctgaccgcctgaacacctccaacaacaccaaagtgcgcatcgaccccatcatcgtcgcgcctgacggttcgctttccggcccggggatggcctgtaccaccgtcgccaaacagacttacgccctgcccgcccgtccgaatctggacggtggggacgccgctggcatgagccagcccgcgcccactgacgttcgcccgatcagcaacagcacgctgacgccggcagacagcgtgggcgcgccggtgaacagcggcggtttcctcggcgcgccgacgcccctgaacaacggcgtgctggagagtagcgaaccagcggcagccgccgcgacggctcctgccgccggcgccacgccaacagcgccagtgaccgcgcctggctccattcagggtaatgtggtgcccgctgcggccaccgccgcagccgctggcgccgtggcggcctcgtcctccgcgacctccagcgccagcggtaattttgttgtccaggtgggcgcagtaagcgaccagacgcgggcgcagcagtatcagcagcgcctgagccagcagttttctgtgccaggccgggtcatgcaaaacggcgcggtctggcgtattcagctgggtccctttgctgataaagcacaggccagcgccgtgcagcagcgcctgcaaagcgaagcgcagctgcagtcctttattactcgcgccaactaa

SEQ ID NO: 44 - DNA, 1287 bp, Klebsiella pneumoniae
  source 1..1287: mol_type = genomic DNA
atggatgatttcaaaccagaagacgatatgaaagccgatcgcaacgatcgtcgtgctggtcgttcccgtcagtcttccgagcgtgatgccgatccgcagatcaattttgacgatgttgatcttgatgccgatgaaggccgtccgacgcgcgctggtaaggcccgtcgcgagcgtgaagaggaagagttcgaagaagaactggatgcgcaagacgaggagatgctcgaagagcagcctgtagagcgtcgtccgcgcaagcgtaaaaaagcgccggccaaaccggcctcccgccagtacatcatgatgggtgtggggattctggtgctgctgctgttgatcgtgggtatcggttccgcactgaaatcgccatcatcttccagccagcagaccgcttccggcgagaagagcattaatctgtctgacgaccagtccgccagcatgcctgctgccggccaggaccagactgccgccgctaacagcacctcacagcaggacgtaacggtaccgcctattgccgcgaacccgacgcagggccaggcagcggttgcgccgcagggccagcagcgtatcgaagttcagggcgatctgaacaatgccttgacccagcagcagggccaactggacggcgccgtggctaactcgacgctgccgactgaaccggctaccgtcgcgccaatccggaatggcgccaatggcaccgcggcgccgcgccaggcgaccgagcgtcagacagcagcgaccccgcgtccggctgaacgtaagcataccgttatcgaagcgaagccgcagtcgaagccacaggccgtggcgaaaacgccggtagaatcgaagccggtccagccgaagcatgttgaaagcacggcgaccaccgctccggcgaaaacgtccgtcagcgaaagcaaaccggtggccaccgctcagagcaaaccgaccacgacgaccgcagcgccagcggcaacggcagctgcggcagcgccggcagcgaagaccgggaagacggcaggtgacgtcagctcaatgaaaactgcgccgtcgggtcactatactctgcagctcagcagctcctctaactacgacaacctcaacaactgggcgaagaaagagaagctggataaatatgttgtctatgaaacgtcgcgtaacggccaaccatggtacgtgctggtgagcggtatctatgcatcgaaagatgaagcgaaacgtgctgtcacctcgctgccggcggacgtgcaggcgaaaaatccatgggcaaaaccgctgcatcaggttcaggctgacctgaaataa

SEQ ID NO: 45 - DNA, 1326 bp, Klebsiella pneumoniae
  source 1..1326: mol_type = genomic DNA
atgtcaaaggcaaccgaacaaaacgacaagcttaaacgagcgatcatcatttcagtcgcgctgcacatcattctgatcgcgctgctgatctggagttcgtttgacgagcatctggatgcctctgccggcggcggcggcggatcgtcgattgatgccgtcatggtcgatccgggggcggtggtaaataactataaccgtcagcaacagcagcaggccagcgcacgtcgcgccgctgaacagcgtgaaaaacaggcgcagcagcaggcggaagagttacgtgagaaacaggcggcggaacaggaacggctgaaacagctcgaacaggagcggctgcaggcgcaggaagcggcgaaagaagcgaaggagcagcagaagcaggctgaagaagcggctgccaaggccgccgcggcggcaaaagccaaagcggacgcacaggcaaaagaagcgcaggaagccgctgccaaagcggccgccgaggcgaaagcgaaggcggatgcccaggcgaaagcggcagaacaggcggcggccaaggcggctgctgacgcgaaaaagcaggccgaagccgctgcagcgaaagccgctgccgaggcgaagaaacaggcggaagctgaagcggcgaaagctgcggccgaggcgcagaagaaagcggaagcggcggctgcgaagaaagcgcaacaggaagcggagaaaaaagcccagcaggaagcggctaagcaggcggcagctgaaaaagcggctgccgaaaaagccgctgagaaagccgccgcgcaaaaagcggccgctgagaaggccgccgccgagaaagccgcagccgctgaaaaagcggcggcagcgaaagcggctgcagcagagaaggctgcagctgataaagcggccaaagcggcagcagcaaaagccgcggcggcgaagaaagcggcggctgcgaaagaagcggacggcgttgacaacctgctcggcgatctgagttctggtaagaatgcgcctaaaacaggcggtggggccaaaggaaacaatgcctccgctgccgggagtggtaatactaaaaacagtgcctcaggggctgatatcaacaactatgccggacagataaaatcggcgattgaaagtaagttttatgacgcatcgtcctatgcgggcaaaacatgtaccttgcgtatcaaacttgctcctgacggcctgttgttaaatatacagtccgaaggtggtgatcctgctctgtgccaggccgctcttgccgcagcccgacaggctaagtttccgaaaccacctagccaggcagtatatgaagtcttcaaaaatgcgccactggacttcaaacctcagtga

SEQ ID NO: 46 - DNA, 1455 bp, Klebsiella pneumoniae
  source 1..1455: mol_type = genomic DNA
atgttttttttaagtattttttacatggagatgacaaaagtgaaattaagcgctctgtttattgccctgattcctctactgggctcgccggttattcatgcagaaactactgctgcgccggttctggaaaatcgcgctgcgcagggagatatcaccactcctggcggcgcgcgtcgtttaacaggcgatcaaaccgaagcgctgcgcgcctcgttaatcaataagccagctaaaaacgttattttgctgattggcgatggcatgggtgattcggaaattaccgctgcgcgaaactatgccgagggggcgggcggtttctttaaaggaattgatgctctgccgttaaccgggcagtacacgcattattcgctggataaaaaaaccgggaaaccggactacgtgaccgactcggcggcctccgccaccgcctggaccaccggcgtgaagacttataacggcgcgctgggcgtcgatattcatgagaatgcgcatcagaccatcctcgagctggcgaaagcggcggggctggccaccggcaacgtttccaccgccgagctgcaggacgccacccccgcggcgttggtagcgcatgtgacatcgcgtaaatgctacggcccgacggtcaccagcgaaaaatgccccagcaatgcgctggaaaaagggggcaaaggctccattaccgaacagctgctgaacgcccgaccggatgtcaccttgggcggcggcgcgaagacctttaccgaaacggcgacggcgggcgagtggcagggcaaaaccctgcgcgagcaggcgcaagcgcgcggctaccagattgtgaccgacgcggcttctcttgccgccgcgacggaagccagtcaggataaaccgctgctgggactctttgccgatggcaatatgccggtacgctgggaagggccgaaggcgtcttatcacggtaatatcgataagccgccggtgacctgtacgccaaacccgaagcgtgacgcctcggtgccgacgctggcgcagatgacggagaaagcgattgacctgctcagtcgcaacgagaaaggtttcttcctgcaagtcgaaggcgcttccatcgataagcaggaccatgcggcgaatccgtgcggccagatcggcgaaacggttgatcttgacgaagcggtgcagaaggcgctggaattcgcgcgaaaagacggtaataccctggtgatcgtcaccgccgaccatgcgcatgccagccagatcatcccggcggatagcaaagccccggggctgacccaggctctgaacacgcacgatggcgcggtgatggtgatgagctacggcaactctgaggaagagtcgatggagcacaccggcacccaactgcgcattgcggcctacggtccgcatgcggctaacgtcgtaggcctgaccgatcagaccgacctgttcacgaccatgaaagctgccctgagtctcaaataa

SEQ ID NO: 47 - DNA, 1608 bp, Klebsiella pneumoniae
  source 1..1608: mol_type = genomic DNA
atgtcactgccgttcaaaccccatattatcgccctgctctgtagcgctggcttactcgcggcggcaggaacactctatgtgcaaagccgaaccccagcgacgatcgctgaaccgcctgcgcagcaagcgccagcgcccgcagcgtcgacgacacagccggtggccgccacttacacccaggcgcaaattgatcagtgggtcgcccctatcgcgctctacccggacagcctgctgtcgcaggtgttgatggcctccacttatcccgacaacgtcctgcaggcggtccagtggtcccaggataaccccgcgatgaaaggggatgcggccgtgcaggcggttgccagccagccgtgggaccctagcgtcaaatctcttgtcgctttccctgccctgctggcgatgatgggcgagaatccgccctgggtggaaaatcttggcaatgcgtttttggcccagccgcatgatgtgatggattcagtgcagcgcctgcgcgccattgcccaacaaaccgggacgctgaaatccacaccgcagcagaaagtgattgtcacccctgccgcaccggtttcagccagcagcagcacggcagcaaccgcaaccgcccacacagcggcgcctgcgcccacgcaggtcattaaaatagagccgaccaatccacaggtggtctatgttcccagctataacccctccaccgtctatggtacctggccgaacagcgcctatccgccggtctatctgccgccccctcccggggagcagtttaccgatagcttcgtcaaaggcttcgggtacagcctcggcgtggccaccacctgggcgctgttcagcagtatcgactgggatgatgatgaccatcaccatcacgatgacgactaccaccacggcgattactcgcataatggcgataacatcaatattaatgtaaataatttcaatcatataacaggagaaaacctgccgggaaaccacgttaactggcagcacaatcctgcctatcgcggacacacaccgtatcccgataatacggtagctcagcgcttccatcagaccaacgtttccggcggactgagcgcgacccaacatgcgccagtcgatcgcgaagcgcagcgccaggcagcgatgacccagctgcagcataacgtaccgacggccacagcgggcaacctggcggcaaacaacgcctcacgcgacgcccagcgtcaggcggcctcggcgcagctgaagcaagccacccaacgcagtaattaccgcggttacgacagtacgccgacccaacagcagcgtcgcgaggcggcaaaaacgcagctgaaaaaccccacgccgcagcaacagcagcgtcgagaagccgccaggagccacgagcagaaccgcacacctcagcagcagcagcgccggcagcagttccagtccgccacgccagcccagcgtcagcagacgctcagccatctgcgcgccaacgcccttagcggcaacgaaagccgcgccccctcctggcaagcgcagcaggaacgaggactgcagagccgccagttttccggcgtaaaccgcgagttacgcgatggcaccagagaacgtctttccgaacaccatgaactgcgtcgccgctaa

SEQ ID NO: 48 - DNA, 1794 bp, Klebsiella pneumoniae
  source 1..1794: mol_type = genomic DNA
atgtttaagtttaaggcttcttatgtcgcactggcggcagtattaacctcgtcggtagtttatgccgaccccacaagctatacgcactcttccggcgccacggttatcgatattgaaaagccgaacgccgccggtgtctcccataacctgtaccgcgacttcaacgtcggcgccaatggcaccatcctcaataacagcggcgatgatgtcagccacagcacatttggcaatatcgcccgcaacaataatctgaccgccggcagcgcttcggtgattttgaacgaggtgacctccaaaaacgccagtagcctgaagggctttatcgaagtcaacggtcagaaagcggatgtggtaatcgccaacccgaacggcatcacctgttccggctgtagctttgttaataccaacaaggctatcctgaccaccggcaaggttaatatgaccgacgatggcgctatcggcagctataccgtaacgggcggcaccctcaccatcggcgaaaatggcatgaacgccgccaacggctatgcggttctgctcgccgacgcgatcaatatcaacggtaaagtgcaggccaacaacgccctggtcagcgcgggcaacttcaccatggataacagctctggctcggtgacctccgctggtaaaaaggccaccctgatccagatgacggttaacccgcagtacagcatcgacgtcagcagccttggcggcattgaggccaacagcatcagcatggtcggcaataacatcggctttggcgtacgtaataaaggctctatcgtcgcgaatagttcgctgcagctcaccagcaacggtaatctgctgaacaaaggcacgatcaaaagcaacggtctgctgagtcaggtcgccaccgcctcgggcatcaccaatgacggtagcatcgccggcgcctattatttaatgctctccagtggcgattatatcgttaacaccggttctctctccggcggccagctgattgccaccgctaacggcaacatcaccaacggcgactcaggcacgatgaccggcaccagtggattaagcctgaccagcggcgggaaaatccgcaacgaagaaaaagcctccctgctgtcaaataaccagattgccgccacggcaatcggtgatttcctcaatgaaggcaaaatcagcgccaaacacaccagcctgacgtttgtcggcgacagctttaaaaacactggcaatattaactctactggccaaaccaccattcagtcgcttaaacaggacggcagcgccaatacgggcgagatctataacctcggcaatatcaccggcgaaaatatcaatctgcagaccaatggcacgctggcgcaaagcagcagtggtcgtattgaggcaaccaacgccattaccgcccacagctactggctgaaccaaaatggttatatgaatgccgccgatatcaccaccgatcacggcgtagtgaataattatggcaatattactgccaaaaatatttcaatcacgacctactcagatatcaccaacgaagggcagatcagcagcaccggcgacctgaccttaaataccaaaaataaaggcgcgatctacaattattcaaccctcagcgcgggcggcaacatgacgttaaccgccaccaaagtggtcaatggtggtaaaagttgcggcatactgggcctggcgaaatgcggcgtcgggacgttaactgctgacaagctggtactgaactcatcgcagaaatatgttagcgacatgggtggaaaacagtatttcaagagcaccgaagtcaacacggtgaaataa

SEQ ID NO: 49 - DNA, 1875 bp, Klebsiella pneumoniae
  source 1..1875: mol_type = genomic DNA
atgatggacaacctacgcacggccgccaacagcgtcgtgctcaagattattttcggtatcattatcgtctcgttcattttgaccggggtgagtggttacctgattggcggtggcaaaaactatgccgcaaaagtgaatggccaggagattggccgtgggcagtttgaaaacgccgtcgccagcgaacgtaaccgtatgcagcagcagcttggcgatcaattctccgagctggcggcgaacgaaaactacatgaaaaccatgcgccagcaggtgctgaaccgcctgatcgatgagtcgcttctggatcagtatgcccgcgagctgggcctcagcatcagcgatgagcaggtgaagcaggcgatcttccagacccaggcgttccagacgaacggtaagttcgacaaccagcgtttcagtggtattgtcgcccagatggggatgaccaccgatcagtacgcccaggcgctgcgtaaccagctgaccacgcagcagctgattaacgccattgcgggtaccgacttcatgctgccgggcgagtccgatcagctggcggcgctggtatctcaacagcgggtggtccgcgaagcgaccatcaacgtaaatgccctggcggcaaaacagaccgccagcgatgaggaaatcaacgccttctggcagcagaatcaggcccgttttatggcgccggagcagttccgcgtcagctacatcaaaatggatgccgccagcatgcaggagagcgcctctgacgaagagattcagtcatggtacgaccagcacaaggatcagttcactcagccgcagcgcaaccgctacagcgtgattcagaccaaaactgaagccgatgcgaaagcggtactggccgagctgcaaaaaggagcggacttcgccacgctggcgaaagaaaaatcgaccgatattatctctgcccgcaacggtggcgatatggggtggatggaagatgcctctaccgtgcctgagctgaaagatgccgggctgaaagagaaaggccagctgtctggcgtgatcaaatcctcggttggcttcctggtagctcgtctggacgacgtccagccggcgcaggtgaagccgctggctgacgtgcgtaatgacattgcggcgaaagtgaagcaggaaaaagcgttggatgcttactacgcgctgcagcagaaggtgagcgatgcggccagcaacgataatgaatcgctggcgagcgcagcgcaggtcgccgggctgaaggtcgtagaaaccggctggtttggccgcgataacctgccggaggagctgaactttaaaccggtcgctgacgctattttcaacggcggtctggtgggtgagaacggcgcgccgggcagcaactccgatatcattaccgttgacggcgatcgcgcttttgttctgcgcattagcgaacacaaagccgaggcggtgaagccgctggccgaagtgaaggcacaggttagcgatatcgttaagcacaataaagcggaacagcaggcgaaactggaggccgacaagctgctggcggcgctgaaagacggcaaaggcgatgaagcgatgaaggcggctggcctgagctttggcgcgccgcagacgctttctcgtaccggccaggatccgctgagccagctggcatttaccctgccgctgccgcagcagggtaaaccggtctacggcgtgggcagcaatatgcaaggcgatgtggtgctggtagcgctggatgaggtgaaagccggcagcatgccggaagagcagaagaaggccatggttcaggggatcacccagaacaatgcccaaatcgctttcgaagcgctgatgagcaacctgcgcaaggcggcgaaaattaagctcggcgacagcatcgaccagcagcagtaa

SEQ ID NO: 50 - DNA, 1974 bp, Klebsiella pneumoniae
  source 1..1974: mol_type = genomic DNA
atgttcaggttaaacccttttatccgggcgggattgtctgcgtccgtcgtatcgttggcgtttccggctctggccgatgtgaatgaagaaacgctggtggtgaccgcctcggccactgaacagaatgtcaaagacgcgccggcgagcatcagcgtcatcacccaacaggatttacaacgcaagcctgttcagaacctgaaagacgtgctgcgcgatgtgcctggggtccagctcaccaacgaaggggataaccgcaagggcgttagcatccgcggtctgagcagcagctataccctgatcctggtcgacggcaagcgcgttaactcgcggaacgccgtcttccgccacaatgacttcgaccttaactggatcccggtggatgctattgagcgtatcgaagtggtgcgcggcccgatgtcctccctctacggctccgatgcgctcggtggggtggtcaacattattaccaaaaaaatcggccagaaatggaccgggacgctgagtgctgataccactattcaggagcaccgcgatcgcggggatacctggaacggccagttcttcaccagcggcccgctgatcgacggcgtacttggaatgaaggcctacggcagcctggcaaaacgcgccaaggacgatccgcagtcatccagtaatgccaccggcgagacgccgcgcatcgagggcttcaccagccgcgatggcaatgttgaattcgcctggacgccgaacgaaaaccacgattttaccgcaggctacggctttgaccgtcaggatcgcgattccgattcccttgaccgcaaccgccttgagcgggagaactactctctgagccataacggccgctgggatatcggcaatagcgagctcaagttctacggcgaaaaggtggataacaaaaatccagggcagagcgggactattacctcggaaagcaatgccatcgacggcaagtatgtcctgccgctgggcatgattaaccagctggtgaccttcggcggcgaatggcgccacgacaaacttaaagatccggtcaacctgagcagcggcggccagtcaacgtcggccagccagtacgccctgtttatcgaagacgaatggcgcatcatcgagccgctggcgctgaccaccggcattcgtatggacgaccatcagacctatggcgatcactggagcccgcgcgcctatctggtgtataacgccaccgataccgtcaccgtcaaaggcggctgggcgacggcgtttaaagccccgtcgctgctgcagcttaaccccgactggaccaccaactcctgccgcggctcgtgcagcatcgtcggtaacccggatctgaaaccggaaaccagcgaaagcttcgagctcggtctctactaccgcggggaagagggctggcttgaaaatgtcgaaggcagcatcaccaccttccagaataatgtcgacgacatgatcgacgttctgcgcacctccagcgccagcgaagcgccgggctacccgaactttgtcggctggaaaactgtcaacggcaagcgcgtgccgatcttccgctatttcaacgtcaacaaagcccgcatcaaaggggtggagacggaggtgaagatcccgtttggcgatgagtggaagctgacggtgaactacacctacaacgatggtcgcgatctgagcaatggcggcgacaaaccgctgcagacgctgccgttccataccgccaacggcacgctcgactggaaaccgctggacgactggtccttctacgtgacggccaactataccggccagcagcgcgcggtgagcgccaccggcaaaacgccgggcggctacaccctgtttgacgttggcgcggcatggcaggtgaccaaaaacgtgaaactgcgctccggggtgcagaacgtgggtgataaagatctgagccgggacgactacagctataccgaagaaggccgtcgctactttatggcggtggattatcgcttctga

SEQ ID NO: 51 - DNA, 2028 bp, Klebsiella pneumoniae
  source 1..2028: mol_type = genomic DNA
atgaacagagccgccacgctgaccctcaacgcgcccctgctgatgctcgtcgctgcgctggcgctttcaacccctttcaccgccggcgccgcgccggcctttcttgattacgcccaacagcaaacccagcaatctcaggcgcaagaaaaaaacgatgccgcaagcgcaaaacaaacacaagaaagccgccagagcgcagataataaaaaaaccggtaccagcacctcacaattacaaaaaagaatcaccagccagcaggcggcgattgcacaaaaagataagcttatacagcaattaaaaaaacagcttgccgctacgccgcaaacggatactgccggagcgaatgagcaagcggcgttgaataagagaattaatgaattacaggtcgccttaagcgccgctactgcagaaaaagaggcattaataaaaaaagcaggcgttgtgcagaataataatctacagcaaagccaggccgcggcgcgtcagcagatccagcaattaacgacgcagattcagcaagccgaagctgaaaataaacgcctcagcgccagctttaccacgcttaataaagataaacacgcgctaatgacccaactggccgcaacggaaaaagagaaacaggccgctcttgagcaggtcaaagcgcttaacgctgacaaacagccgctgacgacccggctggccgccgcggaaaaagagaaacaggccgtcctcgagcaggttaaggcccttaacgccgataaacagtcgctgactattcgcctcgccgctgcggagaaagcgcagcaggccgctgttgaccaggctaaagcgcttaacgctgacaaacagccgctggctacccgactggccgccgcggaaaaagagaaacaggccgtcctcgagcaggttaaggcccttagcgccgataagcagtcgctgactattcgcctcgccgctgcggagaaggcgcagcaggccgctcttgaccaggctaaagcgcttaacgctgacaaacagccgctggcgacccggctggccgccgcggaaaaagagaaacaggccgtcctcgagcaggttaaagcccttaacgccgataagcagtcgctgactattcgcctcgccgctgcggaaaagacgcagcaggctgccctcgatcaggtcaaagcccttaacgccgataaacaatcgctgtccacccggctggccgccgcggataaagcgccgcatggccccgctaacgacgccgctgcgccaaaaaatgagccaccagagatggcggccatagtggcagcctatcgcctgcaggcggataaagacaacgcccagctacggatgaaagaagatgaaatcgaactgctgagaacgcagctttctgtacagtccaaaacgcgcagcggcgagagcgccgccgccaaactcagcgcatcgggagaacagcaggcttatgcgatcggcgcctcgatgggaagcgaggcgctcaacgtccttaccacccgtcgtactcagggagttaccgtcgacgcaggcctggtgctgcagggcatcgaagatgcctttcgcggccagcttcgtctcggagagcaggaacgtaacaaggcgctgtttgatgtgtcgcagcaggtttttcagaacctgaataaaatagagcagaaaaacatcagtgccggcaagaaatatcagcaggcgtttgcgcgcaaaaaagatgtggtctttaaagagggcgtctacagccgcgtcgattacctgggtaaaggaaaaataagcggtaatgacctggttaccgtggtgatcaaagagatgctgacggacgggacggtgatcaacgatatggaagcgaaagatcaggcgcttacgcaaaagctggatgcctatcccccggtgtttcgcgaaccgctgaagcgtctacagaaccacggctccgtgacgctcgtcgtcccgcctgaaaaggcctatggcagtaaaggattaccgccaaaaatcccgccaggcgccaccatggtttattccgtgcggatagtagatagccaacccgagccggcaaaatag

SEQ ID NO: 52 - DNA, 2106 bp, Klebsiella pneumoniae
  source 1..2106: mol_type = genomic DNA
atgaaaatcctgtccgtgcgtcacgccgccctcccggccctgctcttgccgctcattgcggcagcccaggccgctgatgaacaaaccatggtggtgaccgccgcgccaaccacggtttctgaactggatacccccgccgccgtcagcgtggtgaatggggatgagatgcgccaggccgcgccgcgcgtcaatctctctgaatcgctgggcgccgtgccgggcctgcaggtgcagaaccggcaaaactatgcccaggatctgcagctgtcgattcgcggctttggctcgcgctcaacctatggcgtgcgcggactgcgcatctatgtggatggcattccggccaccatgcccgacggccaggggcagacctcaaatattgatatcggcagcgttgacaccattgaggtgctgcgcggccccttctctgccctgtacggtaactcgtccggcggggtgatcaacgtcaccagccagaccggcacccagccgcccaccgtggaagccagcagctactatggcagcttcggcacctggcactacgggatgaaagccactggcgccgttggcgacggcagccacgcaggcgatgtggattacacggtctcaaccaatcgcttcaccacccatggctatcgcgatcacagcggcgcgcgcaaaaatctggcgaacgcccggctgggggtgcgcatcaacgacgtcagtaagctgactctgctgctgaatagcgtggatatcaaagccaatgacgccggtggcctgaccgccgatgaatggcgcgataacccgcgccagtcgccgcgcggcgaccagtataatacccgcaagaatacccgacagacccaggccggcctgcgctatgagcgccagctcagtgcccaggacgatctcagcgttatgatgtacgctggagaacgtgaaaccactcagttccagtcgatcccgcgcgcgccgcagctgaagccgagccatgccggcggggtgatcgaccttacccgtcactaccaggggatcgatacccggctgacccatcgcggagagctgctggtgcccgtcacgctcaccgccggtctcgactacgaaaacatgagcgagcggcgcaaagggtatgaaaactttgtgatggtcaacggcgcgccgcagtatggcgaacagggcgcgctgcgccgtaacgaacgcaacctgatgtggaacgtcgacccctacctgcagacccagtggcagctcactgacaaactctcgctcgatgccggggttcgctacagctcggtatggttcgactcgaacgactactacatcaccccaggcaatggcgatgacagcggtgatgccagctatcacaaatggctgcccgcgggctcgctgaaatatgccctgaccgacgcgtggaacgtctatctttccgccggccgcggcttcgagacgccaaccattaacgaactctcctaccgctccgataaccagagcggcctcaacttcggcctgaaaccctccaccaacgacacggtggagatcggcagcaagacgcggatcggcaatgggctgttcaccgccgccctgttccagaccaataccgataatgagattgtggtcgacagcagcagcggcgggcgcaccagttataaaaacgccggcaagacccgccgtcaggggatggagctggggctggatcagcagtttggcgagagctggcgtctgaaggcggcctggacctggctggacgcgacctatcgcactaacgtctgcgacgacgccagctgcaatggcaatcgcattccggggatcgcgcgcaatatgggctacgcctcctttggctatcagccggagcaaggttggtacgccgggagcgatattcgctatatgagcgatatcatggccaatgacgaaaacaccgccaaagcgccctcctggacggtggttggcctgacgactggctataaatggagctacggcaggatggatatggatctgttcggtcgcatcgacaacctgttcgaccgggagtacgtcgggtctgtcatcgttaacgagtctaacggacgttactacgagcctgccccgggacgtaactacggcatcggcctgaacctcgcctggcgcttcgaataa

SEQ ID NO: 53 - DNA, 2259 bp, Klebsiella pneumoniae
  source 1..2259: mol_type = genomic DNA
atgaaatacacgtctcacttcccgctggggatcgtcattcctctgctcgcctgtagcgtgccgctgcaggcggcagagaacatgaccgaacaatcgacgcctgacgagagcgccgccactgccgaaaatcacgaggagacgatggtcataaccgccgccaggcagaacctgcaggcgccgggcgtgtcgaccatcaccgcagaagagatccgcaaacatccccccgcccgcgatgtgtcggagttaattcgtacgcagcccggggtaaacctgaccggcaactccaccagcgggcagcgcggcaacaaccggcaaattgatatccgtggcatggggcccgagaatacgctggtgctggtcgatggtaaaccggtgaccagccgtaactcggtgcggtatggctggcgcggcgatcgtgactcccgcggcgataccagttgggtgccagcggagatgatcgatcatatcgatgtgatccgcggcccggcggcggcgcgctatggtaatggcgcgatgggcggggtcgtcaacatcgtgaccaaaccgaccacgcgagaatggcacgggtcgtggaatacctatatgaatgctccgcagcaccgtaaagaaggggcgacgaaacgtactaactttagcctcaatggtccgctgtcggacagtgtcagcttcaatctctggggtaatctgagtaaaacccaggccgatgcacaggatattaacgccgggcatgaagcggaacgtaccggttcctacgccggttcttatcccgccggacgtgaaggggtggtgaacaaagatattcacagtaagctgcgctgggagtttgccccgatgcaggccctggagtttgaggccggttacagccgccagggtaatctctatgccggcgacacacaaaacaccaataccagtacgctggtgaagagtatgtacgggaaagagaccaaccgtctctaccggcaaacttacggcgtaacatggaccggcggctgggataatggcgtgaccagcaacagctatgcccagtacgaacacacccgtaactcgcgaatggatgaagggctggcgggcggtacggaagggatcttctccagtagcgagttttcagatatcgatctggccgatgtcctgctacatagtgaagtgaatattccgtttacgctgggggtcgatcagaatctgacgctgggggcagaatggaatcagcagcggatgaaagatggcgtatcgacaacccaggcgctctcttatggcactatcgatggcgtatcggctaccggtcgtagcccgtactccagtgccgagatcttctcgctgtttaccgaagataatatggcgctaacggacagcaccatgctgacacccgctctgcgcttcgatcaccacagcatcgtcggcaataactggagcccctcactgaacctgtctcaggagctgacggacgactggacgctgaagctgggcattgcccgtgcttacaaggcgcctaacctctaccagttgaacccgaactatattctctacagcaacggtcaaggctgttacgccagtagttccgcctgctatctgatggggaatagcgatctgaaagcggagaccagcgttaataaagagattggtcttgagtacaagcatgatggctatcaggcggggatcacctggttccgtaacgactatcacaataagattgagtcagggtatgcggcggtgggtaccgccagcaacggcaccaccaatatctatcagtgggaaaacgtaccaaaggcgttagtggaaggcctggaaggaacgctgaatctgccggtgggggaggcggttaactggagcaataacctgacctggatgctgcagagcaagaataagacgaccggcgaccggctgtcagtgatcccgcagtttaccctgaactcgactttgagctggcaggttcgtgaagatctctccctgcagagcacctttacctggtatggccgacagaaaccaaaacgcttcaattataagggcgaggcggtcagcggcagcgaactaaacgaagtcagcccatacagcattgtcggcctcagtgcgacctgggatgtgaacaaaaatctgagcttcaccagcgggatagataacctgtttgatattcgccactaccgggcagggaatgcgcaaacgaccggcaacgcgacgacgggagcttatctgtatggcgcgggtgccgagacctataacgaatcggggcggaccttctttatgagcgttaatactcatttctga

SEQ ID NO: 54 - DNA, 2286 bp, Klebsiella pneumoniae
  source 1..2286: mol_type = genomic DNA
atggaaaaaaacgcttctctgcctttcggcagtttcaactcattggcattgtttacaggtctgtgtctgggagcctcgccggcagcaggcatcgcagcggaaaattcggtcaaaaatagtgaagagacgctggtagtggaagccgctccgccttcactctactcccccggcgcttccgccgatcccaagttcaataaaccgctggtcgataccacccgcaccatcaccgtgatcccggaacaggtgattaaagatcagggcgtcaccaacctgactgacgccctcaaaaacgttcccggcgtcggggcgttttatgccggggagaatggcagctcaaccaccggggatgccatctttatgcgcggcgtggatacctctaacagcatctatgtggacggcattcgcgacatcggtagcgtgacgcgcgataccttcaatacccagcaggtggaagtcatcaaagggcccgccggcacggactatggccgcagcgcgccctccggctcgatcaatatgatcagcaagcagccgcgccttgactccgggatcgacggctcggccagcatcggcagcgcctggtcgcgccggggcactctcgacctgaaccaggcgtttagcgacaacgctgcgttccgtctgaacctgatgggggaaaaaacgcatgacgctggtcgggaccgcattgaaaacgaacgctatggcatcgcaccgtcgctggccttcggccttgataccccaactcgtctgtatctgaactatctgcacgtccggcagaacaacaccccggatggcgggatccctaccgtcggcctgccgggctattcggcgccttcgccgaagtatgccgcactcaactccgccgggaaggtcgataccagcaatttctatggcaccgactccgattacgataaatctactaccgacagcggtaccctgcgcttcgaacacgatctgacggagaataccaccgtgcgcaataccacccgctggtcgcgagtgaaacaggagtatcttttgaccgcggtgatgggcggcgcgaacaatatcaccgcccccgatatcaatgacgtcaacacctggagctggtcgcgtctggttaataccaaagatgtcagcaaccgtattctgaccaaccagaccaatatcacctcgactttcaatactggctcgataggccatgacgtcagcgccggcgtggagtttacccgggaaaaccagaccaactatggcgttaacgccaggaccgcgccggcggtgaatctctaccatccggtgagcaacctgtcgattggcgggctggacagaaacggggcgaacgccaacggccagaccgataccttcgggatttatgcctttgatacgctgacgctgaccgagcggattgagatcaacggcgggctgcgtctcgacaattaccataccaaatatgacagcgccaccgcctgcggcggcagcggacgcggggctatcgcctgcccgcccggacagtcgaccggcagcccggtcaccactgtcgataccgctaaatccggcaatctggttaactggaaagccggggcgctgtaccgcttaaccgagcagggcaatgtctacgtcaactacgccatctcacagcagccgccgggaggcagcagcttcgccctggccgccagcggcagcggcaacagcgctaaccggaccgactttaagccacagaaggcgaaatccagcgagctgggcaccaagtggcaaatcttcgacaaccgtctgctgctcagcgcggcgttattccgcaccgatattgaaaacgaagtggccgccaacgatgacggaacctggtcgcagtacggcaaaaagcgcgtggaggggtatgaactctccgcgaccggaaacctgaccccggactggacgattatcgccggctacactcagcagcatgcgacagtgacggagggacagaacgttgcacaggatggatcttccgccctggcctacaccccgaaacatgcctttacgctgtggacgcagtatcaggccaccagcgatctgtccgtcggcggcggtgtgcgctatgtcggaagcctgcgccggggcagcgatggtgcagtcggtaccccggatcacaccgagggctactgggttgccgacgccaaactgggctatcgggtcaacagcaacctcgatctgcagctcaatatgtataacctgtttgataccgattacgtggcctccatcaacaagagcggctatcgctatcatccgggcgaaccccggacctttatgctgacggcgaacgtccatttctga

SEQ ID NO: 55 - DNA, 2373 bp, Klebsiella pneumoniae
  source 1..2373: mol_type = genomic DNA
atggcgactatgtacaaatcgactccgtcagcagcatggtgtaaaaaacgcctgctggtgacctctttgtttgcagcaatttatcagacttctgccatcgcagcagatacttccgccgttagcggcgaggcggtggatgacacctcggaacaaatgaccgtcaccgcccccgcgccggtgcagaaagccggtagcgaacacagcatcagcgcccgggagctggagaataaaggcgctaacgatttcggctcaatcatgcgctatgagccgctcatcagcgccaccggggccagcggcggctccggcaacggcaaaagcggcttcgaccgcggaggttacaccggctacaacattcgcggtatggagagcaaccgcgtaggcatcgacgtggacggtatcgcgcaacccaacgccaccggccgcggctacgtcggccgcgccgggctcaacaccttcggcatcggccgcgattatatcgacccgtatatgtacggcagcgttgatatccagtccggcgccacctcgacggaaacggccaacagcgctatcggggggaatgtctccttccgcccgaaatcagcggatgattacctgcgcccgggcaagaccagcgccttcggctaccgcagcggttacgactctgcggatcgcagctggcacaacggggtgaccgtcgccggcggcgatgagttcctgcgcgggattttggtctatagccgccgtgacggccaggaaactgaaaacaacagcggcaccgtcgacgcctacccggcgaactggcactccgatgcttttctggcctccgggatctggcagcctaacgatgagcacaagctgaccagcaccttcgactattaccataaaaccaaccacacccactacgatacctgggactccagcggcaacagcaccatcggcaccgccaaccagaccagccagacccggcgctggggcctgagcctgaaggatgactggacgccgatgaacgactacctcgacagcgtctccacaaaaatctactaccagcataccgaagcccatgactggacttatatgccggacagcgtcacccgcaaaatgcagacggtgaactctaactacgataccgacacctggggcctgcagaccgcgctggcgaaaaccctgggccgccacgatctgagcgccggtttcaacgccagcaccagcaaaacccagcggccgttcagccagtcgccgatccccagcgtttacagcgagatcatgcagccggaggcagacagccgcagctacaccctcggcggctttgtccaggataagatcaacttcgatctcgacagccacaacttcgccgttattcccggcgtgcgcgtggtgcatcaatcgactaagccggaaaatctgtccgatctcgccgccaacagcagcgtgctgagcgaatcgtcggtggcgaatctgtacggcaaaaacagcgatacccaggttctgccgtcgttgaccttccagtacgacctcaccccgcgcctgatgacctacctgcagtaccagcgcggggcgcagttccccaacgccagccagctgtatggctcctggaacctcggctccagctacgccggcagccagcagtatgccctgatcggcaataccgatctgaagacggaaaccagcgataatctcgagtgggggctgaaaggggaagttaccgaaggcatcaccctgcgcacggcgctgttctacaacagctataagaactttatcgcctatacccgctatacccgcgccaacaatccgggccagttcacgaatgtgccgtcgaacatctacaccatttatcaggcggaaaaccgcgataaagcctatatctacggcggtgagattagcaccaaatttaactttggcacctggtttgagcaggtggacggcctgagcgccaccctcgccctcggctatagcgaagggaaatcgaaatccagctacagcggcgataaatacgtcgacctcgacagcgtggcgccaatgaaagccatcgtcggcgtggcgtgggacgatccggcgaaacgctacggcaccgccctgacggcgacctttgtcaaagggaaacaggcgaccgccaccaaccgcgaaagctacagcaacagcggatccgccatcaccgatgccagcagcgactatatgcgcgtgccgggctacggcatgctggactggaccgcgtactggcaggtggcgaaaaacgtgcgcctcaatggcggggtctacaacctcaccgatcgtaaatactgggattacctgagcagccgcaatatcgagaccggcaccaaccaggacgccaacgataaagcgctggcggtgatgccgggccgcacctggcagctgggcgtcaacgtcgacttctga

SEQ ID NO: 56 - DNA, 2430 bp, Klebsiella pneumoniae
  source 1..2430: mol_type = genomic DNA
atggcgatgaaaaagttgctcatagcgtcgctgctgtttagcagcgcgactgtatacggtgctgaagggttcgtggtgaaggacattcatttcgaaggcttgcagcgtgtcgctgttggtgcggccctcctcagtatgccagtgcgtcctggcgatacggtgaccgacgatgatatcagtaacactattcgcgcgctgtttgccactggcaacttcgaggacgtccgcgtcctgcgcgatggtgataccctgctggttcaggtgaaagagcgtccgacgatcgccagcatcactttctccggcaacaagtcggtgaaagatgacatgctcaagcagaaccttgaggcctcaggcgttcgggtgggcgagtcgcttgaccgcacgaccatcgcggatatcgagaagggtcttgaagacttctactacagcgtcggtaaatacagcgccagcgtcaaagcagtcgttacgccgctgccgcgtaaccgtgtcgatttgaagctggtcttccaggaaggcgtctccgcaaaaattcaacagatcaacatcgtcggcaaccatgcgttttcgaccgatgagctgatctcccacttccagctgcgcgatgaggtgccgtggtggaacgtggtcggcgaccgtaaataccagaagcagaagctagcgggcgaccttgaaaccctgcgcagctactacctggatcgcggctatgcccgtttcaacatcgattctacccaggtcagcctgacgccggataagaaagggatctacatcaccgtcaacatcaccgaaggcgatcagtacaagttttccggagtgcaggtgacgggcaacctcgctggccattccgcggaaatcgaagcgctgactaaagttgagccaggcgaactgtacaacggcgcgaaagtgaccaagatggaaaacgacatcaagaaactgttgggtcgttatggttacgcctatccgcgcgtgcagtcgcagccggagatcaacgacagcgataaaaccgttaagctgcacgttaacgtcgacgcaggcaaccgttattacgtgcgtaaaattcgcttcgaaggcaacgacacctctaaagatgccgtactgcgccgcgaaatgcgccagatggaaggcgcatggctgggcagcgacctcgtcgatcagggtaaagaccgtctcaatcgtttaggtttctttgaaacggtggatactgatacccagcgcgtgccgggcagcccggaccaggtcgacgttgtctacaaggtgaaagagcgtaacaccggtagcttcaacttcggtatcggctacggcaccgagagcggcgtcagcttccaggcgggcgttcagcaggataactggttaggtactggctatgctgtcgggatcaacggtaccaaaaacgactaccagacctataccgagctgtcggtgaccaacccgtacttcaccgtagacggtgtaagcctcggcggtcgtgtcttctataatgactttgatgcgaacgatgcggatctgtctgactataccaacaaaagctatggtacagacattacgctgggcttcccggtcaacgaatacaacacgctgcgcgccggcgtcggttatgtgcataactccctgtccaatatgcagccgcaggtggcaatgtggcgttaccttaactcgatgggccagtatccggacaacaccaacgaccggaactcgttcagtgcgaatgacttcaccttcaactacggttggacctataacaagcttgaccgcggcttcttcccaacggaaggttcgcgcgtcaacctgaacggtaaggtgaccattccgggctcagacaacgagtactacaaagcgacgctggataccgcgacctacgtgccgatcgacaacgatcatcagtgggtagtactgggtcgtacgcgctttggttatggcgatggtatcggcggcaaagagatgccgttctatgagaacttctatgccggtggttccagcaccgtgcgtggcttccagtcgaacaccattggtccgaaggcggtgtacttcccggcaagcagtcgtcatgatgatgacgatagttacgataatgaatgtaagagcaccgaatccgcaccgtgtaaatccgatgatgcggtgggcggtaacgcgatggcggtggccagccttgagctgattaccccgacgccgtttattagtgacaaatatgcgaactcggtccgtacttccgtcttctgggatatgggtaccgtatgggatactcactgggattcgagcgcgtacgctggttatccggattacagcgatccgagcaacatccgtatgtctgcgggtattgccgtgcagtggatgtcgccgttggggccgttggtcttctcctacgcccaaccgttcaaaaagtacgatggagacaaagccgaacagttccagtttaacattggtaaaacctggtaa

SEQ ID NO: 57 - DNA, 2691 bp, Klebsiella pneumoniae
  source 1..2691: mol_type = genomic DNA
atgacagatgtgactattaaagcgctggcctcagagattcagacctctgtggatcgcctgatacagcaatttgctgacgcaggcatccgcaaatcggctgatgattctgtgacctcgcaagagaaacaaactttgttgacgcacctgaaccgtgaacacggctcggcgccagacaagctgacgttacagcgtaagacgcgcagtacgttaaatattccaggtaccggtggaaagagtaaatcggtacaaatcgaagtccgcaagaaacgcacctttgtgaaacgcgatccgcaagaggctgaacgcctggccgcggaagagcaggcgcagcgtgaagcggaagagcaggcccgtcgtgaagctgaagaagcagcgaaacgcgaggcgcaattaaaagctgaacgtgaggccgcagaacaagctaaacgtgaagtcgctgataaagcgaaacgtgaagctgcggaaaaagacaaagtgagcaatcaacataccgacgaaatgaccaaaaccgcccaggctgaaaagatccgtcgcgagaacgaagccgcggaattgaagcgcaaatcggaagaagaagcacgccgcaaacttgaagaagaagcgcgccgtgtagcggaagaagcacgccgtatggctgaagaaaacgaaaaaaattggtctgaaacctcagacagcccggaagatagcagcgactatcacgtcaccacatcacagcatgctcgtcaggctgaagatgataacgatcgtgaagtcgaaggcggtcgcggccgtagccgtagcagcaaagcggctcgtccggcgaagaaaggcaacaaacacgctgaatcgaaagctgatcgtgaagaagcccgcgcggccgtgcgcggcggtaaaggcggtaagcaccgtaaaggttccgctctgcagcagggcttccagaagccagcgcaggccgttaaccgtgacgtcgtaatcggcgaaaccatcaccgttggcgaactggctaacaagatggcggtgaaaggttctcaggtcatcaaagcgatgatgaagctgggcgccatggcgaccatcaaccaggtcatcgaccaggaaaccgcacagctggttgccgaagagatgggccacaaagttatcctgcgtcgtgaaaacgaactggaagaagccgtaatgagcgaccgtgacaccggcgcggcggctgaaccgcgcgcaccggtcgtgaccattatgggtcacgttgaccacggtaaaacctcgctgctggactacattcgttctaccaaggttgcctccggcgaagcgggtggtattacccagcacatcggtgcttaccacgtcgaaaccgacaacggcatgatcaccttcctggataccccgggccacgccgcgtttacctccatgcgtgctcgtggcgcgcaggcgacggatatcgtggttctggtggtggcggcagacgacggcgtgatgccgcagactatcgaagctatccagcacgctaaagcggcgcaggtaccggtggtagtggcggtgaacaagatcgataagccagaagccgatccggatcgcgtgaagaacgaactgtcccagtacggcatcctgccggaagagtggggcggcgagagccagttcgtccacgtttccgcgaaagcgggtaccggcatcgacgacctgctggacgcgatcctgctgcaggctgaagttcttgagctgaaagcggtccgcaacggtatggcgagcggcgcggtcatcgaatccttccttgataaaggtcgtggtccggtagctaccgttctggttcgcgaaggtactctgcacaagggcgacattgttctgtgcggcttcgaatatggccgtgtgcgcgcgatgcgtgacgaactgggtcgcgaagtgctggaagcgggtccgtccattccggtggaaatcctcggcctgtccggtgtgccggctgccggtgatgaagtgaccgtagtgcgtgacgagaaaaaagcgcgtgaagtggcgctgtatcgtcagggcaaattccgtgaagttaagctggcgcgtcagcagaaatctaaactggaaaacatgttcgctaacatgaccgaaggcgaagttcacgaagtgaacatcgtactgaaagcggacgtacagggttctgtcgaagcgatttccgattccttactgaaactgtctaccgacgaagtgaaagtgaagatcatcggttccggcgtaggtggtatcaccgaaaccgacgctaccctggcagcagcatccaacgcgattctggttggcttcaacgttcgtgccgatgcctctgcgcgtaaagttatcgaagcggaaagcctggatctgcgttactactccgtcatctataacctgatcgacgaagtgaaagcggcgatgagcggcatgctgtctccggaactgaaacagcagatcatcggtctggctgaagtgcgtgatgtcttcaaatcgccgaaattcggcgccatcgcgggctgtatggttaccgaagggacgatcaaacgtcacaacccaatccgcgttctgcgtgacaacgtggttatctatgaaggcgagctggaatccctgcgccgcttcaaagatgacgttaacgaagtccgtaacggcatggaatgtggtatcggcgtgaagaactacaacgacgttcgcgttggcgatatgatcgaagtgttcgaaatcatcgaaatccagcgtagcatcgattaa

SEQ ID NO: 58 - DNA, 3234 bp, Klebsiella pneumoniae
  source 1..3234: mol_type = genomic DNA
atgaaaagaatgttaatcaacgcaactcagcaggaagagttgcgcgtcgcccttgttgatgggcagcgcctgtacgacctggatatcgaaagccccgggcacgaacagaaaaaagcgaacatctacaaaggcaaaatcacccgcattgaacccagccttgaagccgcgtttgttgattacggcgccgagcgtcatggtttcctccccctcaaagaaatcgcccgcgaatacttccccgccaactacaatgcgcatggtcgtcctaatatcaaagacgtactgcgggaaggtcaggaagttatcgtgcagattgataaagaagaacgcggcaacaaaggcgctgcgctcaccacctttatcagcctcgcgggcagctatctggtactgatgccgaacaacccgcgcgccgggggaatttcccgccgtatcgagggcgacgaccgtaccgaactgaaagaagcgctggcgagcctggagcttccggacggcatgggcctgatcgttcgcaccgctggcgtcggcaaatccgccgaagccctgcagtgggacctgagcttccgcctgaagcactgggaagcgattcagaaagccgcggaaagccgtccggcgccgttcctgatccaccaggaaagcaacgtcattgtccgcgccttccgtgactacctgcgccaggacatcggcgaaatcctgatcgataacccgaaagtgcttgagctggcgcgccagcatatcgccgcgctgggtcgtccggatttcagcagcaaaataaaactgtacaccggtgaaatcccgctgttcagccattatcagatcgaatcgcaaattgagtccgccttccagcgcgaagtgcgcctgccttccggcgggtctatcgttatcgatagcaccgaagcgctgaccgcgatcgatatcaactccgcccgcgccacccgcggcggcgatatcgaagagacagccttcaataccaacctcgaagcggctgacgaaattgcccgccagctgcgtctgcgcgacctcggcggcctgatcgttatcgacttcatcgatatgaccccggttcgccaccagcgcgccgtggagaatcgtctgcgcgaagccgtccgtcaggaccgtgcgcgcattcagatcagccatatttcgcgcttcggcctgctggagatgtcccgtcagcgcctgagcccgtcgctgggcgagtccagccaccacgtctgcccgcgctgctccggcaccggcaccgtgcgtgataacgaatcgctgtcgctctctattctgcgtctgatcgaagaagaagcgctgaaagagaataccaaagaagtccacgccattgttccggtaccgatcgcctcctatctgctgaacgaaaaacgtgccgcagtgagcgctatcgaatcccgtcagggcgatgtgcgcgttattatcgtgccaaacgacgaaatgcaaacgccgcactactccgtcctgcgcgtgcgcaaaggtgaagaaacctcaacgctgagctatctgctgccgaagctgcatgaagaagaaatggcgctgccaggcgacgatgagccggcggagcggaaacgtccggaacagccggccctggccgcttttgtcatgccagatgcgccgccagccccgatgctcgaagagcctgccgccgcgcctgtcgccgcagcggcaccggtcgcggccgccgcaccggcgcagcctggcctgctctcacgcttcttcagcgcgctgaagaatatcttctctggcgccgaagaggccaaaccggctgaagttcaggtcgagaagaaagcggaagaaaaaccggagcgtcagcaggagcgtcgtaaaccgcgcgccaacaaccgccgcgaccgcaacgaccgccgtgataaccgcgacaatcgtgacaaccgcgataaccgtgacaatcgcgacacccgtgcggacaatgccgagggccgtgaaccgcgcgaatcgcgtgaagagaaccgtcgcaaccgtcgcgagaagccgtcgcagaacgtggaagcccgtgatgttcgccaaacctcaggcgacgacgcggagaaagcgaaatcccgtgacgagcagcagccgcgccgcgaacgcacccgccgccgcagtgacgacaaacgtcaggcgcagcaggaagccaaagcgcagactcgcgaagagccggttgtgcaggagacggagcaggaagagcgtgtacaaactctgccgcgtcgtaaaccgcgccagctggcacagaaagtgcgcgttgagtccgctgtcgtcgagccagttgccgagatcgtgccagaagccgtagtggctgaagttatcgctccgcacagcgagccggtgaaagccgagctgccggcaggggtggagagcgtggcggaccaggacgaaaatggcgaatcccgtgaagcgaacggtatgccgcgtcgctcacgtcgctccccgcgtcacctgcgcgtcagcggtcagcgtcgtcgtcgctatcgtgacgaacgctatccgacccagtcgcctatgccgctgaccgtagcctgcgcatcgccggagatggcttccggtaaagtctggatccgctacccggtggttcgtccgcaggatcagcagccggaagaggttcaggttcaggacgccagcgtcgcgaaaactgtcgaggccgtagcggccccggtcgccgtcgttgaaaccgttaccgctgcgccggtcaccgtcgagccggctaccatggaaccagtaaccgctgagccggtagtcgtcgagccggtagcggccgccgagccgctggtcgttgatgctgcggaagttgtcgcgccagcagccgtcgagccagcgcctcaggagccggtcaccgaagcaccggctgtcgaagcgcctcaggctatcgcgccagtgacgctcgacgccgagccggtggtggtagaacctgaagcggtggaaacgacgcctgtcgttgcagcgccagtggaaactatcgccccggtcgcagaaaccgtggagcaagcgccagtgaccgaagcggcccctgccgaaccggtcaaagccgagcccccggtgagcaagccggtcgtagtggcgggtcatcgccatgccaccgcgccaatgacccgtgcgccagctccggactatgtcccggaagcaccgcgtcatagcacctgggtgcgcccgccgttcgcctttgaaggtaaaggcgccgccggtggtcatagcgcgacccataaagccaccgctgaaccgactcgcccacagcccgtcgagtaa

SEQ ID NO: 59 - DNA, 3480 bp, Klebsiella pneumoniae
  source 1..3480: mol_type = genomic DNA
atgcgcaagctctcactaagtttactcacgctgtccctcggcgttgcgctgctgccgttagcgcaggcggcgacgacgcctgcccaggagcatctgctggagcaggtccgcctcggcgaggccagcaatcgtgaagacctggtgcgccagtcgctgtaccgtctggagctgattgatcccaacaacccggagctgattgccgcgcggatgcgctatctgctgcgtcagggggatgccgccggggcgcaaaaagagctggaacgactgacgaagcaggcgccggactccccggagctgaaggcgtcgcgcaatgagatgaaaagcaacaccggcgagggccgccaggcgctgcagcaggcgcgactgctgggcgtggccgggaaggtcgatgaagccatcgccgcctatgaaaaactgtacggcggggtgccggatgacgttgacgtcgccattgagtactggacgctggtggcgcgcctgccggcccgccatagcgaaggcgtcagccagttgaaaaaactgaacgccagcgcgccgggcaacgtcagcctgctgacttcgctggcgaagcagatgttcgccgataacaaaccgcaggaggggttcgcctatctggcggagatggcccgatcggcctcgggacgcggtatcgccgccgatatgtggttcagtgaggtgaaaagcatgccggtgagtaaggccagcgtgcaggcgttgcagcaatttcttctgcagtttcccaccggctcggtggcggcgaacgcccgcgttctgctcgaccaacagcaggcgcagctgcaggatccgactttccgcgcccgctcggaagggctggcggcggtcaagtccgggaataccacgcaggcggtcgcggatctgcagaaagccgttcaggccgacagccgcgacagcgacgcggtgggcgctctcggccaggcctattcccagcgcggcgaccgcgcgcgggcagtggcgcagctcagtaaagcgattgctatggaccctgacagcccgaaccgcggcaagtgggacagcctgctgcaaactaaccgctactggctgctgataaagcagggggataacgccctgaaagccggccagctttcgcaggcgcagaactattatgcccaggcgcagcgggtcgatcgcaccgacagctatgccgtgctggggctgggggacgtcgcggcggcgcgcaaagaggcggcggcggcggagcgctattaccagcaggcgttgcgcctggatcgcggcaataacctggcggtgcgcggcctggccaacctctatcgcgccgaatcgccggagaaagccagcgcctggatcgccggcctccctcccgctcagcggcggagcatcgatgatattgagcgcagcctgactaacgaccggctggagaaacaggcgcaggctctggagagccagggcaactgggcgcaggcggcggaagttcagcgtcggcgcctggcgctggatccggacagcgtctggataacctaccgtctggcgcgggatctggtcagcgccggcgaacgccaggaggccgacgcgctgatgcggacgatggtcaaccgccagccgcaggacgccgaacgggtctacgcctcgggactctacctgtcggggaacgaccaggacgatctggctctggcgcaaatcgccgctctgccgcgcagcgcgtggacggataacattcgtgagctcgaagcgcgtttgcaaagcgaccgggtgctgcgccaggccaaccagctgcgcgacagcggtaacgaagcgcaggcgatcgcccttatccgacagcagcccgcctcggtgcgctatgacctgacgctcgccgactgggcgcagcagcgcggcgacagccagacggcgattgccaactatcagcgggtgctgcgccaggaggccgacaacggcgatgcgcgcctcggccttgcggaagtctacctggccgagggcgataaaccggccgcccgggcgcaggtcatgcagctgaaaggcgcagagaccgaatccatgaacatgcagcggcgggtggcgctggcgcgagctggccttggcgataccgctgacgcgcaacggatttttaatcagattgtgccgcaggcgaaggcgcagccgccctcgatggagagcgcgctggtgctgcgcgatgccgcgcgctttgccacccagagcggggcgccgcagcaggcgctgacgcactaccgggaagctatggtggcctccggcattacccccgcgcagccgcaggataacgatacttttacgcggctgacgcgcaacgacagccatgatgactggctgaagcgcgggatccgcagcgatgccgccgacctttatcgtcagcaggatctgaacgtcaccctggaacatgacttctggggttccagcggcaccggcggctattccgacctgaaggcgcataccaccatgctgcagatggatgctccgctggcggatggccggatgttcttccgcaccgacctggtcaatatggatgccggcagcttttccacccacagcgacgggagctactcgcccagctggggcacctgcggggagatcgcctgtaccagcggcagtaaaaatcagaccgacagcggggccagcgtggcggtcggctggaagaatgacacctggagcggggatatcggcaccacgccgatgggcttcaatgtcgtcgatgtggtgggggggctgagctacagcagcgacgtcgggccggtggggtacacggtcaacgtccaccggcggcctatctccagctcgctgctctcctttggcgggcagaaggacagcagcagccataccggcgccacctggggcggcgtccgcgccgacggcggcggcctgagcctgagctacgatcgcggggaggctcacggcatctggtcctcgctgggcgccgactcgctgaccggtaaaaacgtggcggataactggcgcgtgcgctggatgaccgggtactactacaaggtcatcaacgagaataatcgtcgcgtcaccgtcggcctcaacaatatgatctggcactacgacaaagatctcagcggctacaccctcggccagggcggctattacagcccacaggagtatctctcgttcgccgtgccggtgacctggcgtcagcgcaccgagaactggtcctgggagctcggcgggtcggtgtcatggtcccattcgcgcacccagacgcaagcccgctatccgctgctgaacctgatcccgtccgactaccggcagcgcgccagcgagctgacggaggaggggagcagcagccatggattcggttacaccgccagagcgctggtggagcggcgggtgaccagcaactggttcgtcggcgccgcggtcgatattcagcaggcgaaggattacaccccgagccatgcgctgctttacgtccgctactcggcggccggctggcagggggatctggatatgccgccccagccgctggtgccctacgccgactggtag

SEQ ID NO: 60 - DNA, 4236 bp, Klebsiella pneumoniae
  source 1..4236: mol_type = genomic DNA
atgagccaggaatacaccgaagacaaagaagtcaaactaaccaaactcagcagcgggcgccgactccttgaggcgatgctcatcctttgctccctcttcgccatctggctgatggcggcactactgagctttaacccctcggaccccagctggtcgcaaacggcatggcatgagcctattcataatttaggcggcgcccccggcgcgtggcttgccgataccctctttttcatttttggcgtcatggcctacaccatcccggtgatcatcatcggcggatgctggtttgcctggcggcatcaggaaaacgacgaatacattgattattttgccgtttcccttcgcctcatcggtgcgttagccctgatcctgacctcctgtggtctggcggcgattaacgccgatgatatctggtacttcgcctccggcggggtgatcggcagcctgctgagcaccacgctgcaacccctgctgcacagcagcggcggcaccatcgccctgttgtgtatctgggcggccgggctgacgctgttcaccggctggtcgtgggtcagcattgcggaaaagctgggcggcggcatcctgtccgttctcacctttgccagcaaccgtacccgtcgggatgatacctgggtcgatgaaggcgaatatgaagacgacgaggaagagtacgacgacgaagaggcggccaggccgcaggaatcgcgtcgcgcccgtatcttacgcagcgcgctggcgcggcgtaagcgtctggccgagaagtttaccaaccctatggggcgtaaaaccgacgctgcgcttttctccggcaaacggatggatgacggcgaagaggtggtgcaatacagcgccagcggggcgcctgttgccgccgacgatgtactgttttccggcgccagcgccgcgcgtcccgcagaggatgatgtgctgttctccggcgccagcgccgtgcgcccgggcgatttcgacccttacgatccgttgttgaatggccacagtatcgctgagccggtaagcgcagcggcggcggctacagccgcgccgcaggcgtgggcagaatcaccggtgggccatcacggcgctgcgccagcttatcagccggaagccagctatccgccgcagcaggcctatcagcctgaacccgctccgttccagcaggcctatcagcctgaacccgctccgttccagcaggctgcttatcagccgccagcggggcaaaccgcaccgcaggcgtatcagcctgagccagcgccgtatcaacagccggtttacgatccgcgtgccggtcaacctgcgccgcaggcctatcagcctgagccagcgccgtatcagcagccggcttacgatccgtatgccggtcaacctgcgccgcaggcctatcagcctgaacctgcgccgtatcagcagccggcttacgatccgcatgccggtcaacctgcaccgcaggcctatcagcctgagccagcgccgtatcagcagccggcttacgatccctatgccggtcaacctgcgccgcaggcctatcagccggagccagcgccgtatcagcagccaacttacgatccctatgccggtcagcctgcgcctcagacctatcagcagccggcttacgatccgaatgccggtcagcccgcgccgcagccgtatcagccggagccagcggcgtatcagccgcaaagcgcgccagttcccccaccggagccagagcccgaggtcgtgcaggaggaagtgaaacgtccgccgctctattatttcgaggaagtggaagagaagcgggcgcgcgaacgcgagctgttggcctcctggtatcagccaattcctgagccggaaagtccgattgccactaaaccgctgacgccgccgaccactgcgtccaaaccgccagtggagacaaccgtagtctctgcggtagcggctggggtgcatcaggctaccgccgccagcggcggcgcggcggcagcaacctcgtccactgccgcatccgctgcggctacgccattgttcagcccggcgtccagcggcccaagggttcaggtgaaagagggcatcggtccaaaactaccgcggcccaatcgcgtgcgtgttcctacgcgtcgggaactggcctcctacggcatcaagctaccgtcgcagcgggaggcggaacagcgcgcgcggcaggcggagcgcgatccgcattatgatgatgagctgctctcggatgaggaagcggatgctatggagcaggatgaactggctcgccagttcgccgccacccagcagcagcgctacggtcatcgctgggaagacgataacgcgactgatgacgatgaggccgacgccgcggcggaagcggagctggcgcgtcagtttgccgctacccagcagcagcggtacgctaccgagcagccgccgggcgccaacccgttctcgccggcagattatgaattctcgccgatgaaaacgttggtcaatgacggcccgagcgaaccgctgtttacgccgacgccggaagtccagccgcagcagccggcccagcgctatcaacaaccggcggccgctccgcagcagggttatcaacctgcgcagcatcagccgatacaccatcagcctgtgccgccacagccgcagtcctatccgactgcgtcgcagcccgtacagccgcaacaaccggttgccccgcaggggcatcagcctgccgcccctgcgccgcaggagagcctgatccacccgctgctgatgcgcaatggcgatagtcgaccgctgcaaaagccgaccacgccactgccgtcgctggatctgcttaccccgccgccgagtgaagtcgagccggtggatacctttgctctcgagcagatggcacgcctggtggaagcgcgactcgctgatttccgcattaaagcggatgtggtgaactactcaccggggccggtgatcacccgcttcgaactgaatctggcgcctggcgttaaggccgcacggatctctaacctgtcacgggacctggcgcgatcgctgtcaacggtcgccgtgcgcgtggtggaggtgatcccgggcaaaccgtatgtcgggcttgagctgccgaataaaaaacgccagaccgtctacctgcgtgaagtgctcgacaacgccaagttccgtgataacccatctccgctcaccgtggtgttgggtaaagacatcgctggcgatccggtagtagccgatctggcgaaaatgccgcatctgctggtggccggtaccaccggttccggtaagtctgttggcgtcaacgccatgatcctcagcatgctctacaaggcgcagccggaagatgtgcgtttcattatgatcgacccgaaaatgctcgagctgtcggtctacgaaggaattccgcacctgctgacggaagtggtcaccgacatgaaagacgccgccaatgcgctgcgctggagcgtcaatgagatggagcgccgctacaagctgatgtcggcgctgggcgtgcgtaacctcgcgggctacaacgagaagatcgccgaagccgcgcgcatgggacgtccgatcccggatccgtactggaagcctggcgacagcatggacgccgtacatccggtgctggaaaaactgccgtacatcgtggtgctggtggatgaattcgccgatctgatgatgaccgtcggcaaaaaggtggaagagctgatcgctcgcctggcgcagaaagcgcgcgcggcggggatccacctggtgctggcgacacagcgtccgtcggtagatgttattaccggcctgattaaggccaacatcccgacgcgcatcgcctttaccgtgtcgagtaaaattgactcacgtaccattctcgatcagggcggcgcggaatcgctgctgggtatgggggatatgctttactccgggccgaactctaccacgccggtgcgtgtccacggggcgtttgtgcgcgaccaggaagtccacgccgtggttcaggactggaaagcccgcggtcgcccgcaatatgtggatggcattacctccgacagcgaaagcgaaggcggcggtggcggcttcgacggcggggaagagttggatccgttgttcgatcaggcagtcaactttgtgaccgagaagcgcaaagcgtcgatttccggggttcagcgtcagttccgcatcggctataaccgtgccgcgcgtattatcgaacagatggaagcgcagggtatcgtcagcgagcagggccataacggtaaccgcgaagtgctggcgccgccgccctttgaatga

SEQ ID NO: 61 - RNA, 285 nt, Klebsiella pneumoniae
  source 1..285: mol_type = mRNA
atgaagaagttagctttactctccgccgtaatgacgcttggaatgtcgtcatgggcttttgctgccgacaacccgccgccgccgccggaaaaaggcgcgcagcatcagggtaaaccgccggtgaaaaacggccaacacgaaggtaagcaagcgcaatacaacagaaaacagccacaacgagacggcaaacagccgcagcacgacggtaaacagccgcagcacaacggcaagcagccgccaaaagggagcgagcacagcgggaaaccgctgccgccgaaagcgtaa

SEQ ID NO: 62 - RNA, 354 nt, Klebsiella pneumoniae
  source 1..354: mol_type = mRNA
atgaaacgttacgcaaccgcactgctcttttgcactctgtcgctgaccagcctggccgctcgcgccgatattatcgatgacgcgatcggcaatattcagcaagccattaacgacgcctataaccccggcagcagtcgctccgatgacgacgacagatacgatgacgatggccggtatgatgacgggcgctatcaggggagccgtcagcagagccgtgacagtcagcgccagtatgacgagcggcaacgccagctggacgagcgccgccgccagctggatgaacgccagcgtcagctcgaccgcgatcgtcgtcagttagaaagcgaccagcgtcgtctggatgatagctactga

SEQ ID NO: 63 - RNA, 360 nt, Klebsiella pneumoniae
  source 1..360: mol_type = mRNA
atgttcaggtcactgattctggcagcagtactgctggccgcagggccactggtcgctaacgctggtgaaatcaccctgctgccatcggtaaaattacaaataggcgatcgtgacaattacggtaactactgggacggtggcagctggcgcgaccgtgattactggcgtcgtcactatgaatggcgtgataaccgttggcatcgtcatgacaacggctggcacaaaggctggtacaaaggcagagataaagcctgggagcgcggctatcgtgctggctggaacgaccgcgatgaccaccgcggcggctggggtcgcggcccgggcgggcgcggtcacggtcatggacatggccatcactaa

SEQ ID NO: 64 - RNA, 363 nt, Klebsiella pneumoniae
  source 1..363: mol_type = mRNA
atgaaggaaatcggcttaccgttattgctactgaccgcgctggccagtccggcttttgctgcagactgtcagccaaacggcattggcggctcgttttgcattaacgatgacggtacgactaccgacacggtgcctaacgaagtcaacggcatggatacgtactcgaataatggcggctataccagttccctgcccgatcggtcaggggcggatgaagcactggaaggttcatcgctgtcgacgcagcaaggcgtcggcagcggacagagcgacagtgcgctggcgggtcgcgactggcattcgcccgccaatctgaatgatggcgccgccacctccagtatgagcctgctggataaaccctga

SEQ ID NO: 65 - RNA, 366 nt, Klebsiella pneumoniae
  source 1..366: mol_type = mRNA
atgaatatgaaaaaactgacgacccttttgctcaccgccaccttaggtcttgccagcggcgcggccctggcggcagacaccggcgcccagtccaataatggccaggccaactcttccgcggatgccggtcaggtggcgccggatgcccgtgagaacgtggcgccgaacaacgtggacaatagtcagatcaactctggctctgggggcaccacgggctcgacgatgacccaggataatatgtcgagcaatgaggtacataaaaactcgatgtgtaaagacggccgctgtccggacaccggtaaaaaactggacaacggtggcaatacgacccaagacaacagcaaaaccgacggcaccacccagtaa

SEQ ID NO: 66 - RNA, 369 nt, Klebsiella pneumoniae
  source 1..369: mol_type = mRNA
atgaaacaccgcatcgctctgcttctggtcctgacttcacttagcgccagcgccctagccgcctctccctgccaggaaaaagagcaggatattcaacgagagatcagctacgccgaaaagcatcataatcaaagtcgcattgatgggctaaataccgcgctacgtcaggttcgggaaaactgtagcgacagtaaactcaaagccgatcatcagcaaaaaattgccaaacagcgggaagagatcgctgaacgtcagcgcgatctgcaggaagcccggaagaaaggcgatgcggacaaaattaacaaacgccagcataaactcaatgaagcgcaacaggagttaaaaacgctggagtctcgggattactaa

SEQ ID NO: 67 - RNA, 426 nt, Klebsiella pneumoniae
  source 1..426: mol_type = mRNA
atgcgactcataacacgacacgtgagagaggatattatgaaaaaagcaatgattgcgttatcggctattctggttgcggctccggtttttgctgcgacaacacatgcaacagatgataccgtcgcggcggcgaatgccaacgccaacaccgctaaagagaagctgcatcaggcccagcacgagggcgaagagcagcagctgaaggcgaaacacgccgccgaaggcaagcaggacagcgtcggcagccaggtgagcgaaggcgcgcagaaaacctggaacaagaccaaagaaggcaccgagaaggggtgggataagaccaaagaggtcagtgaaaaaggctggaacgccaccaaatccggtgcggaaaagggctgggataaaaccaaaaccggcgccgaagagttaaaaaataaagtgactgaataa

SEQ ID NO: 68 - RNA, 456 nt, Klebsiella pneumoniae
  source 1..456: mol_type = mRNA
atgaaaaagatgatttctctggcagtaattttatcctgtgtgctgagcgtcccggcctttgccgatggcccgaacgacggccatcgcccggagcagcccacggtgtggcagaacggtccggaccatgacgggcatgcaccgcagggcggacctgacgcgcatcatcagggcgaccatgaccagcgtggcccggatcgcgacggccatgacaaacgcgatctggcacgtcatgagcaggaccatttcgcctggcgcgggaacgatttccgcaaaggccacccggctccggcgccgttccgtggcgatgaatatcgcgtccgcgactggagcgaccgcggcctgccgcccccgccggaaggccatcactggtcctatatcgacggtaactatgtgctgatcgccgcggcgaccgggatcatcacctcgattctggtgagcggcgccctcggccactaa

SEQ ID NO: 69 - RNA, 507 nt, Klebsiella pneumoniae
  source 1..507: mol_type = mRNA
atgaaaaaaccgacatccgccacccgtggcaaatccggccgcaagtcgcgtgaagagttaaatcaggaagctcgcgatcgcaaacggcagaagaaacatcgtggccacgcggcaggcagtcgcgcgaacggcggcgatgcggcttcagcgggtaaaaaacagcgtcaggcgcaagatccgcgcgtgggtagcaaaaaaccgatcccgctgggcgtgagcgaaagcagcgttccagctcccaagcagcataaaccaaagagcgagaaacctatgctttcaccgcaggctgagctggagttgctggagaatgatgagcgcctggacgcgctgctggaacgtctggaagagggcggcaccctgaatgctgaagagcagagctgggtggacgccaaactggatcgcattgatgagctgatgcagcagctcggcctctcttacgatgatgaagatgaagaagaggaagagcgtcaggaagatatgatgcgtctgctgaagggtggaaactaa

SEQ ID NO: 70 - RNA, 690 nt, Klebsiella pneumoniae
  source 1..690: mol_type = mRNA
atggcgagtaagtttcagaaccgtttagtcgggacaatcgtgctggtggcgctgggggtgattatcctgccagggctgctggacgggcagaaaaagcattaccaggatgagtttgccgcgatcccgctggtaccgaaaccaggcgatcgcgatgaaccggatatgttgccggcggcaacccaggcgttgccttcgcaaccgccggaaggggcggcggaagaggtgcgggcgggcgatgccgccgcgccatcgttagatccatcgcgtattccggtgaacagcaacagcttcgatgacgttcaggagccggtggtggccgcgaaaccgcagcccaagccgcagccgaaaccgcagccgcaacagcaggcctcgacgccaacgccgccgccggctaagccacagcagcaacagccaccgcagcagcaggcggccctgccggcgccgaccggcaaagcctatgtggttcagctgggcgcgttgaagaacgccgataaggtgaatgagattgtcggtaaactgcgggcctcgggtttcaaagtctatacgtcgccttcgacgccggtacagggtaaaattacccgcatcctcgtcggcccggatgcgtcaaaagacaagctgaaaggccagctgggcgatctgcagcagatctccgggcttagcggggtggtgatgggcttcaccccgaactga

SEQ ID NO: 71 - RNA, 969 nt, Klebsiella pneumoniae
  source 1..969: mol_type = mRNA
atggcacaacgagattatgtacgccgcagccaaccggcttcttcgcggcgcaaaaagagcacgacccgaagctcaaggaataagcaaagcagccttccggcgatttcaccggcgatggtggcgatcgcggcggctgtgctggtggcctttatcggtggcctctatttcattacgcatcataagaaagaagaagcggaagcgatgcaaaatcgccaggccgccggcaacggcttgccgcccaaaccggaagagcgctggcgctatattaaagagctggaaagccgccagcctggcgtccgcgcgccgaccgaaccgaccgccggtggcgaagtcatgaaaccggaacagctgaccgacgagcagcgccagctgctcgcccagatgcaggccgatatgcgccagcagccgacccagctgaccgaagtgccgtggaacgaacaaacgccggcgcagcgccagcagacgcttcagcgtcagcgtttagcgcagcaacagcagcaggcgcagcagcaacagtgggcgcagactcaggcgcagaccgtccaacagcagccgccgcgcgttcagcagccgaagccggttcagcagcaacagccgaagcagaccgcgtcaaaccagcagccgtaccaggatctgctgcagacgccagcgcataccaataccacgcagccgcgtacccaggccgcggcgccggtaactcgggtggaagaagcgccgaaaaccaccgccgagaagaaagacgatcgtagctggatgatccagtgcggctcttttaaaggcgccgagcaggccgaaaccgtccgcgctcagctggctttcgaagggtttgcttcgcacattaccactaacaacggctggaaccgcgtggttattggcccgttgaaaggcaaagaaagcgccaacgagatgatcacccgcctgaagatggctggacacgcgaactgcattcgtctcgccgccaggggttga

SEQ ID NO: 72 - RNA, 1131 nt, Klebsiella pneumoniae
  source 1..1131: mol_type = mRNA
atgagcgcgggaagcaccaaatttaccgtcagccgtattgcggctctttcactggtttcactctggctggccgggtgtaccaacaccaataatccgcctgcgccggttagctctgccggcggcgccgcctcttccagcaccaactccggcatgctgattacgccgccaccctccggcgtcaagtccgctcctcaggcgcagccgattcagccgatgcagacccagaccattcagccggcgccggtggcgcaggagccggtacagacggtaaatggccggatcgtttacaaccgcaaatatggcgatattccgaaaggtagctataccggcggcagtacctatacggtaaaacgcggcgacacgctattctatatcgcctgggtcaccggcaacgatttccgcgacctggcgcaacgtaacaatatcccggccccgtacgcgctgaacgtggggcaggtactgcaggtcggtaacgcctcaggccagccgatcaccggcgaaaacgccgtttctcaggccagcgcaagagcgagcggcggtgcgacgaccagcacaacttctgcacaaaaatcgaccgcggtggttgcttcacaaccgactattacgtattctgaatcttcaggtgaacagagtgctaccaagatgttgcctaataataaaccagcgaccacaaccacaacggttgtcgcgccggtgacggcaccaacaacggtgagcacaacccagccgactgcaagcagtacgtcaaccagttcgccgatctcagcatggcgctggccgactgatggcaaggttatcgagaactttagcggcgcggaaggcggcaataaaggcatcgatattgcaggcagtaagggacaggctattgtcgcgaccgccgatgggcgcgtcgtctatgccggtaacgcactgcgcggctacggtaatcttattatcatcaaacacaacgatgattacctgagtgcctacgctcataacgataccatgctggttcgggagcaacaggaagtcaaagcggggcagaaaatcgctaccatgggtagcaccggaaccagctcaacaagattacattttgaaattcgttacaaggggaaatccgtcaacccgctgcagtacttaccgcagcgataa

SEQ ID NO: 73 - RNA, 1149 nt, Klebsiella pneumoniae
  source 1..1149: mol_type = mRNA
atgcgtaagcaatggctggggatctgcatagcagcggggctgctggcggcatgttcgagtgatgacgtgcaacaaaaaacggtcagtactccacagccggccgtctgtaatggcccgacggttgagatcagcggcgccgatccgcagtatgaaacgccgaacgccacggcgaatcaggattatgagcgcgacggtaaaagctacaaaatcgttcaggatccggccaactttactcaggccggtttcgcggcgatctatgacgcagaacccaacagcaacctgaccgccagcggcgaagccttcgatccgactcagttgaccgcagcgcacccgacgctgccgatcccgagctacgcgcggatcactaaccttgccaacggacggatgatcgtcgtgcggattaacgatcgcggtccctatggcaacgatcgggtcatctcgctttcccgcgcatccgctgaccgcctgaacacctccaacaacaccaaagtgcgcatcgaccccatcatcgtcgcgcctgacggttcgctttccggcccggggatggcctgtaccaccgtcgccaaacagacttacgccctgcccgcccgtccgaatctggacggtggggacgccgctggcatgagccagcccgcgcccactgacgttcgcccgatcagcaacagcacgctgacgccggcagacagcgtgggcgcgccggtgaacagcggcggtttcctcggcgcgccgacgcccctgaacaacggcgtgctggagagtagcgaaccagcggcagccgccgcgacggctcctgccgccggcgccacgccaacagcgccagtgaccgcgcctggctccattcagggtaatgtggtgcccgctgcggccaccgccgcagccgctggcgccgtggcggcctcgtcctccgcgacctccagcgccagcggtaattttgttgtccaggtgggcgcagtaagcgaccagacgcgggcgcagcagtatcagcagcgcctgagccagcagttttctgtgccaggccgggtcatgcaaaacggcgcggtctggcgtattcagctgggtccctttgctgataaagcacaggccagcgccgtgcagcagcgcctgcaaagcgaagcgcagctgcagtcctttattactcgcgccaactaa

SEQ ID NO: 74 - RNA, 1287 nt, Klebsiella pneumoniae
  source 1..1287: mol_type = mRNA
atggatgatttcaaaccagaagacgatatgaaagccgatcgcaacgatcgtcgtgctggtcgttcccgtcagtcttccgagcgtgatgccgatccgcagatcaattttgacgatgttgatcttgatgccgatgaaggccgtccgacgcgcgctggtaaggcccgtcgcgagcgtgaagaggaagagttcgaagaagaactggatgcgcaagacgaggagatgctcgaagagcagcctgtagagcgtcgtccgcgcaagcgtaaaaaagcgccggccaaaccggcctcccgccagtacatcatgatgggtgtggggattctggtgctgctgctgttgatcgtgggtatcggttccgcactgaaatcgccatcatcttccagccagcagaccgcttccggcgagaagagcattaatctgtctgacgaccagtccgccagcatgcctgctgccggccaggaccagactgccgccgctaacagcacctcacagcaggacgtaacggtaccgcctattgccgcgaacccgacgcagggccaggcagcggttgcgccgcagggccagcagcgtatcgaagttcagggcgatctgaacaatgccttgacccagcagcagggccaactggacggcgccgtggctaactcgacgctgccgactgaaccggctaccgtcgcgccaatccggaatggcgccaatggcaccgcggcgccgcgccaggcgaccgagcgtcagacagcagcgaccccgcgtccggctgaacgtaagcataccgttatcgaagcgaagccgcagtcgaagccacaggccgtggcgaaaacgccggtagaatcgaagccggtccagccgaagcatgttgaaagcacggcgaccaccgctccggcgaaaacgtccgtcagcgaaagcaaaccggtggccaccgctcagagcaaaccgaccacgacgaccgcagcgccagcggcaacggcagctgcggcagcgccggcagcgaagaccgggaagacggcaggtgacgtcagctcaatgaaaactgcgccgtcgggtcactatactctgcagctcagcagctcctctaactacgacaacctcaacaactgggcgaagaaagagaagctggataaatatgttgtctatgaaacgtcgcgtaacggccaaccatggtacgtgctggtgagcggtatctatgcatcgaaagatgaagcgaaacgtgctgtcacctcgctgccggcggacgtgcaggcgaaaaatccatgggcaaaaccgctgcatcaggttcaggctgacctgaaataa

SEQ ID NO: 75 - RNA, 1326 nt, Klebsiella pneumoniae
  source 1..1326: mol_type = mRNA
atgtcaaaggcaaccgaacaaaacgacaagcttaaacgagcgatcatcatttcagtcgcgctgcacatcattctgatcgcgctgctgatctggagttcgtttgacgagcatctggatgcctctgccggcggcggcggcggatcgtcgattgatgccgtcatggtcgatccgggggcggtggtaaataactataaccgtcagcaacagcagcaggccagcgcacgtcgcgccgctgaacagcgtgaaaaacaggcgcagcagcaggcggaagagttacgtgagaaacaggcggcggaacaggaacggctgaaacagctcgaacaggagcggctgcaggcgcaggaagcggcgaaagaagcgaaggagcagcagaagcaggctgaagaagcggctgccaaggccgccgcggcggcaaaagccaaagcggacgcacaggcaaaagaagcgcaggaagccgctgccaaagcggccgccgaggcgaaagcgaaggcggatgcccaggcgaaagcggcagaacaggcggcggccaaggcggctgctgacgcgaaaaagcaggccgaagccgctgcagcgaaagccgctgccgaggcgaagaaacaggcggaagctgaagcggcgaaagctgcggccgaggcgcagaagaaagcggaagcggcggctgcgaagaaagcgcaacaggaagcggagaaaaaagcccagcaggaagcggctaagcaggcggcagctgaaaaagcggctgccgaaaaagccgctgagaaagccgccgcgcaaaaagcggccgctgagaaggccgccgccgagaaagccgcagccgctgaaaaagcggcggcagcgaaagcggctgcagcagagaaggctgcagctgataaagcggccaaagcggcagcagcaaaagccgcggcggcgaagaaagcggcggctgcgaaagaagcggacggcgttgacaacctgctcggcgatctgagttctggtaagaatgcgcctaaaacaggcggtggggccaaaggaaacaatgcctccgctgccgggagtggtaatactaaaaacagtgcctcaggggctgatatcaacaactatgccggacagataaaatcggcgattgaaagtaagttttatgacgcatcgtcctatgcgggcaaaacatgtaccttgcgtatcaaacttgctcctgacggcctgttgttaaatatacagtccgaaggtggtgatcctgctctgtgccaggccgctcttgccgcagcccgacaggctaagtttccgaaaccacctagccaggcagtatatgaagtcttcaaaaatgcgccactggacttcaaacctcagtga

SEQ ID NO: 76 - RNA, 1455 nt, Klebsiella pneumoniae
  source 1..1455: mol_type = mRNA
atgttttttttaagtattttttacatggagatgacaaaagtgaaattaagcgctctgtttattgccctgattcctctactgggctcgccggttattcatgcagaaactactgctgcgccggttctggaaaatcgcgctgcgcagggagatatcaccactcctggcggcgcgcgtcgtttaacaggcgatcaaaccgaagcgctgcgcgcctcgttaatcaataagccagctaaaaacgttattttgctgattggcgatggcatgggtgattcggaaattaccgctgcgcgaaactatgccgagggggcgggcggtttctttaaaggaattgatgctctgccgttaaccgggcagtacacgcattattcgctggataaaaaaaccgggaaaccggactacgtgaccgactcggcggcctccgccaccgcctggaccaccggcgtgaagacttataacggcgcgctgggcgtcgatattcatgagaatgcgcatcagaccatcctcgagctggcgaaagcggcggggctggccaccggcaacgtttccaccgccgagctgcaggacgccacccccgcggcgttggtagcgcatgtgacatcgcgtaaatgctacggcccgacggtcaccagcgaaaaatgccccagcaatgcgctggaaaaagggggcaaaggctccattaccgaacagctgctgaacgcccgaccggatgtcaccttgggcggcggcgcgaagacctttaccgaaacggcgacggcgggcgagtggcagggcaaaaccctgcgcgagcaggcgcaagcgcgcggctaccagattgtgaccgacgcggcttctcttgccgccgcgacggaagccagtcaggataaaccgctgctgggactctttgccgatggcaatatgccggtacgctgggaagggccgaaggcgtcttatcacggtaatatcgataagccgccggtgacctgtacgccaaacccgaagcgtgacgcctcggtgccgacgctggcgcagatgacggagaaagcgattgacctgctcagtcgcaacgagaaaggtttcttcctgcaagtcgaaggcgcttccatcgataagcaggaccatgcggcgaatccgtgcggccagatcggcgaaacggttgatcttgacgaagcggtgcagaaggcgctggaattcgcgcgaaaagacggtaataccctggtgatcgtcaccgccgaccatgcgcatgccagccagatcatcccggcggatagcaaagccccggggctgacccaggctctgaacacgcacgatggcgcggtgatggtgatgagctacggcaactctgaggaagagtcgatggagcacaccggcacccaactgcgcattgcggcctacggtccgcatgcggctaacgtcgtaggcctgaccgatcagaccgacctgttcacgaccatgaaagctgccctgagtctcaaataa

SEQ ID NO: 77 - RNA, 1608 nt, Klebsiella pneumoniae
  source 1..1608: mol_type = mRNA
atgtcactgccgttcaaaccccatattatcgccctgctctgtagcgctggcttactcgcggcggcaggaacactctatgtgcaaagccgaaccccagcgacgatcgctgaaccgcctgcgcagcaagcgccagcgcccgcagcgtcgacgacacagccggtggccgccacttacacccaggcgcaaattgatcagtgggtcgcccctatcgcgctctacccggacagcctgctgtcgcaggtgttgatggcctccacttatcccgacaacgtcctgcaggcggtccagtggtcccaggataaccccgcgatgaaaggggatgcggccgtgcaggcggttgccagccagccgtgggaccctagcgtcaaatctcttgtcgctttccctgccctgctggcgatgatgggcgagaatccgccctgggtggaaaatcttggcaatgcgtttttggcccagccgcatgatgtgatggattcagtgcagcgcctgcgcgccattgcccaacaaaccgggacgctgaaatccacaccgcagcagaaagtgattgtcacccctgccgcaccggtttcagccagcagcagcacggcagcaaccgcaaccgcccacacagcggcgcctgcgcccacgcaggtcattaaaatagagccgaccaatccacaggtggtctatgttcccagctataacccctccaccgtctatggtacctggccgaacagcgcctatccgccggtctatctgccgccccctcccggggagcagtttaccgatagcttcgtcaaaggcttcgggtacagcctcggcgtggccaccacctgggcgctgttcagcagtatcgactgggatgatgatgaccatcaccatcacgatgacgactaccaccacggcgattactcgcataatggcgataacatcaatattaatgtaaataatttcaatcatataacaggagaaaacctgccgggaaaccacgttaactggcagcacaatcctgcctatcgcggacacacaccgtatcccgataatacggtagctcagcgcttccatcagaccaacgtttccggcggactgagcgcgacccaacatgcgccagtcgatcgcgaagcgcagcgccaggcagcgatgacccagctgcagcataacgtaccgacggccacagcgggcaacctggcggcaaacaacgcctcacgcgacgcccagcgtcaggcggcctcggcgcagctgaagcaagccacccaacgcagtaattaccgcggttacgacagtacgccgacccaacagcagcgtcgcgaggcggcaaaaacgcagctgaaaaaccccacgccgcagcaacagcagcgtcgagaagccgccaggagccacgagcagaaccgcacacctcagcagcagcagcgccggcagcagttccagtccgccacgccagcccagcgtcagcagacgctcagccatctgcgcgccaacgcccttagcggcaacgaaagccgcgccccctcctggcaagcgcagcaggaacgaggactgcagagccgccagttttccggcgtaaaccgcgagttacgcgatggcaccagagaacgtctttccgaacaccatgaactgcgtcgccgctaa

SEQ ID NO: 78 - RNA, 1794 nt, Klebsiella pneumoniae
  source 1..1794: mol_type = mRNA
atgtttaagtttaaggcttcttatgtcgcactggcggcagtattaacctcgtcggtagtttatgccgaccccacaagctatacgcactcttccggcgccacggttatcgatattgaaaagccgaacgccgccggtgtctcccataacctgtaccgcgacttcaacgtcggcgccaatggcaccatcctcaataacagcggcgatgatgtcagccacagcacatttggcaatatcgcccgcaacaataatctgaccgccggcagcgcttcggtgattttgaacgaggtgacctccaaaaacgccagtagcctgaagggctttatcgaagtcaacggtcagaaagcggatgtggtaatcgccaacccgaacggcatcacctgttccggctgtagctttgttaataccaacaaggctatcctgaccaccggcaaggttaatatgaccgacgatggcgctatcggcagctataccgtaacgggcggcaccctcaccatcggcgaaaatggcatgaacgccgccaacggctatgcggttctgctcgccgacgcgatcaatatcaacggtaaagtgcaggccaacaacgccctggtcagcgcgggcaacttcaccatggataacagctctggctcggtgacctccgctggtaaaaaggccaccctgatccagatgacggttaacccgcagtacagcatcgacgtcagcagccttggcggcattgaggccaacagcatcagcatggtcggcaataacatcggctttggcgtacgtaataaaggctctatcgtcgcgaatagttcgctgcagctcaccagcaacggtaatctgctgaacaaaggcacgatcaaaagcaacggtctgctgagtcaggtcgccaccgcctcgggcatcaccaatgacggtagcatcgccggcgcctattatttaatgctctccagtggcgattatatcgttaacaccggttctctctccggcggccagctgattgccaccgctaacggcaacatcaccaacggcgactcaggcacgatgaccggcaccagtggattaagcctgaccagcggcgggaaaatccgcaacgaagaaaaagcctccctgctgtcaaataaccagattgccgccacggcaatcggtgatttcctcaatgaaggcaaaatcagcgccaaacacaccagcctgacgtttgtcggcgacagctttaaaaacactggcaatattaactctactggccaaaccaccattcagtcgcttaaacaggacggcagcgccaatacgggcgagatctataacctcggcaatatcaccggcgaaaatatcaatctgcagaccaatggcacgctggcgcaaagcagcagtggtcgtattgaggcaaccaacgccattaccgcccacagctactggctgaaccaaaatggttatatgaatgccgccgatatcaccaccgatcacggcgtagtgaataattatggcaatattactgccaaaaatatttcaatcacgacctactcagatatcaccaacgaagggcagatcagcagcaccggcgacctgaccttaaataccaaaaataaaggcgcgatctacaattattcaaccctcagcgcgggcggcaacatgacgttaaccgccaccaaagtggtcaatggtggtaaaagttgcggcatactgggcctggcgaaatgcggcgtcgggacgttaactgctgacaagctggtactgaactcatcgcagaaatatgttagcgacatgggtggaaaacagtatttcaagagcaccgaagtcaacacggtgaaataa

SEQ ID NO: 79 - RNA, 1875 nt, Klebsiella pneumoniae
  source 1..1875: mol_type = mRNA
atgatggacaacctacgcacggccgccaacagcgtcgtgctcaagattattttcggtatcattatcgtctcgttcattttgaccggggtgagtggttacctgattggcggtggcaaaaactatgccgcaaaagtgaatggccaggagattggccgtgggcagtttgaaaacgccgtcgccagcgaacgtaaccgtatgcagcagcagcttggcgatcaattctccgagctggcggcgaacgaaaactacatgaaaaccatgcgccagcaggtgctgaaccgcctgatcgatgagtcgcttctggatcagtatgcccgcgagctgggcctcagcatcagcgatgagcaggtgaagcaggcgatcttccagacccaggcgttccagacgaacggtaagttcgacaaccagcgtttcagtggtattgtcgcccagatggggatgaccaccgatcagtacgcccaggcgctgcgtaaccagctgaccacgcagcagctgattaacgccattgcgggtaccgacttcatgctgccgggcgagtccgatcagctggcggcgctggtatctcaacagcgggtggtccgcgaagcgaccatcaacgtaaatgccctggcggcaaaacagaccgccagcgatgaggaaatcaacgccttctggcagcagaatcaggcccgttttatggcgccggagcagttccgcgtcagctacatcaaaatggatgccgccagcatgcaggagagcgcctctgacgaagagattcagtcatggtacgaccagcacaaggatcagttcactcagccgcagcgcaaccgctacagcgtgattcagaccaaaactgaagccgatgcgaaagcggtactggccgagctgcaaaaaggagcggacttcgccacgctggcgaaagaaaaatcgaccgatattatctctgcccgcaacggtggcgatatggggtggatggaagatgcctctaccgtgcctgagctgaaagatgccgggctgaaagagaaaggccagctgtctggcgtgatcaaatcctcggttggcttcctggtagctcgtctggacgacgtccagccggcgcaggtgaagccgctggctgacgtgcgtaatgacattgcggcgaaagtgaagcaggaaaaagcgttggatgcttactacgcgctgcagcagaaggtgagcgatgcggccagcaacgataatgaatcgctggcgagcgcagcgcaggtcgccgggctgaaggtcgtagaaaccggctggtttggccgcgataacctgccggaggagctgaactttaaaccggtcgctgacgctattttcaacggcggtctggtgggtgagaacggcgcgccgggcagcaactccgatatcattaccgttgacggcgatcgcgcttttgttctgcgcattagcgaacacaaagccgaggcggtgaagccgctggccgaagtgaaggcacaggttagcgatatcgttaagcacaataaagcggaacagcaggcgaaactggaggccgacaagctgctggcggcgctgaaagacggcaaaggcgatgaagcgatgaaggcggctggcctgagctttggcgcgccgcagacgctttctcgtaccggccaggatccgctgagccagctggcatttaccctgccgctgccgcagcagggtaaaccggtctacggcgtgggcagcaatatgcaaggcgatgtggtgctggtagcgctggatgaggtgaaagccggcagcatgccggaagagcagaagaaggccatggttcaggggatcacccagaacaatgcccaaatcgctttcgaagcgctgatgagcaacctgcgcaaggcggcgaaaattaagctcggcgacagcatcgaccagcagcagtaa

SEQ ID NO: 80 - RNA, 1974 nt, Klebsiella pneumoniae
  source 1..1974: mol_type = mRNA
atgttcaggttaaacccttttatccgggcgggattgtctgcgtccgtcgtatcgttggcgtttccggctctggccgatgtgaatgaagaaacgctggtggtgaccgcctcggccactgaacagaatgtcaaagacgcgccggcgagcatcagcgtcatcacccaacaggatttacaacgcaagcctgttcagaacctgaaagacgtgctgcgcgatgtgcctggggtccagctcaccaacgaaggggataaccgcaagggcgttagcatccgcggtctgagcagcagctataccctgatcctggtcgacggcaagcgcgttaactcgcggaacgccgtcttccgccacaatgacttcgaccttaactggatcccggtggatgctattgagcgtatcgaagtggtgcgcggcccgatgtcctccctctacggctccgatgcgctcggtggggtggtcaacattattaccaaaaaaatcggccagaaatggaccgggacgctgagtgctgataccactattcaggagcaccgcgatcgcggggatacctggaacggccagttcttcaccagcggcccgctgatcgacggcgtacttggaatgaaggcctacggcagcctggcaaaacgcgccaaggacgatccgcagtcatccagtaatgccaccggcgagacgccgcgcatcgagggcttcaccagccgcgatggcaatgttgaattcgcctggacgccgaacgaaaaccacgattttaccgcaggctacggctttgaccgtcaggatcgcgattccgattcccttgaccgcaaccgccttgagcgggagaactactctctgagccataacggccgctgggatatcggcaatagcgagctcaagttctacggcgaaaaggtggataacaaaaatccagggcagagcgggactattacctcggaaagcaatgccatcgacggcaagtatgtcctgccgctgggcatgattaaccagctggtgaccttcggcggcgaatggcgccacgacaaacttaaagatccggtcaacctgagcagcggcggccagtcaacgtcggccagccagtacgccctgtttatcgaagacgaatggcgcatcatcgagccgctggcgctgaccaccggcattcgtatggacgaccatcagacctatggcgatcactggagcccgcgcgcctatctggtgtataacgccaccgataccgtcaccgtcaaaggcggctgggcgacggcgtttaaagccccgtcgctgctgcagcttaaccccgactggaccaccaactcctgccgcggctcgtgcagcatcgtcggtaacccggatctgaaaccggaaaccagcgaaagcttcgagctcggtctctactaccgcggggaagagggctggcttgaaaatgtcgaaggcagcatcaccaccttccagaataatgtcgacgacatgatcgacgttctgcgcacctccagcgccagcgaagcgccgggctacccgaactttgtcggctggaaaactgtcaacggcaagcgcgtgccgatcttccgctatttcaacgtcaacaaagcccgcatcaaaggggtggagacggaggtgaagatcccgtttggcgatgagtggaagctgacggtgaactacacctacaacgatggtcgcgatctgagcaatggcggcgacaaaccgctgcagacgctgccgttccataccgccaacggcacgctcgactggaaaccgctggacgactggtccttctacgtgacggccaactataccggccagcagcgcgcggtgagcgccaccggcaaaacgccgggcggctacaccctgtttgacgttggcgcggcatggcaggtgaccaaaaacgtgaaactgcgctccggggtgcagaacgtgggtgataaagatctgagccgggacgactacagctataccgaagaaggccgtcgctactttatggcggtggattatcgcttctga

SEQ ID NO: 81 - RNA, 2028 nt, Klebsiella pneumoniae
  source 1..2028: mol_type = mRNA
atgaacagagccgccacgctgaccctcaacgcgcccctgctgatgctcgtcgctgcgctggcgctttcaacccctttcaccgccggcgccgcgccggcctttcttgattacgcccaacagcaaacccagcaatctcaggcgcaagaaaaaaacgatgccgcaagcgcaaaacaaacacaagaaagccgccagagcgcagataataaaaaaaccggtaccagcacctcacaattacaaaaaagaatcaccagccagcaggcggcgattgcacaaaaagataagcttatacagcaattaaaaaaacagcttgccgctacgccgcaaacggatactgccggagcgaatgagcaagcggcgttgaataagagaattaatgaattacaggtcgccttaagcgccgctactgcagaaaaagaggcattaataaaaaaagcaggcgttgtgcagaataataatctacagcaaagccaggccgcggcgcgtcagcagatccagcaattaacgacgcagattcagcaagccgaagctgaaaataaacgcctcagcgccagctttaccacgcttaataaagataaacacgcgctaatgacccaactggccgcaacggaaaaagagaaacaggccgctcttgagcaggtcaaagcgcttaacgctgacaaacagccgctgacgacccggctggccgccgcggaaaaagagaaacaggccgtcctcgagcaggttaaggcccttaacgccgataaacagtcgctgactattcgcctcgccgctgcggagaaagcgcagcaggccgctgttgaccaggctaaagcgcttaacgctgacaaacagccgctggctacccgactggccgccgcggaaaaagagaaacaggccgtcctcgagcaggttaaggcccttagcgccgataagcagtcgctgactattcgcctcgccgctgcggagaaggcgcagcaggccgctcttgaccaggctaaagcgcttaacgctgacaaacagccgctggcgacccggctggccgccgcggaaaaagagaaacaggccgtcctcgagcaggttaaagcccttaacgccgataagcagtcgctgactattcgcctcgccgctgcggaaaagacgcagcaggctgccctcgatcaggtcaaagcccttaacgccgataaacaatcgctgtccacccggctggccgccgcggataaagcgccgcatggccccgctaacgacgccgctgcgccaaaaaatgagccaccagagatggcggccatagtggcagcctatcgcctgcaggcggataaagacaacgcccagctacggatgaaagaagatgaaatcgaactgctgagaacgcagctttctgtacagtccaaaacgcgcagcggcgagagcgccgccgccaaactcagcgcatcgggagaacagcaggcttatgcgatcggcgcctcgatgggaagcgaggcgctcaacgtccttaccacccgtcgtactcagggagttaccgtcgacgcaggcctggtgctgcagggcatcgaagatgcctttcgcggccagcttcgtctcggagagcaggaacgtaacaaggcgctgtttgatgtgtcgcagcaggtttttcagaacctgaataaaatagagcagaaaaacatcagtgccggcaagaaatatcagcaggcgtttgcgcgcaaaaaagatgtggtctttaaagagggcgtctacagccgcgtcgattacctgggtaaaggaaaaataagcggtaatgacctggttaccgtggtgatcaaagagatgctgacggacgggacggtgatcaacgatatggaagcgaaagatcaggcgcttacgcaaaagctggatgcctatcccccggtgtttcgcgaaccgctgaagcgtctacagaaccacggctccgtgacgctcgtcgtcccgcctgaaaaggcctatggcagtaaaggattaccgccaaaaatcccgccaggcgccaccatggtttattccgtgcggatagtagatagccaacccgagccggcaaaatag

SEQ ID NO: 82 - RNA, 2106 nt, Klebsiella pneumoniae
  source 1..2106: mol_type = mRNA
atgaaaatcctgtccgtgcgtcacgccgccctcccggccctgctcttgccgctcattgcggcagcccaggccgctgatgaacaaaccatggtggtgaccgccgcgccaaccacggtttctgaactggatacccccgccgccgtcagcgtggtgaatggggatgagatgcgccaggccgcgccgcgcgtcaatctctctgaatcgctgggcgccgtgccgggcctgcaggtgcagaaccggcaaaactatgcccaggatctgcagctgtcgattcgcggctttggctcgcgctcaacctatggcgtgcgcggactgcgcatctatgtggatggcattccggccaccatgcccgacggccaggggcagacctcaaatattgatatcggcagcgttgacaccattgaggtgctgcgcggccccttctctgccctgtacggtaactcgtccggcggggtgatcaacgtcaccagccagaccggcacccagccgcccaccgtggaagccagcagctactatggcagcttcggcacctggcactacgggatgaaagccactggcgccgttggcgacggcagccacgcaggcgatgtggattacacggtctcaaccaatcgcttcaccacccatggctatcgcgatcacagcggcgcgcgcaaaaatctggcgaacgcccggctgggggtgcgcatcaacgacgtcagtaagctgactctgctgctgaatagcgtggatatcaaagccaatgacgccggtggcctgaccgccgatgaatggcgcgataacccgcgccagtcgccgcgcggcgaccagtataatacccgcaagaatacccgacagacccaggccggcctgcgctatgagcgccagctcagtgcccaggacgatctcagcgttatgatgtacgctggagaacgtgaaaccactcagttccagtcgatcccgcgcgcgccgcagctgaagccgagccatgccggcggggtgatcgaccttacccgtcactaccaggggatcgatacccggctgacccatcgcggagagctgctggtgcccgtcacgctcaccgccggtctcgactacgaaaacatgagcgagcggcgcaaagggtatgaaaactttgtgatggtcaacggcgcgccgcagtatggcgaacagggcgcgctgcgccgtaacgaacgcaacctgatgtggaacgtcgacccctacctgcagacccagtggcagctcactgacaaactctcgctcgatgccggggttcgctacagctcggtatggttcgactcgaacgactactacatcaccccaggcaatggcgatgacagcggtgatgccagctatcacaaatggctgcccgcgggctcgctgaaatatgccctgaccgacgcgtggaacgtctatctttccgccggccgcggcttcgagacgccaaccattaacgaactctcctaccgctccgataaccagagcggcctcaacttcggcctgaaaccctccaccaacgacacggtggagatcggcagcaagacgcggatcggcaatgggctgttcaccgccgccctgttccagaccaataccgataatgagattgtggtcgacagcagcagcggcgggcgcaccagttataaaaacgccggcaagacccgccgtcaggggatggagctggggctggatcagcagtttggcgagagctggcgtctgaaggcggcctggacctggctggacgcgacctatcgcactaacgtctgcgacgacgccagctgcaatggcaatcgcattccggggatcgcgcgcaatatgggctacgcctcctttggctatcagccggagcaaggttggtacgccgggagcgatattcgctatatgagcgatatcatggccaatgacgaaaacaccgccaaagcgccctcctggacggtggttggcctgacgactggctataaatggagctacggcaggatggatatggatctgttcggtcgcatcgacaacctgttcgaccgggagtacgtcgggtctgtcatcgttaacgagtctaacggacgttactacgagcctgccccgggacgtaactacggcatcggcctgaacctcgcctggcgcttcgaataa

SEQ ID NO: 83 - RNA, 2259 nt, Klebsiella pneumoniae
  source 1..2259: mol_type = mRNA
atgaaatacacgtctcacttcccgctggggatcgtcattcctctgctcgcctgtagcgtgccgctgcaggcggcagagaacatgaccgaacaatcgacgcctgacgagagcgccgccactgccgaaaatcacgaggagacgatggtcataaccgccgccaggcagaacctgcaggcgccgggcgtgtcgaccatcaccgcagaagagatccgcaaacatccccccgcccgcgatgtgtcggagttaattcgtacgcagcccggggtaaacctgaccggcaactccaccagcgggcagcgcggcaacaaccggcaaattgatatccgtggcatggggcccgagaatacgctggtgctggtcgatggtaaaccggtgaccagccgtaactcggtgcggtatggctggcgcggcgatcgtgactcccgcggcgataccagttgggtgccagcggagatgatcgatcatatcgatgtgatccgcggcccggcggcggcgcgctatggtaatggcgcgatgggcggggtcgtcaacatcgtgaccaaaccgaccacgcgagaatggcacgggtcgtggaatacctatatgaatgctccgcagcaccgtaaagaaggggcgacgaaacgtactaactttagcctcaatggtccgctgtcggacagtgtcagcttcaatctctggggtaatctgagtaaaacccaggccgatgcacaggatattaacgccgggcatgaagcggaacgtaccggttcctacgccggttcttatcccgccggacgtgaaggggtggtgaacaaagatattcacagtaagctgcgctgggagtttgccccgatgcaggccctggagtttgaggccggttacagccgccagggtaatctctatgccggcgacacacaaaacaccaataccagtacgctggtgaagagtatgtacgggaaagagaccaaccgtctctaccggcaaacttacggcgtaacatggaccggcggctgggataatggcgtgaccagcaacagctatgcccagtacgaacacacccgtaactcgcgaatggatgaagggctggcgggcggtacggaagggatcttctccagtagcgagttttcagatatcgatctggccgatgtcctgctacatagtgaagtgaatattccgtttacgctgggggtcgatcagaatctgacgctgggggcagaatggaatcagcagcggatgaaagatggcgtatcgacaacccaggcgctctcttatggcactatcgatggcgtatcggctaccggtcgtagcccgtactccagtgccgagatcttctcgctgtttaccgaagataatatggcgctaacggacagcaccatgctgacacccgctctgcgcttcgatcaccacagcatcgtcggcaataactggagcccctcactgaacctgtctcaggagctgacggacgactggacgctgaagctgggcattgcccgtgcttacaaggcgcctaacctctaccagttgaacccgaactatattctctacagcaacggtcaaggctgttacgccagtagttccgcctgctatctgatggggaatagcgatctgaaagcggagaccagcgttaataaagagattggtcttgagtacaagcatgatggctatcaggcggggatcacctggttccgtaacgactatcacaataagattgagtcagggtatgcggcggtgggtaccgccagcaacggcaccaccaatatctatcagtgggaaaacgtaccaaaggcgttagtggaaggcctggaaggaacgctgaatctgccggtgggggaggcggttaactggagcaataacctgacctggatgctgcagagcaagaataagacgaccggcgaccggctgtcagtgatcccgcagtttaccctgaactcgactttgagctggcaggttcgtgaagatctctccctgcagagcacctttacctggtatggccgacagaaaccaaaacgcttcaattataagggcgaggcggtcagcggcagcgaactaaacgaagtcagcccatacagcattgtcggcctcagtgcgacctgggatgtgaacaaaaatctgagcttcaccagcgggatagataacctgtttgatattcgccactaccgggcagggaatgcgcaaacgaccggcaacgcgacgacgggagcttatctgtatggcgcgggtgccgagacctataacgaatcggggcggaccttctttatgagcgttaatactcatttctga

SEQ ID NO: 84 - RNA, 2286 nt, Klebsiella pneumoniae
  source 1..2286: mol_type = mRNA
atggaaaaaaacgcttctctgcctttcggcagtttcaactcattggcattgtttacaggtctgtgtctgggagcctcgccggcagcaggcatcgcagcggaaaattcggtcaaaaatagtgaagagacgctggtagtggaagccgctccgccttcactctactcccccggcgcttccgccgatcccaagttcaataaaccgctggtcgataccacccgcaccatcaccgtgatcccggaacaggtgattaaagatcagggcgtcaccaacctgactgacgccctcaaaaacgttcccggcgtcggggcgttttatgccggggagaatggcagctcaaccaccggggatgccatctttatgcgcggcgtggatacctctaacagcatctatgtggacggcattcgcgacatcggtagcgtgacgcgcgataccttcaatacccagcaggtggaagtcatcaaagggcccgccggcacggactatggccgcagcgcgccctccggctcgatcaatatgatcagcaagcagccgcgccttgactccgggatcgacggctcggccagcatcggcagcgcctggtcgcgccggggcactctcgacctgaaccaggcgtttagcgacaacgctgcgttccgtctgaacctgatgggggaaaaaacgcatgacgctggtcgggaccgcattgaaaacgaacgctatggcatcgcaccgtcgctggccttcggccttgataccccaactcgtctgtatctgaactatctgcacgtccggcagaacaacaccccggatggcgggatccctaccgtcggcctgccgggctattcggcgccttcgccgaagtatgccgcactcaactccgccgggaaggtcgataccagcaatttctatggcaccgactccgattacgataaatctactaccgacagcggtaccctgcgcttcgaacacgatctgacggagaataccaccgtgcgcaataccacccgctggtcgcgagtgaaacaggagtatcttttgaccgcggtgatgggcggcgcgaacaatatcaccgcccccgatatcaatgacgtcaacacctggagctggtcgcgtctggttaataccaaagatgtcagcaaccgtattctgaccaaccagaccaatatcacctcgactttcaatactggctcgataggccatgacgtcagcgccggcgtggagtttacccgggaaaaccagaccaactatggcgttaacgccaggaccgcgccggcggtgaatctctaccatccggtgagcaacctgtcgattggcgggctggacagaaacggggcgaacgccaacggccagaccgataccttcgggatttatgcctttgatacgctgacgctgaccgagcggattgagatcaacggcgggctgcgtctcgacaattaccataccaaatatgacagcgccaccgcctgcggcggcagcggacgcggggctatcgcctgcccgcccggacagtcgaccggcagcccggtcaccactgtcgataccgctaaatccggcaatctggttaactggaaagccggggcgctgtaccgcttaaccgagcagggcaatgtctacgtcaactacgccatctcacagcagccgccgggaggcagcagcttcgccctggccgccagcggcagcggcaacagcgctaaccggaccgactttaagccacagaaggcgaaatccagcgagctgggcaccaagtggcaaatcttcgacaaccgtctgctgctcagcgcggcgttattccgcaccgatattgaaaacgaagtggccgccaacgatgacggaacctggtcgcagtacggcaaaaagcgcgtggaggggtatgaactctccgcgaccggaaacctgaccccggactggacgattatcgccggctacactcagcagcatgcgacagtgacggagggacagaacgttgcacaggatggatcttccgccctggcctacaccccgaaacatgcctttacgctgtggacgcagtatcaggccaccagcgatctgtccgtcggcggcggtgtgcgctatgtcggaagcctgcgccggggcagcgatggtgcagtcggtaccccggatcacaccgagggctactgggttgccgacgccaaactgggctatcgggtcaacagcaacctcgatctgcagctcaatatgtataacctgtttgataccgattacgtggcctccatcaacaagagcggctatcgctatcatccgggcgaaccccggacctttatgctgacggcgaacgtccatttctga

SEQ ID NO: 85 - RNA, 2373 nt, Klebsiella pneumoniae
  source 1..2373: mol_type = mRNA
atggcgactatgtacaaatcgactccgtcagcagcatggtgtaaaaaacgcctgctggtgacctctttgtttgcagcaatttatcagacttctgccatcgcagcagatacttccgccgttagcggcgaggcggtggatgacacctcggaacaaatgaccgtcaccgcccccgcgccggtgcagaaagccggtagcgaacacagcatcagcgcccgggagctggagaataaaggcgctaacgatttcggctcaatcatgcgctatgagccgctcatcagcgccaccggggccagcggcggctccggcaacggcaaaagcggcttcgaccgcggaggttacaccggctacaacattcgcggtatggagagcaaccgcgtaggcatcgacgtggacggtatcgcgcaacccaacgccaccggccgcggctacgtcggccgcgccgggctcaacaccttcggcatcggccgcgattatatcgacccgtatatgtacggcagcgttgatatccagtccggcgccacctcgacggaaacggccaacagcgctatcggggggaatgtctccttccgcccgaaatcagcggatgattacctgcgcccgggcaagaccagcgccttcggctaccgcagcggttacgactctgcggatcgcagctggcacaacggggtgaccgtcgccggcggcgatgagttcctgcgcgggattttggtctatagccgccgtgacggccaggaaactgaaaacaacagcggcaccgtcgacgcctacccggcgaactggcactccgatgcttttctggcctccgggatctggcagcctaacgatgagcacaagctgaccagcaccttcgactattaccataaaaccaaccacacccactacgatacctgggactccagcggcaacagcaccatcggcaccgccaaccagaccagccagacccggcgctggggcctgagcctgaaggatgactggacgccgatgaacgactacctcgacagcgtctccacaaaaatctactaccagcataccgaagcccatgactggacttatatgccggacagcgtcacccgcaaaatgcagacggtgaactctaactacgataccgacacctggggcctgcagaccgcgctggcgaaaaccctgggccgccacgatctgagcgccggtttcaacgccagcaccagcaaaacccagcggccgttcagccagtcgccgatccccagcgtttacagcgagatcatgcagccggaggcagacagccgcagctacaccctcggcggctttgtccaggataagatcaacttcgatctcgacagccacaacttcgccgttattcccggcgtgcgcgtggtgcatcaatcgactaagccggaaaatctgtccgatctcgccgccaacagcagcgtgctgagcgaatcgtcggtggcgaatctgtacggcaaaaacagcgatacccaggttctgccgtcgttgaccttccagtacgacctcaccccgcgcctgatgacctacctgcagtaccagcgcggggcgcagttccccaacgccagccagctgtatggctcctggaacctcggctccagctacgccggcagccagcagtatgccctgatcggcaataccgatctgaagacggaaaccagcgataatctcgagtgggggctgaaaggggaagttaccgaaggcatcaccctgcgcacggcgctgttctacaacagctataagaactttatcgcctatacccgctatacccgcgccaacaatccgggccagttcacgaatgtgccgtcgaacatctacaccatttatcaggcggaaaaccgcgataaagcctatatctacggcggtgagattagcaccaaatttaactttggcacctggtttgagcaggtggacggcctgagcgccaccctcgccctcggctatagcgaagggaaatcgaaatccagctacagcggcgataaatacgtcgacctcgacagcgtggcgccaatgaaagccatcgtcggcgtggcgtgggacgatccggcgaaacgctacggcaccgccctgacggcgacctttgtcaaagggaaacaggcgaccgccaccaaccgcgaaagctacagcaacagcggatccgccatcaccgatgccagcagcgactatatgcgcgtgccgggctacggcatgctggactggaccgcgtactggcaggtggcgaaaaacgtgcgcctcaatggcggggtctacaacctcaccgatcgtaaatactgggattacctgagcagccgcaatatcgagaccggcaccaaccaggacgccaacgataaagcgctggcggtgatgccgggccgcacctggcagctgggcgtcaacgtcgacttctga

SEQ ID NO: 86 - RNA, 2430 nt, Klebsiella pneumoniae
  source 1..2430: mol_type = mRNA
atggcgatgaaaaagttgctcatagcgtcgctgctgtttagcagcgcgactgtatacggtgctgaagggttcgtggtgaaggacattcatttcgaaggcttgcagcgtgtcgctgttggtgcggccctcctcagtatgccagtgcgtcctggcgatacggtgaccgacgatgatatcagtaacactattcgcgcgctgtttgccactggcaacttcgaggacgtccgcgtcctgcgcgatggtgataccctgctggttcaggtgaaagagcgtccgacgatcgccagcatcactttctccggcaacaagtcggtgaaagatgacatgctcaagcagaaccttgaggcctcaggcgttcgggtgggcgagtcgcttgaccgcacgaccatcgcggatatcgagaagggtcttgaagacttctactacagcgtcggtaaatacagcgccagcgtcaaagcagtcgttacgccgctgccgcgtaaccgtgtcgatttgaagctggtcttccaggaaggcgtctccgcaaaaattcaacagatcaacatcgtcggcaaccatgcgttttcgaccgatgagctgatctcccacttccagctgcgcgatgaggtgccgtggtggaacgtggtcggcgaccgtaaataccagaagcagaagctagcgggcgaccttgaaaccctgcgcagctactacctggatcgcggctatgcccgtttcaacatcgattctacccaggtcagcctgacgccggataagaaagggatctacatcaccgtcaacatcaccgaaggcgatcagtacaagttttccggagtgcaggtgacgggcaacctcgctggccattccgcggaaatcgaagcgctgactaaagttgagccaggcgaactgtacaacggcgcgaaagtgaccaagatggaaaacgacatcaagaaactgttgggtcgttatggttacgcctatccgcgcgtgcagtcgcagccggagatcaacgacagcgataaaaccgttaagctgcacgttaacgtcgacgcaggcaaccgttattacgtgcgtaaaattcgcttcgaaggcaacgacacctctaaagatgccgtactgcgccgcgaaatgcgccagatggaaggcgcatggctgggcagcgacctcgtcgatcagggtaaagaccgtctcaatcgtttaggtttctttgaaacggtggatactgatacccagcgcgtgccgggcagcccggaccaggtcgacgttgtctacaaggtgaaagagcgtaacaccggtagcttcaacttcggtatcggctacggcaccgagagcggcgtcagcttccaggcgggcgttcagcaggataactggttaggtactggctatgctgtcgggatcaacggtaccaaaaacgactaccagacctataccgagctgtcggtgaccaacccgtacttcaccgtagacggtgtaagcctcggcggtcgtgtcttctataatgactttgatgcgaacgatgcggatctgtctgactataccaacaaaagctatggtacagacattacgctgggcttcccggtcaacgaatacaacacgctgcgcgccggcgtcggttatgtgcataactccctgtccaatatgcagccgcaggtggcaatgtggcgttaccttaactcgatgggccagtatccggacaacaccaacgaccggaactcgttcagtgcgaatgacttcaccttcaactacggttggacctataacaagcttgaccgcggcttcttcccaacggaaggttcgcgcgtcaacctgaacggtaaggtgaccattccgggctcagacaacgagtactacaaagcgacgctggataccgcgacctacgtgccgatcgacaacgatcatcagtgggtagtactgggtcgtacgcgctttggttatggcgatggtatcggcggcaaagagatgccgttctatgagaacttctatgccggtggttccagcaccgtgcgtggcttccagtcgaacaccattggtccgaaggcggtgtacttcccggcaagcagtcgtcatgatgatgacgatagttacgataatgaatgtaagagcaccgaatccgcaccgtgtaaatccgatgatgcggtgggcggtaacgcgatggcggtggccagccttgagctgattaccccgacgccgtttattagtgacaaatatgcgaactcggtccgtacttccgtcttctgggatatgggtaccgtatgggatactcactgggattcgagcgcgtacgctggttatccggattacagcgatccgagcaacatccgtatgtctgcgggtattgccgtgcagtggatgtcgccgttggggccgttggtcttctcctacgcccaaccgttcaaaaagtacgatggagacaaagccgaacagttccagtttaacattggtaaaacctggtaa

SEQ ID NO: 87 - RNA, 2691 nt, Klebsiella pneumoniae
  source 1..2691: mol_type = mRNA
atgacagatgtgactattaaagcgctggcctcagagattcagacctctgtggatcgcctgatacagcaatttgctgacgcaggcatccgcaaatcggctgatgattctgtgacctcgcaagagaaacaaactttgttgacgcacctgaaccgtgaacacggctcggcgccagacaagctgacgttacagcgtaagacgcgcagtacgttaaatattccaggtaccggtggaaagagtaaatcggtacaaatcgaagtccgcaagaaacgcacctttgtgaaacgcgatccgcaagaggctgaacgcctggccgcggaagagcaggcgcagcgtgaagcggaagagcaggcccgtcgtgaagctgaagaagcagcgaaacgcgaggcgcaattaaaagctgaacgtgaggccgcagaacaagctaaacgtgaagtcgctgataaagcgaaacgtgaagctgcggaaaaagacaaagtgagcaatcaacataccgacgaaatgaccaaaaccgcccaggctgaaaagatccgtcgcgagaacgaagccgcggaattgaagcgcaaatcggaagaagaagcacgccgcaaacttgaagaagaagcgcgccgtgtagcggaagaagcacgccgtatggctgaagaaaacgaaaaaaattggtctgaaacctcagacagcccggaagatagcagcgactatcacgtcaccacatcacagcatgctcgtcaggctgaagatgataacgatcgtgaagtcgaaggcggtcgcggccgtagccgtagcagcaaagcggctcgtccggcgaagaaaggcaacaaacacgctgaatcgaaagctgatcgtgaagaagcccgcgcggccgtgcgcggcggtaaaggcggtaagcaccgtaaaggttccgctctgcagcagggcttccagaagccagcgcaggccgttaaccgtgacgtcgtaatcggcgaaaccatcaccgttggcgaactggctaacaagatggcggtgaaaggttctcaggtcatcaaagcgatgatgaagctgggcgccatggcgaccatcaaccaggtcatcgaccaggaaaccgcacagctggttgccgaagagatgggccacaaagttatcctgcgtcgtgaaaacgaactggaagaagccgtaatgagcgaccgtgacaccggcgcggcggctgaaccgcgcgcaccggtcgtgaccattatgggtcacgttgaccacggtaaaacctcgctgctggactacattcgttctaccaaggttgcctccggcgaagcgggtggtattacccagcacatcggtgcttaccacgtcgaaaccgacaacggcatgatcaccttcctggataccccgggccacgccgcgtttacctccatgcgtgctcgtggcgcgcaggcgacggatatcgtggttctggtggtggcggcagacgacggcgtgatgccgcagactatcgaagctatccagcacgctaaagcggcgcaggtaccggtggtagtggcggtgaacaagatcgataagccagaagccgatccggatcgcgtgaagaacgaactgtcccagtacggcatcctgccggaagagtggggcggcgagagccagttcgtccacgtttccgcgaaagcgggtaccggcatcgacgacctgctggacgcgatcctgctgcaggctgaagttcttgagctgaaagcggtccgcaacggtatggcgagcggcgcggtcatcgaatccttccttgataaaggtcgtggtccggtagctaccgttctggttcgcgaaggtactctgcacaagggcgacattgttctgtgcggcttcgaatatggccgtgtgcgcgcgatgcgtgacgaactgggtcgcgaagtgctggaagcgggtccgtccattccggtggaaatcctcggcctgtccggtgtgccggctgccggtgatgaagtgaccgtagtgcgtgacgagaaaaaagcgcgtgaagtggcgctgtatcgtcagggcaaattccgtgaagttaagctggcgcgtcagcagaaatctaaactggaaaacatgttcgctaacatgaccgaaggcgaagttcacgaagtgaacatcgtactgaaagcggacgtacagggttctgtcgaagcgatttccgattccttactgaaactgtctaccgacgaagtgaaagtgaagatcatcggttccggcgtaggtggtatcaccgaaaccgacgctaccctggcagcagcatccaacgcgattctggttggcttcaacgttcgtgccgatgcctctgcgcgtaaagttatcgaagcggaaagcctggatctgcgttactactccgtcatctataacctgatcgacgaagtgaaagcggcgatgagcggcatgctgtctccggaactgaaacagcagatcatcggtctggctgaagtgcgtgatgtcttcaaatcgccgaaattcggcgccatcgcgggctgtatggttaccgaagggacgatcaaacgtcacaacccaatccgcgttctgcgtgacaacgtggttatctatgaaggcgagctggaatccctgcgccgcttcaaagatgacgttaacgaagtccgtaacggcatggaatgtggtatcggcgtgaagaactacaacgacgttcgcgttggcgatatgatcgaagtgttcgaaatcatcgaaatccagcgtagcatcgattaa

SEQ ID NO: 88 - RNA, 3234 nt, Klebsiella pneumoniae
  source 1..3234: mol_type = mRNA
atgaaaagaatgttaatcaacgcaactcagcaggaagagttgcgcgtcgcccttgttgatgggcagcgcctgtacgacctggatatcgaaagccccgggcacgaacagaaaaaagcgaacatctacaaaggcaaaatcacccgcattgaacccagccttgaagccgcgtttgttgattacggcgccgagcgtcatggtttcctccccctcaaagaaatcgcccgcgaatacttccccgccaactacaatgcgcatggtcgtcctaatatcaaagacgtactgcgggaaggtcaggaagttatcgtgcagattgataaagaagaacgcggcaacaaaggcgctgcgctcaccacctttatcagcctcgcgggcagctatctggtactgatgccgaacaacccgcgcgccgggggaatttcccgccgtatcgagggcgacgaccgtaccgaactgaaagaagcgctggcgagcctggagcttccggacggcatgggcctgatcgttcgcaccgctggcgtcggcaaatccgccgaagccctgcagtgggacctgagcttccgcctgaagcactgggaagcgattcagaaagccgcggaaagccgtccggcgccgttcctgatccaccaggaaagcaacgtcattgtccgcgccttccgtgactacctgcgccaggacatcggcgaaatcctgatcgataacccgaaagtgcttgagctggcgcgccagcatatcgccgcgctgggtcgtccggatttcagcagcaaaataaaactgtacaccggtgaaatcccgctgttcagccattatcagatcgaatcgcaaattgagtccgccttccagcgcgaagtgcgcctgccttccggcgggtctatcgttatcgatagcaccgaagcgctgaccgcgatcgatatcaactccgcccgcgccacccgcggcggcgatatcgaagagacagccttcaataccaacctcgaagcggctgacgaaattgcccgccagctgcgtctgcgcgacctcggcggcctgatcgttatcgacttcatcgatatgaccccggttcgccaccagcgcgccgtggagaatcgtctgcgcgaagccgtccgtcaggaccgtgcgcgcattcagatcagccatatttcgcgcttcggcctgctggagatgtcccgtcagcgcctgagcccgtcgctgggcgagtccagccaccacgtctgcccgcgctgctccggcaccggcaccgtgcgtgataacgaatcgctgtcgctctctattctgcgtctgatcgaagaagaagcgctgaaagagaataccaaagaagtccacgccattgttccggtaccgatcgcctcctatctgctgaacgaaaaacgtgccgcagtgagcgctatcgaatcccgtcagggcgatgtgcgcgttattatcgtgccaaacgacgaaatgcaaacgccgcactactccgtcctgcgcgtgcgcaaaggtgaagaaacctcaacgctgagctatctgctgccgaagctgcatgaagaagaaatggcgctgccaggcgacgatgagccggcggagcggaaacgtccggaacagccggccctggccgcttttgtcatgccagatgcgccgccagccccgatgctcgaagagcctgccgccgcgcctgtcgccgcagcggcaccggtcgcggccgccgcaccggcgcagcctggcctgctctcacgcttcttcagcgcgctgaagaatatcttctctggcgccgaagaggccaaaccggctgaagttcaggtcgagaagaaagcggaagaaaaaccggagcgtcagcaggagcgtcgtaaaccgcgcgccaacaaccgccgcgaccgcaacgaccgccgtgataaccgcgacaatcgtgacaaccgcgataaccgtgacaatcgcgacacccgtgcggacaatgccgagggccgtgaaccgcgcgaatcgcgtgaagagaaccgtcgcaaccgtcgcgagaagccgtcgcagaacgtggaagcccgtgatgttcgccaaacctcaggcgacgacgcggagaaagcgaaatcccgtgacgagcagcagccgcgccgcgaacgcacccgccgccgcagtgacgacaaacgtcaggcgcagcaggaagccaaagcgcagactcgcgaagagccggttgtgcaggagacggagcaggaagagcgtgtacaaactctgccgcgtcgtaaaccgcgccagctggcacagaaagtgcgcgttgagtccgctgtcgtcgagccagttgccgagatcgtgccagaagccgtagtggctgaagttatcgctccgcacagcgagccggtgaaagccgagctgccggcaggggtggagagcgtggcggaccaggacgaaaatggcgaatcccgtgaagcgaacggtatgccgcgtcgctcacgtcgctccccgcgtcacctgcgcgtcagcggtcagcgtcgtcgtcgctatcgtgacgaacgctatccgacccagtcgcctatgccgctgaccgtagcctgcgcatcgccggagatggcttccggtaaagtctggatccgctacccggtggttcgtccgcaggatcagcagccggaagaggttcaggttcaggacgccagcgtcgcgaaaactgtcgaggccgtagcggccccggtcgccgtcgttgaaaccgttaccgctgcgccggtcaccgtcgagccggctaccatggaaccagtaaccgctgagccggtagtcgtcgagccggtagcggccgccgagccgctggtcgttgatgctgcggaagttgtcgcgccagcagccgtcgagccagcgcctcaggagccggtcaccgaagcaccggctgtcgaagcgcctcaggctatcgcgccagtgacgctcgacgccgagccggtggtggtagaacctgaagcggtggaaacgacgcctgtcgttgcagcgccagtggaaactatcgccccggtcgcagaaaccgtggagcaagcgccagtgaccgaagcggcccctgccgaaccggtcaaagccgagcccccggtgagcaagccggtcgtagtggcgggtcatcgccatgccaccgcgccaatgacccgtgcgccagctccggactatgtcccggaagcaccgcgtcatagcacctgggtgcgcccgccgttcgcctttgaaggtaaaggcgccgccggtggtcatagcgcgacccataaagccaccgctgaaccgactcgcccacagcccgtcgagtaa

SEQ ID NO: 89 - RNA, 3480 nt, Klebsiella pneumoniae
  source 1..3480: mol_type = mRNA
atgcgcaagctctcactaagtttactcacgctgtccctcggcgttgcgctgctgccgttagcgcaggcggcgacgacgcctgcccaggagcatctgctggagcaggtccgcctcggcgaggccagcaatcgtgaagacctggtgcgccagtcgctgtaccgtctggagctgattgatcccaacaacccggagctgattgccgcgcggatgcgctatctgctgcgtcagggggatgccgccggggcgcaaaaagagctggaacgactgacgaagcaggcgccggactccccggagctgaaggcgtcgcgcaatgagatgaaaagcaacaccggcgagggccgccaggcgctgcagcaggcgcgactgctgggcgtggccgggaaggtcgatgaagccatcgccgcctatgaaaaactgtacggcggggtgccggatgacgttgacgtcgccattgagtactggacgctggtggcgcgcctgccggcccgccatagcgaaggcgtcagccagttgaaaaaactgaacgccagcgcgccgggcaacgtcagcctgctgacttcgctggcgaagcagatgttcgccgataacaaaccgcaggaggggttcgcctatctggcggagatggcccgatcggcctcgggacgcggtatcgccgccgatatgtggttcagtgaggtgaaaagcatgccggtgagtaaggccagcgtgcaggcgttgcagcaatttcttctgcagtttcccaccggctcggtggcggcgaacgcccgcgttctgctcgaccaacagcaggcgcagctgcaggatccgactttccgcgcccgctcggaagggctggcggcggtcaagtccgggaataccacgcaggcggtcgcggatctgcagaaagccgttcaggccgacagccgcgacagcgacgcggtgggcgctctcggccaggcctattcccagcgcggcgaccgcgcgcgggcagtggcgcagctcagtaaagcgattgctatggaccctgacagcccgaaccgcggcaagtgggacagcctgctgcaaactaaccgctactggctgctgataaagcagggggataacgccctgaaagccggccagctttcgcaggcgcagaactattatgcccaggcgcagcgggtcgatcgcaccgacagctatgccgtgctggggctgggggacgtcgcggcggcgcgcaaagaggcggcggcggcggagcgctattaccagcaggcgttgcgcctggatcgcggcaataacctggcggtgcgcggcctggccaacctctatcgcgccgaatcgccggagaaagccagcgcctggatcgccggcctccctcccgctcagcggcggagcatcgatgatattgagcgcagcctgactaacgaccggctggagaaacaggcgcaggctctggagagccagggcaactgggcgcaggcggcggaagttcagcgtcggcgcctggcgctggatccggacagcgtctggataacctaccgtctggcgcgggatctggtcagcgccggcgaacgccaggaggccgacgcgctgatgcggacgatggtcaaccgccagccgcaggacgccgaacgggtctacgcctcgggactctacctgtcggggaacgaccaggacgatctggctctggcgcaaatcgccgctctgccgcgcagcgcgtggacggataacattcgtgagctcgaagcgcgtttgcaaagcgaccgggtgctgcgccaggccaaccagctgcgcgacagcggtaacgaagcgcaggcgatcgcccttatccgacagcagcccgcctcggtgcgctatgacctgacgctcgccgactgggcgcagcagcgcggcgacagccagacggcgattgccaactatcagcgggtgctgcgccaggaggccgacaacggcgatgcgcgcctcggccttgcggaagtctacctggccgagggcgataaaccggccgcccgggcgcaggtcatgcagctgaaaggcgcagagaccgaatccatgaacatgcagcggcgggtggcgctggcgcgagctggccttggcgataccgctgacgcgcaacggatttttaatcagattgtgccgcaggcgaaggcgcagccgccctcgatggagagcgcgctggtgctgcgcgatgccgcgcgctttgccacccagagcggggcgccgcagcaggcgctgacgcactaccgggaagctatggtggcctccggcattacccccgcgcagccgcaggataacgatacttttacgcggctgacgcgcaacgacagccatgatgactggctgaagcgcgggatccgcagcgatgccgccgacctttatcgtcagcaggatctgaacgtcaccctggaacatgacttctggggttccagcggcaccggcggctattccgacctgaaggcgcataccaccatgctgcagatggatgctccgctggcggatggccggatgttcttccgcaccgacctggtcaatatggatgccggcagcttttccacccacagcgacgggagctactcgcccagctggggcacctgcggggagatcgcctgtaccagcggcagtaaaaatcagaccgacagcggggccagcgtggcggtcggctggaagaatgacacctggagcggggatatcggcaccacgccgatgggcttcaatgtcgtcgatgtggtgggggggctgagctacagcagcgacgtcgggccggtggggtacacggtcaacgtccaccggcggcctatctccagctcgctgctctcctttggcgggcagaaggacagcagcagccataccggcgccacctggggcggcgtccgcgccgacggcggcggcctgagcctgagctacgatcgcggggaggctcacggcatctggtcctcgctgggcgccgactcgctgaccggtaaaaacgtggcggataactggcgcgtgcgctggatgaccgggtactactacaaggtcatcaacgagaataatcgtcgcgtcaccgtcggcctcaacaatatgatctggcactacgacaaagatctcagcggctacaccctcggccagggcggctattacagcccacaggagtatctctcgttcgccgtgccggtgacctggcgtcagcgcaccgagaactggtcctgggagctcggcgggtcggtgtcatggtcccattcgcgcacccagacgcaagcccgctatccgctgctgaacctgatcccgtccgactaccggcagcgcgccagcgagctgacggaggaggggagcagcagccatggattcggttacaccgccagagcgctggtggagcggcgggtgaccagcaactggttcgtcggcgccgcggtcgatattcagcaggcgaaggattacaccccgagccatgcgctgctttacgtccgctactcggcggccggctggcagggggatctggatatgccgccccagccgctggtgccctacgccgactggtag

SEQ ID NO: 90 - RNA, 4236 nt, Klebsiella pneumoniae
  source 1..4236: mol_type = mRNA
atgagccaggaatacaccgaagacaaagaagtcaaactaaccaaactcagcagcgggcgccgactccttgaggcgatgctcatcctttgctccctcttcgccatctggctgatggcggcactactgagctttaacccctcggaccccagctggtcgcaaacggcatggcatgagcctattcataatttaggcggcgcccccggcgcgtggcttgccgataccctctttttcatttttggcgtcatggcctacaccatcccggtgatcatcatcggcggatgctggtttgcctggcggcatcaggaaaacgacgaatacattgattattttgccgtttcccttcgcctcatcggtgcgttagccctgatcctgacctcctgtggtctggcggcgattaacgccgatgatatctggtacttcgcctccggcggggtgatcggcagcctgctgagcaccacgctgcaacccctgctgcacagcagcggcggcaccatcgccctgttgtgtatctgggcggccgggctgacgctgttcaccggctggtcgtgggtcagcattgcggaaaagctgggcggcggcatcctgtccgttctcacctttgccagcaaccgtacccgtcgggatgatacctgggtcgatgaaggcgaatatgaagacgacgaggaagagtacgacgacgaagaggcggccaggccgcaggaatcgcgtcgcgcccgtatcttacgcagcgcgctggcgcggcgtaagcgtctggccgagaagtttaccaaccctatggggcgtaaaaccgacgctgcgcttttctccggcaaacggatggatgacggcgaagaggtggtgcaatacagcgccagcggggcgcctgttgccgccgacgatgtactgttttccggcgccagcgccgcgcgtcccgcagaggatgatgtgctgttctccggcgccagcgccgtgcgcccgggcgatttcgacccttacgatccgttgttgaatggccacagtatcgctgagccggtaagcgcagcggcggcggctacagccgcgccgcaggcgtgggcagaatcaccggtgggccatcacggcgctgcgccagcttatcagccggaagccagctatccgccgcagcaggcctatcagcctgaacccgctccgttccagcaggcctatcagcctgaacccgctccgttccagcaggctgcttatcagccgccagcggggcaaaccgcaccgcaggcgtatcagcctgagccagcgccgtatcaacagccggtttacgatccgcgtgccggtcaacctgcgccgcaggcctatcagcctgagccagcgccgtatcagcagccggcttacgatccgtatgccggtcaacctgcgccgcaggcctatcagcctgaacctgcgccgtatcagcagccggcttacgatccgcatgccggtcaacctgcaccgcaggcctatcagcctgagccagcgccgtatcagcagccggcttacgatccctatgccggtcaacctgcgccgcaggcctatcagccggagccagcgccgtatcagcagccaacttacgatccctatgccggtcagcctgcgcctcagacctatcagcagccggcttacgatccgaatgccggtcagcccgcgccgcagccgtatcagccggagccagcggcgtatcagccgcaaagcgcgccagttcccccaccggagccagagcccgaggtcgtgcaggaggaagtgaaacgtccgccgctctattatttcgaggaagtggaagagaagcgggcgcgcgaacgcgagctgttggcctcctggtatcagccaattcctgagccggaaagtccgattgccactaaaccgctgacgccgccgaccactgcgtccaaaccgccagtggagacaaccgtagtctctgcggtagcggctggggtgcatcaggctaccgccgccagcggcggcgcggcggcagcaacctcgtccactgccgcatccgctgcggctacgccattgttcagcccggcgtccagcggcccaagggttcaggtgaaagagggcatcggtccaaaactaccgcggcccaatcgcgtgcgtgttcctacgcgtcgggaactggcctcctacggcatcaagctaccgtcgcagcgggaggcggaacagcgcgcgcggcaggcggagcgcgatccgcattatgatgatgagctgctctcggatgaggaagcggatgctatggagcaggatgaactggctcgccagttcgccgccacccagcagcagcgctacggtcatcgctgggaagacgataacgcgactgatgacgatgaggccgacgccgcggcggaagcggagctggcgcgtcagtttgccgctacccagcagcagcggtacgctaccgagcagccgccgggcgccaacccgttctcgccggcagattatgaattctcgccgatgaaaacgttggtcaatgacggcccgagcgaaccgctgtttacgccgacgccggaagtccagccgcagcagccggcccagcgctatcaacaaccggcggccgctccgcagcagggttatcaacctgcgcagcatcagccgatacaccatcagcctgtgccgccacagccgcagtcctatccgactgcgtcgcagcccgtacagccgcaacaaccggttgccccgcaggggcatcagcctgccgcccctgcgccgcaggagagcctgatccacccgctgctgatgcgcaatggcgatagtcgaccgctgcaaaagccgaccacgccactgccgtcgctggatctgcttaccccgccgccgagtgaagtcgagccggtggatacctttgctctcgagcagatggcacgcctggtggaagcgcgactcgctgatttccgcattaaagcggatgtggtgaactactcaccggggccggtgatcacccgcttcgaactgaatctggcgcctggcgttaaggccgcacggatctctaacctgtcacgggacctggcgcgatcgctgtcaacggtcgccgtgcgcgtggtggaggtgatcccgggcaaaccgtatgtcgggcttgagctgccgaataaaaaacgccagaccgtctacctgcgtgaagtgctcgacaacgccaagttccgtgataacccatctccgctcaccgtggtgttgggtaaagacatcgctggcgatccggtagtagccgatctggcgaaaatgccgcatctgctggtggccggtaccaccggttccggtaagtctgttggcgtcaacgccatgatcctcagcatgctctacaaggcgcagccggaagatgtgcgtttcattatgatcgacccgaaaatgctcgagctgtcggtctacgaaggaattccgcacctgctgacggaagtggtcaccgacatgaaagacgccgccaatgcgctgcgctggagcgtcaatgagatggagcgccgctacaagctgatgtcggcgctgggcgtgcgtaacctcgcgggctacaacgagaagatcgccgaagccgcgcgcatgggacgtccgatcccggatccgtactggaagcctggcgacagcatggacgccgtacatccggtgctggaaaaactgccgtacatcgtggtgctggtggatgaattcgccgatctgatgatgaccgtcggcaaaaaggtggaagagctgatcgctcgcctggcgcagaaagcgcgcgcggcggggatccacctggtgctggcgacacagcgtccgtcggtagatgttattaccggcctgattaaggccaacatcccgacgcgcatcgcctttaccgtgtcgagtaaaattgactcacgtaccattctcgatcagggcggcgcggaatcgctgctgggtatgggggatatgctttactccgggccgaactctaccacgccggtgcgtgtccacggggcgtttgtgcgcgaccaggaagtccacgccgtggttcaggactggaaagcccgcggtcgcccgcaatatgtggatggcattacctccgacagcgaaagcgaaggcggcggtggcggcttcgacggcggggaagagttggatccgttgttcgatcaggcagtcaactttgtgaccgagaagcgcaaagcgtcgatttccggggttcagcgtcagttccgcatcggctataaccgtgccgcgcgtattatcgaacagatggaagcgcagggtatcgtcagcgagcagggccataacggtaaccgcgaagtgctggcgccgccgccctttgaatga

SEQ ID NO: 91 - protein, 742 aa, Klebsiella pneumoniae
MNNRIKSLALLVNLGIYGVAFPLSAAETATDDKNSAAEETMVVTAAEQNLQAPGVSTITADEIRKRPPARDVSEIIRTMPGVNLTGNSTSGQRGNNRQIDIRGMGPENTLILIDGKPVTSRNSVRLGWRGERDTRGDTSWVPPEMIERIEVIRGPAAARYGNGAAGGVVNIITKKTGDEWHGSWNTYMNAPEHKDEGSTKRTNFSLSGPLGGDFSFRLFGNLDKTQADAWDINQGHQSERTGIYADTLPAGREGVKNKNIDGLVRWEFAPMQSLEFEAGYSRQGNLYAGDTQNTNSNDLVKENYGKETNRLYRNTYSVTWNGAWDNGVTTSNWAQYERTRNSRKGEGLAGGTEGIFNSNQFTDIDLADVMLHSEVSIPFDYLVNQNLTLGSEWNQQRMKDNASNTQALSGGSIPGYDSTGRSPYSQAEIFSLFAENNMELTDTTMLTPALRFDHHSIVGNNWSPSLNLSQGLWDDFTLKMGIARAYKAPSLYQTNPNYILYSKGQGCYASKDGCYLQGNDDLKAETSINKEIGLEFKRDGWLAGVTWFRNDYRNKIEAGYAPVYQNNKGTDLYQWENVPKAVVEGLEGTLNVPVSETVNWTNNITYMLQSKNKKTGDRLSIIPEYTLNSTLSWQVRDDVSLQSTFTWYGKQEPKKYNYKGQPVTGSEKNEVSPYSILGLSATWDVTKYVSLTGGVDNVFDKRHWRAGNAQTTGGATGTMYGAGAETYNESGRTWYLSVNTHF

SEQ ID NO: 92 - protein, 356 aa, Klebsiella pneumoniae
MKKTAIAIAVALAGFATVAQAAPKDNTWYAGGKLGWSQYHDTGFYGNGFQNNNGPTRNDQLGAGAFGGYQVNPYLGFEMGYDWLGRMAYKGSVDNGAFKAQGVQLTAKLGYPITDDLDIYTRLGGMVWRADSKGNYASTGVSRSEHDTGVSPVFAGGVEWAVTRDIATRLEYQWVNNIGDAGTVGTRPDNGMLSLGVSYRFGQEDAAPVVAPAPAPAPEVATKHFTLKSDVLFNFNKATLKPEGQQALDQLYTQLSNMDPKDGSAVVLGYTDRIGSEAYNQQLSEKRAQSVVDYLVAKGIPAGKISARGMGESNPVTGNTCDNVKARAALIDCLAPDRRVEIEVKGYKEVVTQPAA

SEQ ID NO: 93 - protein, 245 aa, Klebsiella pneumoniae
MDGWQRAFVLHSRPWSETSLMLDVFTEESGRVRLVAKGARSKRSNLKGALQPFTPLLVRFGGRGEVKTLRSAEAVSLALPLSGITLYSGLYVNELISRVLEHETRFSELFFDYLHCIQALAGASGSPEPALRRFELALLGHLGYGVDFLHCAGSGEPVDDTMTYRYREEKGFIASLVIDNNTFTGHHLKALASREFPDVDTLRAAKRFTRIALKPYLGGKPLKSRELFRQFMPARKARADNTNND

SEQ ID NO: 94 - protein, 1649 aa, Klebsiella pneumoniae
MKPFRLAALSLALLTAFSLTGCDDSGTPQASAPAPAADSNPGATAKPDRAQLAALAEKSQGKALTLLDASEVQLDGAATLVLTFSVPLQPDQDFSRSVHLVDKKSGKVDGAWELAPNLKELRLRHLEPKRELIVSVDPTLTALNKATLDKPFEKTLTTRDIAPSVGFASRGSLLPGNVVAGLPVMALNVDNVDVNFFRIKPESLSAFVSQWEYRNSLSNWESDELLKMADLVYSGRFDLNPARNTREKLLLPLSDIKPLQQPGVYVAVMNPAGRYSYSNAATLFTLSDIGVSAHRYHNRLDVFTQSLENGAAQSGIEVQLLNAKGQTLAEAKSDSQGHVTLQTDKEAALLLARKEGQTTLLDLKLPALDLAEFSIAGAPGFSKQFFMFGPRDLYRPGETVILNALLRDSDGKPLAEQPVKLEVVQPDGQVIRSVMSKPVNGLYQFTYPLDSGAATGMWHIRASAGDNQPREWDFHVEDFMPERMALNLTPQAAPVAPDADVTFGVSGAYLYGAPASGNQLQGKLFLRPLRDAVAALPGFQFGDIAEENLSRSLDEVQLTLDEKGHGEVTTSSQWQDSHSPLQVVLQASLLESGGRPVTRTVKQPIWPAEALPGIRPQFTLKDVYDYRTDTTVKQPVVDENSNAAFDIVYADVKGEKKAISGLQVRLIRERRDYYWNWSDSEGWQSQFDQKDLQEGEESLDLQAGQIGKVSFPVEWGSYRLEVKGPDDVVSSVRFWAGYSWQDNSEGAGAARPDRVTMKLDKPSYKPGDTIRLHIAAPAAGKGYAMVESSEGPLWWQEIDVPAEGMDLSIPVDKTWNRHDLYLSTLVVRPGDKSRSATPKRAVGLLHLPLGDENRRLTLALEAPDKMRPNQPLTVKVKASVKEGEAPKQVNVLLSAVDSGVLNITDYATPDPWNAFFGQKRYGADIYDIYGQVIEGQGRVASLRFGGDGDELKRGGKPPVNHVTIVAQQAQPVVLNDQGEGTVTLPIGDFNGELRVMAQAWTADDFGSSEDKVVVAAPVIAELNTPRFLASGDTTRLALDLSNLTDKPQTLQVHLTASGLVTLSEGQLPPVQLAPGARSTLFIPVSALAGFGDGQVNATISGLSLPGETFAPLQKQWKIGVRPAYPAQTVNSGAVLQPGESWQPPAAQSQGFAPQTLQGQLLLSGKPPLNLARYIRELKAYPYGCLEQTASGLFPSLYTSAAQLKALGISGDSDEKRRAAIDVGISRLLQMQLENGGFALWDREGPEEYWLTAYAMDFLVRASEQGYSVPVNAINKGNERLLRYLQEPGLMTVRYSDDAQASRFAAQAYAALVLARQQKAPLGALREIWSRHDQARSGLPLLQLGIALKTMGDAPRGDAALKLAVATPRQDENRWLGDYGSPLRDNALKLALLEENKLLPEVQTQLLSTLSEEAYGQRWLSTQETNALFLAGRTLADLPGSWQAQTSLQAEPLAGDKAQTRNLDGDRLAALQVSNTGSQPLWLRLDSSGYPQSAPQPGGNVLGIERTIFDTQGQQKSLSSLRSGELVLVKLEVTAKRNVPDALVVDLLPAGLELENQNLANSSASLQENGDAVQNLLNQMQQADIQHIEFRDDRFVAAVAVNEGQPVTLVYLARAVTPGTYQVPQPQVESMYAPQWRATGAASGPLTVTP

SEQ ID NO: 95 - DNA, 2229 bp, Klebsiella pneumoniae
  source 1..2229: mol_type = genomic DNA
atgaataacaggatcaaatccctggccttgctggtcaatctgggaatttacggggttgcttttccgttaagcgcagcggaaaccgccaccgacgataaaaacagcgccgctgaagagaccatggtggtcaccgccgccgagcagaacctgcaggcgccgggcgtctccaccatcaccgccgatgagatccgcaaacgccccccggcgcgcgacgtctcggagatcattcgcaccatgccgggggtcaacctgaccggcaactccaccagcggccagcgcggcaacaaccgccagattgatatccgcggcatgggcccggaaaataccctgatcctgatcgacggcaagccggtcaccagccgcaactccgtgcgccttggctggcgcggcgagcgcgacacccgcggcgataccagctgggtgccgccggagatgatcgaacgtatcgaagtgattcgcggcccggccgccgcccgctacggcaacggcgccgccggcggcgtggtgaatatcatcaccaaaaaaaccggcgatgagtggcacggctcatggaacacctatatgaacgccccggagcacaaggatgaaggctccaccaaacgcactaacttcagcctcagcggcccgctgggcggcgattttagcttccgcctgttcggtaacctcgacaaaacgcaggctgacgcctgggatatcaaccagggccatcagtccgagcgtaccgggatctatgccgatactctgccggccgggcgcgaaggggtgaaaaacaaaaacatcgatggtctggtgcgctgggaattcgctccgatgcagtcgctggagtttgaggccggctacagccgccagggcaacctctacgccggcgatacccagaacaccaactccaacgacctggtaaaagagaactacggcaaagagaccaaccgtctgtatcgcaacacctactcggttacctggaacggcgcctgggacaacggggtgaccaccagcaactgggcgcagtacgaacgcacccgcaactcgcgcaaaggcgaaggcctggccggcggcaccgaggggatctttaacagcaaccagttcacggatatcgatctggcggatgtgatgctgcacagcgaagtcagcattcccttcgactatctggttaatcagaacctgacgctgggcagcgagtggaaccagcagcggatgaaggataacgcctccaatacccaggcgctgtcgggcggcagcatcccgggctacgacagcaccggccgcagcccgtactcgcaggcggaaatcttctcgctgttcgctgagaacaacatggagctgaccgacaccaccatgctgaccccggcgctgcgtttcgatcatcacagcatcgtgggcaataactggagcccgtccctcaacctgtcgcagggcctgtgggatgacttcacgctgaagatgggcatcgcccgcgcctataaagcgccgagcctgtatcagaccaacccgaactacattctctacagtaaaggccagggctgctatgccagtaaagacggctgctatctgcagggtaacgacgacttaaaagccgagaccagcatcaacaaagagattggcctcgagtttaaacgcgacggctggctggctggcgtcacctggttccgcaacgactaccgcaacaagattgaagcgggctatgccccggtctatcaaaacaataaaggtaccgatctctaccagtgggaaaacgtgccgaaagcggtggtggaaggtctggaggggacgttgaacgttccggtgagcgagaccgtcaactggaccaacaacatcacctatatgctgcagagtaagaacaaaaagaccggcgatcgtctgtcgattatcccggaatacacgctgaactccaccctgagctggcaggttcgcgatgacgtttcgctgcagtcgaccttcacctggtacggcaagcaggagccgaagaagtacaactacaagggtcaaccggtcaccggcagcgagaagaacgaggttagcccctacagcatcctcggcctgagcgcgacctgggacgtcaccaaatacgtcagtctgaccggcggcgtggataacgtcttcgataagcgccactggcgcgcgggcaacgcccagaccaccgggggcgccaccggcacgatgtacggcgccggcgccgagacctacaatgaatcgggccgcacctggtacctgagcgtcaacacccacttctga

SEQ ID NO: 96 - DNA, 1071 bp, Klebsiella pneumoniae
  source 1..1071: mol_type = genomic DNA
atgaaaaagacagctatcgcgattgcagtggcactggctggcttcgctaccgtagcgcaggccgctccgaaagataacacctggtatgcaggtggtaaactgggttggtcccagtatcacgacaccggtttctacggtaacggtttccagaacaacaacggtccgacccgtaacgatcagcttggtgctggtgcgttcggtggttaccaggttaacccgtacctcggtttcgaaatgggttatgactggctgggccgtatggcatataaaggcagcgttgacaacggtgctttcaaagctcagggcgttcagctgaccgctaaactgggttacccgatcactgacgatctggacatctacacccgtctgggcggcatggtttggcgcgctgactccaaaggcaactacgcttctaccggcgtttcccgtagcgaacacgacactggcgtttccccagtatttgctggcggcgtagagtgggctgttactcgtgacatcgctacccgtctggaataccagtgggttaacaacatcggcgacgcgggcactgtgggtacccgtcctgataacggcatgctgagcctgggcgtttcctaccgcttcggtcaggaagatgctgcaccggttgttgctccggctccggctccggctccggaagtggctaccaagcacttcaccctgaagtctgacgttctgttcaacttcaacaaagctaccctgaaaccggaaggtcagcaggctctggatcagctgtacactcagctgagcaacatggatccgaaagacggttccgctgttgttctgggctacaccgaccgcatcggttccgaagcttacaaccagcagctgtctgagaaacgtgctcagtccgttgttgactacctggttgctaaaggcatcccggctggcaaaatctccgctcgcggcatgggtgaatccaacccggttactggcaacacctgcgacaacgtgaaagctcgcgctgccctgatcgattgcctggctccggatcgtcgtgtagagatcgaagttaaaggctacaaagaagttgtaactcagccggcggcttaa

SEQ ID NO: 97 - DNA, 738 bp, Klebsiella pneumoniae
  source 1..738: mol_type = genomic DNA
gtggatggatggcagcgcgcctttgtcctgcatagccgtccgtggagcgaaaccagcctgatgctggacgtcttcacggaagaatccggtcgcgtgcgcctggttgccaagggcgcacgctccaaacgctctaatctcaaaggcgctttacagccctttacccccttgttggttcgcttcggcgggcgcggcgaagtcaaaaccctgcgcagcgcggaagccgtttcgctggcgctgccgctcagcggcatcaccctctacagcggtctctacgttaacgagcttatctcccgcgtgctggagcacgaaacacgcttctccgaactctttttcgactacctgcactgcattcaggcgctcgccggggccagcggctcgccggagcccgcgctgcggcgttttgagctggcgctgctcgggcacctgggctatggcgtggattttctccactgcgccgggagcggcgagccggtagacgacaccatgacctaccgctatcgtgaagaaaaaggctttattgccagcctggtgattgataacaacacgtttaccgggcaccatttgaaagcattggcgagtcgcgagtttcctgatgttgatacgctgcgcgccgcgaagcgctttacccgtatcgccctgaagccctatctcggcggcaaaccgctcaagagccgggagttgtttcgccagtttatgcccgcgcgaaaggcgcgagccgataatacgaataatgattaa

SEQ ID NO: 98 - DNA, 4950 bp, Klebsiella pneumoniae
  source 1..4950: mol_type = genomic DNA
atgaaaccatttcgtttagcggcgctatcactggcgctccttaccgcattttcattaaccgggtgtgatgacagcggtacaccgcaggcctcggccccggcgccagccgcggatagcaacccaggcgcgacggcaaaaccggaccgcgcccagctggcggcgctggcggaaaaaagccagggtaaagcgctgacgctcctcgacgcctcggaggtgcaactggatggcgccgcgaccctggtgctgacgttttccgtcccgctgcagccggaccaggatttctcccgcagcgtgcatctggtcgacaagaaaagcggtaaggtggacggtgcctgggagctggcgccgaatctgaaagagctgcgtctgcgtcaccttgagccaaagcgcgagctgattgtctcggtcgacccgacgttgaccgccctgaacaaggcgacgctcgacaaaccgtttgagaagaccctgaccacccgcgatatcgcaccgagcgttgggtttgccagccgcggctcgctgctgccgggcaatgtggtcgccgggctgccggtgatggcgcttaacgtcgataatgtcgacgttaatttcttccgcattaagccggaatcgctctccgcctttgtcagtcagtgggaataccgtaactcgttaagcaactgggaatctgatgaactgctgaagatggcggatctggtctacagcggccgcttcgatctcaacccggcgcgtaatactcgcgaaaagctgctgctgccgctgagcgacatcaagccgctccagcagcctggcgtctacgtggcggtcatgaacccggcgggccgttatagctacagcaacgcggcgacgctgtttaccctcagcgatatcggcgtctcggcgcatcgctaccataaccggctggacgtttttacccagagtctggaaaacggcgccgcccagtcgggtattgaggtgcagttgctcaatgccaaagggcagaccctggcggaagcgaaaagcgacagccagggccacgtcaccctgcaaaccgataaagaagccgcgctgctgctggcgcgcaaagaggggcagaccacgctcctcgacctcaagctgccggcgctggatctggcggaattctctatcgcaggcgcgcccggctttagcaaacagttctttatgtttggcccgcgcgatctctatcgtcccggcgaaacggtgatccttaatgcgctgttgcgcgatagcgacggcaagccgctggcggagcagccggtgaaactggaggtggttcagccggacggccaggtgatccgctcggtgatgagtaaaccagtgaatggcctgtaccagtttacttatccgctcgacagcggcgcggctaccggcatgtggcacattcgcgccagcgccggcgacaaccagccgcgcgaatgggatttccacgttgaagactttatgccggaacgcatggccctgaacctgacgccgcaggccgcgccagtcgcgccggatgccgacgtcacctttggcgttagcggcgcttatctctacggcgcgccggccagcggcaaccagctgcagggcaagctgttcctgcgcccgctgcgcgacgccgtcgctgcgctgccgggcttccagttcggcgatattgcggaagaaaacctctcccgtagtctggatgaagtccagctgacgctggacgaaaaaggccatggcgaagtgaccacctcaagccagtggcaggatagccattcgccgctgcaggtggtgctgcaggccagcctgctggaatccggcggtcgcccggtgacccgtacggttaagcagccgatctggcccgccgaggcgctgccggggatccgcccgcaatttacgctgaaggacgtttacgattaccgcaccgataccaccgtgaaacagccggtggtggatgaaaacagtaacgccgcgtttgatatcgtctatgccgacgtcaaaggcgagaaaaaagcgatctccggtctccaggtgcggctgatccgcgagaggcgcgactactactggaactggtccgacagcgaaggctggcagtcgcagttcgatcaaaaagatctgcaggagggcgaggagtcgctggacctgcaggccggccagatcggcaaagtgagcttcccggtggagtggggatcctatcgcctggaggtgaaaggaccggacgacgtggtcagcagcgtgcgcttctgggcgggctacagctggcaggataacagcgaaggcgccggcgcggcgcgtcctgaccgggtcaccatgaagctggataaaccgagctataaaccgggcgacaccatcaggctgcacatcgccgccccggcggcgggtaaaggctacgcaatggtggaatccagcgaagggccgctgtggtggcaggaaattgacgtcccggcggaggggatggacctgtcgatcccggtggataaaacctggaatcgccacgacctctacctcagcacgctggtggttcgtcccggcgataaatcgcgctcggcaacgccgaaacgcgcggtgggcctgctgcatctgccgctgggcgatgaaaaccgtcgcctgacgctggcgctggaggccccggacaaaatgcgccccaaccagccgctgacggtgaaagtgaaagccagcgtcaaagagggcgaggcgccgaaacaggtcaatgtcctgctctcggcggtggatagcggggtactgaatatcaccgattacgctaccccggatccgtggaacgccttctttggccagaagcgttacggcgcagatatctatgacatctacggccaggtgatcgaagggcagggacgtgtggccagcctgcgcttcggcggcgatggcgacgagttaaaacgcggcggcaaaccgccggttaaccacgtgacgatcgtcgcccagcaggcgcagccggtggtattgaacgatcagggcgaaggcacggtgacgttacccattggcgattttaacggcgagctgcgggtgatggcccaggcgtggacggcggatgacttcggcagcagcgaagataaagtggtggtggccgccccggtcatcgccgagctgaacactccgcgcttcctcgccagcggcgatactacgcgtctggcgctggatctcagcaatctgaccgataaaccgcaaaccctgcaggttcaccttaccgccagcgggctggtaacgttgagcgagggtcaactgccgccggtacagctggcgccgggggcgcgcagcacgctgtttataccagtgagcgcgctggcgggcttcggcgatggtcaggttaatgccaccatcagcggcctgagcctgccgggcgaaaccttcgccccgctgcaaaaacagtggaaaatcggcgtgcgtccggcgtacccggcacagaccgtcaacagcggcgcagtgctgcagccaggtgaaagctggcagccgccggcggcgcagagccagggctttgccccgcaaacgctgcagggccaactgctgctcagcggtaaaccaccgttgaatctcgcccgctatatccgcgagctgaaagcctatccttacggctgccttgagcagaccgccagcggcctgttcccgtccctgtataccagcgcggcgcagctaaaggcgctgggcattagcggggacagcgacgaaaaacgccgcgcggccatcgatgtcggtatttcacgcctgctgcagatgcagctggaaaacggcggctttgcgctatgggatagagaggggccggaagagtactggctgaccgcctacgccatggacttcctcgtgcgcgccagcgagcagggctatagcgtaccggtcaacgccattaataaaggcaacgagcgcctgctgcgctatctgcaggagccgggcctgatgaccgtgcgctacagcgacgacgcccaggctagccgctttgccgcccaggcatacgccgcgctggtgctggcgcgtcagcaaaaagcgccgctcggcgccttgcgtgaaatctggagccgtcacgaccaggcgcgatccggcttgccgctgctgcagctcggcattgcgctgaaaactatgggcgatgcgccgcgcggcgacgccgccctgaagctggcggttgccaccccgcgccaggatgagaatcgttggctgggcgattacggtagcccgctgcgcgataacgccctgaagctggcgctgctggaagagaataagctgctgccggaggtgcaaactcagctgttgagcacgctgtcggaagaggcctatggccagcgctggctgtcgacccaggaaaccaacgccttgttccttgccggacgtacgctggcggacctgccgggcagctggcaggcgcaaacctcgctgcaggcggagccgctggccggcgacaaagcgcaaacccgcaacctcgacggcgatcggctggcggcgctgcaggtgagtaataccggcagtcagccgctgtggctgcgtctcgacagcagcggctatccgcaaagcgcgccgcagccgggcggtaacgtcctggggattgaacgcactattttcgatacccaggggcagcagaaatcgctgtcttcactgcgcagcggcgaactggtgctcgtcaagctggaggtgaccgccaaacgcaacgtgcctgacgcgctggtggttgatctgttgccagccggtctggagctggaaaaccagaatctggccaacagcagcgccagcctgcaggagaatggcgacgcggtgcaaaacctgcttaaccagatgcagcaggcggatattcagcatatcgagttccgcgatgaccgtttcgtcgccgccgtcgctgtcaatgaaggccagccggtgacgctggtctatctggcgcgggcggtgacgccgggcacttatcaggtcccgcagccgcaggtggaatcgatgtatgcgccgcagtggcgggcgacaggagcggccagcggtccgctcaccgtgacgccgtaa

SEQ ID NO: 99 - RNA, 2229 nt, Klebsiella pneumoniae
  source 1..2229: mol_type = mRNA
atgaataacaggatcaaatccctggccttgctggtcaatctgggaatttacggggttgcttttccgttaagcgcagcggaaaccgccaccgacgataaaaacagcgccgctgaagagaccatggtggtcaccgccgccgagcagaacctgcaggcgccgggcgtctccaccatcaccgccgatgagatccgcaaacgccccccggcgcgcgacgtctcggagatcattcgcaccatgccgggggtcaacctgaccggcaactccaccagcggccagcgcggcaacaaccgccagattgatatccgcggcatgggcccggaaaataccctgatcctgatcgacggcaagccggtcaccagccgcaactccgtgcgccttggctggcgcggcgagcgcgacacccgcggcgataccagctgggtgccgccggagatgatcgaacgtatcgaagtgattcgcggcccggccgccgcccgctacggcaacggcgccgccggcggcgtggtgaatatcatcaccaaaaaaaccggcgatgagtggcacggctcatggaacacctatatgaacgccccggagcacaaggatgaaggctccaccaaacgcactaacttcagcctcagcggcccgctgggcggcgattttagcttccgcctgttcggtaacctcgacaaaacgcaggctgacgcctgggatatcaaccagggccatcagtccgagcgtaccgggatctatgccgatactctgccggccgggcgcgaaggggtgaaaaacaaaaacatcgatggtctggtgcgctgggaattcgctccgatgcagtcgctggagtttgaggccggctacagccgccagggcaacctctacgccggcgatacccagaacaccaactccaacgacctggtaaaagagaactacggcaaagagaccaaccgtctgtatcgcaacacctactcggttacctggaacggcgcctgggacaacggggtgaccaccagcaactgggcgcagtacgaacgcacccgcaactcgcgcaaaggcgaaggcctggccggcggcaccgaggggatctttaacagcaaccagttcacggatatcgatctggcggatgtgatgctgcacagcgaagtcagcattcccttcgactatctggttaatcagaacctgacgctgggcagcgagtggaaccagcagcggatgaaggataacgcctccaatacccaggcgctgtcgggcggcagcatcccgggctacgacagcaccggccgcagcccgtactcgcaggcggaaatcttctcgctgttcgctgagaacaacatggagctgaccgacaccaccatgctgaccccggcgctgcgtttcgatcatcacagcatcgtgggcaataactggagcccgtccctcaacctgtcgcagggcctgtgggatgacttcacgctgaagatgggcatcgcccgcgcctataaagcgccgagcctgtatcagaccaacccgaactacattctctacagtaaaggccagggctgctatgccagtaaagacggctgctatctgcagggtaacgacgacttaaaagccgagaccagcatcaacaaagagattggcctcgagtttaaacgcgacggctggctggctggcgtcacctggttccgcaacgactaccgcaacaagattgaagcgggctatgccccggtctatcaaaacaataaaggtaccgatctctaccagtgggaaaacgtgccgaaagcggtggtggaaggtctggaggggacgttgaacgttccggtgagcgagaccgtcaactggaccaacaacatcacctatatgctgcagagtaagaacaaaaagaccggcgatcgtctgtcgattatcccggaatacacgctgaactccaccctgagctggcaggttcgcgatgacgtttcgctgcagtcgaccttcacctggtacggcaagcaggagccgaagaagtacaactacaagggtcaaccggtcaccggcagcgagaagaacgaggttagcccctacagcatcctcggcctgagcgcgacctgggacgtcaccaaatacgtcagtctgaccggcggcgtggataacgtcttcgataagcgccactggcgcgcgggcaacgcccagaccaccgggggcgccaccggcacgatgtacggcgccggcgccgagacctacaatgaatcgggccgcacctggtacctgagcgtcaacacccacttctga

SEQ ID NO: 100 - RNA, 1071 nt, Klebsiella pneumoniae
  source 1..1071: mol_type = mRNA
atgaaaaagacagctatcgcgattgcagtggcactggctggcttcgctaccgtagcgcaggccgctccgaaagataacacctggtatgcaggtggtaaactgggttggtcccagtatcacgacaccggtttctacggtaacggtttccagaacaacaacggtccgacccgtaacgatcagcttggtgctggtgcgttcggtggttaccaggttaacccgtacctcggtttcgaaatgggttatgactggctgggccgtatggcatataaaggcagcgttgacaacggtgctttcaaagctcagggcgttcagctgaccgctaaactgggttacccgatcactgacgatctggacatctacacccgtctgggcggcatggtttggcgcgctgactccaaaggcaactacgcttctaccggcgtttcccgtagcgaacacgacactggcgtttccccagtatttgctggcggcgtagagtgggctgttactcgtgacatcgctacccgtctggaataccagtgggttaacaacatcggcgacgcgggcactgtgggtacccgtcctgataacggcatgctgagcctgggcgtttcctaccgcttcggtcaggaagatgctgcaccggttgttgctccggctccggctccggctccggaagtggctaccaagcacttcaccctgaagtctgacgttctgttcaacttcaacaaagctaccctgaaaccggaaggtcagcaggctctggatcagctgtacactcagctgagcaacatggatccgaaagacggttccgctgttgttctgggctacaccgaccgcatcggttccgaagcttacaaccagcagctgtctgagaaacgtgctcagtccgttgttgactacctggttgctaaaggcatcccggctggcaaaatctccgctcgcggcatgggtgaatccaacccggttactggcaacacctgcgacaacgtgaaagctcgcgctgccctgatcgattgcctggctccggatcgtcgtgtagagatcgaagttaaaggctacaaagaagttgtaactcagccggcggcttaa

SEQ ID NO: 101 - RNA, 738 nt, Klebsiella pneumoniae
  source 1..738: mol_type = mRNA
gtggatggatggcagcgcgcctttgtcctgcatagccgtccgtggagcgaaaccagcctgatgctggacgtcttcacggaagaatccggtcgcgtgcgcctggttgccaagggcgcacgctccaaacgctctaatctcaaaggcgctttacagccctttacccccttgttggttcgcttcggcgggcgcggcgaagtcaaaaccctgcgcagcgcggaagccgtttcgctggcgctgccgctcagcggcatcaccctctacagcggtctctacgttaacgagcttatctcccgcgtgctggagcacgaaacacgcttctccgaactctttttcgactacctgcactgcattcaggcgctcgccggggccagcggctcgccggagcccgcgctgcggcgttttgagctggcgctgctcgggcacctgggctatggcgtggattttctccactgcgccgggagcggcgagccggtagacgacaccatgacctaccgctatcgtgaagaaaaaggctttattgccagcctggtgattgataacaacacgtttaccgggcaccatttgaaagcattggcgagtcgcgagtttcctgatgttgatacgctgcgcgccgcgaagcgctttacccgtatcgccctgaagccctatctcggcggcaaaccgctcaagagccgggagttgtttcgccagtttatgcccgcgcgaaaggcgcgagccgataatacgaataatgattaa

SEQ ID NO: 102 - RNA, 4950 nt, Klebsiella pneumoniae
  source 1..4950: mol_type = mRNA
atgaaaccatttcgtttagcggcgctatcactggcgctccttaccgcattttcattaaccgggtgtgatgacagcggtacaccgcaggcctcggccccggcgccagccgcggatagcaacccaggcgcgacggcaaaaccggaccgcgcccagctggcggcgctggcggaaaaaagccagggtaaagcgctgacgctcctcgacgcctcggaggtgcaactggatggcgccgcgaccctggtgctgacgttttccgtcccgctgcagccggaccaggatttctcccgcagcgtgcatctggtcgacaagaaaagcggtaaggtggacggtgcctgggagctggcgccgaatctgaaagagctgcgtctgcgtcaccttgagccaaagcgcgagctgattgtctcggtcgacccgacgttgaccgccctgaacaaggcgacgctcgacaaaccgtttgagaagaccctgaccacccgcgatatcgcaccgagcgttgggtttgccagccgcggctcgctgctgccgggcaatgtggtcgccgggctgccggtgatggcgcttaacgtcgataatgtcgacgttaatttcttccgcattaagccggaatcgctctccgcctttgtcagtcagtgggaataccgtaactcgttaagcaactgggaatctgatgaactgctgaagatggcggatctggtctacagcggccgcttcgatctcaacccggcgcgtaatactcgcgaaaagctgctgctgccgctgagcgacatcaagccgctccagcagcctggcgtctacgtggcggtcatgaacccggcgggccgttatagctacagcaacgcggcgacgctgtttaccctcagcgatatcggcgtctcggcgcatcgctaccataaccggctggacgtttttacccagagtctggaaaacggcgccgcccagtcgggtattgaggtgcagttgctcaatgccaaagggcagaccctggcggaagcgaaaagcgacagccagggccacgtcaccctgcaaaccgataaagaagccgcgctgctgctggcgcgcaaagaggggcagaccacgctcctcgacctcaagctgccggcgctggatctggcggaattctctatcgcaggcgcgcccggctttagcaaacagttctttatgtttggcccgcgcgatctctatcgtcccggcgaaacggtgatccttaatgcgctgttgcgcgatagcgacggcaagccgctggcggagcagccggtgaaactggaggtggttcagccggacggccaggtgatccgctcggtgatgagtaaaccagtgaatggcctgtaccagtttacttatccgctcgacagcggcgcggctaccggcatgtggcacattcgcgccagcgccggcgacaaccagccgcgcgaatgggatttccacgttgaagactttatgccggaacgcatggccctgaacctgacgccgcaggccgcgccagtcgcgccggatgccgacgtcacctttggcgttagcggcgcttatctctacggcgcgccggccagcggcaaccagctgcagggcaagctgttcctgcgcccgctgcgcgacgccgtcgctgcgctgccgggcttccagttcggcgatattgcggaagaaaacctctcccgtagtctggatgaagtccagctgacgctggacgaaaaaggccatggcgaagtgaccacctcaagccagtggcaggatagccattcgccgctgcaggtggtgctgcaggccagcctgctggaatccggcggtcgcccggtgacccgtacggttaagcagccgatctggcccgccgaggcgctgccggggatccgcccgcaatttacgctgaaggacgtttacgattaccgcaccgataccaccgtgaaacagccggtggtggatgaaaacagtaacgccgcgtttgatatcgtctatgccgacgtcaaaggcgagaaaaaagcgatctccggtctccaggtgcggctgatccgcgagaggcgcgactactactggaactggtccgacagcgaaggctggcagtcgcagttcgatcaaaaagatctgcaggagggcgaggagtcgctggacctgcaggccggccagatcggcaaagtgagcttcccggtggagtggggatcctatcgcctggaggtgaaaggaccggacgacgtggtcagcagcgtgcgcttctgggcgggctacagctggcaggataacagcgaaggcgccggcgcggcgcgtcctgaccgggtcaccatgaagctggataaaccgagctataaaccgggcgacaccatcaggctgcacatcgccgccccggcggcgggtaaaggctacgcaatggtggaatccagcgaagggccgctgtggtggcaggaaattgacgtcccggcggaggggatggacctgtcgatcccggtggataaaacctggaatcgccacgacctctacctcagcacgctggtggttcgtcccggcgataaatcgcgctcggcaacgccgaaacgcgcggtgggcctgctgcatctgccgctgggcgatgaaaaccgtcgcctgacgctggcgctggaggccccggacaaaatgcgccccaaccagccgctgacggtgaaagtgaaagccagcgtcaaagagggcgaggcgccgaaacaggtcaatgtcctgctctcggcggtggatagcggggtactgaatatcaccgattacgctaccccggatccgtggaacgccttctttggccagaagcgttacggcgcagatatctatgacatctacggccaggtgatcgaagggcagggacgtgtggccagcctgcgcttcggcggcgatggcgacgagttaaaacgcggcggcaaaccgccggttaaccacgtgacgatcgtcgcccagcaggcgcagccggtggtattgaacgatcagggcgaaggcacggtgacgttacccattggcgattttaacggcgagctgcgggtgatggcccaggcgtggacggcggatgacttcggcagcagcgaagataaagtggtggtggccgccccggtcatcgccgagctgaacactccgcgcttcctcgccagcggcgatactacgcgtctggcgctggatctcagcaatctgaccgataaaccgcaaaccctgcaggttcaccttaccgccagcgggctggtaacgttgagcgagggtcaactgccgccggtacagctggcgccgggggcgcgcagcacgctgtttataccagtgagcgcgctggcgggcttcggcgatggtcaggttaatgccaccatcagcggcctgagcctgccgggcgaaaccttcgccccgctgcaaaaacagtggaaaatcggcgtgcgtccggcgtacccggcacagaccgtcaacagcggcgcagtgctgcagccaggtgaaagctggcagccgccggcggcgcagagccagggctttgccccgcaaacgctgcagggccaactgctgctcagcggtaaaccaccgttgaatctcgcccgctatatccgcgagctgaaagcctatccttacggctgccttgagcagaccgccagcggcctgttcccgtccctgtataccagcgcggcgcagctaaaggcgctgggcattagcggggacagcgacgaaaaacgccgcgcggccatcgatgtcggtatttcacgcctgctgcagatgcagctggaaaacggcggctttgcgctatgggatagagaggggccggaagagtactggctgaccgcctacgccatggacttcctcgtgcgcgccagcgagcagggctatagcgtaccggtcaacgccattaataaaggcaacgagcgcctgctgcgctatctgcaggagccgggcctgatgaccgtgcgctacagcgacgacgcccaggctagccgctttgccgcccaggcatacgccgcgctggtgctggcgcgtcagcaaaaagcgccgctcggcgccttgcgtgaaatctggagccgtcacgaccaggcgcgatccggcttgccgctgctgcagctcggcattgcgctgaaaactatgggcgatgcgccgcgcggcgacgccgccctgaagctggcggttgccaccccgcgccaggatgagaatcgttggctgggcgattacggtagcccgctgcgcgataacgccctgaagctggcgctgctggaagagaataagctgctgccggaggtgcaaactcagctgttgagcacgctgtcggaagaggcctatggccagcgctggctgtcgacccaggaaaccaacgccttgttccttgccggacgtacgctggcggacctgccgggcagctggcaggcgcaaacctcgctgcaggcggagccgctggccggcgacaaagcgcaaacccgcaacctcgacggcgatcggctggcggcgctgcaggtgagtaataccggcagtcagccgctgtggctgcgtctcgacagcagcggctatccgcaaagcgcgccgcagccgggcggtaacgtcctggggattgaacgcactattttcgatacccaggggcagcagaaatcgctgtcttcactgcgcagcggcgaactggtgctcgtcaagctggaggtgaccgccaaacgcaacgtgcctgacgcgctggtggttgatctgttgccagccggtctggagctggaaaaccagaatctggccaacagcagcgccagcctgcaggagaatggcgacgcggtgcaaaacctgcttaaccagatgcagcaggcggatattcagcatatcgagttccgcgatgaccgtttcgtcgccgccgtcgctgtcaatgaaggccagccggtgacgctggtctatctggcgcgggcggtgacgccgggcacttatcaggtcccgcagccgcaggtggaatcgatgtatgcgccgcagtggcgggcgacaggagcggccagcggtccgctcaccgtgacgccgtaa

SEQ ID NO: 103 - DNA, 10 bp, synthetic construct
  misc_feature 1..10: note = Model DNA fragment
  source 1..10: mol_type = other DNA
attcggaacc

SEQ ID NO: 104 - DNA, 10 bp, synthetic construct
  misc_feature 1..10: note = Model DNA fragment
  source 1..10: mol_type = other DNA
atacgggacc

(19) United States
(12) Patent Application Publication
(10) Pub. No.: US 2025/0205325 A1
(43) Pub. Date: Jun. 26, 2025

(54) Proteins and nucleic acids useful in vaccines targeting Klebsiella pneumoniae

(71) Applicant: Evaxion Biotech A/S, Hørsholm (DK)

(72) Inventors: Niels Iversen Møller, Hørsholm (DK); Andreas Holm Mattsson, Hørsholm (DK)

(21) Appl. No.: 18/999,335

(22) Filed: Dec. 23, 2024

Related U.S. Application Data
(63) Continuation of application No. 17/088,683, filed on Nov. 4, 2020, now Pat. No. 12,226,469.
Application No. 17/088,683 is a continuation of application No. 16/594,987, filed on Oct. 7, 2019, now Pat. No. 10,849,968.
Application No. 16/594,987 is a continuation of application No. 15/542,580, filed on Jul. 10, 2017, now Pat. No. 10,434,162.

(30) Foreign Application Priority Data
Jan. 12, 2015 (EP) 15150819.9

(51) Int. Cl. A61K 39/108; A61K 38/03; A61K 38/16; C07K 14/26; C07K 16/12; C12N 15/70; C12N 15/75; C12N 15/79; G01N 33/569

(52) U.S. Cl. CPC A61K 39/0266; A61K 38/03; A61K 38/164; C07K 14/26; C07K 16/12; C12N 15/79; G01N 33/56916; C07K 2317/24; C07K 2317/54; C07K 2317/55; C07K 2317/622; C12N 15/70; C12N 15/75; G01N 2333/26; G01N 2469/20

(57) ABSTRACT

The present invention relates to proteins and nucleic acids derived from Klebsiella pneumoniae as well as therapeutic and diagnostic uses of the proteins and nucleic acids.

### CROSS REFERENCE TO RELATED APPLICATIONS

In accordance with 37 C.F.R. 1.76, a claim of priority is included in an Application Data Sheet filed concurrently herewith. Accordingly, the present invention claims priority as a continuation of U.S. patent application Ser. No. 17/088,683, entitled “PROTEINS AND NUCLEIC ACIDS USEFUL IN VACCINES TARGETING KLEBSIELLA PNEUMONIAE”, filed Nov. 4, 2020, which is a continuation of U.S. patent application Ser. No. 16/594,987, entitled “PROTEINS AND NUCLEIC ACIDS USEFUL IN VACCINES TARGETING KLEBSIELLA PNEUMONIAE”, filed Oct. 7, 2019, which is a continuation of U.S. patent application Ser. No. 15/542,580, entitled “PROTEINS AND NUCLEIC ACIDS USEFUL IN VACCINES TARGETING KLEBSIELLA PNEUMONIAE”, filed Jul. 10, 2017, now U.S. Pat. No. 10,434,162, issued Oct. 8, 2019, which is a § 371 national stage entry of International Application No. PCT/EP2016/050468, entitled “PROTEINS AND NUCLEIC ACIDS USEFUL IN VACCINES TARGETING KLEBSIELLA PNEUMONIAE”, filed Jan. 12, 2016, which claims the benefit of the priority of European Patent Application No. 15150819.9, entitled “PROTEINS AND NUCLEIC ACIDS USEFUL IN VACCINES TARGETING KLEBSIELLA PNEUMONIAE”, filed Jan. 12, 2015, the entire contents of each are incorporated herein by reference.

### REFERENCE TO AN ELECTRONIC SEQUENCE LISTING

The contents of the electronic sequence listing (6318U.000041.xml; Size: 215 KB and Date of Creation: Dec. 20, 2024) is herein incorporated by reference in its entirety.

### FIELD OF THE INVENTION

The present invention relates to the field of antimicrobial prophylaxis and therapy. In particular the present invention relates to novel proteins and polynucleotides derived from Klebsiella pneumoniae. The invention further relates to vectors comprising the polynucleotides, transformed host organisms expressing the polynucleotides, antibodies (mono- or polyclonal) specific for the polypeptides as well as diagnostic, prophylactic and therapeutic uses and methods. Finally, also methods of preparation are part of the invention.

### BACKGROUND OF THE INVENTION

Bacterial infections are in most instances successfully treated by administration of antibiotics to patients in need thereof. However, due to careless or thoughtless use of powerful antibiotics, many pathological germs become resistant against antibiotics over time. One threatening example is Klebsiella pneumoniae.

The genus Klebsiella belongs to the family Enterobacteriaceae and is divided into at least 4 species. They are gram-negative, capsulated, oxidase-negative, non-motile, straight rods. They are facultative anaerobes, having both a respiratory and fermentative metabolism. Most strains can use citrate and glucose as their sole carbon source. Some strains can fix nitrogen. They are commonly found in the intestines, clinical samples, soil, water and grains. The species Klebsiella pneumoniae can be divided into 3 subspecies; pneumoniae, ozaenae and rhinoscleromatis (Orskov, I. 1984. Genus V. Klebsiella Trevisan 1885, 105, Krieg and Holt (editors) in Bergey's Manual of Systematic Bacteriology, 1:461-465). Klebsiella pneumoniae is the most common gram-negative pathogen causing community acquired pneumonia (Carpenter, J., et al, 1990. Rev Infect, Dis. 12:672-682). Klebsiella is also responsible for an estimated 8% of all nosocomial infections (Sahly, H. and Podschun, R., 1997. Clin. Diagn. Lab. Immunol. 4:393-399).

K. pneumoniae is an opportunistic pathogen that is associated with pneumonia, septicemia, meningitis, endocarditis, ventriculitis, and infections of urinary tract and wounds. These diseases are both nosocomial and community acquired, K. pneumoniae also plays a large role in two major nonrheumatoid arthritic diseases, Ankylosing Spondylitis and Reiter's Syndrome (Schwimmbeck, P. and Oldstone, M., 1989. Current Topics in Microbiology and Immunology. 145:45-56.). Despite available antibiotics, observed mortality rates for pneumonia are approximately 50%, but when bacteremic K. pneumoniae occurs in alcoholics, the mortality rises to almost 100% (Sahly, H. and Podschun, R., 1997. Clin. Diagn. Lab. Immunol, 4:393-399). The overall mortality rate for Klebsiella bacteremia in one study was 37% and has ranged in others from 25% to 55% (Watanakunakron, C. and Jura, J., 1991. Scand. J. Infect. Dis. 23:399-405).

Incidence of K. pneumoniae meningitis is on the rise. A study of 3377 cases of Bacterial meningitis in 1948, found only 7 were K. pneumoniae. In 1957, K. pneumonia accounted for 1.5% of all cases of meningitis. In an eleven-year study, from 1981 to 1991, 13% of culture proven bacterial meningitis cases were K. pneumoniae. There was an increase occurrence of K. pneumoniae meningitis within this study with 7% occurrence in the first 6 years, and 16% occurrence in the last S years (Tang, L-M and Chen, S-T., 1994. Scand. J. Infect, Dis. 26:95-102).

In recent years, Klebsiella strains have become multi-resistant to many antibiotics. In the 1970's, the resistance was mainly to aminoglycoside antibiotics. Since 1982, some Klebsiella strains have become resistant to the extended-spectrum cephalosporins (Sahly, H. and Podschun, R., 1997. Clin. Diagn. Lab. Immunol. 4:393-399). Resistance to the extended-spectrum cephalosporins among clinical isolates of Klebsiella in France and England has been reported at 14 to 16% (Sirot, D. 1995 J. Antimicrob. Chemother. 36:19-34). Since Klebsiella is a good recipient for R factors, resistance has been gained to β-lactams, tetracycline, chloramphenicols, ceftazidime, sulfonamides and trimethoprim. Today, almost all strains of Klebsiella are resistant to ampicillin. (Orskov, I. 1984. Genus V. Klebsiella Trevisan 1885, 105, Krieg and Holt (editors) In Bergey's Manual of Systematic Bacteriology, 1:461-465).

Microbial fermentation is an Important way to convert renewable resources to products of biological and industrial importance. K. pneumoniae has been used to convert simple sugars to the commodity chemicals 1,3-propanediol and 1,2-propanediol. These products have been made by fed-batch fermentation of glycerol by K. pneumoniae. (Cameron, D. et al, 1998. Biotechnol. Prog. 14:116-125). Genes from the 1,3-propanediol pathway of K. pneumoniae have recently been cloned and expressed into both E. coli and S. cerevisiae. Metabolic engineering of these genes can significantly improve the product yield and productivity (Cameron, D. et al, 1998. Biotechnol, Prog. 14:116-125).

With K. pneumoniae playing the lead role, the Klebsiella genus is becoming an increasingly important pathogen. Over the past 10 years, discovery of multi-drug resistant strains has emphasized the importance of this genus. Furthermore, Klebsiella is considered to be a model for systemic infections caused by capsulated bacteria.

Vaccination is considered to be a very effective method of preventing infectious diseases in human and veterinary health care. Vaccination is the administration of immunogenically effective amounts of antigenic material (the vaccine) to produce immunity to a disease/disease-causing pathogenic agent. Vaccines have contributed to the eradication of smallpox, the near eradication of polio, and the control of a variety of diseases, including rubella, measles, mumps, chickenpox, typhoid fever.

Before “the genomic era”, vaccines were based on killed or live attenuated, microorganisms, or parts purified from them. Subunit vaccines are considered as a modern upgrade of these types of vaccine, as the subunit vaccines contain one or more protective antigens, which are more or less the weak spot of the pathogen. Hence, in order to develop subunit vaccines, it is critical to identify the proteins, which are important for inducing protection and to eliminate others.

An antigen is said to be protective if it is able to induce protection from subsequent challenge by a disease-causing infectious agent in an appropriate animal model following immunization.

The empirical approach to subunit vaccine development, which includes several steps, begins with pathogen cultivation, followed by purification into components, and then testing of antigens for protection, Apart from being time and labour consuming, this approach has several limitations that can lead to failure. It is not possible to develop vaccines using this approach for microorganisms, which cannot easily be cultured and only allows for the identification of the antigens, which can be obtained in sufficient quantities. The empirical approach has a tendency to focus on the most abundant proteins, which in some cases are not immuno-protective. In other cases, the antigen expressed during in vivo infection is not expressed during in vitro cultivation. Furthermore, antigen discovery by use of the empirical approach demands an extreme amount of proteins in order to discover the protective antigens, which are like finding needles in the haystack. This renders it a very expensive approach, and it limits the vaccine development around diseases, which is caused by pathogens with a large genome or disease areas, which perform badly in a cost-effective perspective.

### OBJECT OF THE INVENTION

It is an object of embodiments of the invention to provide K. pneumoniae derived antigenic polypeptides that may serve as constituents in vaccines against K, pneumoniae infections and in diagnosis of K. pneumoniae infections. It is also an object to provide nucleic acids, vectors, transformed cells, vaccine compositions, and other useful means for molecular cloning as well as for therapy and diagnosis with relevance for K. pneumoniae.

### SUMMARY OF THE INVENTION

It has been found by the present inventor(s) that K. pneumoniae, in particular drug resistant K. pneumoniae, expresses a number of hitherto unknown putatively surface exposed proteins which are candidates as vaccine targets as well as candidates as immunizing agents for preparation of antibodies that target K. pneumoniae.

So, in a first aspect the present invention relates to a polypeptide comprising
a) an amino acid sequence selected from the group consisting of any one of SEQ ID NOS: 1-30, 93, and 94, or
b) an amino acid sequence consisting of at least or exactly or at most 5 contiguous amino acid residues from any one of SEQ ID NOs: 1-30, 93, and 94, or
c) an amino acid sequence having a sequence identity of at least 60% with the amino acid sequence of a),
d) an amino acid sequence having a sequence identity of at least 60% with the amino acid sequence of b), or
e) an assembly of amino acids derived from any one of SEQ ID NOs: 1-30, 93, and 94, which has essentially the same 3D conformation as in the protein from which said assembly is derived so as to constitute a 8-cell epitope, said polypeptide being antigenic in a mammal.

In a second aspect, the invention relates to an isolated nucleic acid fragment, which comprises
i) a nucleotide sequence encoding a polypeptide of the invention, or
ii) a nucleotide sequence consisting of any one of SEQ ID NOS: 31-90 and 95-102.
iii) a nucleotide sequence consisting of at least or exactly or at most 10 consecutive nucleotides in any one of SEQ ID NOS: 31-90 and 95-102,
iv) a nucleotide sequence having a sequence identity of at least 60% with the nucleotide sequence in i) or ii),
v) a nucleotide sequence having a sequence identity of at least 60% with the nucleotide sequence in iii),
vi) a nucleotide sequence complementary to the nucleotide sequence in i)-v), or
vii) a nucleotide sequence which hybridizes under stringent conditions with the nucleotide sequence in i)-vi).

In a third aspect, the invention relates to a vector comprising the nucleic acid of the invention, such as a cloning vector or an expression vector.

In fourth aspect, the invention relates to a cell which is transformed so as to carry the vector of the invention.

In a fifth aspect, the invention relates to a pharmaceutical composition comprising a polypeptide of the invention, a nucleic acid fragment of the invention, a vector of the invention, or a transformed cell of the invention, and a pharmaceutically acceptable carrier, vehicle or diluent.

In a sixth aspect, the invention relates to a method for inducing immunity in an animal by administering at least once an immunogenically effective amount of a polypeptide of the invention, a nucleic acid fragment of the invention, a vector of the invention, a transformed cell of the invention, or a pharmaceutical composition of the fifth aspect of the invention so as to induce adaptive immunity against K. pneumoniae in the animal.

In a seventh and eighth aspect, the invention relates to 1) a polyclonal antibody in which the antibodies specifically bind to at least one polypeptide of the invention, and which is essentially free from antibodies binding specifically to other K. pneumoniae polypeptides, and to 2) an isolated monoclonal antibody or antibody analogue which binds specifically to a polypeptide of the invention. In a related ninth aspect, the invention relates to a pharmaceutical composition comprising such a polyclonal or monoclonal antibody and a pharmaceutically acceptable carrier, vehicle or diluent.

In a 10^{th} aspect, the invention relates to a method for prophylaxis, treatment or amelioration of infection with K. pneumoniae, comprising administering a therapeutically effective amount of an antibody of the 7^{th} or 8^{th} aspect of the invention or a pharmaceutical composition of the eighth aspect to an individual in need thereof.

In an 11^{th} aspect, the invention relates to a method for determining, quantitatively or qualitatively, the presence of K. pneumoniae, in particular the presence of K. pneumoniae, In a sample, the method comprising contacting the sample with an antibody of aspects 8 or 9 of the invention and detecting the presence of antibody bound to material in the sample.

In an 12^{th} aspect of the invention is provided a method for determining, quantitatively or qualitatively, the presence of antibodies specific for K. pneumoniae, In particular the presence of antibodies specific for K. pneumoniae, in a sample, the method comprising contacting the sample with a polypeptide of the invention and detecting the presence of antibody that specifically bind said polypeptide.

In a 13^{th} aspect, the invention relates to a method for determining, quantitatively or qualitatively, the presence of a nucleic acid characteristic of K. pneumoniae, in particular the presence of a nucleic acid characteristic of K. pneumoniae, In a sample, the method comprising contacting the sample with a nucleic acid fragment of the invention and detecting the presence of nucleic acid in the sample that hybridizes to said nucleic acid fragment.

In a 14^{th} aspect, the invention relates to a method for the preparation of the polypeptide of the invention, comprising
culturing a transformed cell of the present invention, which is capable of expressing the nucleic acid of the invention, under conditions that facilitate that the transformed cell expresses the nucleic acid fragment of the invention, which encodes a polypeptide of the invention, and subsequently recovering said polypeptide, or
preparing said polypeptide by means of solid or liquid phase peptide synthesis.

In a 15^{th} aspect, the invention relates to a method for determining whether a substance, such as an antibody, is potentially useful for treating infection with K, pneumoniae, the method comprising contacting the polypeptide of the invention with the substance and subsequently establishing whether the substance has at least one of the following characteristics:
1) the ability to bind specifically to said polypeptide,
2) the ability to compeed with said polypeptide for specific binding to a ligand/receptor, and
3) the ability to specifically inactivate said polypeptide.

Finally, in a 16^{th} aspect, the invention relates to a method for determining whether a substance, such as a nucleic acid, is potentially useful for treating infection with K. pneumoniae, the method comprising contacting the substance with the nucleic acid fragment of claim of the invention and subsequently establishing whether the substance has either the ability to
1) bind specifically to the nucleic acid fragment, or
2) bind specifically to a nucleic acid that hybridizes specifically with the nucleic acid fragment.

### LEGENDS TO THE FIGURE

FIGS. 1-6 show Kaplan-Meier survival plots in groups of mice subjected to challenge infection with K. pneumoniae, i.e. survival plot corresponding to the data in tables 1-6 herein.

FIGS. 1 and 3-6 show the survival plots from experiments 1 and 3-6, respectively, where mice received the infectious challenge via the intranasal route, whereas FIG. 2 shows the survival plots for mice challenged via the intraperitoneal route.

### DETAILED DISCLOSURE OF THE INVENTION

### Definitions

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring amino acid sequence or nucleic acid sequence, respectively

The term “amino acid sequence” s the order in which amino acid residues, connected by peptide bonds, lie in the chain in peptides and proteins.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

“Sequence identity” is in the context of the present invention determined by comparing 2 optimally aligned sequences of equal length (e.g. DNA, RNA or amino acid) according to the following formula: (N_{ref}−N_{dif})·100/N_{ref}, wherein N_{ref} is the number of residues in one of the 2 sequences and N_{dif} is the number of residues which are non-identical in the two sequences when they are aligned over their entire lengths and in the same direction. So, two sequences 5′-ATTCGGAACC-3′ (SEQ ID No: 103) and 5′-ATACGGGACC-3′ (SEQ ID No: 104) will provide the sequence identity 80% (N_{ref}=10 and N_{dif}=2).

An “assembly of amino acids” means two or more amino acids bound together by physical or chemical means.

The “3D conformation” is the 3 dimensional structure of a biomolecule such as a protein. In monomeric polypeptides/proteins, the 3D conformation is also termed “the tertiary structure” and denotes the relative locations in 3 dimensional space of the amino acid residues forming the polypeptide.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immungon by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule prese

An “immunogen” is a substance of matter which is capable of inducing an adaptive immune response in a host, whose immune system is confronted with the immunogen. As such, Immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigene determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Hybridization under “stringent conditions” is herein defined as hybridization performed under conditions by which a probe will hybridize to its target sequence, to a detectably greater degree than to other sequences. Stringent conditions are target sequence-dependent and will differ depending on the structure of the polynucleotide. By controlling the stringency of the hybridization and/or washing conditions, target sequences can be identified which are 100% complementary to a probe (homologous probing). Alternatively, stringency conditions can be adjusted to allow some mismatching in sequences so that lower degrees of similarity are detected (heterologous probing). Specificity is typically the function of post-hybridization washes, the critical factors being the ionic strength and temperature of the final wash solution.

Generally, stringent wash temperature conditions are selected to be about 5° C. to about 2° C. lower than the melting point (Tm) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tm, which is also called the melting temperature, Formulas are available in the art for the determination of melting temperatures.

The term “animal” is in the present context in general intended to denote an animal species (preferably mammalian), such as Homo sapiens, Canis domesticus, etc, and not just one single animal. However, the term also denotes a population of such an animal species, since it is important that the individuals immunized according to the method of the invention substantially all will mount an immune response against the immunogen of the present invention.

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanised antibodies, altered antibodies, univalent antibodies, Fab proteins, and single domain antibodies.

“Specific binding” denotes binding between two substances which goes beyond binding of either substance to randomly chosen substances and also goes beyond simple association between substances that tend to aggregate because they share the same overall hydrophobicity or hydrophilicity. As such, specific binding usually involves a combination of electrostatic and other interactions between two conformationally complementary areas on the two substances, meaning that the substances can “recognize” each other in a complex mixture.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage-both these infectious agents are capable of introducing a heterologous nucleic acid sequence

The term “expression vector” refers to a vector containing a nucleic acid sequence coding for at least part of a gene product capable of being transcribed. In some cases, when the transcription product is an mRNA molecule, this is in turn translated into a protein, polypeptide, or peptide.

### SPECIFIC EMBODIMENTS OF THE INVENTION

### The Polypeptides of the Invention

In some embodiments the at least or exactly 5 contiguous amino acids referred to in option b) In the definition of the first aspect of the invention constitute at least or exactly 5, at least or exactly or at most 6, at least or exactly or at most 7, at least or exactly or at most 8, at least or exactly or at most 9, at least or exactly or at most 10, at least or exactly or at most 11, at least or exactly or at most 12, at least or exactly or at most 13, at least or exactly or at most 14, at least or exactly or at most 15, at least or exactly or at most 16, at least or exactly or at most 17, at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27 at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, and at least or exactly or at most 93 contiguous amino acid residues.

The number of contiguous amino acids can be higher, for all of SEQ ID NOS: 2-30. Another way to phrase this is that for each of SEQ ID NOS: 1-30, 93, and 94, the number of the contiguous amino acid residues is at least or exactly or at most N-n, where N is the length of the sequence ID in question and n is any integer between N—S and O; that is, the at least 5 contiguous amino acids can be at least any number between 5 and the length of the reference sequence minus one, in increments of one. Consequently:

Insofar as embodiment b relates to SEQ ID NO: 2-30, 93, and 94, the at least 5 contiguous amino acids referred to in option b) in the definition of the first aspect of the invention may also constitute at least or exactly or at most 94, at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, and at least or exactly or at most 116 contiguous amino acid residues.

Insofar as embodiment b relates to SEQ ID NO: 3-30, 93, and 94, the at least 5 contiguous amino acids referred to in option b) in the definition of the first aspect of the invention may also constitute at least or exactly or at most 117, or at least or exactly or at most 118 contiguous amino acid residues.

Insofar as embodiment b relates to SEQ ID NO: 4-30, 93, and 94, the at least 5 contiguous amino acids referred to in option b) In the definition of the first aspect of the invention may also constitute at least or exactly or at most 119 contiguous amino acid residues.

Insofar as embodiment b relates to SEQ ID NO: 5-30, 93, and 94, the at least 5 contiguous amino acids referred to in option b) in the definition of the first aspect of the invention may also constitute at least or exactly or at most 120 contiguous amino acid residues.

Insofar as embodiment b relates to SEQ ID NO: 6-30, 93, and 94, the at least 5 contiguous amino acids referred to in option b) in the definition of the first aspect of the invention may also constitute at least or exactly or at most 121 contiguous amino acid residues.

Insofar as embodiment b relates to SEQ ID NO: 7-30, 93, and 94, the at least 5 contiguous amino acids referred to in option b) in the definition of the first aspect of the invention may also constitute at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139, or at least or exactly or at most 140 contiguous amino acid residues.

Insofar as embodiment b relates to SEQ ID NO: 8-30, 93, and 94, the at least 5 contiguous amino acids referred to in option b) in the definition of the first aspect of the invention may also constitute at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, or at least or exactly or at most 150 contiguous amino acid residues.

Insofar as embodiment b relates to SEQ ID NO: 9-30, 93, and 94, the at least 5 contiguous amino acids referred to in option b) in the definition of the first aspect of the invention may also constitute at least or exactly or at most 151, at least or exactly or at most 152, at least or exactly or at most 153, at least or exactly or at most 154, at least or exactly or at most 155, at least or exactly or at most 156, at least or exactly or at most 157, at least or exactly or at most 158, at least or exactly or at most 159, at least or exactly or at most 160, at least or exactly or at most 161, at least or exactly or at most 162, at least or exactly or at most 163, at least or exactly or at most 164, at least or exactly or at most 165, at least or exactly or at most 166, or at least or exactly or at most 167 contiguous amino acid residues.

Insofar as embodiment b relates to SEQ ID NO: 10-30, 93, and 94, the at least 5 contiguous amino acids referred to in option b) in the definition of the first aspect of the invention may also constitute at least or exactly or at most 168, at least or exactly or at most 169, at least or exactly or at most 170, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, or at least or exactly or at most 192 at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, or at least or exactly or at most 208 at least or exactly or at most 209, at least or exactly or at most 210, at least or exactly or at most 211, at least or exactly or at most 212, at least or exactly or at most 213, at least or exactly or at most 214, at least or exactly or at most 215, at least or exactly or at most 216, at least or exactly or at most 217, at least or exactly or at most 218, at least or exactly or at most 219, at least or exactly or at most 220, at least or exactly or at most 221, at least or exactly or at most 222, at least or exactly or at most 223, at least or exactly or at most 224, at least or exactly or at most 225, at least or exactly or at most 226, at least or exactly or at most 227, or at least or exactly or at most 228 contiguous amino acid residues.

Insofar as embodiment b relates to SEQ ID NO: 11-30, 93, and 94, the at least 5 contiguous amino acids referred to in option b) in the definition of the first aspect of the invention may also constitute at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, or at least or exactly or at most 241, at least or exactly or at most 242, at least or exactly or at most 243, at least or exactly or at most 244, or at least or exactly or at most 245 contiguous amino acid residues.

Insofar as embodiment b relates to SEQ ID NO: 11-30 and 94, the at least 5 contiguous amino acids referred to in option b) in the definition of the first aspect of the invention may also constitute at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 261, at least or exactly or at most 262, at least or exactly or at most 263, at least or exactly or at most 264, at least or exactly or at most 265, at least or exactly or at most 266, at least or exactly or at most 267, at least or exactly or at most 268, at least or exactly or at most 269, at least or exactly or at most 270, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, at least or exactly or at most 285, at least or exactly or at most 286, at least or exactly or at most 287, at least or exactly or at most 288, at least or exactly or at most 289, at least or exactly or at most 290, at least or exactly or at most 291, at least or exactly or at most 292, at least or exactly or at most 293, at least or exactly or at most 294, at least or exactly or at most 295, at least or exactly or at most 296, at least or exactly or at most 297, at least or exactly or at most 298, at least or exactly or at most 299, at least or exactly or at most 300, at least or exactly or at most 301, at least or exactly or at most 302, at least or exactly or at most 303, at least or exactly or at most 304, at least or exactly or at most 305, at least or exactly or at most 306, at least or exactly or at most 307, at least or exactly or at most 308, at least or exactly or at most 309, at least or exactly or at most 310, at least or exactly or at most 311, at least or exactly or at most 312, at least or exactly or at most 313, at least or exactly or at most 314, at least or exactly or at most 315, at least or exactly or at most 316, at least or exactly or at most 317, at least or exactly or at most 318, at least or exactly or at most 319, at least or exactly or at most 320, or at least or exactly or at most 321 contiguous amino acid residues.

Insofar as embodiment b relates to SEQ ID NO: 12-30 and 94, the at least S contiguous amino acids referred to in option b) in the definition of the first aspect of the invention may also constitute at least or exactly or at most 322, at least or exactly or at most 323, at least or exactly or at most 324, at least or exactly or at most 325, or at least or exactly or at most 326, at least or exactly or at most 327, at least or exactly or at most 328, at least or exactly or at most 329, at least or exactly or at most 330, at least or exactly or at most 331, at least of exactly or at most 332, at least or exactly or at most 333, at least or exactly or at most 334, at least or exactly or at most 335, at least or exactly or at most 336, at least or exactly or at most 337, at least or exactly or at most 338, at least or exactly or at most 339, at least or exactly or at most 340, at least or exactly or at most 341, at least or exactly or at most 342, at least or exactly or at most 343, at least or exactly or at most 344, at least or exactly or at most 345, at least or exactly or at most 346, at least or exactly or at most 347, at least or exactly or at most 348, at least or exactly or at most 349, at least or exactly or at most 350, at least or exactly or at most 351, at least or exactly or at most 352, at least or exactly or at most 353, at least or exactly or at most 354, at least or exactly or at most 355, at least or exactly or at most 356 at least or exactly or at most 357, at least or exactly or at most 358, at least or exactly or at most 359, at least or exactly or at most 360, at least or exactly or at most 361, at least or exactly or at most 362, at least or exactly or at most 363, at least or exactly or at most 364, at least or exactly or at most 365, at least or exactly or at most 366, at least or exactly or at most 367, at least or exactly or at most 368, at least or exactly or at most 369, at least or exactly or at most 370, at least or exactly or at most 371, at least or exactly or at most 372, at least or exactly or at most 373, at least or exactly or at most 374, or at least or exactly or at most 375 contiguous amino acid residues.

Insofar as embodiment b relates to SEQ ID NO: 13-30 and 94, the at least 5 contiguous amino acids referred to in option b) in the definition of the first aspect of the invention may also constitute at least or exactly or at most 376, at least or exactly or at most 377, at least or exactly or at most 378, at least or exactly or at most 379, at least or exactly or at most 380, or at least or exactly or at most 381 contiguous amino acid residues.

Insofar as embodiment b relates to SEQ ID NO: 14-30 and 94, the at least 5 contiguous amino acids referred to in option b) in the definition of the first aspect of the invention may also constitute at least or exactly or at most 382, at least or exactly or at most 383, at least or exactly or at most 384, at least or exactly or at most 385, at least or exactly or at most 386, at least or exactly or at most 387, at least or exactly or at most 388, at least or exactly or at most 389, at least or exactly or at most 390, at least or exactly or at most 391, at least or exactly or at most 392, at least or exactly or at most 393, at least or exactly or at most 394, at least or exactly or at most 395, at least or exactly or at most 396, at least or exactly or at most 397, at least or exactly or at most 398, at least or exactly or at most 399, at least or exactly or at most 400, at least or exactly or at most 401, at least or exactly or at most 402, at least or exactly or at most 403, at least or exactly or at most 404, at least or exactly or at most 405, at least or exactly or at most 406, at least or exactly or at most 407, at least or exactly or at most 408, at least or exactly or at most 409, at least or exactly or at most 410, at least or exactly or at most 411, at least or exactly or at most 412, at least or exactly or at most 413, at least or exactly or at most 414, at least or exactly or at most 415, at least or exactly or at most 416, at least or exactly or at most 417, at least or exactly or at most 418, at least or exactly or at most 419, at least or exactly or at most 420, at least or exactly or at most 421, at least or exactly or at most 422, at least or exactly or at most 423, at least or exactly or at most 424, at least or exactly or at most 425, at least or exactly or at most 426, or at least or exactly or at most 427 contiguous amino acid residues.

Insofar as embodiment b relates to SEQ ID NO: 15-30 and 94, the at least 5 contiguous amino acids referred to in option b) In the definition of the first aspect of the invention may also constitute at least or exactly or at most 428, at least or exactly or at most 429, at least or exactly or at most 430, at least or exactly or at most 431, at least or exactly or at most 432, at least or exactly or at most 433, at least or exactly or at most 434, at least or exactly or at most 435, at least or exactly or at most 436, at least or exactly or at most 437, at least or exactly or at most 438, at least or exactly or at most 439, or at least or exactly or at most 440 contiguous amino acid residues.

Insofar as embodiment b relates to SEQ ID NO: 16-30 and 94, the at least 5 contiguous amino acids referred to in option b) in the definition of the first aspect of the invention may also constitute at least or exactly or at most 441, at least or exactly or at most 442, at least or exactly or at most 443, at least or exactly or at most 444, at least or exactly or at most 445, at least or exactly or at most 446, at least or exactly or at most 447, at least or exactly or at most 448, at least or exactly or at most 449, at least or exactly or at most 450, at least or exactly or at most 451, at least or exactly or at most 452, at least or exactly or at most 453, at least or exactly or at most 454, at least or exactly or at most 455, at least or exactly or at most 456, at least or exactly or at most 457, at least or exactly or at most 458, at least or exactly or at most 459, at least or exactly or at most 460, at least or exactly or at most 461, at least or exactly or at most 462, at least or exactly or at most 463, at least or exactly or at most 464, at least or exactly or at most 465, at least or exactly or at most 466, at least or exactly or at most 467, at least or exactly or at most 468, at least or exactly or at most 469, at least or exactly or at most 470, at least or exactly or at most 471, at least or exactly or at most 472, at least or exactly or at most 473, at least or exactly or at most 474, at least or exactly or at most 475, at least or exactly or at most 476, at least or exactly or at most 477, at least or exactly or at most 478, at least or exactly or at most 479, at least or exactly or at most 480, at least or exactly or at most 481, at least or exactly or at most 482, or at least or exactly or at most 483 contiguous amino acid residues.

Insofar as embodiment b relates to SEQ ID NO: 17-30 and 94, the at least 5 contiguous amino acids referred to in option b) in the definition of the first aspect of the invention may also constitute at least or exactly or at most 484, at least or exactly or at most 485, at least or exactly or at most 486, at least or exactly or at most 487, at least or exactly or at most 488, at least or exactly or at most 489, at least or exactly or at most 490, at least or exactly or at most 491, at least or exactly or at most 492, at least or exactly or at most 493, or at least or exactly or at most 494 at least or exactly or at most 495, at least or exactly or at most 496, at least or exactly or at most 497, at least or exactly or at most 498, at least or exactly or at most 499, at least or exactly or at most 500, at least or exactly or at most 501, at least or exactly or at most 502, at least or exactly or at most 503, at least or exactly or at most 504, at least or exactly or at most 505, at least or exactly or at most 506, at least or exactly or at most 507, at least or exactly or at most 508 at least or exactly or at most 509, at least or exactly or at most 510, at least or exactly or at most 511, at least or exactly or at most 512, at least or exactly or at most 513, at least or exactly or at most 514, at least or exactly or at most 515, at least or exactly or at most 516, at least or exactly or at most 517, at least or exactly or at most 518, at least or exactly or at most 519, at least or exactly or at most 520, at least or exactly or at most 521, at least or exactly or at most 522, at least or exactly or at most 523, at least or exactly or at most 524, at least or exactly or at most 525, at least or exactly or at most 526, at least or exactly or at most 527, at least or exactly or at most 528, at least or exactly or at most 529, at least or exactly or at most 530, at least or exactly or at most 531, at least or exactly or at most 532, at least or exactly or at most 533, or at least or exactly or at most 534 contiguous amino acid residues.

Insofar as embodiment b relates to SEQ ID NO: 18-30 and 94, the at least 5 contiguous amino acids referred to in option b) in the definition of the first aspect of the invention may also constitute at least or exactly or at most 535, at least or exactly or at most 536, at least or exactly or at most 537, at least or exactly or at most 538, at least or exactly or at most 539, at least or exactly or at most 540, at least or exactly or at most 541, at least or exactly or at most 542, at least or exactly or at most 543, at least or exactly or at most 544, at least or exactly or at most 545, at least or exactly or at most 546, at least or exactly or at most 547, at least or exactly or at most 548, at least or exactly or at most 549, at least or exactly or at most 550, at least or exactly or at most 551, at least or exactly or at most 552, at least or exactly or at most 553, at least or exactly or at most 554, at least or exactly or at most 555, at least or exactly or at most 556, at least or exactly or at most 557, at least or exactly or at most 558, at least or exactly or at most 559, at least or exactly or at most 560, at least or exactly or at most 561, at least or exactly or at most 562, or at least or exactly or at most 563, 427 at least or exactly or at most 564, at least or exactly or at most 565, at least or exactly or at most 566, at least or exactly or at most 567, at least or exactly or at most 568, at least or exactly or at most 569, at least or exactly or at most 570, at least or exactly or at most 571, at least or exactly or at most 572, at least or exactly or at most 57.3, at least or exactly or at most 574, at least or exactly or at most 575, at least or exactly or at most 576, at least or exactly or at most 577, at least or exactly or at most 578, at least or exactly or at most 579, at least or exactly or at most 580, at least or exactly or at most 581, at least or exactly or at most 582, at least or exactly or at most 583, at least or exactly or at most 584, at least or exactly or at most 585, at least or exactly or at most 586, at least or exactly or at most 587, at least or exactly or at most 588, at least or exactly or at most 589, at least or exactly or at most 590, at least or exactly or at most 591, at least or exactly or at most 592, at least or exactly or at most 593, at least or exactly or at most 594, at least or exactly or at most 595, or at least or exactly or at most 596 contiguous amino acid residues.

Insofar as embodiment b relates to SEQ ID NO: 19-30 and 94, the at least 5 contiguous amino acids referred to in option b) in the definition of the first aspect of the invention may also constitute at least or exactly or at most 597, at least or exactly or at most 598, at least or exactly or at most 599, at least or exactly or at most 600, at least or exactly or at most 601, at least or exactly or at most 602, at least or exactly or at most 603, at least or exactly or at most 604, at least or exactly or at most 605, at least or exactly or at most 606, at least or exactly or at most 607, at least or exactly or at most 608, at least or exactly or at most 609, at least or exactly or at most 610, at least or exactly or at most 611, at least or exactly or at most 612, at least or exactly or at most 613, at least or exactly or at most 614, at least or exactly or at most 615, at least or exactly or at most 616, at least or exactly or at most 617, at least or exactly or at most 618, at least or exactly or at most 619, at least or exactly or at most 620, at least or exactly or at most 621, at least or exactly or at most 622, or at least or exactly or at most 623 contiguous amino acid residues.

Insofar as embodiment b relates to SEQ ID NO: 20-30 and 94, the at least 5 contiguous amino acids referred to in option b) in the definition of the first aspect of the invention may also constitute at least or exactly or at most 624, at least or exactly or at most 625, at least or exactly or at most 626, at least or exactly or at most 627, at least or exactly or at most 628, at least or exactly or at most 629, at least or exactly or at most 630, at least or exactly or at most 631, at least or exactly or at most 632, at least or exactly or at most 633, at least or exactly or at most 634, at least or exactly or at most 635, at least or exactly or at most 636, at least or exactly or at most 637, at least or exactly or at most 638, at least or exactly or at most 639, at least or exactly or at most 640, at least or exactly or at most 641, at least or exactly or at most 642, at least or exactly or at most 643, at least or exactly or at most 644, at least or exactly or at most 645, at least or exactly or at most 646, at least or exactly or at most 647, at least or exactly or at most 648, at least or exactly or at most 649, at least or exactly or at most 650, at least or exactly or at most 651, at least or exactly or at most 652, at least or exactly or at most 653, at least or exactly or at most 654, at least or exactly or at most 655, or at least or exactly or at most 656 contiguous amino acid residues.

Insofar as embodiment b relates to SEQ ID NO: 21-30 and 94, the at least 5 contiguous amino acids referred to in option b) in the definition of the first aspect of the invention may also constitute at least or exactly or at most 657, at least or exactly or at most 658, at least or exactly or at most 659, at least or exactly or at most 660, at least or exactly or at most 661, at least or exactly or at most 662, at least or exactly or at most 663, at least or exactly or at most 664, at least or exactly or at most 665, at least or exactly or at most 666, at least or exactly or at most 667, at least or exactly or at most 668, at least or exactly or at most 669, at least or exactly or at most 670, at least or exactly or at most 671, at least or exactly or at most 672, at least or exactly or at most 673, or at least or exactly or at most 674 contiguous amino acid residues.

Insofar as embodiment b relates to SEQ ID NO: 22-30 and 94, the at least 5 contiguous amino acids referred to in option b) in the definition of the first aspect of the invention may also constitute at least or exactly or at most 675, at least or exactly or at most 676, at least or exactly or at most 677, at least or exactly or at most 678, at least or exactly or at most 679, at least or exactly or at most 680, at least or exactly or at most 681, at least or exactly or at most 682, at least or exactly or at most 683, at least or exactly or at most 684, at least or exactly or at most 685, at least or exactly or at most 686, at least or exactly or at most 687, at least or exactly or at most 688, at least or exactly or at most 689, at least or exactly or at most 690, at least or exactly or at most 691, at least or exactly or at most 692, at least or exactly or at most 693, at least or exactly or at most 694, at least or exactly or at most 695, at least or exactly or at most 696, at least or exactly or at most 697, at least or exactly or at most 698, at least or exactly or at most 699, or at least or exactly or at most 700 contiguous amino acid residues.

Insofar as embodiment b relates to SEQ ID NO: 23-30 and 94, the at least S contiguous amino acids referred to in option b) in the definition of the first aspect of the invention may also constitute at least or exactly or at most 701, at least or exactly or at most 702, at least or exactly or at most 703, at least or exactly or at most 704, at least or exactly or at most 705, at least or exactly or at most 706, at least or exactly or at most 707, at least or exactly or at most 708, at least or exactly or at most 709, at least or exactly or at most 710, at least or exactly or at most 711, at least or exactly or at most 712, at least or exactly or at most 713, at least or exactly or at most 714, at least or exactly or at most 715, at least or exactly or at most 716, at least or exactly or at most 717, at least or exactly or at most 718, at least or exactly or at most 719, at least or exactly or at most 720, at least or exactly or at most 721, at least or exactly or at most 722, at least or exactly or at most 723, at least or exactly or at most 724, at least or exactly or at most 725, at least or exactly or at most 726, at least or exactly or at most 727, at least or exactly or at most 728, at least or exactly or at most 729, at least or exactly or at most 730, at least or exactly or at most 731, at least or exactly or at most 732, at least or exactly or at most 733, at least or exactly or at most 734, at least or exactly or at most 735, at least or exactly or at most 736, at least or exactly or at most 737, at least or exactly or at most 738, at least or exactly or at most 739, at least or exactly or at most 740, at least or exactly or at most 741, at least or exactly or at most 742, at least or exactly or at most 743, at least or exactly or at most 744, at least or exactly or at most 745, at least or exactly or at most 746, at least or exactly or at most 747, at least or exactly or at most 748, at least or exactly or at most 749, at least or exactly or at most: 750, or at least or exactly or at most 751 contiguous amino acid residues.

Insofar as embodiment b relates to SEQ ID NO: 24-30 and 94, the at least 5 contiguous amino acids referred to in option b) In the definition of the first aspect of the invention may also constitute at least or exactly or at most 752, at least or exactly or at most 753, at least or exactly or at most 754, at least or exactly or at most 755, at least or exactly or at most 756, at least or exactly or at most 757, at least or exactly or at most 758, at least or exactly or at most 759, or at least or exactly or at most 760 contiguous amino acid residues.

Insofar as embodiment b relates to SEQ ID NO: 25-30 and 94, the at least 5 contiguous amino acids referred to in option b) in the definition of the first aspect of the invention may also constitute at least or exactly or at most 761, at least or exactly or at most 762, at least or exactly or at most 763, at least or exactly or at most 764, at least or exactly or at most 765, at least or exactly or at most 766, at least or exactly or at most 767, at least or exactly or at most 768, at least or exactly or at most 769, at least or exactly or at most 770, at least or exactly or 10 at most 771, at least or exactly or at most 772, at least or exactly or at most 773, at least or exactly or at most 774, at least or exactly or at most 775, at least or exactly or at most 776, at least or exactly or at most 777, at least or exactly or at most 778, at least or exactly or at most 779, at least or exactly or at most 780, at least or exactly or at most 781, at least or exactly or at most 782, at least or exactly or at most 783, at least or exactly or at most 784, at least or exactly or at most 785, at least or exactly or at most 786, at least or exactly or at most 787, at least or exactly or at most 788, or at least or exactly or at most 789 contiguous amino acid residues.

Insofar as embodiment b relates to SEQ ID NO: 26-30 and 94, the at least 5 contiguous amino acids referred to in option b) in the definition of the first aspect of the invention may also constitute at least or exactly or at most 790, at least or exactly or at most 791, at least or exactly or at most 792, at least or exactly or at most 793, at least or exactly or at most 794, at least or exactly or at most 795, at least or exactly or at most 796, at least or exactly or at most 797, at least or exactly or at most 798, at least or exactly or at most 799, at least or exactly or at most 800, at least or exactly or at most 801, at least or exactly or at most 802, at least or exactly or at most 803, at least or exactly or at most 804, at least or exactly or at most 805, at least or exactly or at most 806, at least or exactly or at most 807, or at least or exactly or at most 808 contiguous amino acid residues.

Insofar as embodiment b relates to SEQ ID NO: 27-30 and 94, the at least 5 contiguous amino acids referred to in option b) in the definition of the first aspect of the invention may also constitute at least or exactly or at most 809, at least or exactly or at most 810, at least or exactly or at most 811, at least or exactly or at most 812, at least or exactly or at most 813, at least or exactly or at most 815, at least or exactly or at most 816, at least or exactly or at most 817, at least or exactly or at most 8.18, at least or exactly or at most 819, at least or exactly or at most 820, at least or exactly or at most 821, at least or exactly or at most 811, at least or exactly or at most 823, at least or exactly or at most 824, at least or exactly or at most 825, at least or exactly or at most 826, at least or exactly or at most 827, at least or exactly or at most 828, at least or exactly or at most 829, at least or exactly or at most 830, at least or exactly or at most 831, at least or exactly or at most 832, at least or exactly or at most 833, at least or exactly or at most 834, at least or exactly or at most 835, at least or exactly or at most 836, at least or exactly or at most 837, at least or exactly or at most 838, at least or exactly or at most 839, at least or exactly or at most 840, at least or exactly or at most 841, at least or exactly or at most 842, at least or exactly or at most 843, at least or exactly or at most 844, at least or exactly or at most 845, at least or exactly or at most 846, at least or exactly or at most 847, at least or exactly or at most 848, at least or exactly or at most 849, at least or exactly or at most 850, at least or exactly or at most 851, at least or exactly or at most 852, at least or exactly or at most 853, at least or exactly or at most 854, at least or exactly or at most 855, at least or exactly or at most 856, at least or exactly or at most 857, at least or exactly or at most 858, at least or exactly or at most 859, at least or exactly or at most 860, at least or exactly or at most 861, at least or exactly or at most 862, at least or exactly or at most 863, at least or exactly or at most 864, at least or exactly or at most 865, at least or exactly or at most 866, at least or exactly or at most 867, at least or exactly or at most 868, at least or exactly or at most 869, at least or exactly or at most 870, at least or exactly or at most 871, at least or exactly or at most 872, at least or exactly or at most 873, at least or exactly or at most 874, at least or exactly or at most 875, at least or exactly or at most 876, at least or exactly or at most 877, at least or exactly or at most 878, at least or exactly or at most 879, at least or exactly or at most 880, at least or exactly or at most 881, at least or exactly or at most 882, at least or exactly or at most 883, at least or exactly or at most 884, at least or exactly or at most 885, at least or exactly or at most 886, at least or exactly or at most 887, at least or exactly or at most 888, at least or exactly or at most 889, at least or exactly or at most 890, at least or exactly or at most 891, at least or exactly or at most 892, at least or exactly or at most 893, at least or exactly or at most 894, or at least or exactly or at most 895 contiguous amino acid residues.

Insofar as embodiment b relates to SEQ ID NO: 28-30 and 94, the at least 5 contiguous amino acids referred to in option b) in the definition of the first aspect of the invention may also constitute at least or exactly or at most 896, at least or exactly or at most 897, at least or exactly or at most 898, at least or exactly or at most 899, at least or exactly or at most 900, at least or exactly or at most 901, at least or exactly or at most 902, at least or exactly or at most 903, at least or exactly or at most 904, at least or exactly or at most 905, at least or exactly or at most 906, at least or exactly or at most 907, at least or exactly or at most 908, at least or exactly or at most 909, at least or exactly or at most 910, at least or exactly or at most 911, at least or exactly or at most 912, at least or exactly or at most 913, at least or exactly or at most 914, at least or exactly or at most 915, at least or exactly or at most 916, at least or exactly or at most 917, at least or exactly or at most 918, at least or exactly or at most 919, at least or exactly or at most 920, at least or exactly or at most 921, at least or exactly or at most 922, at least or exactly or at most 923, at least or exactly or at most 924, at least or exactly or at most 925, at least or exactly or at most 926, at least or exactly or at most 927, at least or exactly or at most 928, at least or exactly or at most 929, at least or exactly or at most 930, at least or exactly or at most 931, at least or exactly or at most 932, at least or exactly or at most 933, at least or exactly or at most 934, at least or exactly or at most 935, at least or exactly or at most 936, at least or exactly or at most 937, at least or exactly or at most 938, at least or exactly or at most 939, at least or exactly or at most 940, at least or exactly or at most 941, at least or exactly or at most 942, at least or exactly or at most 943, at least or exactly or at most 944, at least or exactly or at most 945, at least or exactly or at most 946, at least or exactly or at most 947, at least or exactly or at most 948, at least or exactly or at most 949, at least or exactly or at most 950, at least or exactly or at most 951, at least or exactly or at most 952, at least or exactly or at most 953, at least or exactly or at most 954, at least or exactly or at most 955, at least or exactly or at most 956, at least or exactly or at most 957, at least or exactly or at most 958, at least or exactly or at most 959, at least or exactly or at most 960, at least or exactly or at most 961, at least or exactly or at most 962, at least or exactly or at most 963, at least or exactly or at most 964, at least or exactly or at most 965, at least or exactly or at most 966, at least or exactly or at most 967, at least or exactly or at most 968, at least or exactly or at most 969, at least or exactly or at most 970, at least or exactly or at most 971, at least or exactly or at most 972, at least or exactly or at most 973, at least or exactly or at most 974, at least or exactly or at most 975, at least or exactly or at most 976, at least or exactly or at most 977, at least or exactly or at most 978, at least or exactly or at most 979, at least or exactly or at most 980, at least or exactly or at most 981, at least or exactly or at most 982, at least or exactly or at most 983, at least or exactly or at most 984, at least or exactly or at most 985, at least or exactly or at most 986, at least or exactly or at most 987, at least or exactly or at most 988, at least or exactly or at most 989, at least or exactly or at most 990, at least or exactly or at most 991, at least or exactly or at most 992, at least or exactly or at most 993, at least or exactly or at most 994, at least or exactly or at most 995, at least or exactly or at most 996, at least or exactly or at most 997, at least or exactly or at most 998, at least or exactly or at most 999, at least or exactly or at most 1000, at least or exactly or at most 1001, at least or exactly or at most 1002, at least or exactly or at most 1003, at least or exactly or at most 1004, at least or exactly or at most 1005, at least or exactly or at most 1006, at least or exactly or at most 1007, at least or exactly or at most 1008, at least or exactly or at most 1009, at least or exactly or at most 1010, at least or exactly or at most 1011, at least or exactly or at most 1012, at least or exactly or at most 1013, at least or exactly or at most 1014, at least or exactly or at most 1015, at least or exactly or at most 1016, at least or exactly or at most 1017, at least or exactly or at most 1018, at least or exactly or at most 1019, at least or exactly or at most 1020, at least or exactly or at most 1021, at least or exactly or at most 1022, at least or exactly or at most 1023, at least or exactly or at most 1024, at least or exactly or at most 1025, at least or exactly or at most 1026, at least or exactly or at most 1027, at least or exactly or at most 1028, at least or exactly or at most 1029, at least or exactly or at most 1030, at least or exactly or at most 1031, at least or exactly or at most 1032, at least or exactly or at most 1033, at least or exactly or at most 1034, at least or exactly or at most 1035, at least or exactly or at most 1036, at least or exactly or at most 1037, at least or exactly or at most 1038, at least or exactly or at most 1039, at least or exactly or at most 1040, at least or exactly or at most 1041, at least or exactly or at most 1042, at least or exactly or at most 1043, at least or exactly or at most 1044, at least or exactly or at most 1045, at least or exactly or at most 1046, at least or exactly or at most 1047, at least or exactly or at most 1048, at least or exactly or at most 1049, at least or exactly or at most 1050, at least or exactly or at most 1051, at least or exactly or at most 1052, at least or exactly or at most 1053, at least or exactly or at most 1054, at least or exactly or at most 1055, at least or exactly or at most 1056, at least or exactly or at most 1057, at least or exactly or at most 1058, at least or exactly or at most 1059, at least or exactly or at most 1060, at least or exactly or at most 1061, at least or exactly or at most 1062, at least or exactly or at most 1063, at least or exactly or at most 1064, at least or exactly or at most 1065, at least or exactly or at most 1066, at least or exactly or at most 1067, at least or exactly or at most 1068, at least or exactly or at most 1069, at least or exactly or at most 1070, at least or exactly or at most 1071, at least or exactly or at most 1072, at least or exactly or at most 1073, at least or exactly or at most 1074, at least or exactly or at most 1075, or at least or exactly or at most 1076 contiguous amino acid residues.

Insofar as embodiment b relates to SEQ ID NO: 29-30 and 94, the at least 5 contiguous amino acids referred to in option b) in the definition of the first aspect of the invention may also constitute at least or exactly or at most 1077, at least or exactly or at most 1078, at least or exactly or at most 1079, at least or exactly or at most 1080, at least or exactly or at most 1081, at least or exactly or at most 1082, at least or exactly or at most 1083, at least or exactly or at most 1084, at least or exactly or at most 1085, at least or exactly or at most 1086, at least or exactly or at most 1087, at least or exactly or at most 1088, at least or exactly or at most 1089, at least or exactly or at most 1090, at least or exactly or at most 1091, at least or exactly or at most 1092, at least or exactly or at most 1093, at least or exactly or at most 1094, at least or exactly or at most 1095, at least or exactly or at most 1096, at least or exactly or at most 1097, at least or exactly or at most 1098, at least or exactly or at most 1099, at least or exactly or at most 1100, at least or exactly or at most 1101, at least or exactly or at most 1102, at least or exactly or at most 1103, at least or exactly or at most 1104, at least or exactly or at most 1105, at least or exactly or at most 1106, at least or exactly or at most 1107, at least or exactly or at most 1108, at least or exactly or at most 1109, at least or exactly or at most 1110, at least or exactly or at most 1111, at least or exactly or at most 1112, at least or exactly or at most 1113, at least or exactly or at most 1114, at least or exactly or at most 1115, at least or exactly or at most 1116, at least or exactly or at most 1117, at least or exactly or at most 1118, at least or exactly or at most 1119, at least or exactly or at most 1120, at least or exactly or at most 1121, at least or exactly or at most 1122, at least or exactly or at most 1123, at least or exactly or at most 1124, at least or exactly or at most 1125, at least or exactly or at most 1126, at least or exactly or at most 1127, at least or exactly or at most 1128, at least or exactly or at most 1129, at least or exactly or at most 1130, at least or exactly or at most 1131, at least or exactly or at most 1132, at least or exactly or at most 1133, at least or exactly or at most 1134, at least or exactly or at most 1135, at least or exactly or at most 1136, at least or exactly or at most 1137, at least or exactly or at most 1138, at least or exactly or at most 1139, at least or exactly or at most 1140, at least or exactly or at most 1141, at least or exactly or at most 1142, at least or exactly or at most 1143, at least or exactly or at most 1144, at least or exactly or at most 1145, at least or exactly or at most 1146, at least or exactly or at most 1147, at least or exactly or at most 1148, at least or exactly or at most 1149, at least or exactly or at most 1150, at least or exactly or at most 1151, at least or exactly or at most 1152, at least or exactly or at most 1153, at least or exactly or at most 1154, at least or exactly or at most 1155, at least or exactly or at most 1156, at least or exactly or at most 1157, or at least or exactly or at most 1158 contiguous amino acid residues.

Insofar as embodiment b relates to SEQ ID NO: 30 and 94, the at least 5 contiguous amino acids referred to in option b) in the definition of the first aspect of the invention may also constitute at least or exactly or at most 1159, at least or exactly or at most 1160, at least or exactly or at most 1161, at least or exactly or at most 1162, at least or exactly or at most 1163, at least or exactly or at most 1164, at least or exactly or at most 1165, at least or exactly or at most 1166, at least or exactly or at most 1167, at least or exactly or at most 1168, at least or exactly or at most 1169, at least or exactly or at most 1170, at least or exactly or at most 1171, at least or exactly or at most 1172, at least or exactly or at most 1173, at least or exactly or at most 1174, at least or exactly or at most 1175, at least or exactly or at most 1176, at least or exactly or at most 1177, at least or exactly or at most 1178, at least or exactly or at most 1179, at least or exactly or at most 1180, at least or exactly or at most 1181, at least or exactly or at most 1182, at least or exactly or at most 1183, at least or exactly or at most 1184, at least or exactly or at most 1185, at least or exactly or at most 1186, at least or exactly or at most 1187, at least or exactly or at most 1188, at least or exactly or at most 1189, at least or exactly or at most 1190, at least or exactly or at most 1.191, at least or exactly or at most 1192, at least or exactly or at most 1193, at least or exactly or at most 1194, at least or exactly or at most 1195, at least or exactly or at most 1196, at least or exactly or at most 1197, at least or exactly or at most 1198, at least or exactly or at most 1199, at least or exactly or at most 1200, at least or exactly or at most 1201, at least or exactly or at most 1202, at least or exactly or at most 1203, at least or exactly or at most 1204, at least or exactly or at most 1205, at least or exactly or at most 1206, at least or exactly or at most 1207, at least or exactly or at most 1208, at least or exactly or at most 1209, at least or exactly or at most 1210, at least or exactly or at most 1211, at least or exactly or at most 1212, at least or exactly or at most 1213, at least or exactly or at most 1214, at least or exactly or at most 1215, at least or exactly or at most 1216, at least or exactly or at most 1217, at least or exactly or at most 1218, at least or exactly or at most 1219, at least or exactly or at most 1220, at least or exactly or at most 1221, at least or exactly or at most 1222, at least or exactly or at most 1223, at least or exactly or at most 1224, at least or exactly or at most 1225, at least or exactly or at most 1226, at least or exactly or at most 1227, at least or exactly or at most 1228, at least or exactly or at most 1229, at least or exactly or at most 1230, at least or exactly or at most 1231, at least or exactly or at most 1232, at least or exactly or at most 1233, at least or exactly or at most 1234, at least or exactly or at most 1235, at least or exactly or at most 1236, at least or exactly or at most 1237, at least or exactly or at most 1238, at least or exactly or at most 1239, at least or exactly or at most 1240, at least or exactly or at most 1241, at least or exactly or at most 1242, at least or exactly or at most 1243, at least or exactly or at most 1244, at least or exactly or at most 1245, at least or exactly or at most 1246, at least or exactly or at most 1247, at least or exactly or at most 1248, at least or exactly or at most 1249, at least or exactly or at most 1250, at least or exactly or at most 1251, at least or exactly or at most 1252, at least or exactly or at most 1253, at least or exactly or at most 1254, at least or exactly or at most 1255, at least or exactly or at most 1256, at least or exactly or at most 1257, at least or exactly or at most 1258, at least or exactly or at most 1259, at least or exactly or at most 1260, at least or exactly or at most 1261, at least or exactly or at most 1262, at least or exactly or at most 1263, at least or exactly or at most 1264, at least or exactly or at most 1265, at least or exactly or at most 1266, at least or exactly or at most 1267, at least or exactly or at most 1268, at least or exactly or at most 1269, at least or exactly or at most 1270, at least or exactly or at most 1271, at least or exactly or at most 1272, at least or exactly or at most 1273, at least or exactly or at most 1274, at least or exactly or at most 1275, at least or exactly or at most 1276, at least or exactly or at most 1277, at least or exactly or at most 1278, at least or exactly or at most 1279, at least or exactly or at most 1280, at least or exactly or at most 1281, at least or exactly or at most 1282, at least or exactly or at most 1283, at least or exactly or at most 1284, at least or exactly or at most 1285, at least or exactly or at most 1286, at least or exactly or at most 1287, at least or exactly or at most 1288, at least or exactly or at most 1289, at least or exactly or at most 1290, at least or exactly or at most 1291, at least or exactly or at most 1292, at least or exactly or at most 1293, at least or exactly or at most 1294, at least or exactly or at most 1295, at least or exactly or at most 1296, at least or exactly or at most 1297, at least or exactly or at most 1298, at least or exactly or at most 1299, at least or exactly or at most 1300, at least or exactly or at most 1301, at least or exactly or at most 1302, at least or exactly or at most 1303, at least or exactly or at most 1304, at least or exactly or at most 1305, at least or exactly or at most 1306, at least or exactly or at most 1307, at least or exactly or at most 1308, at least or exactly or at most 1309, at least or exactly or at most 1310, at least or exactly or at most 1311, at least or exactly or at most 1312, at least or exactly or at most 1313, at least or exactly or at most 1314, at least or exactly or at most 1315, at least or exactly or at most 1316, at least or exactly or at most 1317, at least or exactly or at most 1318, at least or exactly or at most 1319, at least or exactly or at most 1320, at least or exactly or at most 1321, at least or exactly or at most 1322, at least or exactly or at most 1323, at least or exactly or at most 1324, at least or exactly or at most 1325, at least or exactly or at most 1326, at least or exactly or at most 1327, at least or exactly or at most 1328, at least or exactly or at most 1329, at least or exactly or at most 1330, at least or exactly or at most 1331, at least or exactly or at most 1332, at least or exactly or at most 1333, at least or exactly or at most 1334, at least or exactly or at most 1335, at least or exactly or at most 1336, at least or exactly or at most 1337, at least or exactly or at most 1338, at least or exactly or at most 1339, at least or exactly or at most 1340, at least or exactly or at most 1341, at least or exactly or at most 1342, at least or exactly or at most 1343, at least or exactly or at most 1344, at least or exactly or at most 1345, at least or exactly or at most 1346, at least or exactly or at most 1347, at least or exactly or at most 1348, at least or exactly or at most 1349, at least or exactly or at most 1350, at least or exactly or at most 1351, at least or exactly or at most 1352, at least or exactly or at most 1353, at least or exactly or at most 1354, at least or exactly or at most 1355, at least or exactly or at most 1356, at least or exactly or at most 1357, at least or exactly or at most 1358, at least or exactly or at most 1359, at least or exactly or at most 1360, at least or exactly or at most 1361, at least or exactly or at most 1362, at least or exactly or at most 1363, at least or exactly or at most 1364, at least or exactly or at most 1365, at least or exactly or at most 1366, at least or exactly or at most 1367, at least or exactly or at most 1368, at least or exactly or at most 1369, at least or exactly or at most 1370, at least or exactly or at most 1371, at least or exactly or at most 1372, at least or exactly or at most 1373, at least or exactly or at most 1374, at least or exactly or at most 1375, at least or exactly or at most 1376, at least or exactly or at most 1377, at least or exactly or at most 1378, at least or exactly or at most 1379, at least or exactly or at most 1380, at least or exactly or at most 1381, at least or exactly or at most 1382, at least or exactly or at most 1383, at least or exactly or at most 1384, at least or exactly or at most 1385, at least or exactly or at most 1386, at least or exactly or at most 1387, at least or exactly or at most 1388, at least or exactly or at most 1389, at least or exactly or at most 1390, at least or exactly or at most 1391, at least or exactly or at most 1392, at least or exactly or at most 1393, at least or exactly or at most 1394, at least or exactly or at most 1395, at least or exactly or at most 1396, at least or exactly or at most 1397, at least or exactly or at most 1398, at least or exactly or at most 1399, at least or exactly or at most 1400, at least or exactly or at most 1401, at least or exactly or at most 1402, at least or exactly or at most 1403, at least or exactly or at most 1404, at least or exactly or at most 1405, at least or exactly or at most 1406, at least or exactly or at most 1407, at least or exactly or at most 1408, at least or exactly or at most 1409, at least or exactly or at most 1410, or at least or exactly or at most 1411 contiguous amino acid residues.

Insofar as embodiment b relates to SEQ ID NO: 94, the at least 5 contiguous amino acids referred to in option b) in the definition of the first aspect of the invention may also constitute at least or exactly or at most 1412, at least or exactly or at most 1413, at least or exactly or at most 1414, at least or exactly or at most 1415, at least or exactly or at most 1416, at least or exactly or at most 1417, at least or exactly or at most 1418, at least or exactly or at most 1419, at least or exactly or at most 1420, at least or exactly or at most 1421, at least or exactly or at most 1422, at least or exactly or at most 1423, at least or exactly or at most 1424, at least or exactly or at most 1425, at least or exactly or at most 1426, at least or exactly or at most 1427, at least or exactly or at most 1428, at least or exactly or at most 1429, at least or exactly or at most 1430, at least or exactly or at most 1431, at least or exactly or at most 1432, at least or exactly or at most 1433, at least or exactly or at most 1434, at least or exactly or at most 1435, at least or exactly or at most 1436, at least or exactly or at most 1437, at least or exactly or at most 1438, at least or exactly or at most 1439, at least or exactly or at most 1440, at least or exactly or at most 1441, at least or exactly or at most 1442, at least or exactly or at most 1443, at least or exactly or at most 1444, at least or exactly or at most 1445, at least or exactly or at most 1446, at least or exactly or at most 1447, at least or exactly or at most 1448, at least or exactly or at most 1449, at least or exactly or at most 1450, at least or exactly or at most 1451, at least or exactly or at most 1452, at least or exactly or at most 1453, at least or exactly or at most 1454, at least or exactly or at most 1455, at least or exactly or at most 1456, at least or exactly or at most 1457, at least or exactly or at most 1458, at least or exactly or at most 1459, at least or exactly or at most 1460, at least or exactly or at most 1461, at least or exactly or at most 1462, at least or exactly or at most 1463, at least or exactly or at most 1464, at least or exactly or at most 1465, at least or exactly or at most 1466, at least or exactly or at most 1467, at least or exactly or at most 1468, at least or exactly or at most 1469, at least or exactly or at most 1470, at least or exactly or at most 1471, at least or exactly or at most 1472, at least or exactly or at most 1473, at least or exactly or at most 1474, at least or exactly or at most 1475, at least or exactly or at most 1476, at least or exactly or at most 1477, at least or exactly or at most 1478, at least or exactly or at most 1479, at least or exactly or at most 1480, at least or exactly or at most 1481, at least or exactly or at most 1482, at least or exactly or at most 1483, at least or exactly or at most 1484, at least or exactly or at most 1485, at least or exactly or at most 1486, at least or exactly or at most 1487, at least or exactly or at most 1488, at least or exactly or at most 1489, at least or exactly or at most 1490, at least or exactly or at most 1491, at least or exactly or at most 1492, at least or exactly or at most 1493, at least or exactly or at most 1494, at least or exactly or at most 1495, at least or exactly or at most 1496, at least or exactly or at most 1497, at least or exactly or at most 1498, at least or exactly or at most 1499, at least or exactly or at most 1500, at least or exactly or at most 1501, at least or exactly or at most 1502, at least or exactly or at most 1503, at least or exactly or at most 1504, at least or exactly or at most 1505, at least or exactly or at most 1506, at least or exactly or at most 1507, at least or exactly or at most 1508, at least or exactly or at most 1509, at least or exactly or at most 1510, at least or exactly or at most 1511, at least or exactly or at most 1512, at least or exactly or at most 1513, at least or exactly or at most 1514, at least or exactly or at most 1515, at least or exactly or at most 1516, at least or exactly or at most 1517, at least or exactly or at most 1518, at least or exactly or at most 1519, at least or exactly or at most 1520, at least or exactly or at most 1521, at least or exactly or at most 1522, at least or exactly or at most 1523, at least or exactly or at most 1524, at least or exactly or at most 1525, at least or exactly or at most 1526, at least or exactly or at most 1527, at least or exactly or at most 1528, at least or exactly or at most 1529, at least or exactly or at most 1530, at least or exactly or at most 1531, at least or exactly or at most 1532, at least or exactly or at most 1533, at least or exactly or at most 1534, at least or exactly or at most 1535, at least or exactly or at most 1536, at least or exactly or at most 1537, at least or exactly or at most 1538, at least or exactly or at most 1539, at least or exactly or at most 1540, at least or exactly or at most 1541, at least or exactly or at most 1542, at least or exactly or at most 1543, at least or exactly or at most 1544, at least or exactly or at most 1545, at least or exactly or at most 1546, at least or exactly or at most 1547, at least or exactly or at most 1548, at least or exactly or at most 1549, at least or exactly or at most 1550, at least or exactly or at most 1551, at least or exactly or at most 1552, at least or exactly or at most 1553, at least or exactly or at most 1554, at least or exactly or at most 1555, at least or exactly or at most 1556, at least or exactly or at most 1557, at least or exactly or at most 1558, at least or exactly or at most 1559, at least or exactly or at most 1560, at least or exactly or at most 1561, at least or exactly or at most 1562, at least or exactly or at most 1563, at least or exactly or at most 1564, at least or exactly or at most 1565, at least or exactly or at most 1566, at least or exactly or at most 1567, at least or exactly or at most 1568, at least or exactly or at most 1569, at least or exactly or at most 1570, at least or exactly or at most 1571, at least or exactly or at most 1572, at least or exactly or at most 1573, at least or exactly or at most 1574, at least or exactly or at most 1575, at least or exactly or at most 1576, at least or exactly or at most 1577, at least or exactly or at most 1578, at least or exactly or at most 1579, at least or exactly or at most 1580, at least or exactly or at most 1581, at least or exactly or at most 1582, at least or exactly or at most 1583, at least or exactly or at most 1584, at least or exactly or at most 1585, at least or exactly or at most 1586, at least or exactly or at most 1587, at least or exactly or at most 1588, at least or exactly or at most 1589, at least or exactly or at most 1590, at least or exactly or at most 1591, at least or exactly or at most 1592, at least or exactly or at most 1593, at least or exactly or at most 1594, at least or exactly or at most 1595, at least or exactly or at most 1596, at least or exactly or at most 1597, at least or exactly or at most 1598, at least or exactly or at most 1599, at least or exactly or at most 1600, at least or exactly or at most 1601, at least or exactly or at most 1602, at least or exactly or at most 1603, at least or exactly or at most 1604, at least or exactly or at most 1605, at least or exactly or at most 1606, at least or exactly or at most 1607, at least or exactly or at most 1608, at least or exactly or at most 1609, at least or exactly or at most 1610, at least or exactly or at most 1611 at least or exactly or at most 1612, at least or exactly or at most 1613, at least or exactly or at most 1614, at least or exactly or at most 1615, at least or exactly or at most 1616, at least or exactly or at most 1617, at least or exactly or at most 1618, at least or exactly or at most 1619, at least or exactly or at most 1620, at least or exactly or at most 1621, at least or exactly or at most 1622, at least or exactly or at most 1623, at least or exactly or at most 1624, at least or exactly or at most 1625, at least or exactly or at most 1626, at least or exactly or at most 1627, at least or exactly or at most 1628, at least or exactly or at most 1629, at least or exactly or at most 1630, at least or exactly or at most 1631, at least or exactly or at most 1632, at least or exactly or at most 1633, at least or exactly or at most 1634, at least or exactly or at most 1635, at least or exactly or at most 1636, at least or exactly or at most 1637, at least or exactly or at most 1638, at least or exactly or at most 1639, at least or exactly or at most 1640, at least or exactly or at most 1641, at least or exactly or at most 1642, at least or exactly or at most 1643, at least of exactly or at most 1644, at least or exactly or at most 1645, at least or exactly or at most 1646, at least or exactly or at most 1647, at least or exactly or at most 1648, at least or exactly or at most 1649 amino acid residues.

In some embodiments the invention relates to a polypeptide comprising an amino acid sequence consisting of at most 5 contiguous amino acid residues from any one of SEQ ID NOs 1-30. In these embodiment, the at most 5 contiguous amino acids can, for example, constitute 2, 3, 4 contiguous amino acid residues; preferably 4 contiguous amino acids.

In some embodiments, the polypeptide of the invention also has a sequence identity with the amino acid sequence of a) defined above of at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%. Similarly, the polypeptide of the invention in some embodiments also has a sequence identity with the amino acid sequence of b) defined above of at least 60%, such as at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 63, 64, 65, 66, 67, 68, 69, 70, 71, 72, 73, 74, 75, 76, 77, 78, 79, 80, 81, 82, 83, 84, 85, 86, 87, 88, 89, and 90 in any one of SEQ ID NOs: 1-30, 93, and 94, If the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 91, 92, 93, 94, 95, 96, 97, 98, 99, 100, 101, 102, 103, 104, 105, 106, 107, 108, 109, 110, 111, 112, and 113 in any on of SEQ ID NOs: 2-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 114 and 115 in any one of SEQ ID NOs: 3-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to amino acid residue 116 in any one of SEQ 10 NOs: 4-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any amino acid residue 1.17 in any one of SEQ ID NOS: 5-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to amino acid residue 118 in any one of SEQ ID NOS: 6-30, 93, and 94, If the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 119, 120, 121, 122, 123, 124, 125, 126, 127, 128, 129, 130, 131, 132, 133, 134, 135, 136, and 137 in any one of SEQ ID NOs: 7-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 138, 139, 140, 141, 142, 143, 144, 145, 146, and 147 In any one of SEQ ID NOs: 8-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 148, 149, 150, 151, 152, 153, 154, 155, 156, 157, 158, 159, 160, 161, 162, 163, and 164 in any one of SEQ ID NOs: 9-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 165, 166, 167, 168, 169, 170, 171, 172, 173, 174, 175, 176, 177, 178, 179, 180, 181, 182, 183, 184, 185, 186, 187, 188, 189, 190, 191, or 192 193, 194, 195, 196, 197, 198, 199, 200, 201, 202, 203, 204, 205, 206, 207, or 208 209, 210, 211, 212, 213, 214, 215, 216, 217, 218, 219, 220, 221, 222, 223, 224, and 225 in any one of SEQ ID NOS: 10-30, 93, and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 226, 227, 228, 229, 230, 231, 232, 233, 234, 235, 236, 237, 238, 239, 240, and 241 and 318 in any one of SEQ ID NOs: 11-30, 93 and 94 if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, and 318 in any one of SEQ ID NOS: 11-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 319, 320, 321, 322, 323, 324, 325, 326, 327, 328, 329,330, 331, 332, 333, 334, 335, 336, 337, 338, 339, 340, 341, 342, 343, 344, 345, 346, 347, 348, 349, 350, 351, 352, 353, 354, 355, or 356 357, 358, 359, 360, 361, 362, 363, 364, 365, 366, 367, 368, 369, 370, 371, and 372 in any one of SEQ ID NOS: 12-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 373, 374, 375, 376, 377, and 378 in any one of SEQ ID NOs: 13-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, and 424 in any one of SEQ ID NOs: 14-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 425, 426, 427, 428, 429, 430, 431, 432, 433, 434, 435, 436, and 437 in any one of SEQ ID NOs: 15-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480 in any one of SEQ ID NOS: 16-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 481, 482, 483, 484, 485, 486, 487, 488, 489, 490, 491, 492, 493, 494, 495, 496, 497, 498, 499, 500, 501, 502, 503, 504, 505, 506, 507, 508 509, 510, 511, 512, 513, 514, 515, 516, 517, 518, 519, 520, 521, 522, 523, 524, 525, 526, 527, 528, 529, 530, and 531 in any one of SEQ ID NOS: 17-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, or 563, 427564, 565, 566, 567, 568, 569, 570, 571, 572, 573, 574, 575, 576, 577, 578, 579, 580, 581, 582, 583, 584, SSS, 586, 587, 588, 589, 590, 591, 592, and 593 in any one of SEQ ID NOs: 18-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 594, 595, 596, 597, 598, 599, 600, 601, 602, 603, 604, 605, 606, 607, 608, 609, 610, 611, 612, 613, 614, 615, 616, 617, 618, 619, and 620 in any one of SEQ ID NOS: 19-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, and 653 in any one of SEQ ID NOS: 20-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 654, 655, 656, 657, 658, 659, 660, 661, 662, 663, 664, 665, 666, 667, 668, 669, 670, and 671 in any one of SEQ ID NOS: 21-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 672, 673, 674, 675, 676, 677, 678, 679, 680, 681, 682, 683, 684, 685, 686, 687, 688, 669, 690, 691, 692, 693, 694, 695, 696, and 697 in any one of SEQ ID NOS: 22-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, and 748 in any one of SEQ ID NOS: 23-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 749, 750, 751, 752, 753, 754, 755, 756 and 757 in any one of SEQ ID NOs: 24-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 758, 759, 760, 761, 762, 763, 764, 765, 766, 767, 768, 769, 770, 771, 772, 773, 774, 775, 776, 777, 778, 779, 780, 781, 782, 783, 784, 785 and 786 in any one of SEQ ID NOs: 25-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 787, 788, 789, 790, 791, 792, 793, 794, 795, 796, 797, 798, 799, 800, 801, 802, 803, 804 and 805 in any one of SEQ ID NOS: 26-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 806, 807, 808, 809, 810, 811, 812, 813, 815, 816, 817, 818, 819, 820, 821, 811, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891 and 892 in any one of SEQ ID NOS: 27-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072 and 1073 in any one of SEQ ID NOS: 28-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154 and 115S in any one of SEQ ID NOs: 29-30 and 94, if the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409 and 1410 in SEQ ID NO: 30 or 94, If the length of the at least or exactly or at most 5 amino acid residues so permits—the N-terminal first residue will not be higher numbered than N−L+1, where N is the number of amino acid residues of the reference sequence and L is the number of amino acids defined for option b.

In the embodiments defined by option b) above, the polypeptide of the invention is also one that has at least or exactly or at most 5 contiguous amino acid residues defined for option b) above and also has its N-terminal amino acid residue corresponding to any one of amino acid residues 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, and 1645 in SEQ ID NO: 94, If the length of the at least or exactly or at most 5 amino acid residues so permits—the N-terminal first residue will not be higher numbered than N−L+1, where N is the number of amino acid residues of the reference sequence and L is the number of amino acids defined for option b.

The polypeptide of the invention is in certain embodiments also fused or conjugated to an immunogenic carrier molecule; or, phrased otherwise, the polypeptide of the invention also includes such an immunogenic carrier molecule in addition to the material derived from SEQ ID NOS: 1-30. The immunogenic carrier molecule is a typically polypeptide that induces T-helper lymphocyte responses in a majority of humans, such as immunogenic carrier proteins selected from the group consisting of keyhole limpet hemocyanino or a fragment thereof, tetanus toxoid or a fragment thereof, diphtheria toxoid or a fragment thereof. Other suitable carrier molecules are discussed infra. One further fusion partner which is preferably incorporated is a “His tag”, i.e. a stretch of amino acids, which is rich in or only consists of histidinyl residues so as to facilitate protein purification.

In preferred embodiments, the polypeptide of the invention detailed above is capable of inducing an adaptive immune response against the polypeptide in a mammal, in particular in a human being. Preferably, the adaptive immune response is a protective adaptive immune response against infection with K. pneumoniae . . . . The polypeptide may in these cases induce a humeral and/or a cellular immune response.

A particularly preferred polypeptide of the invention is derived from SEQ ID NO: 6 and is otherwise as defined above.

### Epitopes

SEQ ID NOS: 1-30 include antigenic determinants (epitopes) that are as such recognized by antibodies and/or when bound to MHC molecules by T-cell receptors. For the purposes of the present invention, 8-cell epitopes (i.e. antibody binding epitopes) are of particular relevance.

It is relatively uncomplicated to identify linear 8-cell epitopes-one very simple approach entails that antibodies raised against K. pneumoniae or K. pneumoniae derived proteins disclosed herein are tested for binding to overlapping oligomeric peptides derived from any one of SEQ ID NO: 1-30. Thereby, the regions of the K. pneumoniae polypeptide which are responsible for or contribute to binding to the antibodies can be identified.

Alternatively, or additionally, one can produce mutated versions of the polypeptides of the invention, e.g. version where each single non-alanine residue in any one of SEQ ID NOS: 1-30 are point mutated to alanine—this method also assists in identifying complex assembled B-cell epitopes; this is the case when binding of the same antibody is modified by exchanging amino acids in different areas of the full-length polypeptide.

Also, in silico methods for B-cell epitope prediction can be employed: useful state-of-the-art systems for β-turn prediction is provided in Petersen B et al. (November 2010), Plos One 5 (11): e15079; prediction of linear B-cell epitopes, cf: Larsen J E P et al. (April 2006), Immunome Research, 2:2; prediction of solvent exposed amino acids: Petersen B et al (July 2009), BMC Structural Biology, 9:51.

### The Nucleic Acid Fragments of the Invention

The nucleic acid fragment of the invention referred to above is preferably is a DNA fragment (of a sequence such as SEQ ID NOs: 31-60 and 95-98) or an RNA fragment (of a sequence such as SEQ ID NOS 61-90 and 99-102).

In some embodiments the at least or exactly 10 consecutive nucleotides referred to in option iii) in the definition of the second aspect consists of at least or exactly 10, such as at least or exactly or at most 11, such as at least or exactly or at most 12, at least or exactly or at most 13, at least or exactly or at most 14, at least or exactly or at most 15, at least or exactly or at most 16, at least or exactly or at most 17 at least or exactly or at most 18, at least or exactly or at most 19, at least or exactly or at most 20, at least or exactly or at most 21, at least or exactly or at most 22, at least or exactly or at most 23, at least or exactly or at most 24, at least or exactly or at most 25, at least or exactly or at most 26, at least or exactly or at most 27, at least or exactly or at most 28, at least or exactly or at most 29, at least or exactly or at most 30, at least or exactly or at most 31, at least or exactly or at most 32, at least or exactly or at most 33, at least or exactly or at most 34, at least or exactly or at most 35, at least or exactly or at most 36, at least or exactly or at most 37, at least or exactly or at most 38, at least or exactly or at most 39, at least or exactly or at most 40, at least or exactly or at most 41, at least or exactly or at most 42, at least or exactly or at most 43, at least or exactly or at most 44, at least or exactly or at most 45, at least or exactly or at most 46, at least or exactly or at most 47, at least or exactly or at most 48, at least or exactly or at most 49, at least or exactly or at most 50, at least or exactly or at most 51, at least or exactly or at most 52, at least or exactly or at most 53, at least or exactly or at most 54, at least or exactly or at most 55, at least or exactly or at most 56, at least or exactly or at most 57, at least or exactly or at most 58, at least or exactly or at most 59, at least or exactly or at most 60, at least or exactly or at most 61, at least or exactly or at most 62, at least or exactly or at most 63, at least or exactly or at most 64, at least or exactly or at most 65, at least or exactly or at most 66, at least or exactly or at most 67, at least or exactly or at most 68, at least or exactly or at most 69, at least or exactly or at most 70, at least or exactly or at most 71, at least or exactly or at most 72, at least or exactly or at most 73, at least or exactly or at most 74, at least or exactly or at most 75, at least or exactly or at most 76, at least or exactly or at most 77, at least or exactly or at most 78, at least or exactly or at most 79, at least or exactly or at most 80, at least or exactly or at most 81, at least or exactly or at most 82, at least or exactly or at most 83, at least or exactly or at most 84, at least or exactly or at most 85, at least or exactly or at most 86, at least or exactly or at most 87, at least or exactly or at most 88, at least or exactly or at most 89, at least or exactly or at most 90, at least or exactly or at most 91, at least or exactly or at most 92, at least or exactly or at most 93, at least or exactly or at most 94, at least or exactly or at most 95, at least or exactly or at most 96, at least or exactly or at most 97, at least or exactly or at most 98, at least or exactly or at most 99, at least or exactly or at most 100, at least or exactly or at most 101, at least or exactly or at most 102, at least or exactly or at most 103, at least or exactly or at most 104, at least or exactly or at most 105, at least or exactly or at most 106, at least or exactly or at most 107, at least or exactly or at most 108, at least or exactly or at most 109, at least or exactly or at most 110, at least or exactly or at most 111, at least or exactly or at most 112, at least or exactly or at most 113, at least or exactly or at most 114, at least or exactly or at most 115, at least or exactly or at most 1.16, at least or exactly or at most 117, at least or exactly or at most 118, at least or exactly or at most 119, at least or exactly or at most 120, at least or exactly or at most 121, at least or exactly or at most 122, at least or exactly or at most 123, at least or exactly or at most 124, at least or exactly or at most 125, at least or exactly or at most 126, at least or exactly or at most 127, at least or exactly or at most 128, at least or exactly or at most 129, at least or exactly or at most 130, at least or exactly or at most 131, at least or exactly or at most 132, at least or exactly or at most 133, at least or exactly or at most 134, at least or exactly or at most 135, at least or exactly or at most 136, at least or exactly or at most 137, at least or exactly or at most 138, at least or exactly or at most 139, at least or exactly or at most 140, at least or exactly or at most 141, at least or exactly or at most 142, at least or exactly or at most 143, at least or exactly or at most 144, at least or exactly or at most 145, at least or exactly or at most 146, at least or exactly or at most 147, at least or exactly or at most 148, at least or exactly or at most 149, at least or exactly or at most 150, at least or exactly or at most 151, at least or exactly or at most 152, at least or exactly or at most 153, at least or exactly or at most 154, at least or exactly or at most 155, at least or exactly or at most 156, at least or exactly or at most 1.57, at least or exactly or at most 158, at least or exactly or at most 159, at least or exactly or at most 160, at least or exactly or at most 171, at least or exactly or at most 172, at least or exactly or at most 173, at least or exactly or at most 174, at least or exactly or at most 175, at least or exactly or at most 176, at least or exactly or at most 177, at least or exactly or at most 178, at least or exactly or at most 179, at least or exactly or at most 180, at least or exactly or at most 181, at least or exactly or at most 182, at least or exactly or at most 183, at least or exactly or at most 184, at least or exactly or at most 185, at least or exactly or at most 186, at least or exactly or at most 187, at least or exactly or at most 188, at least or exactly or at most 189, at least or exactly or at most 190, at least or exactly or at most 191, at least or exactly or at most 192, at least or exactly or at most 193, at least or exactly or at most 194, at least or exactly or at most 195, at least or exactly or at most 196, at least or exactly or at most 197, at least or exactly or at most 198, at least or exactly or at most 199, at least or exactly or at most 200, at least or exactly or at most 201, at least or exactly or at most 202, at least or exactly or at most 203, at least or exactly or at most 204, at least or exactly or at most 205, at least or exactly or at most 206, at least or exactly or at most 207, at least or exactly or at most 208, at least or exactly or at most 209, at least or exactly or at most 210, at least or exactly or at most 211, at least or exactly or at most 212, at least or exactly or at most 213, at least or exactly or at most 214, at least or exactly or at most 215, at least or exactly or at most 216, at least or exactly or at most 217, at least or exactly or at most 218, at least or exactly or at most 219, at least or exactly or at most 220, at least or exactly or at most 22.1, at least or exactly or at most 222, at least or exactly or at most 223, at least or exactly or at most 224, at least or exactly or at most 225, at least or exactly or at most 226, at least or exactly or at most 227, at least or exactly or at most 228, at least or exactly or at most 229, at least or exactly or at most 230, at least or exactly or at most 231, at least or exactly or at most 232, at least or exactly or at most 233, at least or exactly or at most 234, at least or exactly or at most 235, at least or exactly or at most 236, at least or exactly or at most 237, at least or exactly or at most 238, at least or exactly or at most 239, at least or exactly or at most 240, at least or exactly or at most 241, at least or exactly or at most 242, at least or exactly or at most 243, at least or exactly or at most 244, at least or exactly or at most 245, at least or exactly or at most 246, at least or exactly or at most 247, at least or exactly or at most 248, at least or exactly or at most 249, at least or exactly or at most 250, at least or exactly or at most 251, at least or exactly or at most 252, at least or exactly or at most 253, at least or exactly or at most 254, at least or exactly or at most 255, at least or exactly or at most 256, at least or exactly or at most 257, at least or exactly or at most 258, at least or exactly or at most 259, at least or exactly or at most 260, at least or exactly or at most 271, at least or exactly or at most 272, at least or exactly or at most 273, at least or exactly or at most 274, at least or exactly or at most 275, at least or exactly or at most 276, at least or exactly or at most 277, at least or exactly or at most 278, at least or exactly or at most 279, at least or exactly or at most 280, at least or exactly or at most 281, at least or exactly or at most 282, at least or exactly or at most 283, at least or exactly or at most 284, and at least or exactly or at most 285 consecutive nucleotides in any one of SEQ ID NOS: 31-60. Longer fragments are contemplated, i.e. fragments having at least 200, at least 300 at least 400, at least 500, at least 600, at least 700, at least 800, at least 900, at least 1000, at least 1500, at least 2000, at least 2500, at least 3000, at least 3500, and at least 4000 consecutive nucleotides from those of SEQ ID NOS: 31-90 and 95-102 that encompass fragments of such lengths.

In some embodiments the at most 10 consecutive nucleotides referred to in option iii) in the definition of the second aspect of the invention constitute 6, 7, 8, 9 or 10 nucleotides.

The nucleic acid fragment of the invention discussed above typically has a sequence identity with the nucleotide sequence defined for i) or ii) above, which is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

The nucleic acid fragment of the invention discussed above may also have a sequence identity with the nucleotide sequence defined for iii) above, which is at least 65%, such as at least 70%, at least 75%, at least 80%, at least 85%, at least 90%, at least 91%, at least 92%, at least 93%, at least 94%, at least 95%, at least 96%, at least 97%, at least 98%, and at least 99%.

The nucleic acid fragment of the invention described above comprises in certain embodiments at least or exactly or at most X distinct nucleic acid sequences each encoding a polypeptide of the invention, where each of said X distinct nucleic acid sequences encodes at least or exactly or at most one immunogenic amino acid sequence present in or derived from any one of SEQ ID NOS; 1-30 and wherein said X distinct nucleic acid sequences together encode immunogenic amino acid sequences present in or derived from at least or exactly or at most X of SEQ ID NOS: 1-30, 93 or 94, wherein X is an integer selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32. In other words, such a nucleic acid fragment encodes several polypeptides of the invention. In some embodiments, the X nucleic acid sequences are expressed as separate encoded proteins and in other embodiments as “pearls on a string”, i.e. fused proteins. In some embodiments, immunogenic amino acid sequences from any one of SEQ ID NOS: 1-30, 93 and 94 are only present in one of said X nucleic acid sequences.

It will be understood that the nucleic acid fragments of the invention may be used for both production, carrier and vaccine purposes—the latter will require that the sequences are included in expression vectors that may lead to production of immunogenic proteins in the host animal receiving the vector.

### The Vectors of the Invention

Vectors of the invention fall into several categories discussed infra. One preferred vector of the invention comprises in operable linkage and in the 5′-3′ direction, an expression control region comprising an enhancer/promoter for driving expression of the nucleic acid fragment defined for option i) above, optionally a signal peptide coding sequence, a nucleotide sequence defined for option i), and optionally a terminator. Hence, such a vector constitutes an expression vector useful for effecting production in cells of the polypeptide of the invention. Since the polypeptides of the invention are bacterial of origin, recombinant production is conveniently effected in bacterial host ceils, so here it is preferred that the expression control region drives expression in prokaryotic cell such as a bacterium, e.g. in E coli. However, if the vector is to drive expression in mammalian cell (as would be the case for a DNA vaccine vector), the expression control region should be adapted to this particular use.

At any rate, certain vectors of the invention are capable of autonomous replication.

Also, the vector of the invention may be one that is capable of being integrated into the genome of a host cell—this is particularly useful if the vector is use in the production of stably transformed cells, where the progeny will also include the genetic information introduced via the vector. Alternatively, vectors incapable of being integrated into the genome of a mammalian host cell are useful in e.g. DNA vaccination.

Typically, the vector of the invention is selected from the group consisting of a virus, such as a attenuated virus (which may in itself be useful as a vaccine agent), a bacteriophage, a plasmid, a minichromosome, and a cosmid.

Particularly interesting vectors are viral vectors (in particular those useful as vaccine agents). These may be selected from the group consisting of a retrovirus vector, such as a lentivirus vector, an adenovirus vector, an adeno-associated virus vector, and a pox virus vector. Certain pox virus vectors are preferred, in particular vaccinia virus vectors. A particularly preferred vaccinia virus vector is a modified vaccinia Ankara (MVA) vector.

A more detailed discussion of vectors of the invention is provided in the following:

Polypeptides of the invention may be encoded by a nucleic acid molecule comprised in a vector. A nucleic acid sequence can be “heterologous,” which means that it is in a context foreign to the cell in which the vector is being introduced, which includes a sequence homologous to a sequence in the cell but in a position within the host cell where it is ordinarily not found. Vectors include naked DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al, 2001; Ausubel et al, 1996, both incorporated herein by reference). In addition to encoding the polypeptides of this invention, a vector of the present invention may encode polypeptide sequences such as a tag or immunogenicity enhancing peptide (e.g. an immunogenic carrier or a fusion partner that stimulates the immune system, such as a cytokine or active fragment thereof). Useful vectors encoding such fusion proteins include pIN vectors (Inouye et al, 1985), vectors encoding a stretch of histidines, and pGEX vectors, for use in generating glutathione S-transferase (GST) soluble fusion proteins for later purification and separation or cleavage.

Vectors of the invention may be used in a host cell to produce a polypeptide of the invention that may subsequently be purified for administration to a subject or the vector may be purified for direct administration to a subject for expression of the protein in the subject (as is the case when administering a nucleic acid vaccine).

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

### 1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural state. Such promoters or enhancers may include promoters or enhancers of other genes, and promoters or enhancers isolated from any other prokaryotic, viral, or eukaryotic cell, and promoters or enhancers not “naturally occurring,” I.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

Naturally, it may be important to employ a promoter and/or enhancer that effectively direct(s) the expression of the DNA segment in the cell type or organism chosen for expression. Those of skill in the art of molecular biology generally know the use of promoters, enhancers, and cell type combinations for protein expression (see Sambrook et al, 2001, incorporated herein by reference), The promoters employed may be constitutive, tissue-specific, or inducible and in certain embodiments may direct high level expression of the introduced DNA segment under specified conditions, such as large-scale production of recombinant proteins or peptides.

Examples of inducible elements, which are regions of a nucleic acid sequence that can be activated in response to a specific stimulus, include but are not limited to Immunoglobulin Heavy Chain (Banerji et al, 1983; Gilles et al, 1983; Grosschedl et al, 1985; Atchinson et al, 1986, 1987; toiler et al, 1987; Weinberger et al, 1984; Kiledjian et al, 1988; Porton et al; 1990), Immunoglobulin Light Chain (Queen et al, 1983; Picard et al, 1984), T Cell Receptor (Luria et al, 1987; Winoto et al, 1989; Redondo et al; 1990), HLA DQα and/or DQβ (Sullivan et al, 1987), β-Interferon (Goodbourn et al, 1986; Fujita et al, 1987; Goodbourn et al, 1988), Interleukin-2 (Greene et al, 1989), Interleukin-2 Receptor (Greene et al, 1989; Lin et al, 1990), MHC Class II 5 (Koch et al, 1989), MHC Class I HLA-DRα (Sherman et al, 1989), β-Actin (Kawamoto et al, 1988; Ng et al; 1989), Muscle Creatine Kinase (MCK) (Jaynes et al, 1988; Horlick et al, 1989; Johnson et al, 1989), Prealbumin (Transthyretin) (Costa et al, 1988), Elastase I (Ornitz et al, 1987), Metallothionein (MTII) (Karin et al, 1987; Culotta et al, 1989), Collagenase (Pinkert et al, 1987; Angel et al, 1987), Albumin (Pinkert et al, 1987; Tranche et al, 1989, 1990), α-Fetoprotein (Godbout et al, 1988; Campere et al, 1989), γ-Globin (Bodine et al, 1987; Perez-Stable et al, 1990), β-Globin (Trudel et al, 1987), c-fos (Cohen et al, 1987), c-HA-ras (Triesman, 1986; Deschamps et al, 1985), Insulin (Ediund et al, 1985), Neural Cell Adhesion Molecule (NCAM) (Hirsh et al, 1990), ol-Antitrypain (Larimer et al, 1990), H28 (TH28) Histone (Hwang et al, 1990), Mouse and/or Type I Collagen (Ripe et al, 1989), Glucose-Regulated Proteins (GRP94 and GRP78) (Chang et al, 1989), Rat Growth Hormone (Larsen et al, 1986), Human Serum Amyloid A (SAA) (Edbrooke et al, 1989), Troponin I (TN I) (Yutzey et al, 1989), Platelet-Derived Growth Factor (PDGF) (Pech et al, 1989), Duchenne Muscular Dystrophy (Klamut et al, 1990), SV40 (Banerji et al, 1981; Moreau et al, 1981; Sleigh et al, 1985; Firak et al, 1986; Herr et al, 1986; Imbra et al, 1986; Kadesch et al, 1986; Wang et al, 1986; Ondek et al, 1987; Kuhl et al, 1987; Schaffner et al, 1988), Polyora (Swartzendruber et al, 1975; Vasseur et al, 1980; Katinka et al, 1980, 1981; Tyndell et al, 1981; Dandolo et al, 1983; de Villiers et al, 1984; Hen et al, 1986; Satake et al, 1988; Campbell et al, 1988), Retroviruses (Kriegler et al, 1982, 1983; Levinson et al, 1982; Kriegler et al, 1983, 1984a, b, 1988; Bosze et al, 1986; Miksicek et al, 1986; Celander et al, 1987; Thiesen et al, 1988; Celander et al, 1988; Choi et al, 1988; Reisman et al, 1989), Papilloma Virus (Campo et al, 1983; Lusky et al, 1983; Spandidos and Wilkie, 1983; Spalholz et al, 1985; Lusky et al, 1986; Cripe et al, 1987; Gloss et al, 1987; Hirochika et al, 1987; Stephens et al, 1987), Hepatitis B Virus (Bulla et al, 1986; Jameel et al, 1986; Shaul et al, 1987; Spandau et al, 1988; Varnice et al, 1988), Human Immunodeficiency Virus (Muesing et al, 1987; Hauber et al, 1988; Jakobovits et al, 1988; Feng et al, 1988; Takebe et al, 1988; Rosen et al, 1988; Berkhout et al, 1989; Laspia et al, 1989; Sharp et al, 1989; Braddock et al, 1989), Cytomegalovirus (CMV) IE (Weber et al, 1984; Boshart et al, 1985; Foecking et al, 1986), Gibbon Ape Leukemia Virus (Holbrook et al, 1987; Quinn et al, 1989).

Inducible Elements include, but are not limited to MT II-Phorbol Ester (TFA)/Heavy metals (Palmiter et al, 1982; Haslinger et al, 1985; Searle et al, 1985; Stuart et al, 1985; Imagawa et al, 1987, Karin et al, 1987; Angel et al, 1987b; McNeall et al, 1989); MMTV (mouse mammary tumor virus)—Glucocorticoids (Huang et al, 1981; Lee et al, 1981; Majors et al, 1983; Chandler et al, 1983; Lee et al, 1984; Ponta et al, 1985; Sakai et al, 1988); β-Interferon—poly(rl)x/poly(rc) (Tavernier et al, 1983); Adenovirus 5 E2-EIA (Imperiale et al, 1984); Collagenase—Phorbol Ester (TPA) (Angel et al, 1987a); Stromelysin—Phorbol Ester (TPA) (Angel et al, 1987b); SV40—Phorbol Ester (TPA) (Angel et al, 1987b); Murine MX Gene—Interferon, Newcastle Disease Virus (Hug et al, 1988); GRP78 Gene—A23187 (Resendez et al, 1988); α-2-Macroglobulin—IL-6 (Kunz et al, 1989); Vimentin-Serum (Rittling et al, 1989); MHC Class I Gene H-2κb—Interferon (Blanar et al, 1989); HSP70—E1A/SV40 Large T Antigen (Taylor et al, 1989, 1990a, 1990b); Proliferin-Phorbol Ester/TPA (Mordacq et al, 1989); Tumor Necrosis Factor-PMA (Hensel et al, 1989); and Thyroid Stimulating Hormonea Gene—Thyroid Hormone (Chatterjee et al, 1989).

Also contemplated as useful in the present invention are the dectin-1 and dectin-2 promoters. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In various embodiments, the human cytomegalovirus (CMV) immediate early gene promoter, the SV40 early promoter, and the Rous sarcoma virus long terminal repeat can be used to obtain high level expression of a related polynucleotide to this invention. The use of other viral or mammalian cellular or bacterial phage promoters, which are well known in the art, to achieve expression of polynucleotides is contemplated as well.

In embodiments in which a vector is administered to a subject for expression of the protein, it is contemplated that a desirable promoter for use with the vector is one that is not down-regulated by cytokines or one that is strong enough that even if down-regulated, it produces an effective amount of the protein/polypeptide of the current invention in a subject to elicit an immune response, Non-limiting examples of these are CMV IE and RSV LTR. In other embodiments, a promoter that is up-regulated in the presence of cytokines is employed. The MHC I promoter increases expression in the presence of IFN-γ.

Tissue specific promoters can be used, particularly if expression is in cells in which expression of an antigen is desirable, such as dendritic cells or macrophages. The mammalian MHC I and MHC II promoters are examples of such tissue-specific promoters. 2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic and may be operable in bacteria or mammalian cells. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation, Multiple genes can be efficiently expressed using a single promoter/enhancer to transcribe a single message (see U.S. Pat. Nos. 5,925,565 and 5,935,819, herein incorporated by reference).

### 2. Multiple Cloning Sites

Vectors can include a multiple cloning site (MCS), which is a nucleic acid region that contains multiple restriction enzyme sites, any of which can be used in conjunction with standard recombinant technology to digest the vector, (See Carbonelli et al, 1999, Levenson et al, 1998, and Cocea, 1997, incorporated herein by reference.) Frequently, a vector is linearized or fragmented using a restriction enzyme that cuts within the MCS to enable exogenous sequences to be ligated to the vector, Techniques involving restriction enzymes and ligation reactions are well known to those of skill in the art of recombinant technology,

### 3. Splicing Sites

Most transcribed eukaryotic RNA molecules will undergo RNA splicing to remove introns from the primary transcripts. If relevant in the context of vectors of the present invention, vectors containing genomic eukaryotic sequences may require donor and/or acceptor splicing sites to ensure proper processing of the transcript for protein expression. (See Chandler et al, 1997, incorporated herein by reference.)

### 4. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the bovine growth hormone terminator or viral termination sequences, such as the SV40 terminator. In certain embodiments, the termination signal may be a lack of transcribable or translatable sequence, such as due to a sequence truncation.

### 5. Polyadenylation Signals

In expression, particularly eukaryotic expression (as is relevant in nucleic acid vaccination), one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

### 6. Origins of Replication

In order to propagate a vector in a host cell, it may contain one or more origins of replication sites (often termed “on”), which is a specific nucleic acid sequence at which replication is initiated. Alternatively an autonomously replicating sequence (ARS) can be employed if the host ceil is yeast.

### 7. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, markers that confer resistance to neomycin, puromycin, hygromycin, DHFR, GPT, zeocin or histidinol are useful selectable markers. In addition to markers conferring a phenotype that allows for the discrimination of transformants based on the implementation of conditions, other types of markers including screenable markers such as GFP for colorimetric analysis. Alternatively, screenable enzymes such as herpes simplex virus thymidine kinase (tk) or chloramphenicol acetyltransferase (CAT) may be utilized. One of skill in the art would also know how to employ immunologic markers that can be used in conjunction with FACS analysis. The marker used is not believed to be important, so long as it is capable of being expressed simultaneously with the nucleic acid encoding a protein of the invention. Further examples of selectable and screenable markers are well known to one of skill in the art.

### The Transformed Cells of the Invention

Transformed cells of the invention are useful as organisms for producing the polypeptide of the invention, but also as simple “containers” of nucleic acids and vectors of the invention.

Certain transformed cells of the invention are capable of replicating the nucleic acid fragment defined for option i) of the second aspect of the invention. Preferred transformed cells of the invention are capable of expressing the nucleic acid fragment defined for option i).

For recombinant production it is convenient, but not a prerequisite that the transformed cell according is prokaryotic, such as a bacterium, but generally both prokaryotic cells and eukaryotic cells may be used.

Suitable prokaryotic cells are bacterial cells selected from the group consisting of Escherichia (such as E. coli), Bacillus (e.g. Bacillus subtilis), Salmonella, and Mycobacterium (preferably non-pathogenic, e.g. M. bovis BCG).

Eukaryotic cells can be in the form of yeasts (such as Saccharomyces cerevisiae) and protozoans. Alternatively, the transformed eukaryotic cells are derived from a multicellular organism such as a fungus, an insect cell, a plant cell, or a mammalian cell.

For production purposes, it is advantageous that the transformed cell of the invention is is stably transformed by having the nucleic acid defined above for option i) stably integrated into its genome, and in certain embodiments it is also preferred that the transformed cell secretes or carries on its surface the polypeptide of the invention, since this facilitates recovery of the polypeptides produced. A particular version of this embodiment is one where the transformed cell is a bacterium and secretion of the polypeptide of the invention is into the periplasmic space.

As noted above, stably transformed cells are preferred—these i.a. allows that cell lines comprised of transformed cells as defined herein may be established—such cell lines are particularly preferred aspects of the invention.

Further details on cells and cell lines are presented in the following:

Suitable cells for recombinant nucleic acid expression of the nucleic acid fragments of the present invention are prokaryotes and eukaryotes. Examples of prokaryotic cells include E. coli; members of the Staphylococcus genus, such as S. epidermidis; members of the Lactobacillus genus, such as L. plantarum; members of the Lactococcus genus, such as L. lactis; members of the Bacillus genus, such as B. subtilis; members of the Corynebacterium genus such as C. glutamicum; and members of the Pseudomonas genus such as Ps, fluorescens. Examples of eukaryotic cells include mammalian cells; insect cells; yeast cells such as members of the Saccharomyces genus (e.g. S. cerevisiae), members of the Pichia genus (e.g. P. pastoris), members of the Hansenula genus (e.g. H. polymorpha), members of the Kluyveromyces genus (e.g. K. lactis or K. fragilis) and members of the Schizosaccharomyces genus (e.g. S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2 nd Edition, Cold Spring Harbor Laboratory Press, 1989.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector, A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org) or from other depository institutions such as Deutsche Sammlung vor Microorganismen und Zelikulturen (DSM). An appropriate host can be determined by one of skill in the art based on the vector backbone and the desired result. A plasmid or cosmid, for example, can be introduced into a prokaryote host cell for replication of many vectors or expression of encoded proteins, Bacterial cells used as host cells for vector replication and/or expression include Staphylococcus strains, DH5α, JMI 09, and KC8, as well as a number of commercially available bacterial hosts such as SURE® Competent Cells and SOLOP ACK™ Gold Cells (STRATAGENE®, La Jolla, CA). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include Hela, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides,

### Expression Systems

Numerous expression systems exist that comprise at least a part or all of the compositions discussed above, Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ Baculovirus expression system from CLONTECH®

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### Amplification of Nucleic Acids

Nucleic acids used as a template for amplification may be isolated from cells, tissues or other samples according to standard methodologies (Sambrook et al, 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to sequences of genes identified herein are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Belius, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Alternative methods for amplification of target nucleic acid sequences that may be used in the practice of the present invention are disclosed in U.S. Pat. Nos. 5,843,650, 5,846,709, 5,846,783, 5,849,548, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

### Methods of Gene Transfer

Suitable methods for nucleic acid delivery to effect expression of compositions of the present invention are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al, 1987); by liposome mediated transfection (Nicolau and Sene, 1982; Fraley et al, 1979; Nicolau et al, 1987; Wong et al, 1980; Kaneda et al, 1989; Kato et al, 1991); by microprojectile bombardment (PCT Application Nos, WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al, 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al, 1993; U.S. Pat. Nos. 4,684,611 and 4,952,500, each incorporated herein by reference); by desiccation/inhibition mediated DNA uptake (Potrykus et al, 1985). Through the application of techniques such as these, organelle(s), cell(s), tissue(s) or organism(s) may be stably or transiently transformed.

The Antibodies of the Invention—and their Production/Isolation

Antibodies directed against the proteins of the invention are useful for affinity chromatography, immunoassays, and for distinguishing/Identifying Klebsiella proteins as well as for passive immunisation and therapy.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antiserum is obtained by bleeding the immunized animal into a glass or plastic container, incubating the blood at 25 C for one hour, followed by incubating at 4 C for 2-18 hours. The serum is recovered by centrifugation (eg. 1,000 g for 10 minutes). About 20-50 ml per bleed may be obtained from rabbits.

Monoclonal antibodies are prepared using the standard method of Kohler & Milstein [Nature (1975) 256:495-96], or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells expressing membrane-bound immunoglobulin specific for the antigen bind to the plate, and are not rinsed away with the rest of the suspension. Resulting 8-cells, or all dissociated spleen cells, are then induced to fuse with myeloma cells to form hybridomas, and are cultured in a selective I aedium (elg. hypexanthine, aminopterin, thymidine medium, “HAT”). The resulting hybridomas are plated by limiting dilution, and are assayed for production of antibodies, which bind specifically to the immunizing antigen (and which do not bind to unrelated antigens). The selected MAb-secreting hybridomas are then cultured either in vitro (eg. in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly 32p and 125I), electron-dense reagents, enzymes, and ligands having specific binding partners. Enzymes are typically detected by their activity. For example, horseradish peroxidase is usually detected by its ability to convert 3,3′, 5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, 1151 may serve as a radioactive label or as an electron-dense reagent. HRP may serve as enzyme or as antigen for a MAb. Further, one may combine various labels for desired effect. For example, MAbs and avidin also require labels in the practice of this invention; thus, one might label a MAb with biotin, and detect its presence with avidin labeled with, 125I, or with an anti-biotin MAb labeled with HRP. Other permutations and possibilities will be readily apparent to those of ordinary skill in the art, and are considered as equivalents within the scope of the instant invention.

According to the invention, the isolated monoclonal antibody or antibody analogue is preferably a monoclonal antibody selected from a multi-domain antibody such as a murine antibody, a chimeric antibody such as a humanized antibody, a fully human antibody, and single-domain antibody of a llama or a camel, or which is an antibody analogue selected from a fragment of an antibody such as an Fab or an F(ab′)₂, an scFV; cf. also the definition of the term “antibody” presented above.

### Compositions of the Invention: Vaccines

Pharmaceutical compositions, in particular vaccines, according to the invention may either be prophylactic (ie. to prevent infection) or therapeutic (ie, to treat disease after infection).

Such vaccines comprise immunising antigen(s), Immunogen(s), polypeptide(s), protein(s) or nucleic acid(s), usually in combination with “pharmaceutically acceptable carriers”, which include any carrier that does not itself induce the production of antibodies harmful to the individual receiving the composition.

In some embodiments of the invention, the pharmaceutical compositions such as vaccines include merely one single antigen, immunogen, polypeptide, protein, nucleic acid or vector of the invention, but in other embodiments, the pharmaceutical compositions comprise “cocktails” of the antigens or of the immunogens or of the polypeptides or of the protein or of the nucleic acids or of the vectors of the invention.

In particularly interesting embodiments, the pharmaceutical composition is an MVA vector mentioned herein, which encodes and can effect expression of at least 2 nucleic acid fragments of the invention.

Another interesting embodiment of a pharmaceutical composition comprises RNA as the active principle, i.e. at least one mRNA encoding a polypeptide of the invention.

An embodiment of a pharmaceutical composition of the invention comprises Y or at least Y or at most Y distinct polypeptides of the invention described above, where each of said Y or at least Y or at most Y distinct polypeptides comprises an immunogenic amino acid sequence present in or derived from any one of SEQ ID NOs: 1-30 and wherein said Y or at least Y or at most Y distinct polypeptides together comprise immunogenic amino acid sequences present in or derived from Y or at least Y or at most Y of SEQ ID NOs: 1-30, 93 or 94, wherein Y is an integer selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

Another embodiment of the pharmaceutical composition of the invention comprises Z or at least Z or at most Z distinct nucleic acid molecules (such as DNA and RNA) each encoding a polypeptide of the invention, where each of said Z or at least Z or at most Z distinct nucleic acid molecules encodes an immunogenic amino acid sequence present in or derived from any one of SEQ ID NOS: 1-30, 93, or 94 and wherein said at Z or least Z distinct nucleic acid molecules together encode immunogenic amino acid sequences present in or derived from Z or at least Z or at most Z of SEQ ID NOS: 1-30, 93, or 94, wherein Z is an integer selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles.

Such carriers are well known to those of ordinary skill in the art, Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, etc. pathogen, cf. the description of immunogenic carriers supra.

The pharmaceutical compositions of the invention thus typically contain an immunological adjuvant, which is commonly an aluminium based adjuvant or one of the other adjuvants described in the following:

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 In Vaccine design; the subunit and adjuvant approach, eds. Powell & Newman, Plenum Press 1995), containing 5% Squalene, 0.5% TWEEN 80, and 0.5% Span 85 (optionally containing various amounts of MTP-PE (see below), although not required) formulated into submicron particles using a microfluidizer such as Model 110Y microfluidizer (Microfluidics, Newton, MA), (b) SAF, containing 10% Squalane, 0.4% TWEEN 80, 5% pluronic-blocked polymer L121, and thr-MDP (see below) either microfluidized into a submicron emulsion or vortexed to generate a larger particle size emulsion, and (c) Ribi adjuvant system (RAS), (Ribi Immunochem, Hamilton, MT) containing 2% Squalene, 0.2% TWEEN 80, and one or more bacterial cell wall components from the group consisting of monophosphoryl lipid A (MPL), trehalose dimycolate (TDM), and cell wall skeleton (CWS), preferably MPL+CWS (Detox™) (3) saponin adjuvants such as STIMULON™ (Cambridge Bioscience, Worcester, MA) may be used or particles generated therefrom such as ISCOMs (immunostimulating complexes); (4) Complete Freund's Adjuvant (CFA) and incomplete Freund's Adjuvant (IFA); (5) cytokines, such as interleukins (eg. IL-1, IL-2, IL-4, IL-S, IL-6, IL-7, IL-12, etc.), interferons (eg. gamma interferon), macrophage colony stimulating factor (M-CSF), tumor necrosis factor (TNF), etc.; and (6) other substances that act as immunostimulating agents to enhance the effectiveness of the composition. Alum and MF59™ª adjuvants are preferred.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2″-2′-dipalmitoyl-sn-glycero-3-hydroxyphosphoryloxy)-ethylamine (MTP-PE), etc.

The immunogenic compositions (eg, the immunising antigen or immunogen or polypeptide or protein or nucleic acid, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the antigenic or immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (eg. WO98/20734). Additional formulations suitable for other modes of administration include oral and pulmonary formulations, suppositories, and transdermal applications. In the case of nucleic acid vaccination, also the intravenous or intraarterial routes may be applicable.

Dosage treatment may be a single dose schedule or a multiple dose schedule. The vaccine may be administered in conjunction with other immunoregulatory agents.

As an alternative to protein-based vaccines, DNA vaccination (also termed nucleic acid vaccination or gene vaccination) may be used [eg. Robinson & Torres (1997) Seminars in Immunol 9:271-283; Donnelly et al. (1997) Avnu Rev Immunol 15:617-648; later herein].

A further aspect of the invention is as mentioned above the recognition that combination vaccines can be provided, wherein 2 or more antigens disclosed herein are combined to enhance the immune response by the vaccinated animal, including to optimize initial immune response and duration of immunity. For the purposes of this aspect of the invention, multiple antigenic fragments derived from the same, longer protein can also be used, such as the use of a combination of different lengths of polypeptide sequence fragments from one protein.

Thus, embodiments of the invention relate to a composition (or the use as a vaccine thereof) comprising 2 distinct (i.e. non-identical) proteinaceous immunogens disclosed herein wherein the first of said immunogens is SEQ ID NO: 1 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 2 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 3 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 4 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 5 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 6 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 7 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 8 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 9 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 10 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 11 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, and 16 or in combination with a variant or fragment disclosed herein of any one of SEQ ID NOs: 1, 2, 3, 4, 5, 6, 7, 8, 9, 10, 11, 12, 13, 14, 15, and 16.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 12 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 13 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 14 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 3, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 15 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 3, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 16 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 17 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 18 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 19 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 20 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 21 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-Identical) proteinaceaous immunogens disclosed herein wherein the first of said immunogens is SEQ ID NO: 22 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 23 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 24 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ 10 NO: 25 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 26 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-Identical) proteinaceaous immunogens disclosed herein wherein the first of said immunogens is SEQ ID NO: 27 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 28 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 29 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 30 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 93 or a variant or fragment thereof disclosed herein in combination with a proteinaceous immunogen selected from any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94.

Also, embodiments of the invention relate to a composition (or the use as a vaccine thereof) comprising 2 distinct (i.e. non-identical) proteinaceaous immunogens disclosed herein wherein the first of said immunogens is SEQ ID NO: 94 or a variant or fragment thereof disclosed herein in combination with a proteinaceous immunogen selected from any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94 or in combination with a variant or fragment disclosed herein of any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 93, and 94,

### Treatment Methods of the Invention

The method of the sixth aspect of the invention generally relates to induction of immunity and as such also entails method that relate to treatment, prophylaxis and amelioration of disease.

When Immunization methods entail that a polypeptide of the invention or a composition comprising such a polypeptide is administered the animal (e.g. the human) typically receives between 0.5 and 5,000 μg of the polypeptide of the invention per administration.

In preferred embodiments of the sixth aspect, the immuniation scheme includes that the animal (e.g. the human) receives a priming administration and one or more booster administrations.

Preferred embodiments of the 6^{th} aspect of the invention comprise that the administration is for the purpose of inducing protective immunity against K. pneumoniae. In this embodiment it is particularly preferred that the protective immunity is effective in reducing the risk of attracting infection with K. pneumoniae or is effective in treating or ameliorating infection with K. pneumoniae.

As mentioned herein, the preferred vaccines of the invention induce humoral immunity, so it is preferred that the administration is for the purpose of inducing antibodies specific for K. pneumoniae and wherein said antibodies or 8-lymphocytes producing said antibodies are subsequently recovered from the animal.

But, as also mentioned the method of the 6th aspect may also be useful in antibody production, so in other embodiments the administration is for the purpose of inducing antibodies specific for K. pneumoniae and wherein B-lymphocytes producing said antibodies are subsequently recovered from the animal and used for preparation of monoclonal antibodies.

Pharmaceutical compositions can as mentioned above comprise polypeptides, antibodies, or nucleic acids of the invention. The pharmaceutical compositions will comprise a therapeutically effective amount thereof.

The term “therapeutically effective amount” or “prophylactically effective amount” as used herein refers to an amount of a therapeutic agent to treat, amellorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. Reference is however made to the ranges for dosages of immunologically effective amounts of polypeptides, cf. above.

However, the effective amount for a given situation can be determined by routine experimentation and is within the judgement of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub, Co., N. J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

As is apparent from the claims, the invention also relates to related embodiments to the treatment and prophylaxis disclosed herein: the invention also includes embodiments where
the polypeptide of the invention is for use as a pharmaceutical, in particular for use as a pharmaceutical in the treatment, prophylaxis or amelioration of infection with K. pneumoniae;
the nucleic acid fragment of the invention or the vector of the invention is for use as a pharmaceutical, in particular for use as a pharmaceutical in the treatment, prophylaxis or amelioration of infection with K. pneumoniae;
the transformed cell of the invention is for use as a pharmaceutical, in particular for use as a pharmaceutical in the treatment, prophylaxis or amelioration of infection with K. pneumoniae.
the antibody, antibody fragment or antibody analogue of the invention is for use as a pharmaceutical, in particular for use as a pharmaceutical in the treatment, prophylaxis or amelioration of infection with K. pneumoniae.

### Sequence Information

The sequence listing included sets forth the sequences of polypeptides and nucleic acids of the present invention. For easy reference, the sequences are presented in the following:

The polypeptides of the invention have or derive from the following amino acid sequences:

SEQ ID NO: 1
MKKLALLSAVMTLGMSSWAFAADNPPPPPEKGAQHQGKPPVKNGQHEGKQAQYNRKQPQRDGKQPQH
DGKQPQHNGKQPPKGSEHSGKPLPPKA
SEQ ID NO: 2
MKRYATALLFCTLSLTSLAARADIIDDAIGNIQQAINDAYNPGSSRSDDDDRYDDDGRYDDGRYQGSRQ
QSRDSQRQYDERQRQLDERRRQLDERQRQLDRDRRQLESDQRRLDDSY
SEQ ID NO: 3
MFRSLILAAVLLAAGPLVANAGEITLLPSVKLQIGDRDNYGNYWDGGSWRDRDYWRRHYEWRDNRWHR
HDNGWHKGWYKGRDKAWERGYRAGWNDRDDHRGGWGRGPGGRGHGHGHGHH
SEQ ID NO: 4
MKEIGLPLLLLTALASPAFAADCQPNGIGGSFCINDDGTTTDTVPNEVNGMDTYSNNGGYTSSLPDRSGA
DEALEGSSLSTQQGVGSGQSDSALAGRDWHSPANLNDGAATSSMSLLDKP
SEQ ID NO: 5
MNMKKLTTLLLTATLGLASGAALAADTGAQSNNGQANSSADAGQVAPDARENVAPNNVDNSQINSGSG
GTTGSTMTQDNMSSNEVHKNSMCKDGRCPDTGKKLDNGGNTTQDNSKTDGTTQ
SEQ ID NO: 6
MKHRIALLLVLTSLSASALAASPCQEKEQDIQREISYAEKHHNQSRIDGLNTALRQVRENCSDSKLKADH
QQKIAKQREEIAERQRDLQEARKKGDADKINKRQHKLNEAQQELKTLESRDY
SEQ ID NO: 7
MRLITRHVREDIMKKAMIALSAlLVAAPVFAATTHATDDTVAAANANANTAKEKLHQAQHEGEEQQLKAK
HAAEGKQDSVGSQVSEGAQKTWNKTKEGTEKGWDKTKEVSEKGWNATKSGAEKGWDKTKTGAEELK
NKVTE
SEQ ID NO: 8
MKKMISLAVLLSCVLSVPAFADGPIVDGHRPEQPTVWQNGPDHDGHAPQGGPDAHHQGDHDQRGPDRD
GHDKRDLARHEQDHFAWRGNDFRKGHPAPAPFRGDEYRVRDWSDRGLPPPPEGHHWSYIDGNYVLIAA
ATGIITSILVSGALGH
SEQ ID NO: 9
MKKPTSATRGKSGRKSREELNQEARDRKRQKKHRGHAAGSRANGGDAASAGKKQRQAQDPRVGSKKP
IPLGVSESSVPAPKQHKPKSEKPMLSPQAELELLENDERLDALLERLEEGGTLNAEEQSWVDAKLDRIDEL
MQQLGLSYDDEDEEEEERQEDMMRLLKGGIN
SEQ ID NO: 10
MASKFQNRLVGTIVLVALGVIILPGLLDGQKKHYQDEFAAIPLVPKPGDRDEPDMLPAATQALPSQPPEGA
AEEVRAGDAAAPSLDPSRIPVINSNSFDDVQEPVVAAKPQPKPQPKPQPQQQASTPTPPPAKPQQQQPPQ
QQAALPAPTGKAYVVQLGALKNADKVNEIVGKLRASGFKVYTSPSTPVQGKITRILVGPDASKDKLKGQL
GDLQQISGLSGVVMGFTPN
SEQ ID NO: 11
MAQRDYVRRSQPASSRRKKSTTRSSRNKQSSLPAISPAMVAIAAAVLVAFIGGLYFITHHKKEEAEAMQN
RQAAGNGLPPKPEERWRYIKELESRQPGVRAPTEPTAGGEVMKPEQLTDEQRQLLAQMQADMRQQPTQL
TEVPWNEQTPAQRQQTLQRQRLAQQQQQAQQQQWAQTQAQTVQQQPPRVQQPKPVQQQQPKQTAS
NQQPYQDLLQTPAHTNTTQPRTQAAAPVTRVEEAPKTTAEKKDDRSWMIQCGSFKGAEQAETVRAQLAF
EGFASHITTNNGWNRVVIGPLKGKESANEMITRLKMAGHANCIRLAARG
SEQ ID NO: 12
MSAGSTKFTVSRIAALSLVSLWLAGCTNTNNPPAPVSSAGGAASSSTNSGMLITPPPSGVKSAPQAQPIQ
PMQTQTIQPAPVAQEPVQTVNGRIVYNIRKYGDIPKGSYTGGSTYTVKRGDTLFYIAWVTGNDFRDLAQRN
NIPAPYALNVGQVLQVGNASGQPITGENAVSQASARASGGATTSTTSAQKSTAVVASQPTITYSESSGEQ
SATKMLPNNKPATTTTTVVAPVTAPTTVSTTQPTASSTSTSSPISAWRWPTDGKVIENFSGAEGGNKGIDI
AGSKGQAIVATADGRVVYAGNALRGYGNLIIIKHNDDYLSAYAHNDTMLVREQQEVKAGQKIATMGSTG
TSSTRLHFEIRYKGKSVNPLQYLPQR
SEQ ID NO: 13
MRKQWLGICIAAGLLAACSSDDVQQKTVSTPQPAVCNGPTVEISGADPQYETPNATANQDYERDGKSYK
IVQDPANFTQAGFAAIYDAEPNSNLTASGEAFDPTQLTAAHPTLPIPSYARITNLANGRMIVVRINDRGPYG
NDRVISLSRASADRLNTSNNTKVRIDPIIVAPDGSLSGPGMACTTVAKQTYALPARPNLDGGDAAGMSQP
APTDVRPISNSTLTPADSVGAPVNSGGFLGAPTPLNNGVLESSEPAAAAATAPAAGATPTAPVTAPGSIQG
NVVPAAATAAAAGAVAASSSATSSASGNFVVQVGAVSDQTRAQQYQQRLSQQFSVPGRVMQNGAVWR
IQLGPFADKAQASAVQQRLQSEAQLQSFITRAN
SEQ ID NO: 14
MDDFKPEDDMKADRNDRRAGRSRQSSERDADPQINFDDVDLDADEGRPTRAGKARREREEEEFEEELD
AQDEEMLEEQPVERRPRKRKKAPAKPASRQYIMMGVGILVLLLLIVGIGSALKSPSSSSQQTASGEKSINL
SDDQSASMPAAGQDQTAAANSTSQQDVTVPPIAANPTQGQAAVAPQGQQRIEVQGDLNNALTQQQGQ
LDGAVANSTLPTEPATVAPIRNGANGTAAPRQATERQTAATPRPAERKHTVIEAKPQSKPQAVAKTPVESK
PVQPKHVESTATTAPAKTSVSESKPVATAQSKPTTTTAAPAATAAAAAPAAKTGKTAGDVSSMKTAPSGH
YTLQLSSSSNYDNLNNWAKKEKLDKYVVYETSRNGQPWYVLVSGIYASKDEAKRAVTSLPADVQAKNPW
AKPLHQVQADLK
SEQ ID NO: 15
MSKATEQNDKLKRAIIISVALHIILIALLIWSSFDEHLDASAGGGGGSSIDAVMVDPGAVVNNYNRQQQQ
QASARRAAEQREKQAQQQAEELREKQAAEQERLKQLEQERLQAQEAAKEAKEQQKQAEEAAAKAAAAA
KAKADAQAKEAQEAAAKAAAEAKAKADAQAKAAEQAAAKAAADAKKQAEAAAAKAAAEAKKQAEAEAA
KAAAEAQKKAEAAAAKKAQQEAEKKAQQEAAKQAAAEKAAAEKAAEKAAAQKAAAEKAAAEKAAAAEK
AAAAKAAAAEKAAADKAAKAAAAKAAAAKKAAAAKEADGVDNLLGDLSSGKNAPKTGGGAKGNNASAA
GSGINTKNSASGADININYAGQIKSAIESKFYDASSYAGKTCTLRIKLAPDGLLLNIQSEGGDPALCQAALAA
ARQAKFPKPPSQAVYEVFKNAPLDFKPQ
SEQ ID NO: 16
MFFLSIFYMEMTKVKLSALFIALIPLLGSPVIHAETTAAPVLENRAAQGDITTPGGARRLTGDQTEALRASLI
NKPAKNVILLIGDGMGDSEITAARNYAEGAGGFFKGIDALPLTGQYTHYSLDKKTGKPDYVTDSAASATA
WTTGVKTYNGALGVDIHENAHQTILELAKAAGLATGNVSTAELQDATPAALVAHVISRKCYGPTVTSEKC
PSNALEKGGKGSITEQLLNARPDVTLGGGAKTFTETATAGEWQGKTLREQAQARGYQIVTDAASLAAATE
ASQDKPLLGLFADGNMPVRWEGPKASYHGNIDKPPVTCTPNPKRDASVPTLAQMTEKAIDLLSRNEKGFF
LQVEGASIDKQDHAANPCGQIGETVDLDEAVQKALEFARKDGNTLVIVTADHAHASQIIPADSKAPGLTQ
ALNTHDGAVMVMSYGNSEEESMEHTGTQLRIAAYGPHAANVVGLTDQTDLFTTMKAALSLK
SEQ ID NO: 17
MSLPFKPHIIALLCSAGLLAAAGTLYVQSRTPATIAEPPAQQAPAPAASTTQPVAATYTQAQIDQWVAPIAL
YPDSLLSQVLMASTYPDINVLQAVQWSQDNIPAMKGDAAVQAVASQPWDPSVKSLVAFPALLAMMGENPP
WVENLGNAFLAQPHDVMDSVQRLRAIAQQTGTLKSTPQQKVIVTPAAPVSASSSTAATATAHTAAPAPTQ
VIKIEPTNPQVVYVPSYNPSTVYGTWPNSAYPPVYLPPPPGEQFTDSFVKGFGYSLGVATTWALFSSIDWD
DDDHHHHDDDYHHGDYSHNGDNININVNNFNHITGENLPGNHVNWQHNPAYRGHTPYPDNTVAQRFH
QTNVSGGLSATQHAPVDREAQRQAAMTQLQHNVPTATAGNLAANNASRDAQRQAASAQLKQATQRSN
YRGYDSTPTQQQRREAAKTQLKNPTPQQQQRREAARSHEQNRTPQQQQRRQQFQSATPAQRQQTLSHL
RANALSGNESRAPSWQAQQERGLQSRQFSGVNRELRDGTRERLSEHHELRRR
SEQ ID NO: 18
MFKFKASYVALAAVLTSSVVYADPTSYTHSSGATVIDIEKPNAAGVSHNLYRDFNVGANGTILNNSGDDV
SHSTFGNTARNNNLTAGSASVILNEVTSKNASSLKGFIEVNGQKADVVIANPNGITCSGCSFVNTNKAILT
TGKVNMTDDGAIGSYTVTGGTLTIGENGMNAANGYAVLLADAININGKVQANNALVSAGNFTMDNSSG
SVTSAGKKATLIQMTVNPQYSIDVSSLGGIEANSISMVGNNIGFGVRNKGSIVANSSLQLTSNGNLLNKG
TIKSNGLLSQVATASGITNDGSIAGAYYLMLSSGDYIVNTGSLSGGQLIATANGNITNGDSGTMTGTSGL
SLTSGGKIRNEEKASLLSNNQIAATAIGDFLNEGKISAKHTSLTFVGDSFKNTGNINSTGQTTIQSLKQDG
SANTGEIYNLGNITGENINLQTNGTLAQSSSGRIEATNAITAHSYWLNQNGYIvINAADITTDHGVVNNYG
NITAKNISITTYSDITNEGQISSTGDLTLNTKNKGAIYNYSTLSAGGNMTLTATKVVNGGKSCGILGLAKC
GVGTLTADKLVLNSSQKYVSDMGGKQYFKSTEVNTVK
SEQ ID NO: 19
MMDNLRTAANSVVLKIIFGIIIVSFILTGVSGYLIGGGKNYAAKVNGQEIGRGQFENAVASERNRMQQQL
GDQFSELAANENYMKTMRQQVLNRLIDESLLDQYARELGLSISDEQVKQAIFQTQAFQTNGKFDNQRFS
GIVAQMGMTTDQYAQALRNQLTTQQLINAIAGTDFMLPGESDQLAALVSQQRVVREATINVNALAAKQT
ASDEEINAFWQQNQARFMAPEQFRVSYIKMDAASMQESASDEEIQSWYDQHKDQFTQPQRNRYSVIQT
KTEADAKAVLAELQKGADFATLAKEKSTDIISARNGGDMGWMEDASTVPELKDAGLKEKGQLSGVIKSS
VGFLVARLDDVQPAQVKPLADVRNDIAAKVKQEKALDAYYALQQKVSDAASNDNESLASAAQVAGLKVV
ETGWFGRDNLPEELNFKPVADAIFNGGLVGENGAPGSNSDIITVDGDRAFVLRISEHKAEAVKPLAEVKA
QVSDIVKHNKAEQQAKLEADKLLAALKDGKGDEAMKAAGLSFGAPQTLSRTGQDPLSQLAFTLPLPQQG
KPVYGVGSNMQGDVVLVALDEVKAGSMPEEQKKAMVQGITQNNAQIAFEALMSNLRKAAKIKLGDSIDQ
QQ
SEQ ID NO: 20
MFRLNPFIRAGLSASVVSLAFPALADVNEETLVVTASATEQNVKDAPASISVITQQDLQRKPVQNLKDVLR
DVPGVQLTNEGDNRKGVSIRGLSSSYTLILVDGKRVNSRNAVFRHNDFDLNWIPVDAIERIEVVRGPMSS
LYGSDALGGVVNIITKKIGQKWTGTLSADTTIQEHRDRGDTWNGQFFTSGPLIDGVLGMKAYGSLAKRA
KDDPQSSSNATGETPRIEGFTSRDGNVEFAWTPNENHDFTAGYGFDRQDRDSDSLDRNRLERENYSLSH
NGRWDIGNSELKFYGEKVDNKNPGQSGTITSESNAIDGKYVLPLGMINQLVTFGGEWRHDKLKDPVNLS
SGGQSTSASQYALFIEDEWRIIEPLALTTGIRVIDDHQTYGDHWSPRAYLVYNATDTVTVKGGWATAFKA
PSLLQLNPDWTTNSCRGSCSIVGNPDLKPETSESFELGLYYRGEEGWLENVEGSITTFQNNVDDMIDVLR
TSSASEAPGYPNFVGWKTVNGKRVPIFRYFNVNKARIKGVETEVKIPFGDEWKLTVNYTYNDGRDLSNGG
DKPLQTLPFHTANGTLDWKPLDDWSFYVTANYTGQQRAVSATGKTPGGYTLFDVGAAWQVTKNVKLRS
GVQNVGDKDLSRDDYSYTEEGRRYFMAVDYRF
SEQ ID NO: 21
MNRAATLTLNAPLLIALVAALALSTPFTAGAAPAFLDYAQQQTQQSQAQEKNDAASAKQTQESRQSADNK
KTGTSTSQLQKRITSQQAAIAQKDKLIQQLKKQLAATPQTDTAGANEQAALNKRINELQVALSAATAEKE
ALIKKAGVVQNNNLQQSQAAARQQIQQLTTQIQQAEAENKRLSASFTTLNKDKHALMTQLAATEKEKQA
ALEQVKALNADKQPLTTRLAAAEKEKQAVLEQVKALNADKQSLTIRLAAAEKAQQAAVDQAKALNADKQP
LATRLAAAEKEKQAVLEQVKALSADKQSLTIRLAAAEKAQQAALDQAKALNADKQPLATRLAAAEKEKQA
VLEQVKALNADKQSLTIRLAAAEKTQQAALDQVKALNADKQSLSTRLAAADKAPHGPANDAAAPKNEPPE
MAAIVAAYRLQADKDNAQLRIv1KEDEIELLRTQLSVQSKTRSGESAAAKLSASGEQQAYAIGASMGSEAL
NVLTTRRTQGVTVDAGLVLQGIEDAFRGQLRLGEQERNKALFDVSQQVFQNLNKIEQKNISAGKKYQQA
FARKKDVVFKEGVYSRVDYLGKGKISGNDLVTVVIKEMLTDGTVINDMEAKDQALTQKLDAYPPVFREPL
KRLQNHGSVTLVVPPEKAYGSKGLPPKIPPGATMVYSVRIVDSQPEPAK
SEQ ID NO: 22
MKILSVRHAALPALLLPLIAAAQAADEQTMVVTAAPTTVSELDTPAAVSVVNGDEMRQAAPRVNILSESLGA
VPGLQVQNRQNYAQDLQLSIRGFGSRSTYGVRGLRIYVDGIPATMPDGQGQTSNIDIGSVDTIEVLRGPF
SALYGNSSGGVINVTSQTGTQPPTVEASSYYGSFGTWHYGMKATGAVGDGSHAGDVDYTVSTNRFTTH
GYRDHSGARKNLANARLGVRINDVSKLTLLLNSVDIKANDAGGLTADEWRDNPRQSPRGDQYNTRKNT
RQTQAGLRYERQLSAQDDLSVMMYAGERETTQFQSIPRAPQLKPSHAGGVIDLTRHYQGIDTRLTHRGEL
LVPVTLTAGLDYENMSERRKGYENFVMVNGAPQYGEQGALRRNERNLMWNVDPYLQTQWQLTDKLSLD
AGVRYSSVWFDSNDYYITPGNGDDSGDASYHKWLPAGSLKYALTDAWNVYLSAGRGFETPTINELSYRS
DNQSGLNFGLKPSTNDTVEIGSKTRIGNGLFTAALFQTNTDNEIVVDSSSGGRTSYKNAGKTRRQGMEL
GLDQQFGESWRLKAAWTWLDATYRTNVCDDASCNGNRIPGIARNMGYASFGYQPEQGWYAGSDIRYM
SDIMANDENTAKAPSWTVVGLTTGYKWSYGRMDMDLFGRIDNLFDREYVGSVIVNESNGRYYEPAPGRN
YGIGLNLAWRFE
SEQ ID NO: 23
MKYTSHFPLGIVIPLLACSVPLQAAENMTEQSTPDESAATAENHEETMVITAARQNLQAPGVSTITAEEIRK
HPPARDVSELIRTQPGVNLTGNSTSGQRGIVNRQIDIRGMGPENTLVLVDGKPVTSRNSVRYGWRGDRD
SRGDTSWVPAEMIDHIDVIRGPAAARYGNGAMGGVVNIVTKPTTREWHGSWNTYMNAPQHRKEGATK
RTNFSLNGPLSDSVSFNLWGNLSKTQADAQDINAGHEAERTGSYAGSYPAGREGVVNKDIHSKLRWEFA
PMQALEFEAGYSRQGNLYAGDTQNTNTSTLVKSMYGKETNRLYRQTYGVTWTGGWDNGVTSNSYAQYE
HTRNSRMDEGLAGGTEGIFSSSEFSDIDLADVLLHSEVNIPFTLGVDQNLTLGAEWNQQRMKDGVSTTQ
ALSYGTIDGVSATGRSPYSSAEIFSLFTEDNMALTDSTMLTPALRFDHHSIVGNNWSPSLNLSQELTDDW
TLKLGIARAYKAPNLYQLNPNYILYSNGQGCYASSSACYLMGNSDLKAETSVNKEIGLEYKHDGYQAGIT
WFRNDYHNKIESGYAAVGTASNGTTNIYQWENVPKALVEGLEGTLNLPVGEAVNWSNNLTWMLQSKNK
TTGDRLSVIPQFTLNSTLSWQVREDLSLQSTFTWYGRQKPKRFNYKGEAVSGSELNEVSPYSIVGLSATW
DVNKNLSFTSGIDNLFDIRHYRAGNAQTTGNATTGAYLYGAGAETYNESGRTFFMSVNTHF
SEQ ID NO: 24
MEKNASLPFGSFNSLALFTGLCLGASPAAGIAAENSVKNSEETLVVEAAPPSLYSPGASADPKFNKPLVDT
TRTITVIPEQVIKDQGVTNLTDALKNVPGVGAFYAGENGSSTTGDAIFMRGVDTSNSIYVDGIRDIGSVTR
DTFNTQQVEVIKGPAGTDYGRSAPSGSINMISKQPRLDSGIDGSASIGSAWSRRGTLDLNQAFSDNAAF
RLNLMGEKTHDAGRDRIENERYGIAPSLAFGLDTPTRLYLNYLHVRQNNTPDGGIPTVGLPGYSAPSPKYA
ALNSAGKVDTSNFYGTDSDYDKSTTDSGTLRFEHDLTENTTVRNTMWSRVKQEYLLTAVMGGANNITA
PDINDVNTWSWSRLVNTKDVSNRILTNQTNITSTFNTGSIGHDVSAGVEFTRENQTNYGVNARTAPAVN
LYHPVSNLSIGGLDRNGANANGQTDTFGIYAFDTLTLTERIEINGGLRLDNYHTKYDSATACGGSGRGAIA
CPPGQSTGSPVTTVDTAKSGNLVNWKAGALYRLTEQGNVYVNYAISQQPPGGSSFALAASGSGNSANRT
DFKPQKAKSSELGTKWQIFDNRLLLSAALFRTDIENEVAANDDGTWSQYGKKRVEGYELSATGNLTPDW
TIIAGYTQQHATVIEGQNVAQDGSSALAYTPKHAFTLWTQYQATSDLSVGGGVRYVGSLRRGSDGAVGT
PDHTEGYWVADAKLGYRVNSNILDLQLNMYNLFDTDYVASINKSGYRYHPGEPRTFMLTANVHF
SEQ ID NO: 25
MATMYKSTPSAAWCKKRLLVTSLFAAIYQTSAIAADTSAVSGEAVDDTSEQMTVTAPAPVQKAGSEHSIS
ARELENKGANDFGSIMRYEPLISATGASGGSGNGKSGFDRGGYTGYNIRGMESNRVGIDVDGIAQPNAT
GRGYVGRAGLNTFGIGRDYIDPYMYGSVDIQSGATSTETANSAIGGNVSFRPKSADDYLRPGKTSAFGYR
SGYDSADRSWHNGVTVAGGDEFLRGILVYSRRDGQETENNSGTVDAYPANWHSDAFLASGIWQPNDE
HKLTSTFDYYHKTNHTHYDTWDSSGNSTIGTANQTSQTRRWGLSLKDDWTPMNDYLDSVSTKIYYQHT
EAHDWTYMPDSVTRKMQTVNSNYDTDTWGLQTALAKTLGRHDLSAGFNASTSKTQRPFSQSPIPSVYSE
IMQPEADSRSYTLGGFVQDKINFDLDSHNFAVIPGVRVVHQSTKPENLSDLAANSSVLSESSVANLYGKN
SDTQVLPSLTFQYDLTPRLMTYLQYQRGAQFPNASQLYGSWNLGSSYAGSQQYALIGNTDLKTETSDNLE
WGLKGEVTEGITLRTALFYNSYKNFIAYTRYTRANNPGQFTNVPSNIYTIYQAENRDKAYIYGGEISTKFNF
GTWFEQVDGLSATLALGYSEGKSKSSYSGDKYVDLDSVAPMKAIVGVAWDDPAKRYGTALTATFVKGK
QATATNRESYSNSGSAITDASSDYMRVPGYGMLDWTAYWQVAKNVRLNGGVYNLTDRKYWDYLSSRNI
ETGTNQDANDKALAVMPGRTWQLGVNVDF
SEQ ID NO: 26
MAMKKLLIASLLFSSATVYGAEGFVVKDIHFEGLQRVAVGAALLSMPVRPGDTVTDDDISNTIRALFATGN
FEDVRVLRDGDTLLVQVKERPTIASITFSGNKSVKDDMLKQNLEASGVRVGESLDRTTIADIEKGLEDFYY
SVGKYSASVKAVVTPLPRNRVDLKLVFQEGVSAKIQQINIVGNHAFSTDELISHFQLRDEVPWWNVVGD
RKYQKQKLAGDLETLRSYYLDRGYARFNIDSTQVSLTPDKKGIYITVNITEGDQYKFSGVQVTGNILAGHS
AEIEALTKVEPGELYNGAKVTKMENDIKKLLGRYGYAYPRVQSQPEINDSDKTVKLHVNVDAGNRYYVRKI
RFEGNDTSKDAVLRREMRQMEGAWLGSDLVDQGKDRLNRLGFFETVDTDTQRVPGSPDQVDVVYKVKE
RNTGSFNFGIGYGTESGVSFQAGVQQDNWLGTGYAVGINGTKNDYQTYTELSVTNPYFTVDGVSLGGRV
FYNDFDANDADLSDYTNKSYGTDITLGFPVNEYNTLRAGVGYVHNSLSNMQPQVAMWRYLNSMGQYPD
NTNDRNSFSANDFTFNYGWTYNKLDRGFFPTEGSRVNLNGKVTIPGSDNEYYKATLDTATYVPIDNDHQ
WVVLGRTRFGYGDGIGGKEMPFYENFYAGGSSTVRGFQSNTIGPKAVYFPASSRHDDDDSYDNECKSTE
SAPCKSDDAVGGNAMAVASLELITPTPFISDKYANSVRTSVFWDMGTVWDTHWDSSAYAGYPDYSDPS
NIRMSAGIAVQWMSPLGPLVFSYAQPFKKYDGDKAEQFQFNIGKTW
SEQ ID NO: 27
MTDVTIKALASEIQTSVDRLIQQFADAGIRKSADDSVTSQEKQTLLTHLNREHGSAPDKLTLQRKTRSTLN
IPGTGGKSKSVQIEVRKKRTFVKRDPQEAERLAAEEQAQREAEEQARREAEEAAKREAQLKAEREAAEQA
KREVADKAKREAAEKDKVSNQHTDEMTKTAQAEKIRRENEAAELKRKSEEEARRKLEEEARRVAEEARR
MAEENEKNWSETSDSPEDSSDYHVTTSQHARQAEDDNDREVEGGRGRSRSSKAARPAKKGNKHAESK
ADREEARAAVRGGKGGKHRKGSALQQGFQKPAQAVNRDVVIGETITVGELANKMAVKGSQVIKAMMKL
GAMATINQVIDQETAQLVAEEMGHKVILRRENELEEAVMSDRDTGAAAEPRAPVVTIMGHVDHGKTSLL
DYIRSTKVASGEAGGITQHIGAYHVETDNGMITFLDTPGHAAFTSMRARGAQATDIVVLVVAADDGVMP
QTIEAIQHAKAAQVPVVVAVNKIDKPEADPDRVKNELSQYGILPEEWGGESQFVHVSAKAGTGIDDLLDA
ILLQAEVLELKAVRNGMASGAVIESFLDKGRGPVATVLVREGTLHKGDIVLCGFEYGRVRAMRDELGREVL
EAGPSIPVEILGLSGVPAAGDEVTVVRDEKKAREVALYRQGKFREVKLARQQKSKLENMFANMTEGEVHE
VNIVLKADVQGSVEAISDSLLKLSTDEVKVKIIGSGVGGITETDATLAAASNAILVGFNVRADASARKVIEA
ESLDLRYYSVIYNLIDEVKAAMSGMLSPELKQQIIGLAEVRDVFKSPKFGAIAGCMVTEGTIKRHNPIRVLR
DNVVIYEGELESLRRFKDDVNEVRNGMECGIGVKNYNDVRVGDMIEVFEIIEIQRSID
SEQ ID NO: 28
MKRMLINATQQEELRVALVDGQRLYDLDIESPGHEQKKANIYKGKITRIEPSLEAAFVDYGAERHGFLPLK
EIAREYFPANYNAHGRPNIKDVLREGQEVIVQMKEERGNKGAALTTFISLAGSYLVLMPNNPRAGGISRRI
EGDDRTELKEALASLELPDGMGLIVRTAGVGKSAEALQWDLSFRLKHWEAIQKAAESRPAPFLIHQESNV
IVRAFRDYLRQDIGEILIDNPKVLELARQHIAALGRPDFSSKIKLYTGEIPLFSHYQIESQIESAFQREVRLPS
GGSIVIDSTEALTAIDINSARATRGGDIEETAFNTNLEAADEIARQLRLRDLGGLIVIDFIDMTPVRHQRAV
ENRLREAVRQDRARIQISHISRFGLLEMSRQRLSPSLGESSHHVCPRCSGTGTVRDNESLSLSILRLIEEE
ALKENTKEVHAIVPVPIASYLLNEKRAAVSAIESRQGDVRVIIVPNDEMQTPHYSVLRVRKGEETSTLSYLL
PKLHEEEMALPGDDEPAERKRPEQPALAAFVMPDAPPAPMLEEPAAAPVAAAAPVAAAAPAQPGLLSRFFS
ALKNIFSGAEEAKPAEVQVEKKAEEKPERQQERRKPRANNRRDRNDRRDNRDNRDNRDNRDNRDTRAD
NAEGREPRESREENRRNRREKPSQNVEARDVRQTSGDDAEKAKSRDEQQPRRERTRRRSDDKRQAQQ
EAKAQTREEPVVQETEQEERVQTLPRRKPRQLAQKVRVESAVVEPVAEIVPEAVVAEVIAPHSEPVKAELP
AGVESVADQDENGESREANGMPRRSRRSPRHLRVSGQRRRRYRDERYPTQSPMPLTVACASPEMASGK
VWIRYPVVRPQDQQPEEVQVQDASVAKTVEAVAAPVAVVETVTAAPVTVEPATMEPVTAEPVVVEPVAAA
EPLVVDAAEVVAPAAVEPAPQEPVTEAPAVEAPQAIAPVTLDAEPVVVEPEAVETTPVVAAPVETIAPVAETV
EQAPVTEAAPAEPVKAEPPVSKPVVVAGHRHATAPMTRAPAPDYVPEAPRHSTWVRPPFAFEGKGAAGGH
SATHKATAEPTRPQPVE
SEQ ID NO: 29
MRKLSLSLLILSLGVALLPLAQAATTPAQEHLLEQVRLGEASNREDLVRQSLYRLELIDPNNPELIAARMRY
LLRQGDAAGAQKELERLTKQAPDSPELKASRNIEMKSNTGEGRQALQQARLLGVAGKVDEAIAAYEKLYG
GVPDDVDVAIEYWTLVARLPARHSEGVSQLKKLNASAPGNVSLLTSLAKQMFADNIKPQEGFAYLAEMAR
SASGRGIAADMWFSEVKSMPVSKASVQALQQFLLQFPTGSVAANARVLLDQQQAQLQDPTFRARSEGLA
AVKSGNTTQAVADLQKAVQADSRDSDAVGALGQAYSQRGDRARAVAQLSKAIAMDPDSPNIRGKWDSL
LQTNRYWLLIKQGDNALKAGQLSQAQNYYAQAQRVDRTDSYAVLGLGDVAAARKEAAAAERYYQQALRL
DRGNNLAVRGLANLYRAESPEKASAWIAGLPPAQRRSIDDIERSLTNDRLEKQAQALESQGNWAQAAEV
QRRRLALDPDSVWITYRLARDLVSAGERQEADALMRTMVNRQPQDAERVYASGLYLSGNDQDDLALAQI
AALPRSAWTDNIRELEARLQSDRVLRQANQLRDSGNEAQAIALIRQQPASVRYDLTLADWAQQRGDSQT
AIANYQRVLRQEADNGDARLGLAEVYLAEGDKPAARAQVMQLKGAETESMNMQRRVALARAGLGDTAD
AQRIFNQIVPQAKAQPPSMESALVLRDAARFATQSGAPQQALTHYREAMVASGITPAQPQDNDTFTRLTR
NDSHDDWLKRGIRSDAADLYRQQDLNVTLEHDFWGSSGTGGYSDLKAHTIMLQMDAPLADGRMFFRT
DLVNMDAGSFSTHSDGSYSPSWGTCGEIACTSGSKNQTDSGASVAVGWKNDTWSGDIGTTPMGFNVV
DVVGGLSYSSDVGPVGYTVNVHRRPISSSLLSFGGQKDSSSHTGATWGGVRADGGGLSLSYDRGEAHG
IWSSLGADSLTGKNVADNWRVRWMTGYYYKVINENNRRVTVGLNNMIWHYDKDLSGYTLGQGGYYSP
QEYLSFAVPVTWRQRTENWSWELGGSVSWSHSRTQTQARYPLLNLIPSDYRQRASELTEEGSSSHGFGY
TARALVERRVTSNWFVGAAVDIQQAKDYTPSHALLYVRYSAAGWQGDLDMPPQPLVPYADW
SEQ ID NO: 30
MSQEYTEDKEVKLTKLSSGRRLLEAMLILCSLFAIWLMAALLSFNPSDPSWSQTAWHEPIHNLGGAPGAW
LADTLFFIFGVMAYTIPVIIIGGCWFAWRHQENDEYIDYFAVSLRLIGALALILTSCGLAAINADDIWYFASG
GVIGSLLSTTLQPLLHSSGGTIALLCIWAAGLTLFTGWSWVSIAEKLGGGILSVLTFASNRTRRDDTWVDE
GEYEDDEEEYDDEEAARPQESRRARILRSALARRKRLAEKFTNPMGRKTDAALFSGKRMDDGEEVVQYS
ASGAPVAADDVLFSGASAARPAEDDVLFSGASAVRPGDFDPYDPLLNGHSIAEPVSAAAAATAAPQAWA
ESPVGHHGAAPAYQPEASYPPQQAYQPEPAPFQQAYQPEPAPFQQAAYQPPAGQTAPQAYQPEPAPYQQP
VYDPRAGQPAPQAYQPEPAPYQQPAYDPYAGQPAPQAYQPEPAPYQQPAYDPHAGQPAPQAYQPEPAPY
QQPAYDPYAGQPAPQAYQPEPAPYQQPTYDPYAGQPAPQTYQQPAYDPNAGQPAPQPYQPEPAAYQPQS
APVPPPEPEPEVVQEEVKRPPLYYFEEVEEKRARERELLASWYQPIPEPESPIATKPLTPPTTASKPPVETTVV
SAVAAGVHQATAASGGAAAATSSTAASAAATPLFSPASSGPRVQVKEGIGPKLPRPNRVRVPTRRELASY
GIKLPSQREAEQRARQAERDPHYDDELLSDEEADAMEQDELARQFAATQQQRYGHRWEDDNATDDDEA
DAAAEAELARQFAATQQQRYATEQPPGANPFSPADYEFSPMKTLVNDGPSEPLFTPTPEVQPQQPAQRYQ
QPAAAPQQGYQPAQHQPIHHQPVPPQPQSYPTASQPVQPQQPVAPQGHQPAAPAPQESLIHPLLMRNIGD
SRPLQKPTTPLPSLDLLTPPPSEVEPVDTFALEQMARLVEARLADFRIKADVVNYSPGPVITRFELNLAPGVK
AARISNLSRDLARSLSTVAVRVVEVIPGKPYVGLELPNKKRQTVYLREVLDNAKFRDNPSPLTVVLGKDIA
GDPVVADLAKMPHLLVAGTTGSGKSVGVNAMILSMLYKAQPEDVRFIMIDPKMLELSVYEGIPHLLTEVVT
DMKDAANALRWSVNEMERRYKLMSALGVRNLAGYNEKIAEAARMGRPIPDPYWKPGDSMDAVHPVLEK
LPYIVVLVDEFADLMMTVGKKVEELIARLAQKARAAGIHLVLATQRPSVDVITGLIKANIPTRIAFTVSSKID
SRTILDQGGAESLLGMGDMLYSGPNSTTPVRVHGAFVRDQEVHAVVQDWKARGRPQYVDGITSDSESE
GGGGGFDGGEELDPLFDQAVNFVTEKRKASISGVQRQFRIGYNRAARIIEQMEAQGIVSEQGHNGNREV
LAPPPF

The nucleic acid fragments of the present invention have or derive from the following sequences:

SEQ ID NO: 31
ATGAAGAAGTTAGCTTTACTCTCCGCCGTAATGACGCTTGGAATGTCGTCATGGGCTTTTGCTGCCGA
CAACCCGCCGCCGCCGCCGGAAAAAGGCGCGCAGCATCAGGGTAAACCGCCGGTGAAAAACGGCCA
ACACGAAGGTAAGCAAGCGCAATACAACAGAAAACAGCCACAACGAGACGGCAAACAGCCGCAGCAC
GACGGTAAACAGCCGCAGCACAACGGCAAGCAGCCGCCAAAAGGGAGCGAGCACAGCGGGAAACCG
CTGCCGCCGAAAGCGTAA
SEQ ID NO: 32
ATGAAACGTTACGCAACCGCACTGCTCTTTTGCACTCTGTCGCTGACCAGCCTGGCCGCTCGCGCCGA
TATTATCGATGACGCGATCGGCAATATTCAGCAAGCCATTAACGACGCCTATAACCCCGGCAGCAGTC
GCTCCGATGACGACGACAGATACGATGACGATGGCCGGTATGATGACGGGCGCTATCAGGGGAGCC
GTCAGCAGAGCCGTGACAGTCAGCGCCAGTATGACGAGCGGCAACGCCAGCTGGACGAGCGCCGCC
GCCAGCTGGATGAACGCCAGCGTCAGCTCGACCGCGATCGTCGTCAGTTAGAAAGCGACCAGCGTCG
TCTGGATGATAGCTACTGA
SEQ ID NO: 33
ATGTTCAGGTCACTGATTCTGGCAGCAGTACTGCTGGCCGCAGGGCCACTGGTCGCTAACGCTGGTG
AAATCACCCTGCTGCCATCGGTAAAATTACAAATAGGCGATCGTGACAATTACGGTAACTACTGGGAC
GGTGGCAGCTGGCGCGACCGTGATTACTGGCGTCGTCACTATGAATGGCGTGATAACCGTTGGCATC
GTCATGACAACGGCTGGCACAAAGGCTGGTACAAAGGCAGAGATAAAGCCTGGGAGCGCGGCTATC
GTGCTGGCTGGAACGACCGCGATGACCACCGCGGCGGCTGGGGTCGCGGCCCGGGCGGGCGCGGT
CACGGTCATGGACATGGCCATCACTAA
SEQ ID NO: 34
ATGAAGGAAATCGGCTTACCGTTATTGCTACTGACCGCGCTGGCCAGTCCGGCTTTTGCTGCAGACTG
TCAGCCAAACGGCATTGGCGGCTCGTTTTGCATTAACGATGACGGTACGACTACCGACACGGTGCCTA
ACGAAGTCAACGGCATGGATACGTACTCGAATAATGGCGGCTATACCAGTTCCCTGCCCGATCGGTCA
GGGGCGGATGAAGCACTGGAAGGTTCATCGCTGTCGACGCAGCAAGGCGTCGGCAGCGGACAGAGC
GACAGTGCGCTGGCGGGTCGCGACTGGCATTCGCCCGCCAATCTGAATGATGGCGCCGCCACCTCCA
GTATGAGCCTGCTGGATAAACCCTGA
SEQ ID NO: 35
ATGAATATGAAAAAACTGACGACCCTTTTGCTCACCGCCACCTTAGGTCTTGCCAGCGGCGCGGCCCT
GGCGGCAGACACCGGCGCCCAGTCCAATAATGGCCAGGCCAACTCTTCCGCGGATGCCGGTCAGGT
GGCGCCGGATGCCCGTGAGAACGTGGCGCCGAACAACGTGGACAATAGTCAGATCAACTCTGGCTCT
GGGGGCACCACGGGCTCGACGATGACCCAGGATAATATGTCGAGCAATGAGGTACATAAAAACTCGA
TGTGTAAAGACGGCCGCTGTCCGGACACCGGTAAAAAACTGGACAACGGTGGCAATACGACCCAAGA
CAACAGCAAAACCGACGGCACCACCCAGTAA
SEQ ID NO: 36
ATGAAACACCGCATCGCTCTGCTTCTGGTCCTGACTTCACTTAGCGCCAGCGCCCTAGCCGCCTCTCC
CTGCCAGGAAAAAGAGCAGGATATTCAACGAGAGATCAGCTACGCCGAAAAGCATCATAATCAAAGTC
GCATTGATGGGCTAAATACCGCGCTACGTCAGGTTCGGGAAAACTGTAGCGACAGTAAACTCAAAGC
CGATCATCAGCAAAAAATTGCCAAACAGCGGGAAGAGATCGCTGAACGTCAGCGCGATCTGCAGGAA
GCCCGGAAGAAAGGCGATGCGGACAAAATTAACAAACGCCAGCATAAACTCAATGAAGCGCAACAGG
AGTTAAAAACGCTGGAGTCTCGGGATTACTAA.
SEQ ID NO: 37
ATGCGACTCATAACACGACACGTGAGAGAGGATATTATGAAAAAAGCAATGATTGCGTTATCGGCTAT
TCTGGTTGCGGCTCCGGTTTTTGCTGCGACAACACATGCAACAGATGATACCGTCGCGGCGGCGAAT
GCCAACGCCAACACCGCTAAAGAGAAGCTGCATCAGGCCCAGCACGAGGGCGAAGAGCAGCAGCTG
AAGGCGAAACACGCCGCCGAAGGCAAGCAGGACAGCGTCGGCAGCCAGGTGAGCGAAGGCGCGCA
GAAAACCTGGAACAAGACCAAAGAAGGCACCGAGAAGGGGTGGGATAAGACCAAAGAGGTCAGTGA
AAAAGGCTGGAACGCCACCAAATCCGGTGCGGAAAAGGGCTGGGATAAAACCAAAACCGGCGCCGA
AGAGTTAAAAAATAAAGTGACTGAATAA
SEQ ID NO: 38
ATGAAAAAGATGATTTCTCTGGCAGTAATTTTATCCTGTGTGCTGAGCGTCCCGGCCTTTGCCGATGG
CCCGAACGACGGCCATCGCCCGGAGCAGCCCACGGTGTGGCAGAACGGTCCGGACCATGACGGGCA
TGCACCGCAGGGCGGACCTGACGCGCATCATCAGGGCGACCATGACCAGCGTGGCCCGGATCGCGA
CGGCCATGACAAACGCGATCTGGCACGTCATGAGCAGGACCATTTCGCCTGGCGCGGGAACGATTTC
CGCAAAGGCCACCCGGCTCCGGCGCCGTTCCGTGGCGATGAATATCGCGTCCGCGACTGGAGCGAC
CGCGGCCTGCCGCCCCCGCCGGAAGGCCATCACTGGTCCTATATCGACGGTAACTATGTGCTGATCG
CCGCGGCGACCGGGATCATCACCTCGATTCTGGTGAGCGGCGCCCTCGGCCACTAA
SEQ ID NO: 39
ATGAAAAAACCGACATCCGCCACCCGTGGCAAATCCGGCCGCAAGTCGCGTGAAGAGTTAAATCAGG
AAGCTCGCGATCGCAAACGGCAGAAGAAACATCGTGGCCACGCGGCAGGCAGTCGCGCGAACGGCG
GCGATGCGGCTTCAGCGGGTAAAAAACAGCGTCAGGCGCAAGATCCGCGCGTGGGTAGCAAAAAAC
CGATCCCGCTGGGCGTGAGCGAAAGCAGCGTTCCAGCTCCCAAGCAGCATAAACCAAAGAGCGAGAA
ACCTATGCTTTCACCGCAGGCTGAGCTGGAGTTGCTGGAGAATGATGAGCGCCTGGACGCGCTGCTG
GAACGTCTGGAAGAGGGCGGCACCCTGAATGCTGAAGAGCAGAGCTGGGTGGACGCCAAACTGGAT
CGCATTGATGAGCTGATGCAGCAGCTCGGCCTCTCTTACGATGATGAAGATGAAGAAGAGGAAGAGC
GTCAGGAAGATATGATGCGTCTGCTGAAGGGTGGAAACTAA
SEQ ID NO: 40
ATGGCGAGTAAGTTTCAGAACCGTTTAGTCGGGACAATCGTGCTGGTGGCGCTGGGGGTGATTATCC
TGCCAGGGCTGCTGGACGGGCAGAAAAAGCATTACCAGGATGAGTTTGCCGCGATCCCGCTGGTACC
GAAACCAGGCGATCGCGATGAACCGGATATGTTGCCGGCGGCAACCCAGGCGTTGCCTTCGCAACCG
CCGGAAGGGGCGGCGGAAGAGGTGCGGGCGGGCGATGCCGCCGCGCCATCGTTAGATCCATCGCG
TATTCCGGTGAACAGCAACAGCTTCGATGACGTTCAGGAGCCGGTGGTGGCCGCGAAACCGCAGCCC
AAGCCGCAGCCGAAACCGCAGCCGCAACAGCAGGCCTCGACGCCAACGCCGCCGCCGGCTAAGCCA
CAGCAGCAACAGCCACCGCAGCAGCAGGCGGCCCTGCCGGCGCCGACCGGCAAAGCCTATGTGGTT
CAGCTGGGCGCGTTGAAGAACGCCGATAAGGTGAATGAGATTGTCGGTAAACTGCGGGCCTCGGGTT
TCAAAGTCTATACGTCGCCTTCGACGCCGGTACAGGGTAAAATTACCCGCATCCTCGTCGGCCCGGAT
GCGTCAAAAGACAAGCTGAAAGGCCAGCTGGGCGATCTGCAGCAGATCTCCGGGCTTAGCGGGGTG
GTGATGGGCTTCACCCCGAACTGA
SEQ ID NO: 41
ATGGCACAACGAGATTATGTACGCCGCAGCCAACCGGCTTCTTCGCGGCGCAAAAAGAGCACGACCC
GAAGCTCAAGGAATAAGCAAAGCAGCCTTCCGGCGATTTCACCGGCGATGGTGGCGATCGCGGCGG
CTGTGCTGGTGGCCTTTATCGGTGGCCTCTATTTCATTACGCATCATAAGAAAGAAGAAGCGGAAGCG
ATGCAAAATCGCCAGGCCGCCGGCAACGGCTTGCCGCCCAAACCGGAAGAGCGCTGGCGCTATATTA
AAGAGCTGGAAAGCCGCCAGCCTGGCGTCCGCGCGCCGACCGAACCGACCGCCGGTGGCGAAGTCA
TGAAACCGGAACAGCTGACCGACGAGCAGCGCCAGCTGCTCGCCCAGATGCAGGCCGATATGCGCC
AGCAGCCGACCCAGCTGACCGAAGTGCCGTGGAACGAACAAACGCCGGCGCAGCGCCAGCAGACGC
TTCAGCGTCAGCGTTTAGCGCAGCAACAGCAGCAGGCGCAGCAGCAACAGTGGGCGCAGACTCAGG
CGCAGACCGTCCAACAGCAGCCGCCGCGCGTTCAGCAGCCGAAGCCGGTTCAGCAGCAACAGCCGA
AGCAGACCGCGTCAAACCAGCAGCCGTACCAGGATCTGCTGCAGACGCCAGCGCATACCAATACCAC
GCAGCCGCGTACCCAGGCCGCGGCGCCGGTAACTCGGGTGGAAGAAGCGCCGAAAACCACCGCCGA
GAAGAAAGACGATCGTAGCTGGATGATCCAGTGCGGCTCTTTTAAAGGCGCCGAGCAGGCCGAAACC
GTCCGCGCTCAGCTGGCTTTCGAAGGGTTTGCTTCGCACATTACCACTAACAACGGCTGGAACCGCGT
GGTTATTGGCCCGTTGAAAGGCAAAGAAAGCGCCAACGAGATGATCACCCGCCTGAAGATGGCTGGA
CACGCGAACTGCATTCGTCTCGCCGCCAGGGGTTGA
SEQ ID NO: 42
ATGAGCGCGGGAAGCACCAAATTTACCGTCAGCCGTATTGCGGCTCTTTCACTGGTTTCACTCTGGCT
GGCCGGGTGTACCAACACCAATAATCCGCCTGCGCCGGTTAGCTCTGCCGGCGGCGCCGCCTCTTCC
AGCACCAACTCCGGCATGCTGATTACGCCGCCACCCTCCGGCGTCAAGTCCGCTCCTCAGGCGCAGC
CGATTCAGCCGATGCAGACCCAGACCATTCAGCCGGCGCCGGTGGCGCAGGAGCCGGTACAGACGG
TAAATGGCCGGATCGTTTACAACCGCAAATATGGCGATATTCCGAAAGGTAGCTATACCGGCGGCAGT
ACCTATACGGTAAAACGCGGCGACACGCTATTCTATATCGCCTGGGTCACCGGCAACGATTTCCGCGA
CCTGGCGCAACGTAACAATATCCCGGCCCCGTACGCGCTGAACGTGGGGCAGGTACTGCAGGTCGGT
AACGCCTCAGGCCAGCCGATCACCGGCGAAAACGCCGTTTCTCAGGCCAGCGCAAGAGCGAGCGGC
GGTGCGACGACCAGCACAACTTCTGCACAAAAATCGACCGCGGTGGTTGCTTCACAACCGACTATTAC
GTATTCTGAATCTTCAGGTGAACAGAGTGCTACCAAGATGTTGCCTAATAATAAACCAGCGACCACAA
CCACAACGGTTGTCGCGCCGGTGACGGCACCAACAACGGTGAGCACAACCCAGCCGACTGCAAGCA
GTACGTCAACCAGTTCGCCGATCTCAGCATGGCGCTGGCCGACTGATGGCAAGGTTATCGAGAACTTT
AGCGGCGCGGAAGGCGGCAATAAAGGCATCGATATTGCAGGCAGTAAGGGACAGGCTATTGTCGCG
ACCGCCGATGGGCGCGTCGTCTATGCCGGTAACGCACTGCGCGGCTACGGTAATCTTATTATCATCAA
ACACAACGATGATTACCTGAGTGCCTACGCTCATAACGATACCATGCTGGTTCGGGAGCAACAGGAAG
TCAAAGCGGGGCAGAAAATCGCTACCATGGGTAGCACCGGAACCAGCTCAACAAGATTACATTTTGAA
ATTCGTTACAAGGGGAAATCCGTCAACCCGCTGCAGTACTTACCGCAGCGATAA
SEQ ID NO: 43
ATGCGTAAGCAATGGCTGGGGATCTGCATAGCAGCGGGGCTGCTGGCGGCATGTTCGAGTGATGAC
GTGCAACAAAAAACGGTCAGTACTCCACAGCCGGCCGTCTGTAATGGCCCGACGGTTGAGATCAGCG
GCGCCGATCCGCAGTATGAAACGCCGAACGCCACGGCGAATCAGGATTATGAGCGCGACGGTAAAA
GCTACAAAATCGTTCAGGATCCGGCCAACTTTACTCAGGCCGGTTTCGCGGCGATCTATGACGCAGAA
CCCAACAGCAACCTGACCGCCAGCGGCGAAGCCTTCGATCCGACTCAGTTGACCGCAGCGCACCCGA
CGCTGCCGATCCCGAGCTACGCGCGGATCACTAACCTTGCCAACGGACGGATGATCGTCGTGCGGAT
TAACGATCGCGGTCCCTATGGCAACGATCGGGTCATCTCGCTTTCCCGCGCATCCGCTGACCGCCTGA
ACACCTCCAACAACACCAAAGTGCGCATCGACCCCATCATCGTCGCGCCTGACGGTTCGCTTTCCGGC
CCGGGGATGGCCTGTACCACCGTCGCCAAACAGACTTACGCCCTGCCCGCCCGTCCGAATCTGGACG
GTGGGGACGCCGCTGGCATGAGCCAGCCCGCGCCCACTGACGTTCGCCCGATCAGCAACAGCACGC
TGACGCCGGCAGACAGCGTGGGCGCGCCGGTGAACAGCGGCGGTTTCCTCGGCGCGCCGACGCCCC
TGAACAACGGCGTGCTGGAGAGTAGCGAACCAGCGGCAGCCGCCGCGACGGCTCCTGCCGCCGGCG
CCACGCCAACAGCGCCAGTGACCGCGCCTGGCTCCATTCAGGGTAATGTGGTGCCCGCTGCGGCCAC
CGCCGCAGCCGCTGGCGCCGTGGCGGCCTCGTCCTCCGCGACCTCCAGCGCCAGCGGTAATTTTGTT
GTCCAGGTGGGCGCAGTAAGCGACCAGACGCGGGCGCAGCAGTATCAGCAGCGCCTGAGCCAGCAG
TTTTCTGTGCCAGGCCGGGTCATGCAAAACGGCGCGGTCTGGCGTATTCAGCTGGGTCCCTTTGCTG
ATAAAGCACAGGCCAGCGCCGTGCAGCAGCGCCTGCAAAGCGAAGCGCAGCTGCAGTCCTTTATTAC
TCGCGCCAACTAA
SEQ ID NO: 44
ATGGATGATTTCAAACCAGAAGACGATATGAAAGCCGATCGCAACGATCGTCGTGCTGGTCGTTCCCG
TCAGTCTTCCGAGCGTGATGCCGATCCGCAGATCAATTTTGACGATGTTGATCTTGATGCCGATGAAG
GCCGTCCGACGCGCGCTGGTAAGGCCCGTCGCGAGCGTGAAGAGGAAGAGTTCGAAGAAGAACTGG
ATGCGCAAGACGAGGAGATGCTCGAAGAGCAGCCTGTAGAGCGTCGTCCGCGCAAGCGTAAAAAAG
CGCCGGCCAAACCGGCCTCCCGCCAGTACATCATGATGGGTGTGGGGATTCTGGTGCTGCTGCTGTT
GATCGTGGGTATCGGTTCCGCACTGAAATCGCCATCATCTTCCAGCCAGCAGACCGCTTCCGGCGAG
AAGAGCATTAATCTGTCTGACGACCAGTCCGCCAGCATGCCTGCTGCCGGCCAGGACCAGACTGCCG
CCGCTAACAGCACCTCACAGCAGGACGTAACGGTACCGCCTATTGCCGCGAACCCGACGCAGGGCCA
GGCAGCGGTTGCGCCGCAGGGCCAGCAGCGTATCGAAGTTCAGGGCGATCTGAACAATGCCTTGAC
CCAGCAGCAGGGCCAACTGGACGGCGCCGTGGCTAACTCGACGCTGCCGACTGAACCGGCTACCGT
CGCGCCAATCCGGAATGGCGCCAATGGCACCGCGGCGCCGCGCCAGGCGACCGAGCGTCAGACAGC
AGCGACCCCGCGTCCGGCTGAACGTAAGCATACCGTTATCGAAGCGAAGCCGCAGTCGAAGCCACAG
GCCGTGGCGAAAACGCCGGTAGAATCGAAGCCGGTCCAGCCGAAGCATGTTGAAAGCACGGCGACC
ACCGCTCCGGCGAAAACGTCCGTCAGCGAAAGCAAACCGGTGGCCACCGCTCAGAGCAAACCGACCA
CGACGACCGCAGCGCCAGCGGCAACGGCAGCTGCGGCAGCGCCGGCAGCGAAGACCGGGAAGACG
GCAGGTGACGTCAGCTCAATGAAAACTGCGCCGTCGGGTCACTATACTCTGCAGCTCAGCAGCTCCTC
TAACTACGACAACCTCAACAACTGGGCGAAGAAAGAGAAGCTGGATAAATATGTTGTCTATGAAACGT
CGCGTAACGGCCAACCATGGTACGTGCTGGTGAGCGGTATCTATGCATCGAAAGATGAAGCGAAACG
TGCTGTCACCTCGCTGCCGGCGGACGTGCAGGCGAAAAATCCATGGGCAAAACCGCTGCATCAGGTT
CAGGCTGACCTGAAATAA
SEQ ID NO: 45
ATGTCAAAGGCAACCGAACAAAACGACAAGCTTAAACGAGCGATCATCATTTCAGTCGCGCTGCACAT
CATTCTGATCGCGCTGCTGATCTGGAGTTCGTTTGACGAGCATCTGGATGCCTCTGCCGGCGGCGGC
GGCGGATCGTCGATTGATGCCGTCATGGTCGATCCGGGGGCGGTGGTAAATAACTATAACCGTCAGC
AACAGCAGCAGGCCAGCGCACGTCGCGCCGCTGAACAGCGTGAAAAACAGGCGCAGCAGCAGGCGG
AAGAGTTACGTGAGAAACAGGCGGCGGAACAGGAACGGCTGAAACAGCTCGAACAGGAGCGGCTGC
AGGCGCAGGAAGCGGCGAAAGAAGCGAAGGAGCAGCAGAAGCAGGCTGAAGAAGCGGCTGCCAAG
GCCGCCGCGGCGGCAAAAGCCAAAGCGGACGCACAGGCAAAAGAAGCGCAGGAAGCCGCTGCCAAA
GCGGCCGCCGAGGCGAAAGCGAAGGCGGATGCCCAGGCGAAAGCGGCAGAACAGGCGGCGGCCAA
GGCGGCTGCTGACGCGAAAAAGCAGGCCGAAGCCGCTGCAGCGAAAGCCGCTGCCGAGGCGAAGAA
ACAGGCGGAAGCTGAAGCGGCGAAAGCTGCGGCCGAGGCGCAGAAGAAAGCGGAAGCGGCGGCTG
CGAAGAAAGCGCAACAGGAAGCGGAGAAAAAAGCCCAGCAGGAAGCGGCTAAGCAGGCGGCAGCTG
AAAAAGCGGCTGCCGAAAAAGCCGCTGAGAAAGCCGCCGCGCAAAAAGCGGCCGCTGAGAAGGCCG
CCGCCGAGAAAGCCGCAGCCGCTGAAAAAGCGGCGGCAGCGAAAGCGGCTGCAGCAGAGAAGGCT
GCAGCTGATAAAGCGGCCAAAGCGGCAGCAGCAAAAGCCGCGGCGGCGAAGAAAGCGGCGGCTGC
GAAAGAAGCGGACGGCGTTGACAACCTGCTCGGCGATCTGAGTTCTGGTAAGAATGCGCCTAAAACA
GGCGGTGGGGCCAAAGGAAACAATGCCTCCGCTGCCGGGAGTGGTAATACTAAAAACAGTGCCTCAG
GGGCTGATATCAACAACTATGCCGGACAGATAAAATCGGCGATTGAAAGTAAGTTTTATGACGCATCG
TCCTATGCGGGCAAAACATGTACCTTGCGTATCAAACTTGCTCCTGACGGCCTGTTGTTAAATATACAG
TCCGAAGGTGGTGATCCTGCTCTGTGCCAGGCCGCTCTTGCCGCAGCCCGACAGGCTAAGTTTCCGA
AACCACCTAGCCAGGCAGTATATGAAGTCTTCAAAAATGCGCCACTGGACTTCAAACCTCAGTGA
SEQ ID NO: 46
ATGTTTTTTTTAAGTATTTTTTACATGGAGATGACAAAAGTGAAATTAAGCGCTCTGTTTATTGCCCTGA
TTCCTCTACTGGGCTCGCCGGTTATTCATGCAGAAACTACTGCTGCGCCGGTTCTGGAAAATCGCGCT
GCGCAGGGAGATATCACCACTCCTGGCGGCGCGCGTCGTTTAACAGGCGATCAAACCGAAGCGCTGC
GCGCCTCGTTAATCAATAAGCCAGCTAAAAACGTTATTTTGCTGATTGGCGATGGCATGGGTGATTCG
GAAATTACCGCTGCGCGAAACTATGCCGAGGGGGCGGGCGGTTTCTTTAAAGGAATTGATGCTCTGC
CGTTAACCGGGCAGTACACGCATTATTCGCTGGATAAAAAAACCGGGAAACCGGACTACGTGACCGA
CTCGGCGGCCTCCGCCACCGCCTGGACCACCGGCGTGAAGACTTATAACGGCGCGCTGGGCGTCGA
TATTCATGAGAATGCGCATCAGACCATCCTCGAGCTGGCGAAAGCGGCGGGGCTGGCCACCGGCAAC
GTTTCCACCGCCGAGCTGCAGGACGCCACCCCCGCGGCGTTGGTAGCGCATGTGACATCGCGTAAAT
GCTACGGCCCGACGGTCACCAGCGAAAAATGCCCCAGCAATGCGCTGGAAAAAGGGGGCAAAGGCT
CCATTACCGAACAGCTGCTGAACGCCCGACCGGATGTCACCTTGGGCGGCGGCGCGAAGACCTTTAC
CGAAACGGCGACGGCGGGCGAGTGGCAGGGCAAAACCCTGCGCGAGCAGGCGCAAGCGCGCGGCT
ACCAGATTGTGACCGACGCGGCTTCTCTTGCCGCCGCGACGGAAGCCAGTCAGGATAAACCGCTGCT
GGGACTCTTTGCCGATGGCAATATGCCGGTACGCTGGGAAGGGCCGAAGGCGTCTTATCACGGTAAT
ATCGATAAGCCGCCGGTGACCTGTACGCCAAACCCGAAGCGTGACGCCTCGGTGCCGACGCTGGCG
CAGATGACGGAGAAAGCGATTGACCTGCTCAGTCGCAACGAGAAAGGTTTCTTCCTGCAAGTCGAAG
GCGCTTCCATCGATAAGCAGGACCATGCGGCGAATCCGTGCGGCCAGATCGGCGAAACGGTTGATCT
TGACGAAGCGGTGCAGAAGGCGCTGGAATTCGCGCGAAAAGACGGTAATACCCTGGTGATCGTCACC
GCCGACCATGCGCATGCCAGCCAGATCATCCCGGCGGATAGCAAAGCCCCGGGGCTGACCCAGGCT
CTGAACACGCACGATGGCGCGGTGATGGTGATGAGCTACGGCAACTCTGAGGAAGAGTCGATGGAG
CACACCGGCACCCAACTGCGCATTGCGGCCTACGGTCCGCATGCGGCTAACGTCGTAGGCCTGACCG
ATCAGACCGACCTGTTCACGACCATGAAAGCTGCCCTGAGTCTCAAATAA
SEQ ID NO: 47
ATGTCACTGCCGTTCAAACCCCATATTATCGCCCTGCTCTGTAGCGCTGGCTTACTCGCGGCGGCAGG
AACACTCTATGTGCAAAGCCGAACCCCAGCGACGATCGCTGAACCGCCTGCGCAGCAAGCGCCAGCG
CCCGCAGCGTCGACGACACAGCCGGTGGCCGCCACTTACACCCAGGCGCAAATTGATCAGTGGGTCG
CCCCTATCGCGCTCTACCCGGACAGCCTGCTGTCGCAGGTGTTGATGGCCTCCACTTATCCCGACAAC
GTCCTGCAGGCGGTCCAGTGGTCCCAGGATAACCCCGCGATGAAAGGGGATGCGGCCGTGCAGGCG
GTTGCCAGCCAGCCGTGGGACCCTAGCGTCAAATCTCTTGTCGCTTTCCCTGCCCTGCTGGCGATGAT
GGGCGAGAATCCGCCCTGGGTGGAAAATCTTGGCAATGCGTTTTTGGCCCAGCCGCATGATGTGATG
GATTCAGTGCAGCGCCTGCGCGCCATTGCCCAACAAACCGGGACGCTGAAATCCACACCGCAGCAGA
AAGTGATTGTCACCCCTGCCGCACCGGTTTCAGCCAGCAGCAGCACGGCAGCAACCGCAACCGCCCA
CACAGCGGCGCCTGCGCCCACGCAGGTCATTAAAATAGAGCCGACCAATCCACAGGTGGTCTATGTT
CCCAGCTATAACCCCTCCACCGTCTATGGTACCTGGCCGAACAGCGCCTATCCGCCGGTCTATCTGCC
GCCCCCTCCCGGGGAGCAGTTTACCGATAGCTTCGTCAAAGGCTTCGGGTACAGCCTCGGCGTGGCC
ACCACCTGGGCGCTGTTCAGCAGTATCGACTGGGATGATGATGACCATCACCATCACGATGACGACTA
CCACCACGGCGATTACTCGCATAATGGCGATAACATCAATATTAATGTAAATAATTTCAATCATATAAC
AGGAGAAAACCTGCCGGGAAACCACGTTAACTGGCAGCACAATCCTGCCTATCGCGGACACACACCG
TATCCCGATAATACGGTAGCTCAGCGCTTCCATCAGACCAACGTTTCCGGCGGACTGAGCGCGACCCA
ACATGCGCCAGTCGATCGCGAAGCGCAGCGCCAGGCAGCGATGACCCAGCTGCAGCATAACGTACC
GACGGCCACAGCGGGCAACCTGGCGGCAAACAACGCCTCACGCGACGCCCAGCGTCAGGCGGCCTC
GGCGCAGCTGAAGCAAGCCACCCAACGCAGTAATTACCGCGGTTACGACAGTACGCCGACCCAACAG
CAGCGTCGCGAGGCGGCAAAAACGCAGCTGAAAAACCCCACGCCGCAGCAACAGCAGCGTCGAGAA
GCCGCCAGGAGCCACGAGCAGAACCGCACACCTCAGCAGCAGCAGCGCCGGCAGCAGTTCCAGTCC
GCCACGCCAGCCCAGCGTCAGCAGACGCTCAGCCATCTGCGCGCCAACGCCCTTAGCGGCAACGAAA
GCCGCGCCCCCTCCTGGCAAGCGCAGCAGGAACGAGGACTGCAGAGCCGCCAGTTTTCCGGCGTAA
ACCGCGAGTTACGCGATGGCACCAGAGAACGTCTTTCCGAACACCATGAACTGCGTCGCCGCTAA
SEQ ID NO: 48
ATGTTTAAGTTTAAGGCTTCTTATGTCGCACTGGCGGCAGTATTAACCTCGTCGGTAGTTTATGCCGAC
CCCACAAGCTATACGCACTCTTCCGGCGCCACGGTTATCGATATTGAAAAGCCGAACGCCGCCGGTGT
CTCCCATAACCTGTACCGCGACTTCAACGTCGGCGCCAATGGCACCATCCTCAATAACAGCGGCGATG
ATGTCAGCCACAGCACATTTGGCAATATCGCCCGCAACAATAATCTGACCGCCGGCAGCGCTTCGGTG
ATTTTGAACGAGGTGACCTCCAAAAACGCCAGTAGCCTGAAGGGCTTTATCGAAGTCAACGGTCAGAA
AGCGGATGTGGTAATCGCCAACCCGAACGGCATCACCTGTTCCGGCTGTAGCTTTGTTAATACCAACA
AGGCTATCCTGACCACCGGCAAGGTTAATATGACCGACGATGGCGCTATCGGCAGCTATACCGTAAC
GGGCGGCACCCTCACCATCGGCGAAAATGGCATGAACGCCGCCAACGGCTATGCGGTTCTGCTCGCC
GACGCGATCAATATCAACGGTAAAGTGCAGGCCAACAACGCCCTGGTCAGCGCGGGCAACTTCACCA
TGGATAACAGCTCTGGCTCGGTGACCTCCGCTGGTAAAAAGGCCACCCTGATCCAGATGACGGTTAA
CCCGCAGTACAGCATCGACGTCAGCAGCCTTGGCGGCATTGAGGCCAACAGCATCAGCATGGTCGGC
AATAACATCGGCTTTGGCGTACGTAATAAAGGCTCTATCGTCGCGAATAGTTCGCTGCAGCTCACCAG
CAACGGTAATCTGCTGAACAAAGGCACGATCAAAAGCAACGGTCTGCTGAGTCAGGTCGCCACCGCC
TCGGGCATCACCAATGACGGTAGCATCGCCGGCGCCTATTATTTAATGCTCTCCAGTGGCGATTATAT
CGTTAACACCGGTTCTCTCTCCGGCGGCCAGCTGATTGCCACCGCTAACGGCAACATCACCAACGGC
GACTCAGGCACGATGACCGGCACCAGTGGATTAAGCCTGACCAGCGGCGGGAAAATCCGCAACGAA
GAAAAAGCCTCCCTGCTGTCAAATAACCAGATTGCCGCCACGGCAATCGGTGATTTCCTCAATGAAGG
CAAAATCAGCGCCAAACACACCAGCCTGACGTTTGTCGGCGACAGCTTTAAAAACACTGGCAATATTA
ACTCTACTGGCCAAACCACCATTCAGTCGCTTAAACAGGACGGCAGCGCCAATACGGGCGAGATCTAT
AACCTCGGCAATATCACCGGCGAAAATATCAATCTGCAGACCAATGGCACGCTGGCGCAAAGCAGCA
GTGGTCGTATTGAGGCAACCAACGCCATTACCGCCCACAGCTACTGGCTGAACCAAAATGGTTATATG
AATGCCGCCGATATCACCACCGATCACGGCGTAGTGAATAATTATGGCAATATTACTGCCAAAAATATT
TCAATCACGACCTACTCAGATATCACCAACGAAGGGCAGATCAGCAGCACCGGCGACCTGACCTTAAA
TACCAAAAATAAAGGCGCGATCTACAATTATTCAACCCTCAGCGCGGGCGGCAACATGACGTTAACCG
CCACCAAAGTGGTCAATGGTGGTAAAAGTTGCGGCATACTGGGCCTGGCGAAATGCGGCGTCGGGAC
GTTAACTGCTGACAAGCTGGTACTGAACTCATCGCAGAAATATGTTAGCGACATGGGTGGAAAACAGT
ATTTCAAGAGCACCGAAGTCAACACGGTGAAATAA
SEQ ID NO: 49
ATGATGGACAACCTACGCACGGCCGCCAACAGCGTCGTGCTCAAGATTATTTTCGGTATCATTATCGT
CTCGTTCATTTTGACCGGGGTGAGTGGTTACCTGATTGGCGGTGGCAAAAACTATGCCGCAAAAGTGA
ATGGCCAGGAGATTGGCCGTGGGCAGTTTGAAAACGCCGTCGCCAGCGAACGTAACCGTATGCAGCA
GCAGCTTGGCGATCAATTCTCCGAGCTGGCGGCGAACGAAAACTACATGAAAACCATGCGCCAGCAG
GTGCTGAACCGCCTGATCGATGAGTCGCTTCTGGATCAGTATGCCCGCGAGCTGGGCCTCAGCATCA
GCGATGAGCAGGTGAAGCAGGCGATCTTCCAGACCCAGGCGTTCCAGACGAACGGTAAGTTCGACAA
CCAGCGTTTCAGTGGTATTGTCGCCCAGATGGGGATGACCACCGATCAGTACGCCCAGGCGCTGCGT
AACCAGCTGACCACGCAGCAGCTGATTAACGCCATTGCGGGTACCGACTTCATGCTGCCGGGCGAGT
CCGATCAGCTGGCGGCGCTGGTATCTCAACAGCGGGTGGTCCGCGAAGCGACCATCAACGTAAATGC
CCTGGCGGCAAAACAGACCGCCAGCGATGAGGAAATCAACGCCTTCTGGCAGCAGAATCAGGCCCGT
TTTATGGCGCCGGAGCAGTTCCGCGTCAGCTACATCAAAATGGATGCCGCCAGCATGCAGGAGAGCG
CCTCTGACGAAGAGATTCAGTCATGGTACGACCAGCACAAGGATCAGTTCACTCAGCCGCAGCGCAA
CCGCTACAGCGTGATTCAGACCAAAACTGAAGCCGATGCGAAAGCGGTACTGGCCGAGCTGCAAAAA
GGAGCGGACTTCGCCACGCTGGCGAAAGAAAAATCGACCGATATTATCTCTGCCCGCAACGGTGGCG
ATATGGGGTGGATGGAAGATGCCTCTACCGTGCCTGAGCTGAAAGATGCCGGGCTGAAAGAGAAAG
GCCAGCTGTCTGGCGTGATCAAATCCTCGGTTGGCTTCCTGGTAGCTCGTCTGGACGACGTCCAGCC
GGCGCAGGTGAAGCCGCTGGCTGACGTGCGTAATGACATTGCGGCGAAAGTGAAGCAGGAAAAAGC
GTTGGATGCTTACTACGCGCTGCAGCAGAAGGTGAGCGATGCGGCCAGCAACGATAATGAATCGCTG
GCGAGCGCAGCGCAGGTCGCCGGGCTGAAGGTCGTAGAAACCGGCTGGTTTGGCCGCGATAACCTG
CCGGAGGAGCTGAACTTTAAACCGGTCGCTGACGCTATTTTCAACGGCGGTCTGGTGGGTGAGAACG
GCGCGCCGGGCAGCAACTCCGATATCATTACCGTTGACGGCGATCGCGCTTTTGTTCTGCGCATTAGC
GAACACAAAGCCGAGGCGGTGAAGCCGCTGGCCGAAGTGAAGGCACAGGTTAGCGATATCGTTAAG
CACAATAAAGCGGAACAGCAGGCGAAACTGGAGGCCGACAAGCTGCTGGCGGCGCTGAAAGACGGC
AAAGGCGATGAAGCGATGAAGGCGGCTGGCCTGAGCTTTGGCGCGCCGCAGACGCTTTCTCGTACC
GGCCAGGATCCGCTGAGCCAGCTGGCATTTACCCTGCCGCTGCCGCAGCAGGGTAAACCGGTCTACG
GCGTGGGCAGCAATATGCAAGGCGATGTGGTGCTGGTAGCGCTGGATGAGGTGAAAGCCGGCAGCA
TGCCGGAAGAGCAGAAGAAGGCCATGGTTCAGGGGATCACCCAGAACAATGCCCAAATCGCTTTCGA
AGCGCTGATGAGCAACCTGCGCAAGGCGGCGAAAATTAAGCTCGGCGACAGCATCGACCAGCAGCA
GTAA
SEQ ID NO: 50
ATGTTCAGGTTAAACCCTTTTATCCGGGCGGGATTGTCTGCGTCCGTCGTATCGTTGGCGTTTCCGGC
TCTGGCCGATGTGAATGAAGAAACGCTGGTGGTGACCGCCTCGGCCACTGAACAGAATGTCAAAGAC
GCGCCGGCGAGCATCAGCGTCATCACCCAACAGGATTTACAACGCAAGCCTGTTCAGAACCTGAAAG
ACGTGCTGCGCGATGTGCCTGGGGTCCAGCTCACCAACGAAGGGGATAACCGCAAGGGCGTTAGCAT
CCGCGGTCTGAGCAGCAGCTATACCCTGATCCTGGTCGACGGCAAGCGCGTTAACTCGCGGAACGCC
GTCTTCCGCCACAATGACTTCGACCTTAACTGGATCCCGGTGGATGCTATTGAGCGTATCGAAGTGGT
GCGCGGCCCGATGTCCTCCCTCTACGGCTCCGATGCGCTCGGTGGGGTGGTCAACATTATTACCAAA
AAAATCGGCCAGAAATGGACCGGGACGCTGAGTGCTGATACCACTATTCAGGAGCACCGCGATCGCG
GGGATACCTGGAACGGCCAGTTCTTCACCAGCGGCCCGCTGATCGACGGCGTACTTGGAATGAAGGC
CTACGGCAGCCTGGCAAAACGCGCCAAGGACGATCCGCAGTCATCCAGTAATGCCACCGGCGAGACG
CCGCGCATCGAGGGCTTCACCAGCCGCGATGGCAATGTTGAATTCGCCTGGACGCCGAACGAAAACC
ACGATTTTACCGCAGGCTACGGCTTTGACCGTCAGGATCGCGATTCCGATTCCCTTGACCGCAACCGC
CTTGAGCGGGAGAACTACTCTCTGAGCCATAACGGCCGCTGGGATATCGGCAATAGCGAGCTCAAGT
TCTACGGCGAAAAGGTGGATAACAAAAATCCAGGGCAGAGCGGGACTATTACCTCGGAAAGCAATGC
CATCGACGGCAAGTATGTCCTGCCGCTGGGCATGATTAACCAGCTGGTGACCTTCGGCGGCGAATGG
CGCCACGACAAACTTAAAGATCCGGTCAACCTGAGCAGCGGCGGCCAGTCAACGTCGGCCAGCCAGT
ACGCCCTGTTTATCGAAGACGAATGGCGCATCATCGAGCCGCTGGCGCTGACCACCGGCATTCGTAT
GGACGACCATCAGACCTATGGCGATCACTGGAGCCCGCGCGCCTATCTGGTGTATAACGCCACCGAT
ACCGTCACCGTCAAAGGCGGCTGGGCGACGGCGTTTAAAGCCCCGTCGCTGCTGCAGCTTAACCCCG
ACTGGACCACCAACTCCTGCCGCGGCTCGTGCAGCATCGTCGGTAACCCGGATCTGAAACCGGAAAC
CAGCGAAAGCTTCGAGCTCGGTCTCTACTACCGCGGGGAAGAGGGCTGGCTTGAAAATGTCGAAGGC
AGCATCACCACCTTCCAGAATAATGTCGACGACATGATCGACGTTCTGCGCACCTCCAGCGCCAGCGA
AGCGCCGGGCTACCCGAACTTTGTCGGCTGGAAAACTGTCAACGGCAAGCGCGTGCCGATCTTCCGC
TATTTCAACGTCAACAAAGCCCGCATCAAAGGGGTGGAGACGGAGGTGAAGATCCCGTTTGGCGATG
AGTGGAAGCTGACGGTGAACTACACCTACAACGATGGTCGCGATCTGAGCAATGGCGGCGACAAACC
GCTGCAGACGCTGCCGTTCCATACCGCCAACGGCACGCTCGACTGGAAACCGCTGGACGACTGGTCC
TTCTACGTGACGGCCAACTATACCGGCCAGCAGCGCGCGGTGAGCGCCACCGGCAAAACGCCGGGC
GGCTACACCCTGTTTGACGTTGGCGCGGCATGGCAGGTGACCAAAAACGTGAAACTGCGCTCCGGGG
TGCAGAACGTGGGTGATAAAGATCTGAGCCGGGACGACTACAGCTATACCGAAGAAGGCCGTCGCTA
CTTTATGGCGGTGGATTATCGCTTCTGA
SEQ ID NO: 51
ATGAACAGAGCCGCCACGCTGACCCTCAACGCGCCCCTGCTGATGCTCGTCGCTGCGCTGGCGCTTT
CAACCCCTTTCACCGCCGGCGCCGCGCCGGCCTTTCTTGATTACGCCCAACAGCAAACCCAGCAATCT
CAGGCGCAAGAAAAAAACGATGCCGCAAGCGCAAAACAAACACAAGAAAGCCGCCAGAGCGCAGATA
ATAAAAAAACCGGTACCAGCACCTCACAATTACAAAAAAGAATCACCAGCCAGCAGGCGGCGATTGCA
CAAAAAGATAAGCTTATACAGCAATTAAAAAAACAGCTTGCCGCTACGCCGCAAACGGATACTGCCGG
AGCGAATGAGCAAGCGGCGTTGAATAAGAGAATTAATGAATTACAGGTCGCCTTAAGCGCCGCTACT
GCAGAAAAAGAGGCATTAATAAAAAAAGCAGGCGTTGTGCAGAATAATAATCTACAGCAAAGCCAGGC
CGCGGCGCGTCAGCAGATCCAGCAATTAACGACGCAGATTCAGCAAGCCGAAGCTGAAAATAAACGC
CTCAGCGCCAGCTTTACCACGCTTAATAAAGATAAACACGCGCTAATGACCCAACTGGCCGCAACGGA
AAAAGAGAAACAGGCCGCTCTTGAGCAGGTCAAAGCGCTTAACGCTGACAACAGCCGCTGACGACC
CGGCTGGCCGCCGCGGAAAAAGAGAAACAGGCCGTCCTCGAGCAGGTTAAGGCCCTTAACGCCGAT
AAACAGTCGCTGACTATTCGCCTCGCCGCTGCGGAGAAAGCGCAGCAGGCCGCTGTTGACCAGGCTA
AAGCGCTTAACGCTGACAAACAGCCGCTGGCTACCCGACTGGCCGCCGCGGAAAAAGAGAAACAGG
CCGTCCTCGAGCAGGTTAAGGCCCTTAGCGCCGATAAGCAGTCGCTGACTATTCGCCTCGCCGCTGC
GGAGAAGGCGCAGCAGGCCGCTCTTGACCAGGCTAAAGCGCTTAACGCTGACAAACAGCCGCTGGC
GACCCGGCTGGCCGCCGCGGAAAAAGAGAAACAGGCCGTCCTCGAGCAGGTTAAAGCCCTTAACGC
CGATAAGCAGTCGCTGACTATTCGCCTCGCCGCTGCGGAAAAGACGCAGCAGGCTGCCCTCGATCAG
GTCAAAGCCCTTAACGCCGATAAACAATCGCTGTCCACCCGGCTGGCCGCCGCGGATAAAGCGCCGC
ATGGCCCCGCTAACGACGCCGCTGCGCCAAAAAATGAGCCACCAGAGATGGCGGCCATAGTGGCAG
CCTATCGCCTGCAGGCGGATAAAGACAACGCCCAGCTACGGATGAAAGAAGATGAAATCGAACTGCT
GAGAACGCAGCTTTCTGTACAGTCCAAAACGCGCAGCGGCGAGAGCGCCGCCGCCAAACTCAGCGCA
TCGGGAGAACAGCAGGCTTATGCGATCGGCGCCTCGATGGGAAGCGAGGCGCTCAACGTCCTTACCA
CCCGTCGTACTCAGGGAGTTACCGTCGACGCAGGCCTGGTGCTGCAGGGCATCGAAGATGCCTTTCG
CGGCCAGCTTCGTCTCGGAGAGCAGGAACGTAACAAGGCGCTGTTTGATGTGTCGCAGCAGGTTTTT
CAGAACCTGAATAAAATAGAGCAGAAAAACATCAGTGCCGGCAAGAAATATCAGCAGGCGTTTGCGC
GCAAAAAAGATGTGGTCTTTAAAGAGGGCGTCTACAGCCGCGTCGATTACCTGGGTAAAGGAAAAAT
AAGCGGTAATGACCTGGTTACCGTGGTGATCAAAGAGATGCTGACGGACGGGACGGTGATCAACGAT
ATGGAAGCGAAAGATCAGGCGCTTACGCAAAAGCTGGATGCCTATCCCCCGGTGTTTCGCGAACCGC
TGAAGCGTCTACAGAACCACGGCTCCGTGACGCTCGTCGTCCCGCCTGAAAAGGCCTATGGCAGTAA
AGGATTACCGCCAAAAATCCCGCCAGGCGCCACCATGGTTTATTCCGTGCGGATAGTAGATAGCCAAC
CCGAGCCGGCAAAATAG
SEQ ID NO: 52
ATGAAAATCCTGTCCGTGCGTCACGCCGCCCTCCCGGCCCTGCTCTTGCCGCTCATTGCGGCAGCCCA
GGCCGCTGATGAACAAACCATGGTGGTGACCGCCGCGCCAACCACGGTTTCTGAACTGGATACCCCC
GCCGCCGTCAGCGTGGTGAATGGGGATGAGATGCGCCAGGCCGCGCCGCGCGTCAATCTCTCTGAA
TCGCTGGGCGCCGTGCCGGGCCTGCAGGTGCAGAACCGGCAAAACTATGCCCAGGATCTGCAGCTG
TCGATTCGCGGCTTTGGCTCGCGCTCAACCTATGGCGTGCGCGGACTGCGCATCTATGTGGATGGCA
TTCCGGCCACCATGCCCGACGGCCAGGGGCAGACCTCAAATATTGATATCGGCAGCGTTGACACCAT
TGAGGTGCTGCGCGGCCCCTTCTCTGCCCTGTACGGTAACTCGTCCGGCGGGGTGATCAACGTCACC
AGCCAGACCGGCACCCAGCCGCCCACCGTGGAAGCCAGCAGCTACTATGGCAGCTTCGGCACCTGG
CACTACGGGATGAAAGCCACTGGCGCCGTTGGCGACGGCAGCCACGCAGGCGATGTGGATTACACG
GTCTCAACCAATCGCTTCACCACCCATGGCTATCGCGATCACAGCGGCGCGCGCAAAAATCTGGCGA
ACGCCCGGCTGGGGGTGCGCATCAACGACGTCAGTAAGCTGACTCTGCTGCTGAATAGCGTGGATAT
CAAAGCCAATGACGCCGGTGGCCTGACCGCCGATGAATGGCGCGATAACCCGCGCCAGTCGCCGCG
CGGCGACCAGTATAATACCCGCAAGAATACCCGACAGACCCAGGCCGGCCTGCGCTATGAGCGCCAG
CTCAGTGCCCAGGACGATCTCAGCGTTATGATGTACGCTGGAGAACGTGAAACCACTCAGTTCCAGTC
GATCCCGCGCGCGCCGCAGCTGAAGCCGAGCCATGCCGGCGGGGTGATCGACCTTACCCGTCACTA
CCAGGGGATCGATACCCGGCTGACCCATCGCGGAGAGCTGCTGGTGCCCGTCACGCTCACCGCCGG
TCTCGACTACGAAAACATGAGCGAGCGGCGCAAAGGGTATGAAAACTTTGTGATGGTCAACGGCGCG
CCGCAGTATGGCGAACAGGGCGCGCTGCGCCGTAACGAACGCAACCTGATGTGGAACGTCGACCCC
TACCTGCAGACCCAGTGGCAGCTCACTGACAAACTCTCGCTCGATGCCGGGGTTCGCTACAGCTCGG
TATGGTTCGACTCGAACGACTACTACATCACCCCAGGCAATGGCGATGACAGCGGTGATGCCAGCTAT
CACAAATGGCTGCCCGCGGGCTCGCTGAAATATGCCCTGACCGACGCGTGGAACGTCTATCTTTCCG
CCGGCCGCGGCTTCGAGACGCCAACCATTAACGAACTCTCCTACCGCTCCGATAACCAGAGCGGCCT
CAACTTCGGCCTGAAACCCTCCACCAACGACACGGTGGAGATCGGCAGCAAGACGCGGATCGGCAAT
GGGCTGTTCACCGCCGCCCTGTTCCAGACCAATACCGATAATGAGATTGTGGTCGACAGCAGCAGCG
GCGGGCGCACCAGTTATAAAAACGCCGGCAAGACCCGCCGTCAGGGGATGGAGCTGGGGCTGGATC
AGCAGTTTGGCGAGAGCTGGCGTCTGAAGGCGGCCTGGACCTGGCTGGACGCGACCTATCGCACTA
ACGTCTGCGACGACGCCAGCTGCAATGGCAATCGCATTCCGGGGATCGCGCGCAATATGGGCTACGC
CTCCTTTGGCTATCAGCCGGAGCAAGGTTGGTACGCCGGGAGCGATATTCGCTATATGAGCGATATCA
TGGCCAATGACGAAAACACCGCCAAAGCGCCCTCCTGGACGGTGGTTGGCCTGACGACTGGCTATAA
ATGGAGCTACGGCAGGATGGATATGGATCTGTTCGGTCGCATCGACAACCTGTTCGACCGGGAGTAC
GTCGGGTCTGTCATCGTTAACGAGTCTAACGGACGTTACTACGAGCCTGCCCCGGGACGTAACTACG
GCATCGGCCTGAACCTCGCCTGGCGCTTCGAATAA
SEQ ID NO: 53
ATGAAATACACGTCTCACTTCCCGCTGGGGATCGTCATTCCTCTGCTCGCCTGTAGCGTGCCGCTGCA
GGCGGCAGAGAACATGACCGAACAATCGACGCCTGACGAGAGCGCCGCCACTGCCGAAAATCACGA
GGAGACGATGGTCATAACCGCCGCCAGGCAGAACCTGCAGGCGCCGGGCGTGTCGACCATCACCGC
AGAAGAGATCCGCAAACATCCCCCCGCCCGCGATGTGTCGGAGTTAATTCGTACGCAGCCCGGGGTA
AACCTGACCGGCAACTCCACCAGCGGGCAGCGCGGCAACAACCGGCAAATTGATATCCGTGGCATGG
GGCCCGAGAATACGCTGGTGCTGGTCGATGGTAAACCGGTGACCAGCCGTAACTCGGTGCGGTATG
GCTGGCGCGGCGATCGTGACTCCCGCGGCGATACCAGTTGGGTGCCAGCGGAGATGATCGATCATAT
CGATGTGATCCGCGGCCCGGCGGCGGCGCGCTATGGTAATGGCGCGATGGGCGGGGTCGTCAACAT
CGTGACCAAACCGACCACGCGAGAATGGCACGGGTCGTGGAATACCTATATGAATGCTCCGCAGCAC
CGTAAAGAAGGGGCGACGAAACGTACTAACTTTAGCCTCAATGGTCCGCTGTCGGACAGTGTCAGCT
TCAATCTCTGGGGTAATCTGAGTAAAACCCAGGCCGATGCACAGGATATTAACGCCGGGCATGAAGC
GGAACGTACCGGTTCCTACGCCGGTTCTTATCCCGCCGGACGTGAAGGGGTGGTGAACAAAGATATT
CACAGTAAGCTGCGCTGGGAGTTTGCCCCGATGCAGGCCCTGGAGTTTGAGGCCGGTTACAGCCGCC
AGGGTAATCTCTATGCCGGCGACACACAAAACACCAATACCAGTACGCTGGTGAAGAGTATGTACGG
GAAAGAGACCAACCGTCTCTACCGGCAAACTTACGGCGTAACATGGACCGGCGGCTGGGATAATGGC
GTGACCAGCAACAGCTATGCCCAGTACGAACACACCCGTAACTCGCGAATGGATGAAGGGCTGGCGG
GCGGTACGGAAGGGATCTTCTCCAGTAGCGAGTTTTCAGATATCGATCTGGCCGATGTCCTGCTACAT
AGTGAAGTGAATATTCCGTTTACGCTGGGGGTCGATCAGAATCTGACGCTGGGGGCAGAATGGAATC
AGCAGCGGATGAAAGATGGCGTATCGACAACCCAGGCGCTCTCTTATGGCACTATCGATGGCGTATC
GGCTACCGGTCGTAGCCCGTACTCCAGTGCCGAGATCTTCTCGCTGTTTACCGAAGATAATATGGCGC
TAACGGACAGCACCATGCTGACACCCGCTCTGCGCTTCGATCACCACAGCATCGTCGGCAATAACTGG
AGCCCCTCACTGAACCTGTCTCAGGAGCTGACGGACGACTGGACGCTGAAGCTGGGCATTGCCCGTG
CTTACAAGGCGCCTAACCTCTACCAGTTGAACCCGAACTATATTCTCTACAGCAACGGTCAAGGCTGTT
ACGCCAGTAGTTCCGCCTGCTATCTGATGGGGAATAGCGATCTGAAAGCGGAGACCAGCGTTAATAA
AGAGATTGGTCTTGAGTACAAGCATGATGGCTATCAGGCGGGGATCACCTGGTTCCGTAACGACTATC
ACAATAAGATTGAGTCAGGGTATGCGGCGGTGGGTACCGCCAGCAACGGCACCACCAATATCTATCA
GTGGGAAAACGTACCAAAGGCGTTAGTGGAAGGCCTGGAAGGAACGCTGAATCTGCCGGTGGGGGA
GGCGGTTAACTGGAGCAATAACCTGACCTGGATGCTGCAGAGCAAGAATAAGACGACCGGCGACCG
GCTGTCAGTGATCCCGCAGTTTACCCTGAACTCGACTTTGAGCTGGCAGGTTCGTGAAGATCTCTCCC
TGCAGAGCACCTTTACCTGGTATGGCCGACAGAAACCAAAACGCTTCAATTATAAGGGCGAGGCGGT
CAGCGGCAGCGAACTAAACGAAGTCAGCCCATACAGCATTGTCGGCCTCAGTGCGACCTGGGATGTG
AACAAAAATCTGAGCTTCACCAGCGGGATAGATAACCTGTTTGATATTCGCCACTACCGGGCAGGGAA
TGCGCAAACGACCGGCAACGCGACGACGGGAGCTTATCTGTATGGCGCGGGTGCCGAGACCTATAAC
GAATCGGGGCGGACCTTCTTTATGAGCGTTAATACTCATTTCTGA
SEQ ID NO: 54
ATGGAAAAAAACGCTTCTCTGCCTTTCGGCAGTTTCAACTCATTGGCATTGTTTACAGGTCTGTGTCTG
GGAGCCTCGCCGGCAGCAGGCATCGCAGCGGAAAATTCGGTCAAAAATAGTGAAGAGACGCTGGTA
GTGGAAGCCGCTCCGCCTTCACTCTACTCCCCCGGCGCTTCCGCCGATCCCAAGTTCAATAAACCGCT
GGTCGATACCACCCGCACCATCACCGTGATCCCGGAACAGGTGATTAAAGATCAGGGCGTCACCAAC
CTGACTGACGCCCTCAAAAACGTTCCCGGCGTCGGGGCGTTTTATGCCGGGGAGAATGGCAGCTCAA
CCACCGGGGATGCCATCTTTATGCGCGGCGTGGATACCTCTAACAGCATCTATGTGGACGGCATTCG
CGACATCGGTAGCGTGACGCGCGATACCTTCAATACCCAGCAGGTGGAAGTCATCAAAGGGCCCGCC
GGCACGGACTATGGCCGCAGCGCGCCCTCCGGCTCGATCAATATGATCAGCAAGCAGCCGCGCCTTG
ACTCCGGGATCGACGGCTCGGCCAGCATCGGCAGCGCCTGGTCGCGCCGGGGCACTCTCGACCTGA
ACCAGGCGTTTAGCGACAACGCTGCGTTCCGTCTGAACCTGATGGGGGAAAAAACGCATGACGCTGG
TCGGGACCGCATTGAAAACGAACGCTATGGCATCGCACCGTCGCTGGCCTTCGGCCTTGATACCCCA
ACTCGTCTGTATCTGAACTATCTGCACGTCCGGCAGAACAACACCCCGGATGGCGGGATCCCTACCGT
CGGCCTGCCGGGCTATTCGGCGCCTTCGCCGAAGTATGCCGCACTCAACTCCGCCGGGAAGGTCGAT
ACCAGCAATTTCTATGGCACCGACTCCGATTACGATAAATCTACTACCGACAGCGGTACCCTGCGCTT
CGAACACGATCTGACGGAGAATACCACCGTGCGCAATACCACCCGCTGGTCGCGAGTGAAACAGGAG
TATCTTTTGACCGCGGTGATGGGCGGCGCGAACAATATCACCGCCCCCGATATCAATGACGTCAACAC
CTGGAGCTGGTCGCGTCTGGTTAATACCAAAGATGTCAGCAACCGTATTCTGACCAACCAGACCAATA
TCACCTCGACTTTCAATACTGGCTCGATAGGCCATGACGTCAGCGCCGGCGTGGAGTTTACCCGGGA
AAACCAGACCAACTATGGCGTTAACGCCAGGACCGCGCCGGCGGTGAATCTCTACCATCCGGTGAGC
AACCTGTCGATTGGCGGGCTGGACAGAAACGGGGCGAACGCCAACGGCCAGACCGATACCTTCGGG
ATTTATGCCTTTGATACGCTGACGCTGACCGAGCGGATTGAGATCAACGGCGGGCTGCGTCTCGACA
ATTACCATACCAAATATGACAGCGCCACCGCCTGCGGCGGCAGCGGACGCGGGGCTATCGCCTGCCC
GCCCGGACAGTCGACCGGCAGCCCGGTCACCACTGTCGATACCGCTAAATCCGGCAATCTGGTTAAC
TGGAAAGCCGGGGCGCTGTACCGCTTAACCGAGCAGGGCAATGTCTACGTCAACTACGCCATCTCAC
AGCAGCCGCCGGGAGGCAGCAGCTTCGCCCTGGCCGCCAGCGGCAGCGGCAACAGCGCTAACCGGA
CCGACTTTAAGCCACAGAAGGCGAAATCCAGCGAGCTGGGCACCAAGTGGCAAATCTTCGACAACCG
TCTGCTGCTCAGCGCGGCGTTATTCCGCACCGATATTGAAAACGAAGTGGCCGCCAACGATGACGGA
ACCTGGTCGCAGTACGGCAAAAAGCGCGTGGAGGGGTATGAACTCTCCGCGACCGGAAACCTGACCC
CGGACTGGACGATTATCGCCGGCTACACTCAGCAGCATGCGACAGTGACGGAGGGACAGAACGTTGC
ACAGGATGGATCTTCCGCCCTGGCCTACACCCCGAAACATGCCTTTACGCTGTGGACGCAGTATCAGG
CCACCAGCGATCTGTCCGTCGGCGGCGGTGTGCGCTATGTCGGAAGCCTGCGCCGGGGCAGCGATG
GTGCAGTCGGTACCCCGGATCACACCGAGGGCTACTGGGTTGCCGACGCCAAACTGGGCTATCGGGT
CAACAGCAACCTCGATCTGCAGCTCAATATGTATAACCTGTTTGATACCGATTACGTGGCCTCCATCAA
CAAGAGCGGCTATCGCTATCATCCGGGCGAACCCCGGACCTTTATGCTGACGGCGAACGTCCATTTCT
GA
SEQ ID NO: 55
ATGGCGACTATGTACAAATCGACTCCGTCAGCAGCATGGTGTAAAAAACGCCTGCTGGTGACCTCTTT
GTTTGCAGCAATTTATCAGACTTCTGCCATCGCAGCAGATACTTCCGCCGTTAGCGGCGAGGCGGTGG
ATGACACCTCGGAACAAATGACCGTCACCGCCCCCGCGCCGGTGCAGAAAGCCGGTAGCGAACACAG
CATCAGCGCCCGGGAGCTGGAGAATAAAGGCGCTAACGATTTCGGCTCAATCATGCGCTATGAGCCG
CTCATCAGCGCCACCGGGGCCAGCGGCGGCTCCGGCAACGGCAAAAGCGGCTTCGACCGCGGAGGT
TACACCGGCTACAACATTCGCGGTATGGAGAGCAACCGCGTAGGCATCGACGTGGACGGTATCGCGC
AACCCAACGCCACCGGCCGCGGCTACGTCGGCCGCGCCGGGCTCAACACCTTCGGCATCGGCCGCG
ATTATATCGACCCGTATATGTACGGCAGCGTTGATATCCAGTCCGGCGCCACCTCGACGGAAACGGCC
AACAGCGCTATCGGGGGGAATGTCTCCTTCCGCCCGAAATCAGCGGATGATTACCTGCGCCCGGGCA
AGACCAGCGCCTTCGGCTACCGCAGCGGTTACGACTCTGCGGATCGCAGCTGGCACAACGGGGTGA
CCGTCGCCGGCGGCGATGAGTTCCTGCGCGGGATTTTGGTCTATAGCCGCCGTGACGGCCAGGAAAC
TGAAAACAACAGCGGCACCGTCGACGCCTACCCGGCGAACTGGCACTCCGATGCTTTTCTGGCCTCC
GGGATCTGGCAGCCTAACGATGAGCACAAGCTGACCAGCACCTTCGACTATTACCATAAAACCAACCA
CACCCACTACGATACCTGGGACTCCAGCGGCAACAGCACCATCGGCACCGCCAACCAGACCAGCCAG
ACCCGGCGCTGGGGCCTGAGCCTGAAGGATGACTGGACGCCGATGAACGACTACCTCGACAGCGTC
TCCACAAAAATCTACTACCAGCATACCGAAGCCCATGACTGGACTTATATGCCGGACAGCGTCACCCG
CAAAATGCAGACGGTGAACTCTAACTACGATACCGACACCTGGGGCCTGCAGACCGCGCTGGCGAAA
ACCCTGGGCCGCCACGATCTGAGCGCCGGTTTCAACGCCAGCACCAGCAAAACCCAGCGGCCGTTCA
GCCAGTCGCCGATCCCCAGCGTTTACAGCGAGATCATGCAGCCGGAGGCAGACAGCCGCAGCTACAC
CCTCGGCGGCTTTGTCCAGGATAAGATCAACTTCGATCTCGACAGCCACAACTTCGCCGTTATTCCCG
GCGTGCGCGTGGTGCATCAATCGACTAAGCCGGAAAATCTGTCCGATCTCGCCGCCAACAGCAGCGT
GCTGAGCGAATCGTCGGTGGCGAATCTGTACGGCAAAAACAGCGATACCCAGGTTCTGCCGTCGTTG
ACCTTCCAGTACGACCTCACCCCGCGCCTGATGACCTACCTGCAGTACCAGCGCGGGGCGCAGTTCC
CCAACGCCAGCCAGCTGTATGGCTCCTGGAACCTCGGCTCCAGCTACGCCGGCAGCCAGCAGTATGC
CCTGATCGGCAATACCGATCTGAAGACGGAAACCAGCGATAATCTCGAGTGGGGGCTGAAAGGGGAA
GTTACCGAAGGCATCACCCTGCGCACGGCGCTGTTCTACAACAGCTATAAGAACTTTATCGCCTATAC
CCGCTATACCCGCGCCAACAATCCGGGCCAGTTCACGAATGTGCCGTCGAACATCTACACCATTTATC
AGGCGGAAAACCGCGATAAAGCCTATATCTACGGCGGTGAGATTAGCACCAAATTTAACTTTGGCACC
TGGTTTGAGCAGGTGGACGGCCTGAGCGCCACCCTCGCCCTCGGCTATAGCGAAGGGAAATCGAAAT
CCAGCTACAGCGGCGATAAATACGTCGACCTCGACAGCGTGGCGCCAATGAAAGCCATCGTCGGCGT
GGCGTGGGACGATCCGGCGAAACGCTACGGCACCGCCCTGACGGCGACCTTTGTCAAAGGGAAACA
GGCGACCGCCACCAACCGCGAAAGCTACAGCAACAGCGGATCCGCCATCACCGATGCCAGCAGCGA
CTATATGCGCGTGCCGGGCTACGGCATGCTGGACTGGACCGCGTACTGGCAGGTGGCGAAAAACGT
GCGCCTCAATGGCGGGGTCTACAACCTCACCGATCGTAAATACTGGGATTACCTGAGCAGCCGCAAT
ATCGAGACCGGCACCAACCAGGACGCCAACGATAAAGCGCTGGCGGTGATGCCGGGCCGCACCTGG
CAGCTGGGCGTCAACGTCGACTTCTGA
SEQ ID NO: 56
ATGGCGATGAAAAAGTTGCTCATAGCGTCGCTGCTGTTTAGCAGCGCGACTGTATACGGTGCTGAAG
GGTTCGTGGTGAAGGACATTCATTTCGAAGGCTTGCAGCGTGTCGCTGTTGGTGCGGCCCTCCTCAG
TATGCCAGTGCGTCCTGGCGATACGGTGACCGACGATGATATCAGTAACACTATTCGCGCGCTGTTTG
CCACTGGCAACTTCGAGGACGTCCGCGTCCTGCGCGATGGTGATACCCTGCTGGTTCAGGTGAAAGA
GCGTCCGACGATCGCCAGCATCACTTTCTCCGGCAACAAGTCGGTGAAAGATGACATGCTCAAGCAG
AACCTTGAGGCCTCAGGCGTTCGGGTGGGCGAGTCGCTTGACCGCACGACCATCGCGGATATCGAGA
AGGGTCTTGAAGACTTCTACTACAGCGTCGGTAAATACAGCGCCAGCGTCAAAGCAGTCGTTACGCC
GCTGCCGCGTAACCGTGTCGATTTGAAGCTGGTCTTCCAGGAAGGCGTCTCCGCAAAAATTCAACAGA
TCAACATCGTCGGCAACCATGCGTTTTCGACCGATGAGCTGATCTCCCACTTCCAGCTGCGCGATGAG
GTGCCGTGGTGGAACGTGGTCGGCGACCGTAAATACCAGAAGCAGAAGCTAGCGGGCGACCTTGAA
ACCCTGCGCAGCTACTACCTGGATCGCGGCTATGCCCGTTTCAACATCGATTCTACCCAGGTCAGCCT
GACGCCGGATAAGAAAGGGATCTACATCACCGTCAACATCACCGAAGGCGATCAGTACAAGTTTTCCG
GAGTGCAGGTGACGGGCAACCTCGCTGGCCATTCCGCGGAAATCGAAGCGCTGACTAAAGTTGAGCC
AGGCGAACTGTACAACGGCGCGAAAGTGACCAAGATGGAAAACGACATCAAGAAACTGTTGGGTCGT
TATGGTTACGCCTATCCGCGCGTGCAGTCGCAGCCGGAGATCAACGACAGCGATAAAACCGTTAAGC
TGCACGTTAACGTCGACGCAGGCAACCGTTATTACGTGCGTAAAATTCGCTTCGAAGGCAACGACACC
TCTAAAGATGCCGTACTGCGCCGCGAAATGCGCCAGATGGAAGGCGCATGGCTGGGCAGCGACCTC
GTCGATCAGGGTAAAGACCGTCTCAATCGTTTAGGTTTCTTTGAAACGGTGGATACTGATACCCAGCG
CGTGCCGGGCAGCCCGGACCAGGTCGACGTTGTCTACAAGGTGAAAGAGCGTAACACCGGTAGCTTC
AACTTCGGTATCGGCTACGGCACCGAGAGCGGCGTCAGCTTCCAGGCGGGCGTTCAGCAGGATAACT
GGTTAGGTACTGGCTATGCTGTCGGGATCAACGGTACCAAAAACGACTACCAGACCTATACCGAGCT
GTCGGTGACCAACCCGTACTTCACCGTAGACGGTGTAAGCCTCGGCGGTCGTGTCTTCTATAATGACT
TTGATGCGAACGATGCGGATCTGTCTGACTATACCAACAAAAGCTATGGTACAGACATTACGCTGGGC
TTCCCGGTCAACGAATACAACACGCTGCGCGCCGGCGTCGGTTATGTGCATAACTCCCTGTCCAATAT
GCAGCCGCAGGTGGCAATGTGGCGTTACCTTAACTCGATGGGCCAGTATCCGGACAACACCAACGAC
CGGAACTCGTTCAGTGCGAATGACTTCACCTTCAACTACGGTTGGACCTATAACAAGCTTGACCGCGG
CTTCTTCCCAACGGAAGGTTCGCGCGTCAACCTGAACGGTAAGGTGACCATTCCGGGCTCAGACAAC
GAGTACTACAAAGCGACGCTGGATACCGCGACCTACGTGCCGATCGACAACGATCATCAGTGGGTAG
TACTGGGTCGTACGCGCTTTGGTTATGGCGATGGTATCGGCGGCAAAGAGATGCCGTTCTATGAGAA
CTTCTATGCCGGTGGTTCCAGCACCGTGCGTGGCTTCCAGTCGAACACCATTGGTCCGAAGGCGGTG
TACTTCCCGGCAAGCAGTCGTCATGATGATGACGATAGTTACGATAATGAATGTAAGAGCACCGAATC
CGCACCGTGTAAATCCGATGATGCGGTGGGCGGTAACGCGATGGCGGTGGCCAGCCTTGAGCTGATT
ACCCCGACGCCGTTTATTAGTGACAAATATGCGAACTCGGTCCGTACTTCCGTCTTCTGGGATATGGG
TACCGTATGGGATACTCACTGGGATTCGAGCGCGTACGCTGGTTATCCGGATTACAGCGATCCGAGC
AACATCCGTATGTCTGCGGGTATTGCCGTGCAGTGGATGTCGCCGTTGGGGCCGTTGGTCTTCTCCTA
CGCCCAACCGTTCAAAAAGTACGATGGAGACAAAGCCGAACAGTTCCAGTTTAACATTGGTAAAACCT
GGTAA
SEQ ID NO: 57
ATGACAGATGTGACTATTAAAGCGCTGGCCTCAGAGATTCAGACCTCTGTGGATCGCCTGATACAGCA
ATTTGCTGACGCAGGCATCCGCAAATCGGCTGATGATTCTGTGACCTCGCAAGAGAAACAAACTTTGT
TGACGCACCTGAACCGTGAACACGGCTCGGCGCCAGACAAGCTGACGTTACAGCGTAAGACGCGCAG
TACGTTAAATATTCCAGGTACCGGTGGAAAGAGTAAATCGGTACAAATCGAAGTCCGCAAGAAACGCA
CCTTTGTGAAACGCGATCCGCAAGAGGCTGAACGCCTGGCCGCGGAAGAGCAGGCGCAGCGTGAAG
CGGAAGAGCAGGCCCGTCGTGAAGCTGAAGAAGCAGCGAAACGCGAGGCGCAATTAAAAGCTGAAC
GTGAGGCCGCAGAACAAGCTAAACGTGAAGTCGCTGATAAAGCGAAACGTGAAGCTGCGGAAAAAGA
CAAAGTGAGCAATCAACATACCGACGAAATGACCAAAACCGCCCAGGCTGAAAAGATCCGTCGCGAG
AACGAAGCCGCGGAATTGAAGCGCAAATCGGAAGAAGAAGCACGCCGCAAACTTGAAGAAGAAGCG
CGCCGTGTAGCGGAAGAAGCACGCCGTATGGCTGAAGAAAACGAAAAAAATTGGTCTGAAACCTCAG
ACAGCCCGGAAGATAGCAGCGACTATCACGTCACCACATCACAGCATGCTCGTCAGGCTGAAGATGA
TAACGATCGTGAAGTCGAAGGCGGTCGCGGCCGTAGCCGTAGCAGCAAAGCGGCTCGTCCGGCGAA
GAAAGGCAACAAACACGCTGAATCGAAAGCTGATCGTGAAGAAGCCCGCGCGGCCGTGCGCGGCGG
TAAAGGCGGTAAGCACCGTAAAGGTTCCGCTCTGCAGCAGGGCTTCCAGAAGCCAGCGCAGGCCGTT
AACCGTGACGTCGTAATCGGCGAAACCATCACCGTTGGCGAACTGGCTAACAAGATGGCGGTGAAAG
GTTCTCAGGTCATCAAAGCGATGATGAAGCTGGGCGCCATGGCGACCATCAACCAGGTCATCGACCA
GGAAACCGCACAGCTGGTTGCCGAAGAGATGGGCCACAAAGTTATCCTGCGTCGTGAAAACGAACTG
GAAGAAGCCGTAATGAGCGACCGTGACACCGGCGCGGCGGCTGAACCGCGCGCACCGGTCGTGACC
ATTATGGGTCACGTTGACCACGGTAAAACCTCGCTGCTGGACTACATTCGTTCTACCAAGGTTGCCTC
CGGCGAAGCGGGTGGTATTACCCAGCACATCGGTGCTTACCACGTCGAAACCGACAACGGCATGATC
ACCTTCCTGGATACCCCGGGCCACGCCGCGTTTACCTCCATGCGTGCTCGTGGCGCGCAGGCGACGG
ATATCGTGGTTCTGGTGGTGGCGGCAGACGACGGCGTGATGCCGCAGACTATCGAAGCTATCCAGCA
CGCTAAAGCGGCGCAGGTACCGGTGGTAGTGGCGGTGAACAAGATCGATAAGCCAGAAGCCGATCC
GGATCGCGTGAAGAACGAACTGTCCCAGTACGGCATCCTGCCGGAAGAGTGGGGCGGCGAGAGCCA
GTTCGTCCACGTTTCCGCGAAAGCGGGTACCGGCATCGACGACCTGCTGGACGCGATCCTGCTGCAG
GCTGAAGTTCTTGAGCTGAAAGCGGTCCGCAACGGTATGGCGAGCGGCGCGGTCATCGAATCCTTCC
TTGATAAAGGTCGTGGTCCGGTAGCTACCGTTCTGGTTCGCGAAGGTACTCTGCACAAGGGCGACATT
GTTCTGTGCGGCTTCGAATATGGCCGTGTGCGCGCGATGCGTGACGAACTGGGTCGCGAAGTGCTGG
AAGCGGGTCCGTCCATTCCGGTGGAAATCCTCGGCCTGTCCGGTGTGCCGGCTGCCGGTGATGAAGT
GACCGTAGTGCGTGACGAGAAAAAAGCGCGTGAAGTGGCGCTGTATCGTCAGGGCAAATTCCGTGAA
GTTAAGCTGGCGCGTCAGCAGAAATCTAAACTGGAAAACATGTTCGCTAACATGACCGAAGGCGAAG
TTCACGAAGTGAACATCGTACTGAAAGCGGACGTACAGGGTTCTGTCGAAGCGATTTCCGATTCCTTA
CTGAAACTGTCTACCGACGAAGTGAAAGTGAAGATCATCGGTTCCGGCGTAGGTGGTATCACCGAAA
CCGACGCTACCCTGGCAGCAGCATCCAACGCGATTCTGGTTGGCTTCAACGTTCGTGCCGATGCCTCT
GCGCGTAAAGTTATCGAAGCGGAAAGCCTGGATCTGCGTTACTACTCCGTCATCTATAACCTGATCGA
CGAAGTGAAAGCGGCGATGAGCGGCATGCTGTCTCCGGAACTGAAACAGCAGATCATCGGTCTGGCT
GAAGTGCGTGATGTCTTCAAATCGCCGAAATTCGGCGCCATCGCGGGCTGTATGGTTACCGAAGGGA
CGATCAAACGTCACAACCCAATCCGCGTTCTGCGTGACAACGTGGTTATCTATGAAGGCGAGCTGGAA
TCCCTGCGCCGCTTCAAAGATGACGTTAACGAAGTCCGTAACGGCATGGAATGTGGTATCGGCGTGA
AGAACTACAACGACGTTCGCGTTGGCGATATGATCGAAGTGTTCGAAATCATCGAAATCCAGCGTAGC
ATCGATTAA
SEQ ID NO: 58
ATGAAAAGAATGTTAATCAACGCAACTCAGCAGGAAGAGTTGCGCGTCGCCCTTGTTGATGGGCAGC
GCCTGTACGACCTGGATATCGAAAGCCCCGGGCACGAACAGAAAAAAGCGAACATCTACAAAGGCAA
AATCACCCGCATTGAACCCAGCCTTGAAGCCGCGTTTGTTGATTACGGCGCCGAGCGTCATGGTTTCC
TCCCCCTCAAAGAAATCGCCCGCGAATACTTCCCCGCCAACTACAATGCGCATGGTCGTCCTAATATC
AAAGACGTACTGCGGGAAGGTCAGGAAGTTATCGTGCAGATTGATAAAGAAGAACGCGGCAACAAAG
GCGCTGCGCTCACCACCTTTATCAGCCTCGCGGGCAGCTATCTGGTACTGATGCCGAACAACCCGCG
CGCCGGGGGAATTTCCCGCCGTATCGAGGGCGACGACCGTACCGAACTGAAAGAAGCGCTGGCGAG
CCTGGAGCTTCCGGACGGCATGGGCCTGATCGTTCGCACCGCTGGCGTCGGCAAATCCGCCGAAGCC
CTGCAGTGGGACCTGAGCTTCCGCCTGAAGCACTGGGAAGCGATTCAGAAAGCCGCGGAAAGCCGTC
CGGCGCCGTTCCTGATCCACCAGGAAAGCAACGTCATTGTCCGCGCCTTCCGTGACTACCTGCGCCA
GGACATCGGCGAAATCCTGATCGATAACCCGAAAGTGCTTGAGCTGGCGCGCCAGCATATCGCCGCG
CTGGGTCGTCCGGATTTCAGCAGCAAAATAAAACTGTACACCGGTGAAATCCCGCTGTTCAGCCATTA
TCAGATCGAATCGCAAATTGAGTCCGCCTTCCAGCGCGAAGTGCGCCTGCCTTCCGGCGGGTCTATC
GTTATCGATAGCACCGAAGCGCTGACCGCGATCGATATCAACTCCGCCCGCGCCACCCGCGGCGGCG
ATATCGAAGAGACAGCCTTCAATACCAACCTCGAAGCGGCTGACGAAATTGCCCGCCAGCTGCGTCT
GCGCGACCTCGGCGGCCTGATCGTTATCGACTTCATCGATATGACCCCGGTTCGCCACCAGCGCGCC
GTGGAGAATCGTCTGCGCGAAGCCGTCCGTCAGGACCGTGCGCGCATTCAGATCAGCCATATTTCGC
GCTTCGGCCTGCTGGAGATGTCCCGTCAGCGCCTGAGCCCGTCGCTGGGCGAGTCCAGCCACCACGT
CTGCCCGCGCTGCTCCGGCACCGGCACCGTGCGTGATAACGAATCGCTGTCGCTCTCTATTCTGCGTC
TGATCGAAGAAGAAGCGCTGAAAGAGAATACCAAAGAAGTCCACGCCATTGTTCCGGTACCGATCGC
CTCCTATCTGCTGAACGAAAAACGTGCCGCAGTGAGCGCTATCGAATCCCGTCAGGGCGATGTGCGC
GTTATTATCGTGCCAAACGACGAAATGCAAACGCCGCACTACTCCGTCCTGCGCGTGCGCAAAGGTG
AAGAAACCTCAACGCTGAGCTATCTGCTGCCGAAGCTGCATGAAGAAGAAATGGCGCTGCCAGGCGA
CGATGAGCCGGCGGAGCGGAAACGTCCGGAACAGCCGGCCCTGGCCGCTTTTGTCATGCCAGATGC
GCCGCCAGCCCCGATGCTCGAAGAGCCTGCCGCCGCGCCTGTCGCCGCAGCGGCACCGGTCGCGGC
CGCCGCACCGGCGCAGCCTGGCCTGCTCTCACGCTTCTTCAGCGCGCTGAAGAATATCTTCTCTGGC
GCCGAAGAGGCCAAACCGGCTGAAGTTCAGGTCGAGAAGAAAGCGGAAGAAAAACCGGAGCGTCAG
CAGGAGCGTCGTAAACCGCGCGCCAACAACCGCCGCGACCGCAACGACCGCCGTGATAACCGCGAC
AATCGTGACAACCGCGATAACCGTGACAATCGCGACACCCGTGCGGACAATGCCGAGGGCCGTGAAC
CGCGCGAATCGCGTGAAGAGAACCGTCGCAACCGTCGCGAGAAGCCGTCGCAGAACGTGGAAGCCC
GTGATGTTCGCCAAACCTCAGGCGACGACGCGGAGAAAGCGAAATCCCGTGACGAGCAGCAGCCGC
GCCGCGAACGCACCCGCCGCCGCAGTGACGACAAACGTCAGGCGCAGCAGGAAGCCAAAGCGCAGA
CTCGCGAAGAGCCGGTTGTGCAGGAGACGGAGCAGGAAGAGCGTGTACAAACTCTGCCGCGTCGTA
AACCGCGCCAGCTGGCACAGAAAGTGCGCGTTGAGTCCGCTGTCGTCGAGCCAGTTGCCGAGATCGT
GCCAGAAGCCGTAGTGGCTGAAGTTATCGCTCCGCACAGCGAGCCGGTGAAAGCCGAGCTGCCGGC
AGGGGTGGAGAGCGTGGCGGACCAGGACGAAAATGGCGAATCCCGTGAAGCGAACGGTATGCCGCG
TCGCTCACGTCGCTCCCCGCGTCACCTGCGCGTCAGCGGTCAGCGTCGTCGTCGCTATCGTGACGAA
CGCTATCCGACCCAGTCGCCTATGCCGCTGACCGTAGCCTGCGCATCGCCGGAGATGGCTTCCGGTA
AAGTCTGGATCCGCTACCCGGTGGTTCGTCCGCAGGATCAGCAGCCGGAAGAGGTTCAGGTTCAGGA
CGCCAGCGTCGCGAAAACTGTCGAGGCCGTAGCGGCCCCGGTCGCCGTCGTTGAAACCGTTACCGCT
GCGCCGGTCACCGTCGAGCCGGCTACCATGGAACCAGTAACCGCTGAGCCGGTAGTCGTCGAGCCG
GTAGCGGCCGCCGAGCCGCTGGTCGTTGATGCTGCGGAAGTTGTCGCGCCAGCAGCCGTCGAGCCA
GCGCCTCAGGAGCCGGTCACCGAAGCACCGGCTGTCGAAGCGCCTCAGGCTATCGCGCCAGTGACG
CTCGACGCCGAGCCGGTGGTGGTAGAACCTGAAGCGGTGGAAACGACGCCTGTCGTTGCAGCGCCA
GTGGAAACTATCGCCCCGGTCGCAGAAACCGTGGAGCAAGCGCCAGTGACCGAAGCGGCCCCTGCC
GAACCGGTCAAAGCCGAGCCCCCGGTGAGCAAGCCGGTCGTAGTGGCGGGTCATCGCCATGCCACC
GCGCCAATGACCCGTGCGCCAGCTCCGGACTATGTCCCGGAAGCACCGCGTCATAGCACCTGGGTGC
GCCCGCCGTTCGCCTTTGAAGGTAAAGGCGCCGCCGGTGGTCATAGCGCGACCCATAAAGCCACCGC
TGAACCGACTCGCCCACAGCCCGTCGAGTAA
SEQ ID NO: 59
ATGCGCAAGCTCTCACTAAGTTTACTCACGCTGTCCCTCGGCGTTGCGCTGCTGCCGTTAGCGCAGGC
GGCGACGACGCCTGCCCAGGAGCATCTGCTGGAGCAGGTCCGCCTCGGCGAGGCCAGCAATCGTGA
AGACCTGGTGCGCCAGTCGCTGTACCGTCTGGAGCTGATTGATCCCAACAACCCGGAGCTGATTGCC
GCGCGGATGCGCTATCTGCTGCGTCAGGGGGATGCCGCCGGGGCGCAAAAAGAGCTGGAACGACTG
ACGAAGCAGGCGCCGGACTCCCCGGAGCTGAAGGCGTCGCGCAATGAGATGAAAAGCAACACCGGC
GAGGGCCGCCAGGCGCTGCAGCAGGCGCGACTGCTGGGCGTGGCCGGGAAGGTCGATGAAGCCAT
CGCCGCCTATGAAAAACTGTACGGCGGGGTGCCGGATGACGTTGACGTCGCCATTGAGTACTGGACG
CTGGTGGCGCGCCTGCCGGCCCGCCATAGCGAAGGCGTCAGCCAGTTGAAAAAACTGAACGCCAGC
GCGCCGGGCAACGTCAGCCTGCTGACTTCGCTGGCGAAGCAGATGTTCGCCGATAACAAACCGCAGG
AGGGGTTCGCCTATCTGGCGGAGATGGCCCGATCGGCCTCGGGACGCGGTATCGCCGCCGATATGT
GGTTCAGTGAGGTGAAAAGCATGCCGGTGAGTAAGGCCAGCGTGCAGGCGTTGCAGCAATTTCTTCT
GCAGTTTCCCACCGGCTCGGTGGCGGCGAACGCCCGCGTTCTGCTCGACCAACAGCAGGCGCAGCT
GCAGGATCCGACTTTCCGCGCCCGCTCGGAAGGGCTGGCGGCGGTCAAGTCCGGGAATACCACGCA
GGCGGTCGCGGATCTGCAGAAAGCCGTTCAGGCCGACAGCCGCGACAGCGACGCGGTGGGCGCTCT
CGGCCAGGCCTATTCCCAGCGCGGCGACCGCGCGCGGGCAGTGGCGCAGCTCAGTAAAGCGATTGC
TATGGACCCTGACAGCCCGAACCGCGGCAAGTGGGACAGCCTGCTGCAAACTAACCGCTACTGGCTG
CTGATAAAGCAGGGGGATAACGCCCTGAAAGCCGGCCAGCTTTCGCAGGCGCAGAACTATTATGCCC
AGGCGCAGCGGGTCGATCGCACCGACAGCTATGCCGTGCTGGGGCTGGGGGACGTCGCGGCGGCG
CGCAAAGAGGCGGCGGCGGCGGAGCGCTATTACCAGCAGGCGTTGCGCCTGGATCGCGGCAATAAC
CTGGCGGTGCGCGGCCTGGCCAACCTCTATCGCGCCGAATCGCCGGAGAAAGCCAGCGCCTGGATC
GCCGGCCTCCCTCCCGCTCAGCGGCGGAGCATCGATGATATTGAGCGCAGCCTGACTAACGACCGGC
TGGAGAAACAGGCGCAGGCTCTGGAGAGCCAGGGCAACTGGGCGCAGGCGGCGGAAGTTCAGCGT
CGGCGCCTGGCGCTGGATCCGGACAGCGTCTGGATAACCTACCGTCTGGCGCGGGATCTGGTCAGC
GCCGGCGAACGCCAGGAGGCCGACGCGCTGATGCGGACGATGGTCAACCGCCAGCCGCAGGACGC
CGAACGGGTCTACGCCTCGGGACTCTACCTGTCGGGGAACGACCAGGACGATCTGGCTCTGGCGCAA
ATCGCCGCTCTGCCGCGCAGCGCGTGGACGGATAACATTCGTGAGCTCGAAGCGCGTTTGCAAAGCG
ACCGGGTGCTGCGCCAGGCCAACCAGCTGCGCGACAGCGGTAACGAAGCGCAGGCGATCGCCCTTA
TCCGACAGCAGCCCGCCTCGGTGCGCTATGACCTGACGCTCGCCGACTGGGCGCAGCAGCGCGGCG
ACAGCCAGACGGCGATTGCCAACTATCAGCGGGTGCTGCGCCAGGAGGCCGACAACGGCGATGCGC
GCCTCGGCCTTGCGGAAGTCTACCTGGCCGAGGGCGATAAACCGGCCGCCCGGGCGCAGGTCATGC
AGCTGAAAGGCGCAGAGACCGAATCCATGAACATGCAGCGGCGGGTGGCGCTGGCGCGAGCTGGCC
TTGGCGATACCGCTGACGCGCAACGGATTTTTAATCAGATTGTGCCGCAGGCGAAGGCGCAGCCGCC
CTCGATGGAGAGCGCGCTGGTGCTGCGCGATGCCGCGCGCTTTGCCACCCAGAGCGGGGCGCCGCA
GCAGGCGCTGACGCACTACCGGGAAGCTATGGTGGCCTCCGGCATTACCCCCGCGCAGCCGCAGGA
TAACGATACTTTTACGCGGCTGACGCGCAACGACAGCCATGATGACTGGCTGAAGCGCGGGATCCGC
AGCGATGCCGCCGACCTTTATCGTCAGCAGGATCTGAACGTCACCCTGGAACATGACTTCTGGGGTTC
CAGCGGCACCGGCGGCTATTCCGACCTGAAGGCGCATACCACCATGCTGCAGATGGATGCTCCGCTG
GCGGATGGCCGGATGTTCTTCCGCACCGACCTGGTCAATATGGATGCCGGCAGCTTTTCCACCCACA
GCGACGGGAGCTACTCGCCCAGCTGGGGCACCTGCGGGGAGATCGCCTGTACCAGCGGCAGTAAAA
ATCAGACCGACAGCGGGGCCAGCGTGGCGGTCGGCTGGAAGAATGACACCTGGAGCGGGGATATCG
GCACCACGCCGATGGGCTTCAATGTCGTCGATGTGGTGGGGGGGCTGAGCTACAGCAGCGACGTCG
GGCCGGTGGGGTACACGGTCAACGTCCACCGGCGGCCTATCTCCAGCTCGCTGCTCTCCTTTGGCGG
GCAGAAGGACAGCAGCAGCCATACCGGCGCCACCTGGGGCGGCGTCCGCGCCGACGGCGGCGGCC
TGAGCCTGAGCTACGATCGCGGGGAGGCTCACGGCATCTGGTCCTCGCTGGGCGCCGACTCGCTGA
CCGGTAAAAACGTGGCGGATAACTGGCGCGTGCGCTGGATGACCGGGTACTACTACAAGGTCATCAA
CGAGAATAATCGTCGCGTCACCGTCGGCCTCAACAATATGATCTGGCACTACGACAAAGATCTCAGCG
GCTACACCCTCGGCCAGGGCGGCTATTACAGCCCACAGGAGTATCTCTCGTTCGCCGTGCCGGTGAC
CTGGCGTCAGCGCACCGAGAACTGGTCCTGGGAGCTCGGCGGGTCGGTGTCATGGTCCCATTCGCG
CACCCAGACGCAAGCCCGCTATCCGCTGCTGAACCTGATCCCGTCCGACTACCGGCAGCGCGCCAGC
GAGCTGACGGAGGAGGGGAGCAGCAGCCATGGATTCGGTTACACCGCCAGAGCGCTGGTGGAGCG
GCGGGTGACCAGCAACTGGTTCGTCGGCGCCGCGGTCGATATTCAGCAGGCGAAGGATTACACCCC
GAGCCATGCGCTGCTTTACGTCCGCTACTCGGCGGCCGGCTGGCAGGGGGATCTGGATATGCCGCC
CCAGCCGCTGGTGCCCTACGCCGACTGGTAG
SEQ ID NO: 60
ATGAGCCAGGAATACACCGAAGACAAAGAAGTCAAACTAACCAAACTCAGCAGCGGGCGCCGACTCC
TTGAGGCGATGCTCATCCTTTGCTCCCTCTTCGCCATCTGGCTGATGGCGGCACTACTGAGCTTTAAC
CCCTCGGACCCCAGCTGGTCGCAAACGGCATGGCATGAGCCTATTCATAATTTAGGCGGCGCCCCCG
GCGCGTGGCTTGCCGATACCCTCTTTTTCATTTTTGGCGTCATGGCCTACACCATCCCGGTGATCATCA
TCGGCGGATGCTGGTTTGCCTGGCGGCATCAGGAAAACGACGAATACATTGATTATTTTGCCGTTTCC
CTTCGCCTCATCGGTGCGTTAGCCCTGATCCTGACCTCCTGTGGTCTGGCGGCGATTAACGCCGATGA
TATCTGGTACTTCGCCTCCGGCGGGGTGATCGGCAGCCTGCTGAGCACCACGCTGCAACCCCTGCTG
CACAGCAGCGGCGGCACCATCGCCCTGTTGTGTATCTGGGCGGCCGGGCTGACGCTGTTCACCGGCT
GGTCGTGGGTCAGCATTGCGGAAAAGCTGGGCGGCGGCATCCTGTCCGTTCTCACCTTTGCCAGCAA
CCGTACCCGTCGGGATGATACCTGGGTCGATGAAGGCGAATATGAAGACGACGAGGAAGAGTACGA
CGACGAAGAGGCGGCCAGGCCGCAGGAATCGCGTCGCGCCCGTATCTTACGCAGCGCGCTGGCGCG
GCGTAAGCGTCTGGCCGAGAAGTTTACCAACCCTATGGGGCGTAAAACCGACGCTGCGCTTTTCTCC
GGCAAACGGATGGATGACGGCGAAGAGGTGGTGCAATACAGCGCCAGCGGGGCGCCTGTTGCCGCC
GACGATGTACTGTTTTCCGGCGCCAGCGCCGCGCGTCCCGCAGAGGATGATGTGCTGTTCTCCGGCG
CCAGCGCCGTGCGCCCGGGCGATTTCGACCCTTACGATCCGTTGTTGAATGGCCACAGTATCGCTGA
GCCGGTAAGCGCAGCGGCGGCGGCTACAGCCGCGCCGCAGGCGTGGGCAGAATCACCGGTGGGCC
ATCACGGCGCTGCGCCAGCTTATCAGCCGGAAGCCAGCTATCCGCCGCAGCAGGCCTATCAGCCTGA
ACCCGCTCCGTTCCAGCAGGCCTATCAGCCTGAACCCGCTCCGTTCCAGCAGGCTGCTTATCAGCCGC
CAGCGGGGCAAACCGCACCGCAGGCGTATCAGCCTGAGCCAGCGCCGTATCAACAGCCGGTTTACGA
TCCGCGTGCCGGTCAACCTGCGCCGCAGGCCTATCAGCCTGAGCCAGCGCCGTATCAGCAGCCGGCT
TACGATCCGTATGCCGGTCAACCTGCGCCGCAGGCCTATCAGCCTGAACCTGCGCCGTATCAGCAGC
CGGCTTACGATCCGCATGCCGGTCAACCTGCACCGCAGGCCTATCAGCCTGAGCCAGCGCCGTATCA
GCAGCCGGCTTACGATCCCTATGCCGGTCAACCTGCGCCGCAGGCCTATCAGCCGGAGCCAGCGCCG
TATCAGCAGCCAACTTACGATCCCTATGCCGGTCAGCCTGCGCCTCAGACCTATCAGCAGCCGGCTTA
CGATCCGAATGCCGGTCAGCCCGCGCCGCAGCCGTATCAGCCGGAGCCAGCGGCGTATCAGCCGCA
AAGCGCGCCAGTTCCCCCACCGGAGCCAGAGCCCGAGGTCGTGCAGGAGGAAGTGAAACGTCCGCC
GCTCTATTATTTCGAGGAAGTGGAAGAGAAGCGGGCGCGCGAACGCGAGCTGTTGGCCTCCTGGTAT
CAGCCAATTCCTGAGCCGGAAAGTCCGATTGCCACTAAACCGCTGACGCCGCCGACCACTGCGTCCA
AACCGCCAGTGGAGACAACCGTAGTCTCTGCGGTAGCGGCTGGGGTGCATCAGGCTACCGCCGCCA
GCGGCGGCGCGGCGGCAGCAACCTCGTCCACTGCCGCATCCGCTGCGGCTACGCCATTGTTCAGCC
CGGCGTCCAGCGGCCCAAGGGTTCAGGTGAAAGAGGGCATCGGTCCAAAACTACCGCGGCCCAATC
GCGTGCGTGTTCCTACGCGTCGGGAACTGGCCTCCTACGGCATCAAGCTACCGTCGCAGCGGGAGGC
GGAACAGCGCGCGCGGCAGGCGGAGCGCGATCCGCATTATGATGATGAGCTGCTCTCGGATGAGGA
AGCGGATGCTATGGAGCAGGATGAACTGGCTCGCCAGTTCGCCGCCACCCAGCAGCAGCGCTACGG
TCATCGCTGGGAAGACGATAACGCGACTGATGACGATGAGGCCGACGCCGCGGCGGAAGCGGAGCT
GGCGCGTCAGTTTGCCGCTACCCAGCAGCAGCGGTACGCTACCGAGCAGCCGCCGGGCGCCAACCC
GTTCTCGCCGGCAGATTATGAATTCTCGCCGATGAAAACGTTGGTCAATGACGGCCCGAGCGAACCG
CTGTTTACGCCGACGCCGGAAGTCCAGCCGCAGCAGCCGGCCCAGCGCTATCAACAACCGGCGGCC
GCTCCGCAGCAGGGTTATCAACCTGCGCAGCATCAGCCGATACACCATCAGCCTGTGCCGCCACAGC
CGCAGTCCTATCCGACTGCGTCGCAGCCCGTACAGCCGCAACAACCGGTTGCCCCGCAGGGGCATCA
GCCTGCCGCCCCTGCGCCGCAGGAGAGCCTGATCCACCCGCTGCTGATGCGCAATGGCGATAGTCGA
CCGCTGCAAAAGCCGACCACGCCACTGCCGTCGCTGGATCTGCTTACCCCGCCGCCGAGTGAAGTCG
AGCCGGTGGATACCTTTGCTCTCGAGCAGATGGCACGCCTGGTGGAAGCGCGACTCGCTGATTTCCG
CATTAAAGCGGATGTGGTGAACTACTCACCGGGGCCGGTGATCACCCGCTTCGAACTGAATCTGGCG
CCTGGCGTTAAGGCCGCACGGATCTCTAACCTGTCACGGGACCTGGCGCGATCGCTGTCAACGGTCG
CCGTGCGCGTGGTGGAGGTGATCCCGGGCAAACCGTATGTCGGGCTTGAGCTGCCGAATAAAAAACG
CCAGACCGTCTACCTGCGTGAAGTGCTCGACAACGCCAAGTTCCGTGATAACCCATCTCCGCTCACCG
TGGTGTTGGGTAAAGACATCGCTGGCGATCCGGTAGTAGCCGATCTGGCGAAAATGCCGCATCTGCT
GGTGGCCGGTACCACCGGTTCCGGTAAGTCTGTTGGCGTCAACGCCATGATCCTCAGCATGCTCTAC
AAGGCGCAGCCGGAAGATGTGCGTTTCATTATGATCGACCCGAAAATGCTCGAGCTGTCGGTCTACG
AAGGAATTCCGCACCTGCTGACGGAAGTGGTCACCGACATGAAAGACGCCGCCAATGCGCTGCGCTG
GAGCGTCAATGAGATGGAGCGCCGCTACAAGCTGATGTCGGCGCTGGGCGTGCGTAACCTCGCGGG
CTACAACGAGAAGATCGCCGAAGCCGCGCGCATGGGACGTCCGATCCCGGATCCGTACTGGAAGCCT
GGCGACAGCATGGACGCCGTACATCCGGTGCTGGAAAAACTGCCGTACATCGTGGTGCTGGTGGATG
AATTCGCCGATCTGATGATGACCGTCGGCAAAAAGGTGGAAGAGCTGATCGCTCGCCTGGCGCAGAA
AGCGCGCGCGGCGGGGATCCACCTGGTGCTGGCGACACAGCGTCCGTCGGTAGATGTTATTACCGG
CCTGATTAAGGCCAACATCCCGACGCGCATCGCCTTTACCGTGTCGAGTAAAATTGACTCACGTACCA
TTCTCGATCAGGGCGGCGCGGAATCGCTGCTGGGTATGGGGGATATGCTTTACTCCGGGCCGAACTC
TACCACGCCGGTGCGTGTCCACGGGGCGTTTGTGCGCGACCAGGAAGTCCACGCCGTGGTTCAGGA
CTGGAAAGCCCGCGGTCGCCCGCAATATGTGGATGGCATTACCTCCGACAGCGAAAGCGAAGGCGG
CGGTGGCGGCTTCGACGGCGGGGAAGAGTTGGATCCGTTGTTCGATCAGGCAGTCAACTTTGTGACC
GAGAAGCGCAAAGCGTCGATTTCCGGGGTTCAGCGTCAGTTCCGCATCGGCTATAACCGTGCCGCGC
GTATTATCGAACAGATGGAAGCGCAGGGTATCGTCAGCGAGCAGGGCCATAACGGTAACCGCGAAGT
GCTGGCGCCGCCGCCCTTTGAATGA
SEQ ID NO: 61
AUGAAGAAGUUAGCUUUACUCUCCGCCGUAAUGACGCUUGGAAUGUCGUCAUGGGCUUUUGCUGC
CGACAACCCGCCGCCGCCGCCGGAAAAAGGCGCGCAGCAUCAGGGUAAACCGCCGGUGAAAAACG
GCCAACACGAAGGUAAGCAAGCGCAAUACAACAGAAAACAGCCACAACGAGACGGCAAACAGCCGC
AGCACGACGGUAAACAGCCGCAGCACAACGGCAAGCAGCCGCCAAAAGGGAGCGAGCACAGCGGG
AAACCGCUGCCGCCGAAAGCGUAA
SEQ ID NO: 62
AUGAAACGUUACGCAACCGCACUGCUCUUUUGCACUCUGUCGCUGACCAGCCUGGCCGCUCGCGC
CGAUAUUAUCGAUGACGCGAUCGGCAAUAUUCAGCAAGCCAUUAACGACGCCUAUAACCCCGGCAG
CAGUCGCUCCGAUGACGACGACAGAUACGAUGACGAUGGCCGGUAUGAUGACGGGCGCUAUCAGG
GGAGCCGUCAGCAGAGCCGUGACAGUCAGCGCCAGUAUGACGAGCGGCAACGCCAGCUGGACGAG
CGCCGCCGCCAGCUGGAUGAACGCCAGCGUCAGCUCGACCGCGAUCGUCGUCAGUUAGAAAGCGA
CCAGCGUCGUCUGGAUGAUAGCUACUGA
SEQ ID NO: 63
AUGUUCAGGUCACUGAUUCUGGCAGCAGUACUGCUGGCCGCAGGGCCACUGGUCGCUAACGCUGG
UGAAAUCACCCUGCUGCCAUCGGUAAAAUUACAAAUAGGCGAUCGUGACAAUUACGGUAACUACUG
GGACGGUGGCAGCUGGCGCGACCGUGAUUACUGGCGUCGUCACUAUGAAUGGCGUGAUAACCGU
UGGCAUCGUCAUGACAACGGCUGGCACAAAGGCUGGUACAAAGGCAGAGAUAAAGCCUGGGAGCG
CGGCUAUCGUGCUGGCUGGAACGACCGCGAUGACCACCGCGGCGGCUGGGGUCGCGGCCCGGGC
GGGCGCGGUCACGGUCAUGGACAUGGCCAUCACUAA
SEQ ID NO: 64
AUGAAGGAAAUCGGCUUACCGUUAUUGCUACUGACCGCGCUGGCCAGUCCGGCUUUUGCUGCAGA
CUGUCAGCCAAACGGCAUUGGCGGCUCGUUUUGCAUUAACGAUGACGGUACGACUACCGACACGG
UGCCUAACGAAGUCAACGGCAUGGAUACGUACUCGAAUAAUGGCGGCUAUACCAGUUCCCUGCCC
GAUCGGUCAGGGGCGGAUGAAGCACUGGAAGGUUCAUCGCUGUCGACGCAGCAAGGCGUCGGCA
GCGGACAGAGCGACAGUGCGCUGGCGGGUCGCGACUGGCAUUCGCCCGCCAAUCUGAAUGAUGGC
GCCGCCACCUCCAGUAUGAGCCUGCUGGAUAAACCCUGA
SEQ ID NO: 65
AUGAAUAUGAAAAAACUGACGACCCUUUUGCUCACCGCCACCUUAGGUCUUGCCAGCGGCGCGGC
CCUGGCGGCAGACACCGGCGCCCAGUCCAAUAAUGGCCAGGCCAACUCUUCCGCGGAUGCCGGUC
AGGUGGCGCCGGAUGCCCGUGAGAACGUGGCGCCGAACAACGUGGACAAUAGUCAGAUCAACUCU
GGCUCUGGGGGCACCACGGGCUCGACGAUGACCCAGGAUAAUAUGUCGAGCAAUGAGGUACAUAA
AAACUCGAUGUGUAAAGACGGCCGCUGUCCGGACACCGGUAAAAAACUGGACAACGGUGGCAAUA
CGACCCAAGACAACAGCAAAACCGACGGCACCACCCAGUAA
SEQ ID NO: 66
AUGAAACACCGCAUCGCUCUGCUUCUGGUCCUGACUUCACUUAGCGCCAGCGCCCUAGCCGCCUC
UCCCUGCCAGGAAAAAGAGCAGGAUAUUCAACGAGAGAUCAGCUACGCCGAAAAGCAUCAUAAUCA
AAGUCGCAUUGAUGGGCUAAAUACCGCGCUACGUCAGGUUCGGGAAAACUGUAGCGACAGUAAAC
UCAAAGCCGAUCAUCAGCAAAAAAUUGCCAAACAGCGGGAAGAGAUCGCUGAACGUCAGCGCGAUC
UGCAGGAAGCCCGGAAGAAAGGCGAUGCGGACAAAAUUAACAAACGCCAGCAUAAACUCAAUGAAG
CGCAACAGGAGUUAAAAACGCUGGAGUCUCGGGAUUACUAA
SEQ ID NO: 67
AUGCGACUCAUAACACGACACGUGAGAGAGGAUAUUAUGAAAAAAGCAAUGAUUGCGUUAUCGGC
UAUUCUGGUUGCGGCUCCGGUUUUUGCUGCGACAACACAUGCAACAGAUGAUACCGUCGCGGCGG
CGAAUGCCAACGCCAACACCGCUAAAGAGAAGCUGCAUCAGGCCCAGCACGAGGGCGAAGAGCAGC
AGCUGAAGGCGAAACACGCCGCCGAAGGCAAGCAGGACAGCGUCGGCAGCCAGGUGAGCGAAGGC
GCGCAGAAAACCUGGAACAAGACCAAAGAAGGCACCGAGAAGGGGUGGGAUAAGACCAAAGAGGU
CAGUGAAAAAGGCUGGAACGCCACCAAAUCCGGUGCGGAAAAGGGCUGGGAUAAAACCAAAACCG
GCGCCGAAGAGUUAAAAAAUAAAGUGACUGAAUAA
SEQ ID NO: 68
AUGAAAAAGAUGAUUUCUCUGGCAGUAAUUUUAUCCUGUGUGCUGAGCGUCCCGGCCUUUGCCGA
UGGCCCGAACGACGGCCAUCGCCCGGAGCAGCCCACGGUGUGGCAGAACGGUCCGGACCAUGACG
GGCAUGCACCGCAGGGCGGACCUGACGCGCAUCAUCAGGGCGACCAUGACCAGCGUGGCCCGGAU
CGCGACGGCCAUGACAAACGCGAUCUGGCACGUCAUGAGCAGGACCAUUUCGCCUGGCGCGGGAA
CGAUUUCCGCAAAGGCCACCCGGCUCCGGCGCCGUUCCGUGGCGAUGAAUAUCGCGUCCGCGACU
GGAGCGACCGCGGCCUGCCGCCCCCGCCGGAAGGCCAUCACUGGUCCUAUAUCGACGGUAACUAU
GUGCUGAUCGCCGCGGCGACCGGGAUCAUCACCUCGAUUCUGGUGAGCGGCGCCCUCGGCCACUA
A
SEQ ID NO: 69
AUGAAAAAACCGACAUCCGCCACCCGUGGCAAAUCCGGCCGCAAGUCGCGUGAAGAGUUAAAUCAG
GAAGCUCGCGAUCGCAAACGGCAGAAGAAACAUCGUGGCCACGCGGCAGGCAGUCGCGCGAACGG
CGGCGAUGCGGCUUCAGCGGGUAAAAAACAGCGUCAGGCGCAAGAUCCGCGCGUGGGUAGCAAAA
AACCGAUCCCGCUGGGCGUGAGCGAAAGCAGCGUUCCAGCUCCCAAGCAGCAUAAACCAAAGAGCG
AGAAACCUAUGCUUUCACCGCAGGCUGAGCUGGAGUUGCUGGAGAAUGAUGAGCGCCUGGACGCG
CUGCUGGAACGUCUGGAAGAGGGCGGCACCCUGAAUGCUGAAGAGCAGAGCUGGGUGGACGCCAA
ACUGGAUCGCAUUGAUGAGCUGAUGCAGCAGCUCGGCCUCUCUUACGAUGAUGAAGAUGAAGAAG
AGGAAGAGCGUCAGGAAGAUAUGAUGCGUCUGCUGAAGGGUGGAAACUAA
SEQ ID NO: 70
AUGGCGAGUAAGUUUCAGAACCGUUUAGUCGGGACAAUCGUGCUGGUGGCGCUGGGGGUGAUUA
UCCUGCCAGGGCUGCUGGACGGGCAGAAAAAGCAUUACCAGGAUGAGUUUGCCGCGAUCCCGCUG
GUACCGAAACCAGGCGAUCGCGAUGAACCGGAUAUGUUGCCGGCGGCAACCCAGGCGUUGCCUUC
GCAACCGCCGGAAGGGGCGGCGGAAGAGGUGCGGGCGGGCGAUGCCGCCGCGCCAUCGUUAGAU
CCAUCGCGUAUUCCGGUGAACAGCAACAGCUUCGAUGACGUUCAGGAGCCGGUGGUGGCCGCGAA
ACCGCAGCCCAAGCCGCAGCCGAAACCGCAGCCGCAACAGCAGGCCUCGACGCCAACGCCGCCGCC
GGCUAAGCCACAGCAGCAACAGCCACCGCAGCAGCAGGCGGCCCUGCCGGCGCCGACCGGCAAAG
CCUAUGUGGUUCAGCUGGGCGCGUUGAAGAACGCCGAUAAGGUGAAUGAGAUUGUCGGUAAACUG
CGGGCCUCGGGUUUCAAAGUCUAUACGUCGCCUUCGACGCCGGUACAGGGUAAAAUUACCCGCAU
CCUCGUCGGCCCGGAUGCGUCAAAAGACAAGCUGAAAGGCCAGCUGGGCGAUCUGCAGCAGAUCU
CCGGGCUUAGCGGGGUGGUGAUGGGCUUCACCCCGAACUGA
SEQ ID NO: 71
AUGGCACAACGAGAUUAUGUACGCCGCAGCCAACCGGCUUCUUCGCGGCGCAAAAAGAGCACGACC
CGAAGCUCAAGGAAUAAGCAAAGCAGCCUUCCGGCGAUUUCACCGGCGAUGGUGGCGAUCGCGGC
GGCUGUGCUGGUGGCCUUUAUCGGUGGCCUCUAUUUCAUUACGCAUCAUAAGAAAGAAGAAGCGG
AAGCGAUGCAAAAUCGCCAGGCCGCCGGCAACGGCUUGCCGCCCAAACCGGAAGAGCGCUGGCGC
UAUAUUAAAGAGCUGGAAAGCCGCCAGCCUGGCGUCCGCGCGCCGACCGAACCGACCGCCGGUGG
CGAAGUCAUGAAACCGGAACAGCUGACCGACGAGCAGCGCCAGCUGCUCGCCCAGAUGCAGGCCG
AUAUGCGCCAGCAGCCGACCCAGCUGACCGAAGUGCCGUGGAACGAACAAACGCCGGCGCAGCGC
CAGCAGACGCUUCAGCGUCAGCGUUUAGCGCAGCAACAGCAGCAGGCGCAGCAGCAACAGUGGGC
GCAGACUCAGGCGCAGACCGUCCAACAGCAGCCGCCGCGCGUUCAGCAGCCGAAGCCGGUUCAGC
AGCAACAGCCGAAGCAGACCGCGUCAAACCAGCAGCCGUACCAGGAUCUGCUGCAGACGCCAGCGC
AUACCAAUACCACGCAGCCGCGUACCCAGGCCGCGGCGCCGGUAACUCGGGUGGAAGAAGCGCCG
AAAACCACCGCCGAGAAGAAAGACGAUCGUAGCUGGAUGAUCCAGUGCGGCUCUUUUAAAGGCGC
CGAGCAGGCCGAAACCGUCCGCGCUCAGCUGGCUUUCGAAGGGUUUGCUUCGCACAUUACCACUA
ACAACGGCUGGAACCGCGUGGUUAUUGGCCCGUUGAAAGGCAAAGAAAGCGCCAACGAGAUGAUC
ACCCGCCUGAAGAUGGCUGGACACGCGAACUGCAUUCGUCUCGCCGCCAGGGGUUGA
SEQ ID NO: 72
AUGAGCGCGGGAAGCACCAAAUUUACCGUCAGCCGUAUUGCGGCUCUUUCACUGGUUUCACUCUG
GCUGGCCGGGUGUACCAACACCAAUAAUCCGCCUGCGCCGGUUAGCUCUGCCGGCGGCGCCGCCU
CUUCCAGCACCAACUCCGGCAUGCUGAUUACGCCGCCACCCUCCGGCGUCAAGUCCGCUCCUCAGG
CGCAGCCGAUUCAGCCGAUGCAGACCCAGACCAUUCAGCCGGCGCCGGUGGCGCAGGAGCCGGUA
CAGACGGUAAAUGGCCGGAUCGUUUACAACCGCAAAUAUGGCGAUAUUCCGAAAGGUAGCUAUAC
CGGCGGCAGUACCUAUACGGUAAAACGCGGCGACACGCUAUUCUAUAUCGCCUGGGUCACCGGCA
ACGAUUUCCGCGACCUGGCGCAACGUAACAAUAUCCCGGCCCCGUACGCGCUGAACGUGGGGCAG
GUACUGCAGGUCGGUAACGCCUCAGGCCAGCCGAUCACCGGCGAAAACGCCGUUUCUCAGGCCAG
CGCAAGAGCGAGCGGCGGUGCGACGACCAGCACAACUUCUGCACAAAAAUCGACCGCGGUGGUUG
CUUCACAACCGACUAUUACGUAUUCUGAAUCUUCAGGUGAACAGAGUGCUACCAAGAUGUUGCCUA
AUAAUAAACCAGCGACCACAACCACAACGGUUGUCGCGCCGGUGACGGCACCAACAACGGUGAGCA
CAACCCAGCCGACUGCAAGCAGUACGUCAACCAGUUCGCCGAUCUCAGCAUGGCGCUGGCCGACU
GAUGGCAAGGUUAUCGAGAACUUUAGCGGCGCGGAAGGCGGCAAUAAAGGCAUCGAUAUUGCAGG
CAGUAAGGGACAGGCUAUUGUCGCGACCGCCGAUGGGCGCGUCGUCUAUGCCGGUAACGCACUGC
GCGGCUACGGUAAUCUUAUUAUCAUCAAACACAACGAUGAUUACCUGAGUGCCUACGCUCAUAACG
AUACCAUGCUGGUUCGGGAGCAACAGGAAGUCAAAGCGGGGCAGAAAAUCGCUACCAUGGGUAGC
ACCGGAACCAGCUCAACAAGAUUACAUUUUGAAAUUCGUUACAAGGGGAAAUCCGUCAACCCGCUG
CAGUACUUACCGCAGCGAUAA
SEQ ID NO: 73
AUGCGUAAGCAAUGGCUGGGGAUCUGCAUAGCAGCGGGGCUGCUGGCGGCAUGUUCGAGUGAUG
ACGUGCAACAAAAAACGGUCAGUACUCCACAGCCGGCCGUCUGUAAUGGCCCGACGGUUGAGAUC
AGCGGCGCCGAUCCGCAGUAUGAAACGCCGAACGCCACGGCGAAUCAGGAUUAUGAGCGCGACGG
UAAAAGCUACAAAAUCGUUCAGGAUCCGGCCAACUUUACUCAGGCCGGUUUCGCGGCGAUCUAUG
ACGCAGAACCCAACAGCAACCUGACCGCCAGCGGCGAAGCCUUCGAUCCGACUCAGUUGACCGCAG
CGCACCCGACGCUGCCGAUCCCGAGCUACGCGCGGAUCACUAACCUUGCCAACGGACGGAUGAUC
GUCGUGCGGAUUAACGAUCGCGGUCCCUAUGGCAACGAUCGGGUCAUCUCGCUUUCCCGCGCAUC
CGCUGACCGCCUGAACACCUCCAACAACACCAAAGUGCGCAUCGACCCCAUCAUCGUCGCGCCUGA
CGGUUCGCUUUCCGGCCCGGGGAUGGCCUGUACCACCGUCGCCAAACAGACUUACGCCCUGCCCG
CCCGUCCGAAUCUGGACGGUGGGGACGCCGCUGGCAUGAGCCAGCCCGCGCCCACUGACGUUCGC
CCGAUCAGCAACAGCACGCUGACGCCGGCAGACAGCGUGGGCGCGCCGGUGAACAGCGGCGGUUU
CCUCGGCGCGCCGACGCCCCUGAACAACGGCGUGCUGGAGAGUAGCGAACCAGCGGCAGCCGCCG
CGACGGCUCCUGCCGCCGGCGCCACGCCAACAGCGCCAGUGACCGCGCCUGGCUCCAUUCAGGGU
AAUGUGGUGCCCGCUGCGGCCACCGCCGCAGCCGCUGGCGCCGUGGCGGCCUCGUCCUCCGCGAC
CUCCAGCGCCAGCGGUAAUUUUGUUGUCCAGGUGGGCGCAGUAAGCGACCAGACGCGGGCGCAGC
AGUAUCAGCAGCGCCUGAGCCAGCAGUUUUCUGUGCCAGGCCGGGUCAUGCAAAACGGCGCGGUC
UGGCGUAUUCAGCUGGGUCCCUUUGCUGAUAAAGCACAGGCCAGCGCCGUGCAGCAGCGCCUGCA
AAGCGAAGCGCAGCUGCAGUCCUUUAUUACUCGCGCCAACUAA
SEQ ID NO: 74
AUGGAUGAUUUCAAACCAGAAGACGAUAUGAAAGCCGAUCGCAACGAUCGUCGUGCUGGUCGUUC
CCGUCAGUCUUCCGAGCGUGAUGCCGAUCCGCAGAUCAAUUUUGACGAUGUUGAUCUUGAUGCCG
AUGAAGGCCGUCCGACGCGCGCUGGUAAGGCCCGUCGCGAGCGUGAAGAGGAAGAGUUCGAAGAA
GAACUGGAUGCGCAAGACGAGGAGAUGCUCGAAGAGCAGCCUGUAGAGCGUCGUCCGCGCAAGCG
UAAAAAAGCGCCGGCCAAACCGGCCUCCCGCCAGUACAUCAUGAUGGGUGUGGGGAUUCUGGUGC
UGCUGCUGUUGAUCGUGGGUAUCGGUUCCGCACUGAAAUCGCCAUCAUCUUCCAGCCAGCAGACC
GCUUCCGGCGAGAAGAGCAUUAAUCUGUCUGACGACCAGUCCGCCAGCAUGCCUGCUGCCGGCCA
GGACCAGACUGCCGCCGCUAACAGCACCUCACAGCAGGACGUAACGGUACCGCCUAUUGCCGCGAA
CCCGACGCAGGGCCAGGCAGCGGUUGCGCCGCAGGGCCAGCAGCGUAUCGAAGUUCAGGGCGAUC
UGAACAAUGCCUUGACCCAGCAGCAGGGCCAACUGGACGGCGCCGUGGCUAACUCGACGCUGCCG
ACUGAACCGGCUACCGUCGCGCCAAUCCGGAAUGGCGCCAAUGGCACCGCGGCGCCGCGCCAGGC
GACCGAGCGUCAGACAGCAGCGACCCCGCGUCCGGCUGAACGUAAGCAUACCGUUAUCGAAGCGA
AGCCGCAGUCGAAGCCACAGGCCGUGGCGAAAACGCCGGUAGAAUCGAAGCCGGUCCAGCCGAAG
CAUGUUGAAAGCACGGCGACCACCGCUCCGGCGAAAACGUCCGUCAGCGAAAGCAAACCGGUGGC
CACCGCUCAGAGCAAACCGACCACGACGACCGCAGCGCCAGCGGCAACGGCAGCUGCGGCAGCGC
CGGCAGCGAAGACCGGGAAGACGGCAGGUGACGUCAGCUCAAUGAAAACUGCGCCGUCGGGUCAC
UAUACUCUGCAGCUCAGCAGCUCCUCUAACUACGACAACCUCAACAACUGGGCGAAGAAAGAGAAG
CUGGAUAAAUAUGUUGUCUAUGAAACGUCGCGUAACGGCCAACCAUGGUACGUGCUGGUGAGCGG
UAUCUAUGCAUCGAAAGAUGAAGCGAAACGUGCUGUCACCUCGCUGCCGGCGGACGUGCAGGCGA
AAAAUCCAUGGGCAAAACCGCUGCAUCAGGUUCAGGCUGACCUGAAAUAA
SEQ ID NO: 75
AUGUCAAAGGCAACCGAACAAAACGACAAGCUUAAACGAGCGAUCAUCAUUUCAGUCGCGCUGCAC
AUCAUUCUGAUCGCGCUGCUGAUCUGGAGUUCGUUUGACGAGCAUCUGGAUGCCUCUGCCGGCGG
CGGCGGCGGAUCGUCGAUUGAUGCCGUCAUGGUCGAUCCGGGGGCGGUGGUAAAUAACUAUAACC
GUCAGCAACAGCAGCAGGCCAGCGCACGUCGCGCCGCUGAACAGCGUGAAAAACAGGCGCAGCAG
CAGGCGGAAGAGUUACGUGAGAAACAGGCGGCGGAACAGGAACGGCUGAAACAGCUCGAACAGGA
GCGGCUGCAGGCGCAGGAAGCGGCGAAAGAAGCGAAGGAGCAGCAGAAGCAGGCUGAAGAAGCGG
CUGCCAAGGCCGCCGCGGCGGCAAAAGCCAAAGCGGACGCACAGGCAAAAGAAGCGCAGGAAGCC
GCUGCCAAAGCGGCCGCCGAGGCGAAAGCGAAGGCGGAUGCCCAGGCGAAAGCGGCAGAACAGGC
GGCGGCCAAGGCGGCUGCUGACGCGAAAAAGCAGGCCGAAGCCGCUGCAGCGAAAGCCGCUGCCG
AGGCGAAGAAACAGGCGGAAGCUGAAGCGGCGAAAGCUGCGGCCGAGGCGCAGAAGAAAGCGGAA
GCGGCGGCUGCGAAGAAAGCGCAACAGGAAGCGGAGAAAAAAGCCCAGCAGGAAGCGGCUAAGCA
GGCGGCAGCUGAAAAAGCGGCUGCCGAAAAAGCCGCUGAGAAAGCCGCCGCGCAAAAAGCGGCCG
CUGAGAAGGCCGCCGCCGAGAAAGCCGCAGCCGCUGAAAAAGCGGCGGCAGCGAAAGCGGCUGCA
GCAGAGAAGGCUGCAGCUGAUAAAGCGGCCAAAGCGGCAGCAGCAAAAGCCGCGGCGGCGAAGAA
AGCGGCGGCUGCGAAAGAAGCGGACGGCGUUGACAACCUGCUCGGCGAUCUGAGUUCUGGUAAGA
AUGCGCCUAAAACAGGCGGUGGGGCCAAAGGAAACAAUGCCUCCGCUGCCGGGAGUGGUAAUACU
AAAAACAGUGCCUCAGGGGCUGAUAUCAACAACUAUGCCGGACAGAUAAAAUCGGCGAUUGAAAGU
AAGUUUUAUGACGCAUCGUCCUAUGCGGGCAAAACAUGUACCUUGCGUAUCAAACUUGCUCCUGA
CGGCCUGUUGUUAAAUAUACAGUCCGAAGGUGGUGAUCCUGCUCUGUGCCAGGCCGCUCUUGCCG
CAGCCCGACAGGCUAAGUUUCCGAAACCACCUAGCCAGGCAGUAUAUGAAGUCUUCAAAAAUGCGC
CACUGGACUUCAAACCUCAGUGA
SEQ ID NO: 76
AUGUUUUUUUUAAGUAUUUUUUACAUGGAGAUGACAAAAGUGAAAUUAAGCGCUCUGUUUAUUGC
CCUGAUUCCUCUACUGGGCUCGCCGGUUAUUCAUGCAGAAACUACUGCUGCGCCGGUUCUGGAAA
AUCGCGCUGCGCAGGGAGAUAUCACCACUCCUGGCGGCGCGCGUCGUUUAACAGGCGAUCAAACC
GAAGCGCUGCGCGCCUCGUUAAUCAAUAAGCCAGCUAAAAACGUUAUUUUGCUGAUUGGCGAUGG
CAUGGGUGAUUCGGAAAUUACCGCUGCGCGAAACUAUGCCGAGGGGGCGGGCGGUUUCUUUAAA
GGAAUUGAUGCUCUGCCGUUAACCGGGCAGUACACGCAUUAUUCGCUGGAUAAAAAAACCGGGAA
ACCGGACUACGUGACCGACUCGGCGGCCUCCGCCACCGCCUGGACCACCGGCGUGAAGACUUAUA
ACGGCGCGCUGGGCGUCGAUAUUCAUGAGAAUGCGCAUCAGACCAUCCUCGAGCUGGCGAAAGCG
GCGGGGCUGGCCACCGGCAACGUUUCCACCGCCGAGCUGCAGGACGCCACCCCCGCGGCGUUGGU
AGCGCAUGUGACAUCGCGUAAAUGCUACGGCCCGACGGUCACCAGCGAAAAAUGCCCCAGCAAUGC
GCUGGAAAAAGGGGGCAAAGGCUCCAUUACCGAACAGCUGCUGAACGCCCGACCGGAUGUCACCU
UGGGCGGCGGCGCGAAGACCUUUACCGAAACGGCGACGGCGGGCGAGUGGCAGGGCAAAACCCU
GCGCGAGCAGGCGCAAGCGCGCGGCUACCAGAUUGUGACCGACGCGGCUUCUCUUGCCGCCGCGA
CGGAAGCCAGUCAGGAUAAACCGCUGCUGGGACUCUUUGCCGAUGGCAAUAUGCCGGUACGCUGG
GAAGGGCCGAAGGCGUCUUAUCACGGUAAUAUCGAUAAGCCGCCGGUGACCUGUACGCCAAACCC
GAAGCGUGACGCCUCGGUGCCGACGCUGGCGCAGAUGACGGAGAAAGCGAUUGACCUGCUCAGUC
GCAACGAGAAAGGUUUCUUCCUGCAAGUCGAAGGCGCUUCCAUCGAUAAGCAGGACCAUGCGGCG
AAUCCGUGCGGCCAGAUCGGCGAAACGGUUGAUCUUGACGAAGCGGUGCAGAAGGCGCUGGAAUU
CGCGCGAAAAGACGGUAAUACCCUGGUGAUCGUCACCGCCGACCAUGCGCAUGCCAGCCAGAUCA
UCCCGGCGGAUAGCAAAGCCCCGGGGCUGACCCAGGCUCUGAACACGCACGAUGGCGCGGUGAUG
GUGAUGAGCUACGGCAACUCUGAGGAAGAGUCGAUGGAGCACACCGGCACCCAACUGCGCAUUGC
GGCCUACGGUCCGCAUGCGGCUAACGUCGUAGGCCUGACCGAUCAGACCGACCUGUUCACGACCA
UGAAAGCUGCCCUGAGUCUCAAAUAA
SEQ ID NO: 77
AUGUCACUGCCGUUCAAACCCCAUAUUAUCGCCCUGCUCUGUAGCGCUGGCUUACUCGCGGCGGC
AGGAACACUCUAUGUGCAAAGCCGAACCCCAGCGACGAUCGCUGAACCGCCUGCGCAGCAAGCGCC
AGCGCCCGCAGCGUCGACGACACAGCCGGUGGCCGCCACUUACACCCAGGCGCAAAUUGAUCAGU
GGGUCGCCCCUAUCGCGCUCUACCCGGACAGCCUGCUGUCGCAGGUGUUGAUGGCCUCCACUUAU
CCCGACAACGUCCUGCAGGCGGUCCAGUGGUCCCAGGAUAACCCCGCGAUGAAAGGGGAUGCGGC
CGUGCAGGCGGUUGCCAGCCAGCCGUGGGACCCUAGCGUCAAAUCUCUUGUCGCUUUCCCUGCCC
UGCUGGCGAUGAUGGGCGAGAAUCCGCCCUGGGUGGAAAAUCUUGGCAAUGCGUUUUUGGCCCA
GCCGCAUGAUGUGAUGGAUUCAGUGCAGCGCCUGCGCGCCAUUGCCCAACAAACCGGGACGCUGA
AAUCCACACCGCAGCAGAAAGUGAUUGUCACCCCUGCCGCACCGGUUUCAGCCAGCAGCAGCACGG
CAGCAACCGCAACCGCCCACACAGCGGCGCCUGCGCCCACGCAGGUCAUUAAAAUAGAGCCGACCA
AUCCACAGGUGGUCUAUGUUCCCAGCUAUAACCCCUCCACCGUCUAUGGUACCUGGCCGAACAGC
GCCUAUCCGCCGGUCUAUCUGCCGCCCCCUCCCGGGGAGCAGUUUACCGAUAGCUUCGUCAAAGG
CUUCGGGUACAGCCUCGGCGUGGCCACCACCUGGGCGCUGUUCAGCAGUAUCGACUGGGAUGAUG
AUGACCAUCACCAUCACGAUGACGACUACCACCACGGCGAUUACUCGCAUAAUGGCGAUAACAUCA
AUAUUAAUGUAAAUAAUUUCAAUCAUAUAACAGGAGAAAACCUGCCGGGAAACCACGUUAACUGGC
AGCACAAUCCUGCCUAUCGCGGACACACACCGUAUCCCGAUAAUACGGUAGCUCAGCGCUUCCAUC
AGACCAACGUUUCCGGCGGACUGAGCGCGACCCAACAUGCGCCAGUCGAUCGCGAAGCGCAGCGC
CAGGCAGCGAUGACCCAGCUGCAGCAUAACGUACCGACGGCCACAGCGGGCAACCUGGCGGCAAA
CAACGCCUCACGCGACGCCCAGCGUCAGGCGGCCUCGGCGCAGCUGAAGCAAGCCACCCAACGCA
GUAAUUACCGCGGUUACGACAGUACGCCGACCCAACAGCAGCGUCGCGAGGCGGCAAAAACGCAG
CUGAAAAACCCCACGCCGCAGCAACAGCAGCGUCGAGAAGCCGCCAGGAGCCACGAGCAGAACCGC
ACACCUCAGCAGCAGCAGCGCCGGCAGCAGUUCCAGUCCGCCACGCCAGCCCAGCGUCAGCAGACG
CUCAGCCAUCUGCGCGCCAACGCCCUUAGCGGCAACGAAAGCCGCGCCCCCUCCUGGCAAGCGCAG
CAGGAACGAGGACUGCAGAGCCGCCAGUUUUCCGGCGUAAACCGCGAGUUACGCGAUGGCACCAG
AGAACGUCUUUCCGAACACCAUGAACUGCGUCGCCGCUAA
SEQ ID NO: 78
AUGUUUAAGUUUAAGGCUUCUUAUGUCGCACUGGCGGCAGUAUUAACCUCGUCGGUAGUUUAUGC
CGACCCCACAAGCUAUACGCACUCUUCCGGCGCCACGGUUAUCGAUAUUGAAAAGCCGAACGCCGC
CGGUGUCUCCCAUAACCUGUACCGCGACUUCAACGUCGGCGCCAAUGGCACCAUCCUCAAUAACAG
CGGCGAUGAUGUCAGCCACAGCACAUUUGGCAAUAUCGCCCGCAACAAUAAUCUGACCGCCGGCAG
CGCUUCGGUGAUUUUGAACGAGGUGACCUCCAAAAACGCCAGUAGCCUGAAGGGCUUUAUCGAAG
UCAACGGUCAGAAAGCGGAUGUGGUAAUCGCCAACCCGAACGGCAUCACCUGUUCCGGCUGUAGC
UUUGUUAAUACCAACAAGGCUAUCCUGACCACCGGCAAGGUUAAUAUGACCGACGAUGGCGCUAUC
GGCAGCUAUACCGUAACGGGCGGCACCCUCACCAUCGGCGAAAAUGGCAUGAACGCCGCCAACGG
CUAUGCGGUUCUGCUCGCCGACGCGAUCAAUAUCAACGGUAAAGUGCAGGCCAACAACGCCCUGG
UCAGCGCGGGCAACUUCACCAUGGAUAACAGCUCUGGCUCGGUGACCUCCGCUGGUAAAAAGGCC
ACCCUGAUCCAGAUGACGGUUAACCCGCAGUACAGCAUCGACGUCAGCAGCCUUGGCGGCAUUGA
GGCCAACAGCAUCAGCAUGGUCGGCAAUAACAUCGGCUUUGGCGUACGUAAUAAAGGCUCUAUCG
UCGCGAAUAGUUCGCUGCAGCUCACCAGCAACGGUAAUCUGCUGAACAAAGGCACGAUCAAAAGCA
ACGGUCUGCUGAGUCAGGUCGCCACCGCCUCGGGCAUCACCAAUGACGGUAGCAUCGCCGGCGCC
UAUUAUUUAAUGCUCUCCAGUGGCGAUUAUAUCGUUAACACCGGUUCUCUCUCCGGCGGCCAGCU
GAUUGCCACCGCUAACGGCAACAUCACCAACGGCGACUCAGGCACGAUGACCGGCACCAGUGGAUU
AAGCCUGACCAGCGGCGGGAAAAUCCGCAACGAAGAAAAAGCCUCCCUGCUGUCAAAUAACCAGAU
UGCCGCCACGGCAAUCGGUGAUUUCCUCAAUGAAGGCAAAAUCAGCGCCAAACACACCAGCCUGAC
GUUUGUCGGCGACAGCUUUAAAAACACUGGCAAUAUUAACUCUACUGGCCAAACCACCAUUCAGUC
GCUUAAACAGGACGGCAGCGCCAAUACGGGCGAGAUCUAUAACCUCGGCAAUAUCACCGGCGAAAA
UAUCAAUCUGCAGACCAAUGGCACGCUGGCGCAAAGCAGCAGUGGUCGUAUUGAGGCAACCAACG
CCAUUACCGCCCACAGCUACUGGCUGAACCAAAAUGGUUAUAUGAAUGCCGCCGAUAUCACCACCG
AUCACGGCGUAGUGAAUAAUUAUGGCAAUAUUACUGCCAAAAAUAUUUCAAUCACGACCUACUCAG
AUAUCACCAACGAAGGGCAGAUCAGCAGCACCGGCGACCUGACCUUAAAUACCAAAAAUAAAGGCG
CGAUCUACAAUUAUUCAACCCUCAGCGCGGGCGGCAACAUGACGUUAACCGCCACCAAAGUGGUCA
AUGGUGGUAAAAGUUGCGGCAUACUGGGCCUGGCGAAAUGCGGCGUCGGGACGUUAACUGCUGA
CAAGCUGGUACUGAACUCAUCGCAGAAAUAUGUUAGCGACAUGGGUGGAAAACAGUAUUUCAAGA
GCACCGAAGUCAACACGGUGAAAUAA
SEQ ID NO: 79
AUGAUGGACAACCUACGCACGGCCGCCAACAGCGUCGUGCUCAAGAUUAUUUUCGGUAUCAUUAU
CGUCUCGUUCAUUUUGACCGGGGUGAGUGGUUACCUGAUUGGCGGUGGCAAAAACUAUGCCGCAA
AAGUGAAUGGCCAGGAGAUUGGCCGUGGGCAGUUUGAAAACGCCGUCGCCAGCGAACGUAACCGU
AUGCAGCAGCAGCUUGGCGAUCAAUUCUCCGAGCUGGCGGCGAACGAAAACUACAUGAAAACCAU
GCGCCAGCAGGUGCUGAACCGCCUGAUCGAUGAGUCGCUUCUGGAUCAGUAUGCCCGCGAGCUGG
GCCUCAGCAUCAGCGAUGAGCAGGUGAAGCAGGCGAUCUUCCAGACCCAGGCGUUCCAGACGAAC
GGUAAGUUCGACAACCAGCGUUUCAGUGGUAUUGUCGCCCAGAUGGGGAUGACCACCGAUCAGUA
CGCCCAGGCGCUGCGUAACCAGCUGACCACGCAGCAGCUGAUUAACGCCAUUGCGGGUACCGACU
UCAUGCUGCCGGGCGAGUCCGAUCAGCUGGCGGCGCUGGUAUCUCAACAGCGGGUGGUCCGCGA
AGCGACCAUCAACGUAAAUGCCCUGGCGGCAAAACAGACCGCCAGCGAUGAGGAAAUCAACGCCUU
CUGGCAGCAGAAUCAGGCCCGUUUUAUGGCGCCGGAGCAGUUCCGCGUCAGCUACAUCAAAAUGG
AUGCCGCCAGCAUGCAGGAGAGCGCCUCUGACGAAGAGAUUCAGUCAUGGUACGACCAGCACAAG
GAUCAGUUCACUCAGCCGCAGCGCAACCGCUACAGCGUGAUUCAGACCAAAACUGAAGCCGAUGCG
AAAGCGGUACUGGCCGAGCUGCAAAAAGGAGCGGACUUCGCCACGCUGGCGAAAGAAAAAUCGAC
CGAUAUUAUCUCUGCCCGCAACGGUGGCGAUAUGGGGUGGAUGGAAGAUGCCUCUACCGUGCCUG
AGCUGAAAGAUGCCGGGCUGAAAGAGAAAGGCCAGCUGUCUGGCGUGAUCAAAUCCUCGGUUGGC
UUCCUGGUAGCUCGUCUGGACGACGUCCAGCCGGCGCAGGUGAAGCCGCUGGCUGACGUGCGUA
AUGACAUUGCGGCGAAAGUGAAGCAGGAAAAAGCGUUGGAUGCUUACUACGCGCUGCAGCAGAAG
GUGAGCGAUGCGGCCAGCAACGAUAAUGAAUCGCUGGCGAGCGCAGCGCAGGUCGCCGGGCUGAA
GGUCGUAGAAACCGGCUGGUUUGGCCGCGAUAACCUGCCGGAGGAGCUGAACUUUAAACCGGUCG
CUGACGCUAUUUUCAACGGCGGUCUGGUGGGUGAGAACGGCGCGCCGGGCAGCAACUCCGAUAUC
AUUACCGUUGACGGCGAUCGCGCUUUUGUUCUGCGCAUUAGCGAACACAAAGCCGAGGCGGUGAA
GCCGCUGGCCGAAGUGAAGGCACAGGUUAGCGAUAUCGUUAAGCACAAUAAAGCGGAACAGCAGG
CGAAACUGGAGGCCGACAAGCUGCUGGCGGCGCUGAAAGACGGCAAAGGCGAUGAAGCGAUGAAG
GCGGCUGGCCUGAGCUUUGGCGCGCCGCAGACGCUUUCUCGUACCGGCCAGGAUCCGCUGAGCCA
GCUGGCAUUUACCCUGCCGCUGCCGCAGCAGGGUAAACCGGUCUACGGCGUGGGCAGCAAUAUGC
AAGGCGAUGUGGUGCUGGUAGCGCUGGAUGAGGUGAAAGCCGGCAGCAUGCCGGAAGAGCAGAA
GAAGGCCAUGGUUCAGGGGAUCACCCAGAACAAUGCCCAAAUCGCUUUCGAAGCGCUGAUGAGCA
ACCUGCGCAAGGCGGCGAAAAUUAAGCUCGGCGACAGCAUCGACCAGCAGCAGUAA
SEQ ID NO: 80
AUGUUCAGGUUAAACCCUUUUAUCCGGGCGGGAUUGUCUGCGUCCGUCGUAUCGUUGGCGUUUCC
GGCUCUGGCCGAUGUGAAUGAAGAAACGCUGGUGGUGACCGCCUCGGCCACUGAACAGAAUGUCA
AAGACGCGCCGGCGAGCAUCAGCGUCAUCACCCAACAGGAUUUACAACGCAAGCCUGUUCAGAACC
UGAAAGACGUGCUGCGCGAUGUGCCUGGGGUCCAGCUCACCAACGAAGGGGAUAACCGCAAGGGC
GUUAGCAUCCGCGGUCUGAGCAGCAGCUAUACCCUGAUCCUGGUCGACGGCAAGCGCGUUAACUC
GCGGAACGCCGUCUUCCGCCACAAUGACUUCGACCUUAACUGGAUCCCGGUGGAUGCUAUUGAGC
GUAUCGAAGUGGUGCGCGGCCCGAUGUCCUCCCUCUACGGCUCCGAUGCGCUCGGUGGGGUGGU
CAACAUUAUUACCAAAAAAAUCGGCCAGAAAUGGACCGGGACGCUGAGUGCUGAUACCACUAUUCA
GGAGCACCGCGAUCGCGGGGAUACCUGGAACGGCCAGUUCUUCACCAGCGGCCCGCUGAUCGACG
GCGUACUUGGAAUGAAGGCCUACGGCAGCCUGGCAAAACGCGCCAAGGACGAUCCGCAGUCAUCC
AGUAAUGCCACCGGCGAGACGCCGCGCAUCGAGGGCUUCACCAGCCGCGAUGGCAAUGUUGAAUU
CGCCUGGACGCCGAACGAAAACCACGAUUUUACCGCAGGCUACGGCUUUGACCGUCAGGAUCGCG
AUUCCGAUUCCCUUGACCGCAACCGCCUUGAGCGGGAGAACUACUCUCUGAGCCAUAACGGCCGC
UGGGAUAUCGGCAAUAGCGAGCUCAAGUUCUACGGCGAAAAGGUGGAUAACAAAAAUCCAGGGCA
GAGCGGGACUAUUACCUCGGAAAGCAAUGCCAUCGACGGCAAGUAUGUCCUGCCGCUGGGCAUGA
UUAACCAGCUGGUGACCUUCGGCGGCGAAUGGCGCCACGACAAACUUAAAGAUCCGGUCAACCUG
AGCAGCGGCGGCCAGUCAACGUCGGCCAGCCAGUACGCCCUGUUUAUCGAAGACGAAUGGCGCAU
CAUCGAGCCGCUGGCGCUGACCACCGGCAUUCGUAUGGACGACCAUCAGACCUAUGGCGAUCACU
GGAGCCCGCGCGCCUAUCUGGUGUAUAACGCCACCGAUACCGUCACCGUCAAAGGCGGCUGGGCG
ACGGCGUUUAAAGCCCCGUCGCUGCUGCAGCUUAACCCCGACUGGACCACCAACUCCUGCCGCGG
CUCGUGCAGCAUCGUCGGUAACCCGGAUCUGAAACCGGAAACCAGCGAAAGCUUCGAGCUCGGUC
UCUACUACCGCGGGGAAGAGGGCUGGCUUGAAAAUGUCGAAGGCAGCAUCACCACCUUCCAGAAU
AAUGUCGACGACAUGAUCGACGUUCUGCGCACCUCCAGCGCCAGCGAAGCGCCGGGCUACCCGAA
CUUUGUCGGCUGGAAAACUGUCAACGGCAAGCGCGUGCCGAUCUUCCGCUAUUUCAACGUCAACA
AAGCCCGCAUCAAAGGGGUGGAGACGGAGGUGAAGAUCCCGUUUGGCGAUGAGUGGAAGCUGAC
GGUGAACUACACCUACAACGAUGGUCGCGAUCUGAGCAAUGGCGGCGACAAACCGCUGCAGACGC
UGCCGUUCCAUACCGCCAACGGCACGCUCGACUGGAAACCGCUGGACGACUGGUCCUUCUACGUG
ACGGCCAACUAUACCGGCCAGCAGCGCGCGGUGAGCGCCACCGGCAAAACGCCGGGCGGCUACAC
CCUGUUUGACGUUGGCGCGGCAUGGCAGGUGACCAAAAACGUGAAACUGCGCUCCGGGGUGCAGA
ACGUGGGUGAUAAAGAUCUGAGCCGGGACGACUACAGCUAUACCGAAGAAGGCCGUCGCUACUUU
AUGGCGGUGGAUUAUCGCUUCUGA
SEQ ID NO: 81
AUGAACAGAGCCGCCACGCUGACCCUCAACGCGCCCCUGCUGAUGCUCGUCGCUGCGCUGGCGCU
UUCAACCCCUUUCACCGCCGGCGCCGCGCCGGCCUUUCUUGAUUACGCCCAACAGCAAACCCAGCA
AUCUCAGGCGCAAGAAAAAAACGAUGCCGCAAGCGCAAAACAAACACAAGAAAGCCGCCAGAGCGC
AGAUAAUAAAAAAACCGGUACCAGCACCUCACAAUUACAAAAAAGAAUCACCAGCCAGCAGGCGGC
GAUUGCACAAAAAGAUAAGCUUAUACAGCAAUUAAAAAAACAGCUUGCCGCUACGCCGCAAACGGA
UACUGCCGGAGCGAAUGAGCAAGCGGCGUUGAAUAAGAGAAUUAAUGAAUUACAGGUCGCCUUAA
GCGCCGCUACUGCAGAAAAAGAGGCAUUAAUAAAAAAAGCAGGCGUUGUGCAGAAUAAUAAUCUAC
AGCAAAGCCAGGCCGCGGCGCGUCAGCAGAUCCAGCAAUUAACGACGCAGAUUCAGCAAGCCGAA
GCUGAAAAUAAACGCCUCAGCGCCAGCUUUACCACGCUUAAUAAAGAUAAACACGCGCUAAUGACC
CAACUGGCCGCAACGGAAAAAGAGAAACAGGCCGCUCUUGAGCAGGUCAAAGCGCUUAACGCUGAC
AAACAGCCGCUGACGACCCGGCUGGCCGCCGCGGAAAAAGAGAAACAGGCCGUCCUCGAGCAGGU
UAAGGCCCUUAACGCCGAUAAACAGUCGCUGACUAUUCGCCUCGCCGCUGCGGAGAAAGCGCAGC
AGGCCGCUGUUGACCAGGCUAAAGCGCUUAACGCUGACAAACAGCCGCUGGCUACCCGACUGGCC
GCCGCGGAAAAAGAGAAACAGGCCGUCCUCGAGCAGGUUAAGGCCCUUAGCGCCGAUAAGCAGUC
GCUGACUAUUCGCCUCGCCGCUGCGGAGAAGGCGCAGCAGGCCGCUCUUGACCAGGCUAAAGCGC
UUAACGCUGACAAACAGCCGCUGGCGACCCGGCUGGCCGCCGCGGAAAAAGAGAAACAGGCCGUC
CUCGAGCAGGUUAAAGCCCUUAACGCCGAUAAGCAGUCGCUGACUAUUCGCCUCGCCGCUGCGGA
AAAGACGCAGCAGGCUGCCCUCGAUCAGGUCAAAGCCCUUAACGCCGAUAAACAAUCGCUGUCCAC
CCGGCUGGCCGCCGCGGAUAAAGCGCCGCAUGGCCCCGCUAACGACGCCGCUGCGCCAAAAAAUG
AGCCACCAGAGAUGGCGGCCAUAGUGGCAGCCUAUCGCCUGCAGGCGGAUAAAGACAACGCCCAG
CUACGGAUGAAAGAAGAUGAAAUCGAACUGCUGAGAACGCAGCUUUCUGUACAGUCCAAAACGCGC
AGCGGCGAGAGCGCCGCCGCCAAACUCAGCGCAUCGGGAGAACAGCAGGCUUAUGCGAUCGGCGC
CUCGAUGGGAAGCGAGGCGCUCAACGUCCUUACCACCCGUCGUACUCAGGGAGUUACCGUCGACG
CAGGCCUGGUGCUGCAGGGCAUCGAAGAUGCCUUUCGCGGCCAGCUUCGUCUCGGAGAGCAGGAA
CGUAACAAGGCGCUGUUUGAUGUGUCGCAGCAGGUUUUUCAGAACCUGAAUAAAAUAGAGCAGAA
AAACAUCAGUGCCGGCAAGAAAUAUCAGCAGGCGUUUGCGCGCAAAAAAGAUGUGGUCUUUAAAG
AGGGCGUCUACAGCCGCGUCGAUUACCUGGGUAAAGGAAAAAUAAGCGGUAAUGACCUGGUUACC
GUGGUGAUCAAAGAGAUGCUGACGGACGGGACGGUGAUCAACGAUAUGGAAGCGAAAGAUCAGGC
GCUUACGCAAAAGCUGGAUGCCUAUCCCCCGGUGUUUCGCGAACCGCUGAAGCGUCUACAGAACC
ACGGCUCCGUGACGCUCGUCGUCCCGCCUGAAAAGGCCUAUGGCAGUAAAGGAUUACCGCCAAAA
AUCCCGCCAGGCGCCACCAUGGUUUAUUCCGUGCGGAUAGUAGAUAGCCAACCCGAGCCGGCAAA
AUAG
SEQ ID NO: 82
AUGAAAAUCCUGUCCGUGCGUCACGCCGCCCUCCCGGCCCUGCUCUUGCCGCUCAUUGCGGCAGC
CCAGGCCGCUGAUGAACAAACCAUGGUGGUGACCGCCGCGCCAACCACGGUUUCUGAACUGGAUA
CCCCCGCCGCCGUCAGCGUGGUGAAUGGGGAUGAGAUGCGCCAGGCCGCGCCGCGCGUCAAUCUC
UCUGAAUCGCUGGGCGCCGUGCCGGGCCUGCAGGUGCAGAACCGGCAAAACUAUGCCCAGGAUCU
GCAGCUGUCGAUUCGCGGCUUUGGCUCGCGCUCAACCUAUGGCGUGCGCGGACUGCGCAUCUAUG
UGGAUGGCAUUCCGGCCACCAUGCCCGACGGCCAGGGGCAGACCUCAAAUAUUGAUAUCGGCAGC
GUUGACACCAUUGAGGUGCUGCGCGGCCCCUUCUCUGCCCUGUACGGUAACUCGUCCGGCGGGG
UGAUCAACGUCACCAGCCAGACCGGCACCCAGCCGCCCACCGUGGAAGCCAGCAGCUACUAUGGCA
GCUUCGGCACCUGGCACUACGGGAUGAAAGCCACUGGCGCCGUUGGCGACGGCAGCCACGCAGGC
GAUGUGGAUUACACGGUCUCAACCAAUCGCUUCACCACCCAUGGCUAUCGCGAUCACAGCGGCGC
GCGCAAAAAUCUGGCGAACGCCCGGCUGGGGGUGCGCAUCAACGACGUCAGUAAGCUGACUCUGC
UGCUGAAUAGCGUGGAUAUCAAAGCCAAUGACGCCGGUGGCCUGACCGCCGAUGAAUGGCGCGAU
AACCCGCGCCAGUCGCCGCGCGGCGACCAGUAUAAUACCCGCAAGAAUACCCGACAGACCCAGGCC
GGCCUGCGCUAUGAGCGCCAGCUCAGUGCCCAGGACGAUCUCAGCGUUAUGAUGUACGCUGGAGA
ACGUGAAACCACUCAGUUCCAGUCGAUCCCGCGCGCGCCGCAGCUGAAGCCGAGCCAUGCCGGCG
GGGUGAUCGACCUUACCCGUCACUACCAGGGGAUCGAUACCCGGCUGACCCAUCGCGGAGAGCUG
CUGGUGCCCGUCACGCUCACCGCCGGUCUCGACUACGAAAACAUGAGCGAGCGGCGCAAAGGGUA
UGAAAACUUUGUGAUGGUCAACGGCGCGCCGCAGUAUGGCGAACAGGGCGCGCUGCGCCGUAACG
AACGCAACCUGAUGUGGAACGUCGACCCCUACCUGCAGACCCAGUGGCAGCUCACUGACAAACUCU
CGCUCGAUGCCGGGGUUCGCUACAGCUCGGUAUGGUUCGACUCGAACGACUACUACAUCACCCCA
GGCAAUGGCGAUGACAGCGGUGAUGCCAGCUAUCACAAAUGGCUGCCCGCGGGCUCGCUGAAAUA
UGCCCUGACCGACGCGUGGAACGUCUAUCUUUCCGCCGGCCGCGGCUUCGAGACGCCAACCAUUA
ACGAACUCUCCUACCGCUCCGAUAACCAGAGCGGCCUCAACUUCGGCCUGAAACCCUCCACCAACG
ACACGGUGGAGAUCGGCAGCAAGACGCGGAUCGGCAAUGGGCUGUUCACCGCCGCCCUGUUCCAG
ACCAAUACCGAUAAUGAGAUUGUGGUCGACAGCAGCAGCGGCGGGCGCACCAGUUAUAAAAACGC
CGGCAAGACCCGCCGUCAGGGGAUGGAGCUGGGGCUGGAUCAGCAGUUUGGCGAGAGCUGGCGU
CUGAAGGCGGCCUGGACCUGGCUGGACGCGACCUAUCGCACUAACGUCUGCGACGACGCCAGCUG
CAAUGGCAAUCGCAUUCCGGGGAUCGCGCGCAAUAUGGGCUACGCCUCCUUUGGCUAUCAGCCGG
AGCAAGGUUGGUACGCCGGGAGCGAUAUUCGCUAUAUGAGCGAUAUCAUGGCCAAUGACGAAAAC
ACCGCCAAAGCGCCCUCCUGGACGGUGGUUGGCCUGACGACUGGCUAUAAAUGGAGCUACGGCAG
GAUGGAUAUGGAUCUGUUCGGUCGCAUCGACAACCUGUUCGACCGGGAGUACGUCGGGUCUGUCA
UCGUUAACGAGUCUAACGGACGUUACUACGAGCCUGCCCCGGGACGUAACUACGGCAUCGGCCUG
AACCUCGCCUGGCGCUUCGAAUAA
SEQ ID NO: 83
AUGAAAUACACGUCUCACUUCCCGCUGGGGAUCGUCAUUCCUCUGCUCGCCUGUAGCGUGCCGCU
GCAGGCGGCAGAGAACAUGACCGAACAAUCGACGCCUGACGAGAGCGCCGCCACUGCCGAAAAUCA
CGAGGAGACGAUGGUCAUAACCGCCGCCAGGCAGAACCUGCAGGCGCCGGGCGUGUCGACCAUCA
CCGCAGAAGAGAUCCGCAAACAUCCCCCCGCCCGCGAUGUGUCGGAGUUAAUUCGUACGCAGCCC
GGGGUAAACCUGACCGGCAACUCCACCAGCGGGCAGCGCGGCAACAACCGGCAAAUUGAUAUCCG
UGGCAUGGGGCCCGAGAAUACGCUGGUGCUGGUCGAUGGUAAACCGGUGACCAGCCGUAACUCG
GUGCGGUAUGGCUGGCGCGGCGAUCGUGACUCCCGCGGCGAUACCAGUUGGGUGCCAGCGGAGA
UGAUCGAUCAUAUCGAUGUGAUCCGCGGCCCGGCGGCGGCGCGCUAUGGUAAUGGCGCGAUGGG
CGGGGUCGUCAACAUCGUGACCAAACCGACCACGCGAGAAUGGCACGGGUCGUGGAAUACCUAUA
UGAAUGCUCCGCAGCACCGUAAAGAAGGGGCGACGAAACGUACUAACUUUAGCCUCAAUGGUCCG
CUGUCGGACAGUGUCAGCUUCAAUCUCUGGGGUAAUCUGAGUAAAACCCAGGCCGAUGCACAGGA
UAUUAACGCCGGGCAUGAAGCGGAACGUACCGGUUCCUACGCCGGUUCUUAUCCCGCCGGACGUG
AAGGGGUGGUGAACAAAGAUAUUCACAGUAAGCUGCGCUGGGAGUUUGCCCCGAUGCAGGCCCUG
GAGUUUGAGGCCGGUUACAGCCGCCAGGGUAAUCUCUAUGCCGGCGACACACAAAACACCAAUACC
AGUACGCUGGUGAAGAGUAUGUACGGGAAAGAGACCAACCGUCUCUACCGGCAAACUUACGGCGU
AACAUGGACCGGCGGCUGGGAUAAUGGCGUGACCAGCAACAGCUAUGCCCAGUACGAACACACCC
GUAACUCGCGAAUGGAUGAAGGGCUGGCGGGCGGUACGGAAGGGAUCUUCUCCAGUAGCGAGUU
UUCAGAUAUCGAUCUGGCCGAUGUCCUGCUACAUAGUGAAGUGAAUAUUCCGUUUACGCUGGGGG
UCGAUCAGAAUCUGACGCUGGGGGCAGAAUGGAAUCAGCAGCGGAUGAAAGAUGGCGUAUCGACA
ACCCAGGCGCUCUCUUAUGGCACUAUCGAUGGCGUAUCGGCUACCGGUCGUAGCCCGUACUCCAG
UGCCGAGAUCUUCUCGCUGUUUACCGAAGAUAAUAUGGCGCUAACGGACAGCACCAUGCUGACAC
CCGCUCUGCGCUUCGAUCACCACAGCAUCGUCGGCAAUAACUGGAGCCCCUCACUGAACCUGUCUC
AGGAGCUGACGGACGACUGGACGCUGAAGCUGGGCAUUGCCCGUGCUUACAAGGCGCCUAACCUC
UACCAGUUGAACCCGAACUAUAUUCUCUACAGCAACGGUCAAGGCUGUUACGCCAGUAGUUCCGCC
UGCUAUCUGAUGGGGAAUAGCGAUCUGAAAGCGGAGACCAGCGUUAAUAAAGAGAUUGGUCUUGA
GUACAAGCAUGAUGGCUAUCAGGCGGGGAUCACCUGGUUCCGUAACGACUAUCACAAUAAGAUUG
AGUCAGGGUAUGCGGCGGUGGGUACCGCCAGCAACGGCACCACCAAUAUCUAUCAGUGGGAAAAC
GUACCAAAGGCGUUAGUGGAAGGCCUGGAAGGAACGCUGAAUCUGCCGGUGGGGGAGGCGGUUA
ACUGGAGCAAUAACCUGACCUGGAUGCUGCAGAGCAAGAAUAAGACGACCGGCGACCGGCUGUCA
GUGAUCCCGCAGUUUACCCUGAACUCGACUUUGAGCUGGCAGGUUCGUGAAGAUCUCUCCCUGCA
GAGCACCUUUACCUGGUAUGGCCGACAGAAACCAAAACGCUUCAAUUAUAAGGGCGAGGCGGUCA
GCGGCAGCGAACUAAACGAAGUCAGCCCAUACAGCAUUGUCGGCCUCAGUGCGACCUGGGAUGUG
AACAAAAAUCUGAGCUUCACCAGCGGGAUAGAUAACCUGUUUGAUAUUCGCCACUACCGGGCAGG
GAAUGCGCAAACGACCGGCAACGCGACGACGGGAGCUUAUCUGUAUGGCGCGGGUGCCGAGACCU
AUAACGAAUCGGGGCGGACCUUCUUUAUGAGCGUUAAUACUCAUUUCUGA
SEQ ID NO: 84
AUGGAAAAAAACGCUUCUCUGCCUUUCGGCAGUUUCAACUCAUUGGCAUUGUUUACAGGUCUGUG
UCUGGGAGCCUCGCCGGCAGCAGGCAUCGCAGCGGAAAAUUCGGUCAAAAAUAGUGAAGAGACGC
UGGUAGUGGAAGCCGCUCCGCCUUCACUCUACUCCCCCGGCGCUUCCGCCGAUCCCAAGUUCAAU
AAACCGCUGGUCGAUACCACCCGCACCAUCACCGUGAUCCCGGAACAGGUGAUUAAAGAUCAGGGC
GUCACCAACCUGACUGACGCCCUCAAAAACGUUCCCGGCGUCGGGGCGUUUUAUGCCGGGGAGAA
UGGCAGCUCAACCACCGGGGAUGCCAUCUUUAUGCGCGGCGUGGAUACCUCUAACAGCAUCUAUG
UGGACGGCAUUCGCGACAUCGGUAGCGUGACGCGCGAUACCUUCAAUACCCAGCAGGUGGAAGUC
AUCAAAGGGCCCGCCGGCACGGACUAUGGCCGCAGCGCGCCCUCCGGCUCGAUCAAUAUGAUCAG
CAAGCAGCCGCGCCUUGACUCCGGGAUCGACGGCUCGGCCAGCAUCGGCAGCGCCUGGUCGCGCC
GGGGCACUCUCGACCUGAACCAGGCGUUUAGCGACAACGCUGCGUUCCGUCUGAACCUGAUGGGG
GAAAAAACGCAUGACGCUGGUCGGGACCGCAUUGAAAACGAACGCUAUGGCAUCGCACCGUCGCU
GGCCUUCGGCCUUGAUACCCCAACUCGUCUGUAUCUGAACUAUCUGCACGUCCGGCAGAACAACAC
CCCGGAUGGCGGGAUCCCUACCGUCGGCCUGCCGGGCUAUUCGGCGCCUUCGCCGAAGUAUGCCG
CACUCAACUCCGCCGGGAAGGUCGAUACCAGCAAUUUCUAUGGCACCGACUCCGAUUACGAUAAAU
CUACUACCGACAGCGGUACCCUGCGCUUCGAACACGAUCUGACGGAGAAUACCACCGUGCGCAAUA
CCACCCGCUGGUCGCGAGUGAAACAGGAGUAUCUUUUGACCGCGGUGAUGGGCGGCGCGAACAAU
AUCACCGCCCCCGAUAUCAAUGACGUCAACACCUGGAGCUGGUCGCGUCUGGUUAAUACCAAAGAU
GUCAGCAACCGUAUUCUGACCAACCAGACCAAUAUCACCUCGACUUUCAAUACUGGCUCGAUAGGC
CAUGACGUCAGCGCCGGCGUGGAGUUUACCCGGGAAAACCAGACCAACUAUGGCGUUAACGCCAG
GACCGCGCCGGCGGUGAAUCUCUACCAUCCGGUGAGCAACCUGUCGAUUGGCGGGCUGGACAGAA
ACGGGGCGAACGCCAACGGCCAGACCGAUACCUUCGGGAUUUAUGCCUUUGAUACGCUGACGCUG
ACCGAGCGGAUUGAGAUCAACGGCGGGCUGCGUCUCGACAAUUACCAUACCAAAUAUGACAGCGC
CACCGCCUGCGGCGGCAGCGGACGCGGGGCUAUCGCCUGCCCGCCCGGACAGUCGACCGGCAGCC
CGGUCACCACUGUCGAUACCGCUAAAUCCGGCAAUCUGGUUAACUGGAAAGCCGGGGCGCUGUAC
CGCUUAACCGAGCAGGGCAAUGUCUACGUCAACUACGCCAUCUCACAGCAGCCGCCGGGAGGCAG
CAGCUUCGCCCUGGCCGCCAGCGGCAGCGGCAACAGCGCUAACCGGACCGACUUUAAGCCACAGA
AGGCGAAAUCCAGCGAGCUGGGCACCAAGUGGCAAAUCUUCGACAACCGUCUGCUGCUCAGCGCG
GCGUUAUUCCGCACCGAUAUUGAAAACGAAGUGGCCGCCAACGAUGACGGAACCUGGUCGCAGUA
CGGCAAAAAGCGCGUGGAGGGGUAUGAACUCUCCGCGACCGGAAACCUGACCCCGGACUGGACGA
UUAUCGCCGGCUACACUCAGCAGCAUGCGACAGUGACGGAGGGACAGAACGUUGCACAGGAUGGA
UCUUCCGCCCUGGCCUACACCCCGAAACAUGCCUUUACGCUGUGGACGCAGUAUCAGGCCACCAGC
GAUCUGUCCGUCGGCGGCGGUGUGCGCUAUGUCGGAAGCCUGCGCCGGGGCAGCGAUGGUGCAG
UCGGUACCCCGGAUCACACCGAGGGCUACUGGGUUGCCGACGCCAAACUGGGCUAUCGGGUCAAC
AGCAACCUCGAUCUGCAGCUCAAUAUGUAUAACCUGUUUGAUACCGAUUACGUGGCCUCCAUCAAC
AAGAGCGGCUAUCGCUAUCAUCCGGGCGAACCCCGGACCUUUAUGCUGACGGCGAACGUCCAUUU
CUGA
SEQ ID NO: 85
AUGGCGACUAUGUACAAAUCGACUCCGUCAGCAGCAUGGUGUAAAAAACGCCUGCUGGUGACCUC
UUUGUUUGCAGCAAUUUAUCAGACUUCUGCCAUCGCAGCAGAUACUUCCGCCGUUAGCGGCGAGG
CGGUGGAUGACACCUCGGAACAAAUGACCGUCACCGCCCCCGCGCCGGUGCAGAAAGCCGGUAGC
GAACACAGCAUCAGCGCCCGGGAGCUGGAGAAUAAAGGCGCUAACGAUUUCGGCUCAAUCAUGCG
CUAUGAGCCGCUCAUCAGCGCCACCGGGGCCAGCGGCGGCUCCGGCAACGGCAAAAGCGGCUUCG
ACCGCGGAGGUUACACCGGCUACAACAUUCGCGGUAUGGAGAGCAACCGCGUAGGCAUCGACGUG
GACGGUAUCGCGCAACCCAACGCCACCGGCCGCGGCUACGUCGGCCGCGCCGGGCUCAACACCUU
CGGCAUCGGCCGCGAUUAUAUCGACCCGUAUAUGUACGGCAGCGUUGAUAUCCAGUCCGGCGCCA
CCUCGACGGAAACGGCCAACAGCGCUAUCGGGGGGAAUGUCUCCUUCCGCCCGAAAUCAGCGGAU
GAUUACCUGCGCCCGGGCAAGACCAGCGCCUUCGGCUACCGCAGCGGUUACGACUCUGCGGAUCG
CAGCUGGCACAACGGGGUGACCGUCGCCGGCGGCGAUGAGUUCCUGCGCGGGAUUUUGGUCUAU
AGCCGCCGUGACGGCCAGGAAACUGAAAACAACAGCGGCACCGUCGACGCCUACCCGGCGAACUG
GCACUCCGAUGCUUUUCUGGCCUCCGGGAUCUGGCAGCCUAACGAUGAGCACAAGCUGACCAGCA
CCUUCGACUAUUACCAUAAAACCAACCACACCCACUACGAUACCUGGGACUCCAGCGGCAACAGCA
CCAUCGGCACCGCCAACCAGACCAGCCAGACCCGGCGCUGGGGCCUGAGCCUGAAGGAUGACUGG
ACGCCGAUGAACGACUACCUCGACAGCGUCUCCACAAAAAUCUACUACCAGCAUACCGAAGCCCAU
GACUGGACUUAUAUGCCGGACAGCGUCACCCGCAAAAUGCAGACGGUGAACUCUAACUACGAUACC
GACACCUGGGGCCUGCAGACCGCGCUGGCGAAAACCCUGGGCCGCCACGAUCUGAGCGCCGGUUU
CAACGCCAGCACCAGCAAAACCCAGCGGCCGUUCAGCCAGUCGCCGAUCCCCAGCGUUUACAGCGA
GAUCAUGCAGCCGGAGGCAGACAGCCGCAGCUACACCCUCGGCGGCUUUGUCCAGGAUAAGAUCA
ACUUCGAUCUCGACAGCCACAACUUCGCCGUUAUUCCCGGCGUGCGCGUGGUGCAUCAAUCGACU
AAGCCGGAAAAUCUGUCCGAUCUCGCCGCCAACAGCAGCGUGCUGAGCGAAUCGUCGGUGGCGAA
UCUGUACGGCAAAAACAGCGAUACCCAGGUUCUGCCGUCGUUGACCUUCCAGUACGACCUCACCCC
GCGCCUGAUGACCUACCUGCAGUACCAGCGCGGGGCGCAGUUCCCCAACGCCAGCCAGCUGUAUG
GCUCCUGGAACCUCGGCUCCAGCUACGCCGGCAGCCAGCAGUAUGCCCUGAUCGGCAAUACCGAU
CUGAAGACGGAAACCAGCGAUAAUCUCGAGUGGGGGCUGAAAGGGGAAGUUACCGAAGGCAUCAC
CCUGCGCACGGCGCUGUUCUACAACAGCUAUAAGAACUUUAUCGCCUAUACCCGCUAUACCCGCGC
CAACAAUCCGGGCCAGUUCACGAAUGUGCCGUCGAACAUCUACACCAUUUAUCAGGCGGAAAACCG
CGAUAAAGCCUAUAUCUACGGCGGUGAGAUUAGCACCAAAUUUAACUUUGGCACCUGGUUUGAGC
AGGUGGACGGCCUGAGCGCCACCCUCGCCCUCGGCUAUAGCGAAGGGAAAUCGAAAUCCAGCUAC
AGCGGCGAUAAAUACGUCGACCUCGACAGCGUGGCGCCAAUGAAAGCCAUCGUCGGCGUGGCGUG
GGACGAUCCGGCGAAACGCUACGGCACCGCCCUGACGGCGACCUUUGUCAAAGGGAAACAGGCGA
CCGCCACCAACCGCGAAAGCUACAGCAACAGCGGAUCCGCCAUCACCGAUGCCAGCAGCGACUAUA
UGCGCGUGCCGGGCUACGGCAUGCUGGACUGGACCGCGUACUGGCAGGUGGCGAAAAACGUGCG
CCUCAAUGGCGGGGUCUACAACCUCACCGAUCGUAAAUACUGGGAUUACCUGAGCAGCCGCAAUA
UCGAGACCGGCACCAACCAGGACGCCAACGAUAAAGCGCUGGCGGUGAUGCCGGGCCGCACCUGG
CAGCUGGGCGUCAACGUCGACUUCUGA
SEQ ID NO: 86
AUGGCGAUGAAAAAGUUGCUCAUAGCGUCGCUGCUGUUUAGCAGCGCGACUGUAUACGGUGCUGA
AGGGUUCGUGGUGAAGGACAUUCAUUUCGAAGGCUUGCAGCGUGUCGCUGUUGGUGCGGCCCUC
CUCAGUAUGCCAGUGCGUCCUGGCGAUACGGUGACCGACGAUGAUAUCAGUAACACUAUUCGCGC
GCUGUUUGCCACUGGCAACUUCGAGGACGUCCGCGUCCUGCGCGAUGGUGAUACCCUGCUGGUUC
AGGUGAAAGAGCGUCCGACGAUCGCCAGCAUCACUUUCUCCGGCAACAAGUCGGUGAAAGAUGAC
AUGCUCAAGCAGAACCUUGAGGCCUCAGGCGUUCGGGUGGGCGAGUCGCUUGACCGCACGACCAU
CGCGGAUAUCGAGAAGGGUCUUGAAGACUUCUACUACAGCGUCGGUAAAUACAGCGCCAGCGUCA
AAGCAGUCGUUACGCCGCUGCCGCGUAACCGUGUCGAUUUGAAGCUGGUCUUCCAGGAAGGCGUC
UCCGCAAAAAUUCAACAGAUCAACAUCGUCGGCAACCAUGCGUUUUCGACCGAUGAGCUGAUCUCC
CACUUCCAGCUGCGCGAUGAGGUGCCGUGGUGGAACGUGGUCGGCGACCGUAAAUACCAGAAGCA
GAAGCUAGCGGGCGACCUUGAAACCCUGCGCAGCUACUACCUGGAUCGCGGCUAUGCCCGUUUCA
ACAUCGAUUCUACCCAGGUCAGCCUGACGCCGGAUAAGAAAGGGAUCUACAUCACCGUCAACAUCA
CCGAAGGCGAUCAGUACAAGUUUUCCGGAGUGCAGGUGACGGGCAACCUCGCUGGCCAUUCCGCG
GAAAUCGAAGCGCUGACUAAAGUUGAGCCAGGCGAACUGUACAACGGCGCGAAAGUGACCAAGAU
GGAAAACGACAUCAAGAAACUGUUGGGUCGUUAUGGUUACGCCUAUCCGCGCGUGCAGUCGCAGC
CGGAGAUCAACGACAGCGAUAAAACCGUUAAGCUGCACGUUAACGUCGACGCAGGCAACCGUUAUU
ACGUGCGUAAAAUUCGCUUCGAAGGCAACGACACCUCUAAAGAUGCCGUACUGCGCCGCGAAAUG
CGCCAGAUGGAAGGCGCAUGGCUGGGCAGCGACCUCGUCGAUCAGGGUAAAGACCGUCUCAAUCG
UUUAGGUUUCUUUGAAACGGUGGAUACUGAUACCCAGCGCGUGCCGGGCAGCCCGGACCAGGUCG
ACGUUGUCUACAAGGUGAAAGAGCGUAACACCGGUAGCUUCAACUUCGGUAUCGGCUACGGCACC
GAGAGCGGCGUCAGCUUCCAGGCGGGCGUUCAGCAGGAUAACUGGUUAGGUACUGGCUAUGCUG
UCGGGAUCAACGGUACCAAAAACGACUACCAGACCUAUACCGAGCUGUCGGUGACCAACCCGUACU
UCACCGUAGACGGUGUAAGCCUCGGCGGUCGUGUCUUCUAUAAUGACUUUGAUGCGAACGAUGCG
GAUCUGUCUGACUAUACCAACAAAAGCUAUGGUACAGACAUUACGCUGGGCUUCCCGGUCAACGAA
UACAACACGCUGCGCGCCGGCGUCGGUUAUGUGCAUAACUCCCUGUCCAAUAUGCAGCCGCAGGU
GGCAAUGUGGCGUUACCUUAACUCGAUGGGCCAGUAUCCGGACAACACCAACGACCGGAACUCGU
UCAGUGCGAAUGACUUCACCUUCAACUACGGUUGGACCUAUAACAAGCUUGACCGCGGCUUCUUC
CCAACGGAAGGUUCGCGCGUCAACCUGAACGGUAAGGUGACCAUUCCGGGCUCAGACAACGAGUA
CUACAAAGCGACGCUGGAUACCGCGACCUACGUGCCGAUCGACAACGAUCAUCAGUGGGUAGUAC
UGGGUCGUACGCGCUUUGGUUAUGGCGAUGGUAUCGGCGGCAAAGAGAUGCCGUUCUAUGAGAA
CUUCUAUGCCGGUGGUUCCAGCACCGUGCGUGGCUUCCAGUCGAACACCAUUGGUCCGAAGGCGG
UGUACUUCCCGGCAAGCAGUCGUCAUGAUGAUGACGAUAGUUACGAUAAUGAAUGUAAGAGCACC
GAAUCCGCACCGUGUAAAUCCGAUGAUGCGGUGGGCGGUAACGCGAUGGCGGUGGCCAGCCUUG
AGCUGAUUACCCCGACGCCGUUUAUUAGUGACAAAUAUGCGAACUCGGUCCGUACUUCCGUCUUC
UGGGAUAUGGGUACCGUAUGGGAUACUCACUGGGAUUCGAGCGCGUACGCUGGUUAUCCGGAUU
ACAGCGAUCCGAGCAACAUCCGUAUGUCUGCGGGUAUUGCCGUGCAGUGGAUGUCGCCGUUGGG
GCCGUUGGUCUUCUCCUACGCCCAACCGUUCAAAAAGUACGAUGGAGACAAAGCCGAACAGUUCCA
GUUUAACAUUGGUAAAACCUGGUAA
SEQ ID NO: 87
AUGACAGAUGUGACUAUUAAAGCGCUGGCCUCAGAGAUUCAGACCUCUGUGGAUCGCCUGAUACA
GCAAUUUGCUGACGCAGGCAUCCGCAAAUCGGCUGAUGAUUCUGUGACCUCGCAAGAGAAACAAA
CUUUGUUGACGCACCUGAACCGUGAACACGGCUCGGCGCCAGACAAGCUGACGUUACAGCGUAAG
ACGCGCAGUACGUUAAAUAUUCCAGGUACCGGUGGAAAGAGUAAAUCGGUACAAAUCGAAGUCCG
CAAGAAACGCACCUUUGUGAAACGCGAUCCGCAAGAGGCUGAACGCCUGGCCGCGGAAGAGCAGG
CGCAGCGUGAAGCGGAAGAGCAGGCCCGUCGUGAAGCUGAAGAAGCAGCGAAACGCGAGGCGCAA
UUAAAAGCUGAACGUGAGGCCGCAGAACAAGCUAAACGUGAAGUCGCUGAUAAAGCGAAACGUGAA
GCUGCGGAAAAAGACAAAGUGAGCAAUCAACAUACCGACGAAAUGACCAAAACCGCCCAGGCuGAA
AAGAUCCGUCGCGAGAACGAAGCCGCGGAAUUGAAGCGCAAAUCGGAAGAAGAAGCACGCCGCAAA
CUUGAAGAAGAAGCGCGCCGUGUAGCGGAAGAAGCACGCCGUAUGGCUGAAGAAAACGAAAAAAA
UUGGUCUGAAACCUCAGACAGCCCGGAAGAUAGCAGCGACUAUCACGUCACCACAUCACAGCAUGC
UCGUCAGGCUGAAGAUGAUAACGAUCGUGAAGUCGAAGGCGGUCGCGGCCGUAGCCGUAGCAGCA
AAGCGGCUCGUCCGGCGAAGAAAGGCAACAAACACGCUGAAUCGAAAGCUGAUCGUGAAGAAGCC
CGCGCGGCCGUGCGCGGCGGUAAAGGCGGUAAGCACCGUAAAGGUUCCGCUCUGCAGCAGGGCU
UCCAGAAGCCAGCGCAGGCCGUUAACCGUGACGUCGUAAUCGGCGAAACCAUCACCGUUGGCGAA
CUGGCUAACAAGAUGGCGGUGAAAGGUUCUCAGGUCAUCAAAGCGAUGAUGAAGCUGGGCGCCAU
GGCGACCAUCAACCAGGUCAUCGACCAGGAAACCGCACAGCUGGUUGCCGAAGAGAUGGGCCACA
AAGUUAUCCUGCGUCGUGAAAACGAACUGGAAGAAGCCGUAAUGAGCGACCGUGACACCGGCGCG
GCGGCUGAACCGCGCGCACCGGUCGUGACCAUUAUGGGUCACGUUGACCACGGUAAAACCUCGCU
GCUGGACUACAUUCGUUCUACCAAGGUUGCCUCCGGCGAAGCGGGUGGUAUUACCCAGCACAUCG
GUGCUUACCACGUCGAAACCGACAACGGCAUGAUCACCUUCCUGGAUACCCCGGGCCACGCCGCG
UUUACCUCCAUGCGUGCUCGUGGCGCGCAGGCGACGGAUAUCGUGGUUCUGGUGGUGGCGGCAG
ACGACGGCGUGAUGCCGCAGACUAUCGAAGCUAUCCAGCACGCUAAAGCGGCGCAGGUACCGGUG
GUAGUGGCGGUGAACAAGAUCGAUAAGCCAGAAGCCGAUCCGGAUCGCGUGAAGAACGAACUGUC
CCAGUACGGCAUCCUGCCGGAAGAGUGGGGCGGCGAGAGCCAGUUCGUCCACGUUUCCGCGAAAG
CGGGUACCGGCAUCGACGACCUGCUGGACGCGAUCCUGCUGCAGGCUGAAGUUCUUGAGCUGAAA
GCGGUCCGCAACGGUAUGGCGAGCGGCGCGGUCAUCGAAUCCUUCCUUGAUAAAGGUCGUGGUCC
GGUAGCUACCGUUCUGGUUCGCGAAGGUACUCUGCACAAGGGCGACAUUGUUCUGUGCGGCUUC
GAAUAUGGCCGUGUGCGCGCGAUGCGUGACGAACUGGGUCGCGAAGUGCUGGAAGCGGGUCCGU
CCAUUCCGGUGGAAAUCCUCGGCCUGUCCGGUGUGCCGGCUGCCGGUGAUGAAGUGACCGUAGU
GCGUGACGAGAAAAAAGCGCGUGAAGUGGCGCUGUAUCGUCAGGGCAAAUUCCGUGAAGUUAAGC
UGGCGCGUCAGCAGAAAUCUAAACUGGAAAACAUGUUCGCUAACAUGACCGAAGGCGAAGUUCAC
GAAGUGAACAUCGUACUGAAAGCGGACGUACAGGGUUCUGUCGAAGCGAUUUCCGAUUCCUUACU
GAAACUGUCUACCGACGAAGUGAAAGUGAAGAUCAUCGGUUCCGGCGUAGGUGGUAUCACCGAAA
CCGACGCUACCCUGGCAGCAGCAUCCAACGCGAUUCUGGUUGGCUUCAACGUUCGUGCCGAUGCC
UCUGCGCGUAAAGUUAUCGAAGCGGAAAGCCUGGAUCUGCGUUACUACUCCGUCAUCUAUAACCU
GAUCGACGAAGUGAAAGCGGCGAUGAGCGGCAUGCUGUCUCCGGAACUGAAACAGCAGAUCAUCG
GUCUGGCUGAAGUGCGUGAUGUCUUCAAAUCGCCGAAAUUCGGCGCCAUCGCGGGCUGUAUGGU
UACCGAAGGGACGAUCAAACGUCACAACCCAAUCCGCGUUCUGCGUGACAACGUGGUUAUCUAUGA
AGGCGAGCUGGAAUCCCUGCGCCGCUUCAAAGAUGACGUUAACGAAGUCCGUAACGGCAUGGAAU
GUGGUAUCGGCGUGAAGAACUACAACGACGUUCGCGUUGGCGAUAUGAUCGAAGUGUUCGAAAUC
AUCGAAAUCCAGCGUAGCAUCGAUUAA
SEQ ID NO: 88
AUGAAAAGAAUGUUAAUCAACGCAACUCAGCAGGAAGAGUUGCGCGUCGCCCUUGUUGAUGGGCA
GCGCCUGUACGACCUGGAUAUCGAAAGCCCCGGGCACGAACAGAAAAAAGCGAACAUCUACAAAGG
CAAAAUCACCCGCAUUGAACCCAGCCUUGAAGCCGCGUUUGUUGAUUACGGCGCCGAGCGUCAUG
GUUUCCUCCCCCUCAAAGAAAUCGCCCGCGAAUACUUCCCCGCCAACUACAAUGCGCAUGGUCGUC
CUAAUAUCAAAGACGUACUGCGGGAAGGUCAGGAAGUUAUCGUGCAGAUUGAUAAAGAAGAACGC
GGCAACAAAGGCGCUGCGCUCACCACCUUUAUCAGCCUCGCGGGCAGCUAUCUGGUACUGAUGCC
GAACAACCCGCGCGCCGGGGGAAUUUCCCGCCGUAUCGAGGGCGACGACCGUACCGAACUGAAAG
AAGCGCUGGCGAGCCUGGAGCUUCCGGACGGCAUGGGCCUGAUCGUUCGCACCGCUGGCGUCGG
CAAAUCCGCCGAAGCCCUGCAGUGGGACCUGAGCUUCCGCCUGAAGCACUGGGAAGCGAUUCAGA
AAGCCGCGGAAAGCCGUCCGGCGCCGUUCCUGAUCCACCAGGAAAGCAACGUCAUUGUCCGCGCC
UUCCGUGACUACCUGCGCCAGGACAUCGGCGAAAUCCUGAUCGAUAACCCGAAAGUGCUUGAGCU
GGCGCGCCAGCAUAUCGCCGCGCUGGGUCGUCCGGAUUUCAGCAGCAAAAUAAAACUGUACACCG
GUGAAAUCCCGCUGUUCAGCCAUUAUCAGAUCGAAUCGCAAAUUGAGUCCGCCUUCCAGCGCGAA
GUGCGCCUGCCUUCCGGCGGGUCUAUCGUUAUCGAUAGCACCGAAGCGCUGACCGCGAUCGAUAU
CAACUCCGCCCGCGCCACCCGCGGCGGCGAUAUCGAAGAGACAGCCUUCAAUACCAACCUCGAAGC
GGCUGACGAAAUUGCCCGCCAGCUGCGUCUGCGCGACCUCGGCGGCCUGAUCGUUAUCGACUUCA
UCGAUAUGACCCCGGUUCGCCACCAGCGCGCCGUGGAGAAUCGUCUGCGCGAAGCCGUCCGUCAG
GACCGUGCGCGCAUUCAGAUCAGCCAUAUUUCGCGCUUCGGCCUGCUGGAGAUGUCCCGUCAGCG
CCUGAGCCCGUCGCUGGGCGAGUCCAGCCACCACGUCUGCCCGCGCUGCUCCGGCACCGGCACCG
UGCGUGAUAACGAAUCGCUGUCGCUCUCUAUUCUGCGUCUGAUCGAAGAAGAAGCGCUGAAAGAG
AAUACCAAAGAAGUCCACGCCAUUGUUCCGGUACCGAUCGCCUCCUAUCUGCUGAACGAAAAACGU
GCCGCAGUGAGCGCUAUCGAAUCCCGUCAGGGCGAUGUGCGCGUUAUUAUCGUGCCAAACGACGA
AAUGCAAACGCCGCACUACUCCGUCCUGCGCGUGCGCAAAGGUGAAGAAACCUCAACGCUGAGCUA
UCUGCUGCCGAAGCUGCAUGAAGAAGAAAUGGCGCUGCCAGGCGACGAUGAGCCGGCGGAGCGGA
AACGUCCGGAACAGCCGGCCCUGGCCGCUUUUGUCAUGCCAGAUGCGCCGCCAGCCCCGAUGCUC
GAAGAGCCUGCCGCCGCGCCUGUCGCCGCAGCGGCACCGGUCGCGGCCGCCGCACCGGCGCAGCC
UGGCCUGCUCUCACGCUUCUUCAGCGCGCUGAAGAAUAUCUUCUCUGGCGCCGAAGAGGCCAAAC
CGGCUGAAGUUCAGGUCGAGAAGAAAGCGGAAGAAAAACCGGAGCGUCAGCAGGAGCGUCGUAAA
CCGCGCGCCAACAACCGCCGCGACCGCAACGACCGCCGUGAUAACCGCGACAAUCGUGACAACCGC
GAUAACCGUGACAAUCGCGACACCCGUGCGGACAAUGCCGAGGGCCGUGAACCGCGCGAAUCGCG
UGAAGAGAACCGUCGCAACCGUCGCGAGAAGCCGUCGCAGAACGUGGAAGCCCGUGAUGUUCGCC
AAACCUCAGGCGACGACGCGGAGAAAGCGAAAUCCCGUGACGAGCAGCAGCCGCGCCGCGAACGC
ACCCGCCGCCGCAGUGACGACAAACGUCAGGCGCAGCAGGAAGCCAAAGCGCAGACUCGCGAAGA
GCCGGUUGUGCAGGAGACGGAGCAGGAAGAGCGUGUACAAACUCUGCCGCGUCGUAAACCGCGCC
AGCUGGCACAGAAAGUGCGCGUUGAGUCCGCUGUCGUCGAGCCAGUUGCCGAGAUCGUGCCAGAA
GCCGUAGUGGCUGAAGUUAUCGCUCCGCACAGCGAGCCGGUGAAAGCCGAGCUGCCGGCAGGGG
UGGAGAGCGUGGCGGACCAGGACGAAAAUGGCGAAUCCCGUGAAGCGAACGGUAUGCCGCGUCGC
UCACGUCGCUCCCCGCGUCACCUGCGCGUCAGCGGUCAGCGUCGUCGUCGCUAUCGUGACGAACG
CUAUCCGACCCAGUCGCCUAUGCCGCUGACCGUAGCCUGCGCAUCGCCGGAGAUGGCUUCCGGUA
AAGUCUGGAUCCGCUACCCGGUGGUUCGUCCGCAGGAUCAGCAGCCGGAAGAGGUUCAGGUUCAG
GACGCCAGCGUCGCGAAAACUGUCGAGGCCGUAGCGGCCCCGGUCGCCGUCGUUGAAACCGUUAC
CGCUGCGCCGGUCACCGUCGAGCCGGCUACCAUGGAACCAGUAACCGCUGAGCCGGUAGUCGUCG
AGCCGGUAGCGGCCGCCGAGCCGCUGGUCGUUGAUGCUGCGGAAGUUGUCGCGCCAGCAGCCGU
CGAGCCAGCGCCUCAGGAGCCGGUCACCGAAGCACCGGCUGUCGAAGCGCCUCAGGCUAUCGCGC
CAGUGACGCUCGACGCCGAGCCGGUGGUGGUAGAACCUGAAGCGGUGGAAACGACGCCUGUCGUU
GCAGCGCCAGUGGAAACUAUCGCCCCGGUCGCAGAAACCGUGGAGCAAGCGCCAGUGACCGAAGC
GGCCCCUGCCGAACCGGUCAAAGCCGAGCCCCCGGUGAGCAAGCCGGUCGUAGUGGCGGGUCAUC
GCCAUGCCACCGCGCCAAUGACCCGUGCGCCAGCUCCGGACUAUGUCCCGGAAGCACCGCGUCAU
AGCACCUGGGUGCGCCCGCCGUUCGCCUUUGAAGGUAAAGGCGCCGCCGGUGGUCAUAGCGCGAC
CCAUAAAGCCACCGCUGAACCGACUCGCCCACAGCCCGUCGAGUAA
SEQ ID NO: 89
AUGCGCAAGCUCUCACUAAGUUUACUCACGCUGUCCCUCGGCGUUGCGCUGCUGCCGUUAGCGCA
GGCGGCGACGACGCCUGCCCAGGAGCAUCUGCUGGAGCAGGUCCGCCUCGGCGAGGCCAGCAAUC
GUGAAGACCUGGUGCGCCAGUCGCUGUACCGUCUGGAGCUGAUUGAUCCCAACAACCCGGAGCUG
AUUGCCGCGCGGAUGCGCUAUCUGCUGCGUCAGGGGGAUGCCGCCGGGGCGCAAAAAGAGCUGG
AACGACUGACGAAGCAGGCGCCGGACUCCCCGGAGCUGAAGGCGUCGCGCAAUGAGAUGAAAAGC
AACACCGGCGAGGGCCGCCAGGCGCUGCAGCAGGCGCGACUGCUGGGCGUGGCCGGGAAGGUCG
AUGAAGCCAUCGCCGCCUAUGAAAAACUGUACGGCGGGGUGCCGGAUGACGUUGACGUCGCCAUU
GAGUACUGGACGCUGGUGGCGCGCCUGCCGGCCCGCCAUAGCGAAGGCGUCAGCCAGUUGAAAAA
ACUGAACGCCAGCGCGCCGGGCAACGUCAGCCUGCUGACUUCGCUGGCGAAGCAGAUGUUCGCCG
AUAACAAACCGCAGGAGGGGUUCGCCUAUCUGGCGGAGAUGGCCCGAUCGGCCUCGGGACGCGGU
AUCGCCGCCGAUAUGUGGUUCAGUGAGGUGAAAAGCAUGCCGGUGAGUAAGGCCAGCGUGCAGGC
GUUGCAGCAAUUUCUUCUGCAGUUUCCCACCGGCUCGGUGGCGGCGAACGCCCGCGUUCUGCUCG
ACCAACAGCAGGCGCAGCUGCAGGAUCCGACUUUCCGCGCCCGCUCGGAAGGGCUGGCGGCGGUC
AAGUCCGGGAAUACCACGCAGGCGGUCGCGGAUCUGCAGAAAGCCGUUCAGGCCGACAGCCGCGA
CAGCGACGCGGUGGGCGCUCUCGGCCAGGCCUAUUCCCAGCGCGGCGACCGCGCGCGGGCAGUG
GCGCAGCUCAGUAAAGCGAUUGCUAUGGACCCUGACAGCCCGAACCGCGGCAAGUGGGACAGCCU
GCUGCAAACUAACCGCUACUGGCUGCUGAUAAAGCAGGGGGAUAACGCCCUGAAAGCCGGCCAGC
UUUCGCAGGCGCAGAACUAUUAUGCCCAGGCGCAGCGGGUCGAUCGCACCGACAGCUAUGCCGUG
CUGGGGCUGGGGGACGUCGCGGCGGCGCGCAAAGAGGCGGCGGCGGCGGAGCGCUAUUACCAGC
AGGCGUUGCGCCUGGAUCGCGGCAAUAACCUGGCGGUGCGCGGCCUGGCCAACCUCUAUCGCGCC
GAAUCGCCGGAGAAAGCCAGCGCCUGGAUCGCCGGCCUCCCUCCCGCUCAGCGGCGGAGCAUCGA
UGAUAUUGAGCGCAGCCUGACUAACGACCGGCUGGAGAAACAGGCGCAGGCUCUGGAGAGCCAGG
GCAACUGGGCGCAGGCGGCGGAAGUUCAGCGUCGGCGCCUGGCGCUGGAUCCGGACAGCGUCUG
GAUAACCUACCGUCUGGCGCGGGAUCUGGUCAGCGCCGGCGAACGCCAGGAGGCCGACGCGCUGA
UGCGGACGAUGGUCAACCGCCAGCCGCAGGACGCCGAACGGGUCUACGCCUCGGGACUCUACCUG
UCGGGGAACGACCAGGACGAUCUGGCUCUGGCGCAAAUCGCCGCUCUGCCGCGCAGCGCGUGGAC
GGAUAACAUUCGUGAGCUCGAAGCGCGUUUGCAAAGCGACCGGGUGCUGCGCCAGGCCAACCAGC
UGCGCGACAGCGGUAACGAAGCGCAGGCGAUCGCCCUUAUCCGACAGCAGCCCGCCUCGGUGCGC
UAUGACCUGACGCUCGCCGACUGGGCGCAGCAGCGCGGCGACAGCCAGACGGCGAUUGCCAACUA
UCAGCGGGUGCUGCGCCAGGAGGCCGACAACGGCGAUGCGCGCCUCGGCCUUGCGGAAGUCUACC
UGGCCGAGGGCGAUAAACCGGCCGCCCGGGCGCAGGUCAUGCAGCUGAAAGGCGCAGAGACCGAA
UCCAUGAACAUGCAGCGGCGGGUGGCGCUGGCGCGAGCUGGCCUUGGCGAUACCGCUGACGCGC
AACGGAUUUUUAAUCAGAUUGUGCCGCAGGCGAAGGCGCAGCCGCCCUCGAUGGAGAGCGCGCUG
GUGCUGCGCGAUGCCGCGCGCUUUGCCACCCAGAGCGGGGCGCCGCAGCAGGCGCUGACGCACUA
CCGGGAAGCUAUGGUGGCCUCCGGCAUUACCCCCGCGCAGCCGCAGGAUAACGAUACUUUUACGC
GGCUGACGCGCAACGACAGCCAUGAUGACUGGCUGAAGCGCGGGAUCCGCAGCGAUGCCGCCGAC
CUUUAUCGUCAGCAGGAUCUGAACGUCACCCUGGAACAUGACUUCUGGGGUUCCAGCGGCACCGG
CGGCUAUUCCGACCUGAAGGCGCAUACCACCAUGCUGCAGAUGGAUGCUCCGCUGGCGGAUGGCC
GGAUGUUCUUCCGCACCGACCUGGUCAAUAUGGAUGCCGGCAGCUUUUCCACCCACAGCGACGGG
AGCUACUCGCCCAGCUGGGGCACCUGCGGGGAGAUCGCCUGUACCAGCGGCAGUAAAAAUCAGAC
CGACAGCGGGGCCAGCGUGGCGGUCGGCUGGAAGAAUGACACCUGGAGCGGGGAUAUCGGCACC
ACGCCGAUGGGCUUCAAUGUCGUCGAUGUGGUGGGGGGGCUGAGCUACAGCAGCGACGUCGGGC
CGGUGGGGUACACGGUCAACGUCCACCGGCGGCCUAUCUCCAGCUCGCUGCUCUCCUUUGGCGGG
CAGAAGGACAGCAGCAGCCAUACCGGCGCCACCUGGGGCGGCGUCCGCGCCGACGGCGGCGGCCU
GAGCCUGAGCUACGAUCGCGGGGAGGCUCACGGCAUCUGGUCCUCGCUGGGCGCCGACUCGCUG
ACCGGUAAAAACGUGGCGGAUAACUGGCGCGUGCGCUGGAUGACCGGGUACUACUACAAGGUCAU
CAACGAGAAUAAUCGUCGCGUCACCGUCGGCCUCAACAAUAUGAUCUGGCACUACGACAAAGAUCU
CAGCGGCUACACCCUCGGCCAGGGCGGCUAUUACAGCCCACAGGAGUAUCUCUCGUUCGCCGUGC
CGGUGACCUGGCGUCAGCGCACCGAGAACUGGUCCUGGGAGCUCGGCGGGUCGGUGUCAUGGUC
CCAUUCGCGCACCCAGACGCAAGCCCGCUAUCCGCUGCUGAACCUGAUCCCGUCCGACUACCGGCA
GCGCGCCAGCGAGCUGACGGAGGAGGGGAGCAGCAGCCAUGGAUUCGGUUACACCGCCAGAGCGC
UGGUGGAGCGGCGGGUGACCAGCAACUGGUUCGUCGGCGCCGCGGUCGAUAUUCAGCAGGCGAA
GGAUUACACCCCGAGCCAUGCGCUGCUUUACGUCCGCUACUCGGCGGCCGGCUGGCAGGGGGAUC
UGGAUAUGCCGCCCCAGCCGCUGGUGCCCUACGCCGACUGGUAG
SEQ ID NO: 90
AUGAGCCAGGAAUACACCGAAGACAAAGAAGUCAAACUAACCAAACUCAGCAGCGGGCGCCGACUC
CUUGAGGCGAUGCUCAUCCUUUGCUCCCUCUUCGCCAUCUGGCUGAUGGCGGCACUACUGAGCUU
UAACCCCUCGGACCCCAGCUGGUCGCAAACGGCAUGGCAUGAGCCUAUUCAUAAUUUAGGCGGCG
CCCCCGGCGCGUGGCUUGCCGAUACCCUCUUUUUCAUUUUUGGCGUCAUGGCCUACACCAUCCCG
GUGAUCAUCAUCGGCGGAUGCUGGUUUGCCUGGCGGCAUCAGGAAAACGACGAAUACAUUGAUUA
UUUUGCCGUUUCCCUUCGCCUCAUCGGUGCGUUAGCCCUGAUCCUGACCUCCUGUGGUCUGGCGG
CGAUUAACGCCGAUGAUAUCUGGUACUUCGCCUCCGGCGGGGUGAUCGGCAGCCUGCUGAGCACC
ACGCUGCAACCCCUGCUGCACAGCAGCGGCGGCACCAUCGCCCUGUUGUGUAUCUGGGCGGCCGG
GCUGACGCUGUUCACCGGCUGGUCGUGGGUCAGCAUUGCGGAAAAGCUGGGCGGCGGCAUCCUG
UCCGUUCUCACCUUUGCCAGCAACCGUACCCGUCGGGAUGAUACCUGGGUCGAUGAAGGCGAAUA
UGAAGACGACGAGGAAGAGUACGACGACGAAGAGGCGGCCAGGCCGCAGGAAUCGCGUCGCGCCC
GUAUCUUACGCAGCGCGCUGGCGCGGCGUAAGCGUCUGGCCGAGAAGUUUACCAACCCUAUGGGG
CGUAAAACCGACGCUGCGCUUUUCUCCGGCAAACGGAUGGAUGACGGCGAAGAGGUGGUGCAAUA
CAGCGCCAGCGGGGCGCCUGUUGCCGCCGACGAUGUACUGUUUUCCGGCGCCAGCGCCGCGCGU
CCCGCAGAGGAUGAUGUGCUGUUCUCCGGCGCCAGCGCCGUGCGCCCGGGCGAUUUCGACCCUUA
CGAUCCGUUGUUGAAUGGCCACAGUAUCGCUGAGCCGGUAAGCGCAGCGGCGGCGGCUACAGCCG
CGCCGCAGGCGUGGGCAGAAUCACCGGUGGGCCAUCACGGCGCUGCGCCAGCUUAUCAGCCGGAA
GCCAGCUAUCCGCCGCAGCAGGCCUAUCAGCCUGAACCCGCUCCGUUCCAGCAGGCCUAUCAGCCU
GAACCCGCUCCGUUCCAGCAGGCUGCUUAUCAGCCGCCAGCGGGGCAAACCGCACCGCAGGCGUA
UCAGCCUGAGCCAGCGCCGUAUCAACAGCCGGUUUACGAUCCGCGUGCCGGUCAACCUGCGCCGC
AGGCCUAUCAGCCUGAGCCAGCGCCGUAUCAGCAGCCGGCUUACGAUCCGUAUGCCGGUCAACCU
GCGCCGCAGGCCUAUCAGCCUGAACCUGCGCCGUAUCAGCAGCCGGCUUACGAUCCGCAUGCCGG
UCAACCUGCACCGCAGGCCUAUCAGCCUGAGCCAGCGCCGUAUCAGCAGCCGGCUUACGAUCCCUA
UGCCGGUCAACCUGCGCCGCAGGCCUAUCAGCCGGAGCCAGCGCCGUAUCAGCAGCCAACUUACG
AUCCCUAUGCCGGUCAGCCUGCGCCUCAGACCUAUCAGCAGCCGGCUUACGAUCCGAAUGCCGGU
CAGCCCGCGCCGCAGCCGUAUCAGCCGGAGCCAGCGGCGUAUCAGCCGCAAAGCGCGCCAGUUCC
CCCACCGGAGCCAGAGCCCGAGGUCGUGCAGGAGGAAGUGAAACGUCCGCCGCUCUAUUAUUUCG
AGGAAGUGGAAGAGAAGCGGGCGCGCGAACGCGAGCUGUUGGCCUCCUGGUAUCAGCCAAUUCCU
GAGCCGGAAAGUCCGAUUGCCACUAAACCGCUGACGCCGCCGACCACUGCGUCCAAACCGCCAGUG
GAGACAACCGUAGUCUCUGCGGUAGCGGCUGGGGUGCAUCAGGCUACCGCCGCCAGCGGCGGCG
CGGCGGCAGCAACCUCGUCCACUGCCGCAUCCGCUGCGGCUACGCCAUUGUUCAGCCCGGCGUCC
AGCGGCCCAAGGGUUCAGGUGAAAGAGGGCAUCGGUCCAAAACUACCGCGGCCCAAUCGCGUGCG
UGUUCCUACGCGUCGGGAACUGGCCUCCUACGGCAUCAAGCUACCGUCGCAGCGGGAGGCGGAAC
AGCGCGCGCGGCAGGCGGAGCGCGAUCCGCAUUAUGAUGAUGAGCUGCUCUCGGAUGAGGAAGC
GGAUGCUAUGGAGCAGGAUGAACUGGCUCGCCAGUUCGCCGCCACCCAGCAGCAGCGCUACGGUC
AUCGCUGGGAAGACGAUAACGCGACUGAUGACGAUGAGGCCGACGCCGCGGCGGAAGCGGAGCUG
GCGCGUCAGUUUGCCGCUACCCAGCAGCAGCGGUACGCUACCGAGCAGCCGCCGGGCGCCAACCC
GUUCUCGCCGGCAGAUUAUGAAUUCUCGCCGAUGAAAACGUUGGUCAAUGACGGCCCGAGCGAAC
CGCUGUUUACGCCGACGCCGGAAGUCCAGCCGCAGCAGCCGGCCCAGCGCUAUCAACAACCGGCG
GCCGCUCCGCAGCAGGGUUAUCAACCUGCGCAGCAUCAGCCGAUACACCAUCAGCCUGUGCCGCC
ACAGCCGCAGUCCUAUCCGACUGCGUCGCAGCCCGUACAGCCGCAACAACCGGUUGCCCCGCAGG
GGCAUCAGCCUGCCGCCCCUGCGCCGCAGGAGAGCCUGAUCCACCCGCUGCUGAUGCGCAAUGGC
GAUAGUCGACCGCUGCAAAAGCCGACCACGCCACUGCCGUCGCUGGAUCUGCUUACCCCGCCGCC
GAGUGAAGUCGAGCCGGUGGAUACCUUUGCUCUCGAGCAGAUGGCACGCCUGGUGGAAGCGCGA
CUCGCUGAUUUCCGCAUUAAAGCGGAUGUGGUGAACUACUCACCGGGGCCGGUGAUCACCCGCUU
CGAACUGAAUCUGGCGCCUGGCGUUAAGGCCGCACGGAUCUCUAACCUGUCACGGGACCUGGCGC
GAUCGCUGUCAACGGUCGCCGUGCGCGUGGUGGAGGUGAUCCCGGGCAAACCGUAUGUCGGGCU
UGAGCUGCCGAAUAAAAAACGCCAGACCGUCUACCUGCGUGAAGUGCUCGACAACGCCAAGUUCCG
UGAUAACCCAUCUCCGCUCACCGUGGUGUUGGGUAAAGACAUCGCUGGCGAUCCGGUAGUAGCCG
AUCUGGCGAAAAUGCCGCAUCUGCUGGUGGCCGGUACCACCGGUUCCGGUAAGUCUGUUGGCGUC
AACGCCAUGAUCCUCAGCAUGCUCUACAAGGCGCAGCCGGAAGAUGUGCGUUUCAUUAUGAUCGA
CCCGAAAAUGCUCGAGCUGUCGGUCUACGAAGGAAUUCCGCACCUGCUGACGGAAGUGGUCACCG
ACAUGAAAGACGCCGCCAAUGCGCUGCGCUGGAGCGUCAAUGAGAUGGAGCGCCGCUACAAGCUG
AUGUCGGCGCUGGGCGUGCGUAACCUCGCGGGCUACAACGAGAAGAUCGCCGAAGCCGCGCGCAU
GGGACGUCCGAUCCCGGAUCCGUACUGGAAGCCUGGCGACAGCAUGGACGCCGUACAUCCGGUGC
UGGAAAAACUGCCGUACAUCGUGGUGCUGGUGGAUGAAUUCGCCGAUCUGAUGAUGACCGUCGGC
AAAAAGGUGGAAGAGCUGAUCGCUCGCCUGGCGCAGAAAGCGCGCGCGGCGGGGAUCCACCUGGU
GCUGGCGACACAGCGUCCGUCGGUAGAUGUUAUUACCGGCCUGAUUAAGGCCAACAUCCCGACGC
GCAUCGCCUUUACCGUGUCGAGUAAAAUUGACUCACGUACCAUUCUCGAUCAGGGCGGCGCGGAA
UCGCUGCUGGGUAUGGGGGAUAUGCUUUACUCCGGGCCGAACUCUACCACGCCGGUGCGUGUCCA
CGGGGCGUUUGUGCGCGACCAGGAAGUCCACGCCGUGGUUCAGGACUGGAAAGCCCGCGGUCGC
CCGCAAUAUGUGGAUGGCAUUACCUCCGACAGCGAAAGCGAAGGCGGCGGUGGCGGCUUCGACGG
CGGGGAAGAGUUGGAUCCGUUGUUCGAUCAGGCAGUCAACUUUGUGACCGAGAAGCGCAAAGCGU
CGAUUUCCGGGGUUCAGCGUCAGUUCCGCAUCGGCUAUAACCGUGCCGCGCGUAUUAUCGAACAG
AUGGAAGCGCAGGGUAUCGUCAGCGAGCAGGGCCAUAACGGUAACCGCGAAGUGCUGGCGCCGCC
GCCCUUUGAAUGA

The polypeptides of the invention can also be designated as follows:

| SEQ ID NO: | Designation |
|---|---|
| 1 | KP1_2999 |
| 2 | KP1_0355 |
| 3 | KP1_3995 |
| 4 | KP1_3023 |
| 5 | KP1_1709 |
| 6 | KP1_4823 |
| 7 | KP1_1443 |
| 8 | KP1_1444 |
| 9 | KP1_0026 |
| 10 | KP1_3952 |
| 11 | KP1_0090 |
| 12 | KP1_4374 |
| 13 | KP1_1620 |
| 14 | KP1_5089 |
| 15 | KP1_1701 |
| 16 | KP1_1186 |
| 17 | KP1_0947 |
| 18 | KP1_1632 |
| 19 | KP1_1274 |
| 20 | KP1_3820 |
| 21 | KP1_0953 |
| 22 | KP1_2972 |
| 23 | KP1_0306 |
| 24 | KP1_2284 |
| 25 | KP1_4356 |
| 26 | KP1_1032 |
| 27 | KP1_4886 |
| 28 | KP1_2074 |
| 29 | KP1_5221 |
| 30 | KP1_1891 |
| 91 | KP1_1547 |
| 92 | KP1_1958 |
| 93 | KP1_4144 |
| 94 | KP1_4102 |

When designating a fragment of one of these proteins, this is done using the nomenclature KP1_XXXX-A-p1-p2, where XXXX is any of the 4 digit numbers following “KP1” In the table above, and p1 and p2 are the start and end amino acids relative to the entire sequence of the protein. For instance KP1_1891-50-200 is the fragment of KP1 1891 that has the amino acid sequence defined by residues 50 to 200 of KP1_1891, i.e. a (poly) peptide having the amino acid sequence of SEQ ID NO: 30, residues 50-200.

### Example

### Challenge Studies in Mice

1. Challenge Study (Klebsiella pneumonia—Intranasal (IN) Model):
Challenge strain: Klebsiella pneumoniae NTUH-K2044 (Wu K M et al. (2009), J. Bacteriol, 191:4492-4501).
Mouse strain: BalbC/ByJ (inbred)
Dose: 25 μg in each immunization.
Immunization route: 3×subcutaneous
Immunization Interval: 14 days
Inoculation route: intranasal (IN)
End point: Lethal challenge
Adjuvant choice: Priming immunization used Alum+IFA (incomplete Freund's adjuvant); 1^{st} and 2^{nd} booster immunizations used alum only
Bleeds: Bleed mouse 4 days before challenge for subsequent ELISA test.
Trial type: Double blinded
No of mice per group: At most 16 mice (see tables 1-6 for details)
Monitoring period: 7 days
   2. Challenge Study (Klebsiella pneumonia—Intraperitoneal (IP Model)):
Challenge strain: Klebsiella pneumoniae NTUH-K2044)
Mouse strain: NMRI (outbred)
Dose: 25 μg in each immunization.
Immunization route: 3×subcutaneous
Immunization interval: 14 days
Inoculation route: intraperitoneal (IP)
End point: Lethal challenge
Adjuvant choice: Priming immunization used Alum+IFA (incomplete Freund's adjuvant); 1^{st} and 2^{nd} booster immunizations used alum only
Bleeds: Bleed mouse 4 days before challenge for subsequent ELISA test.
Trial type: Double blinded
No of mice per group: At most 16 mice (see tables 1-6 for details)
Monitoring period: 7 days

A total 6 experiments was carried out. Only experiment 2 utilised the IP model whereas the remaining experiments utilised the IN model.

A number of the tested proteins proved insoluble in saline. Instead they were solubilized in 4 M urea to avoid precipitation before adding aluminum hydroxide. After adding aluminum hydroxide the urea is removed completely. The remaining proteins are solubilized in physiological saline.

The protocol for immunization is described below.

### 1st Immunization:

25 μg protein (per mice) are mixed with 100 μl aluminum hydroxide (Alhydrogel 2.0%, Brenntag) per 125 μg protein and incubated with end-over-end rotation for 1 hour.
The compositions is centrifuged at 1000 rpm for 2 minutes and the supernatant is removed
The alu-beads are washed once (slowly) in 0.9% (0.15 M) NaCl to remove excess urea
Freund's incomplete adjuvant (Sigma) is added 1:1 (v/v) and the resulting mixture is vortexed thoroughly for 1 hour.
Subsequently the final composition is injected subcutaneously
   2^{nd} and 3^{rd} Immunization
The mice receive booster immunization with 2 weeks interval, using the same amount of protein mixed with aluminum hydroxide and physiological saline solution.
Subsequently the final composition is injected subcutaneously

The following table A lists the immunogens used in each of the immunization studies in 6 different experiments as well as the p-value for the survival in each group when compared to mice vaccinated with phosphate buffered saline alone.

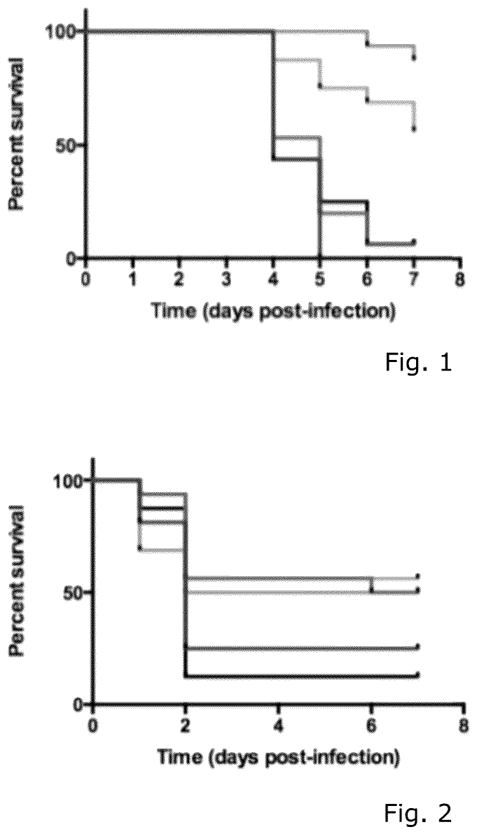

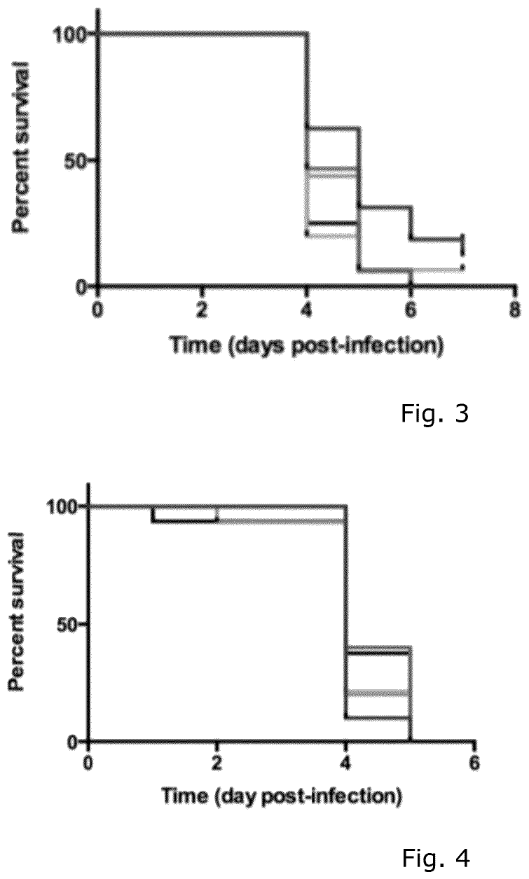

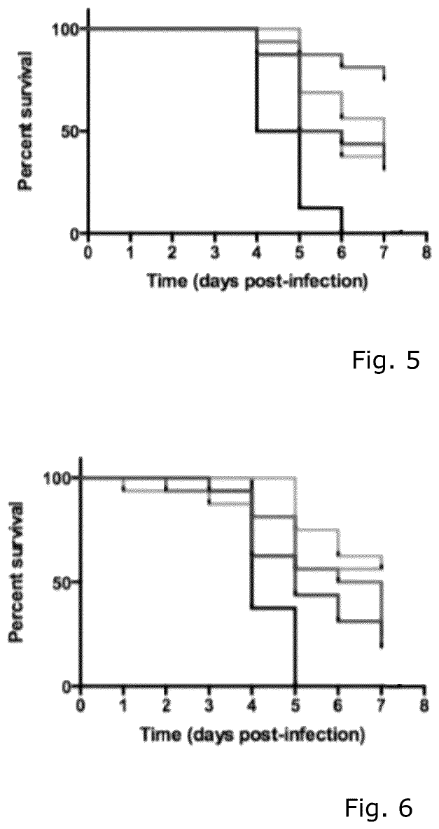

The corresponding survival plots are shown in FIGS. 1-6, which show the results for Experiments 1-6, respectively. The survival data are also indicated for each group of mice in each experiment in Tables 1-6.

TABLE A
Kaplan-Meier survival curve overview

| Challenge: | | P-value | |
|---|---|---|---|
| EXPERIMENT # 1 | | | |
| (IN model) | Immunization agent - Protein ID(s) | Log rank test | Fisher's exact test |
| Group 1 vs PBS | (KP1_1547-26-742; NCBI-ProteinID: BAH62300) | 0.2321 | 0.5000 |
| | SEQ ID NO: 91, residues 26-742 | | |
| | Positive control | | |
| Group 2 vs PBS | (KP1_1958-22-356, NCBI-ProteinID: BAH62679) | 0.9346 | 0.7419 |
| | SEQ ID NO: 92, residues 22-356 | | |
| | Postive control | | |
| Group 3 vs PBS | (KP1_4144-1-245, NCBI-ProteinID: BAH64685) | 0.0004 | 0.0030 |
| | SEQ ID NO: 93, residues 1-245 | | |
| | Postive control | | |
| Group 4 vs PBS | (KP1_1632-23-597 + KP1_1632-276-597 + KP1_1632-23-275) | <0.0001 | <0.0001 |
| | Mixture of 3 peptides: SEQ ID NO: 18, residues 23-597 + SEQ ID NO: 18 | | |
| | residues 276-597 + SEQ ID NO: 18, residues 23-275 | | |
| EXPERIMENT # 2 | | | |
| (IP model) | Protein ID | Log rank test | Fisher's exact test |
| Group 1 vs PBS | (KP1_5089-121-428 + KP1_0947-1-535 + KP1_0947-21-535) | 0.6869 | 0.32700 |
| | Mixture of 3 peptides: SEQ ID NO: 14, residues 121-428 + SEQ ID NO: | | |
| | 17, residues 1-535 + SEQ ID NO: 17, residues 21-535 | | |
| Group 2 vs PBS | (KP1_1620-1-382 + KP1_1620-21-382 + KP1_4374-27-376) | 0.0249 | 0.02690 |
| | Mixture of 3 peptides: SEQ ID NO: 13, residues 1-382 + SEQ ID NO: 13, | | |
| | residues 21-382 + SEQ ID NO: 12, residues 27-376 | | |
| Group 3 vs PBS | (KP1_0953-31-500 + KP_0953-31-675 + KP1_0355-22-117) | 0.0153 | 0.01170 |
| | Mixture of 3 peptides: SEQ ID NO: 21, residues 31-500 + SEQ ID NO: 21, | | |
| | residues 31-675 + SEQ ID NO: 2, residues 22-117. | | |
| Group 4 vs PBS | (KP1_4356-35-790 + KP1_4356-450-790 + KP1_4356-35-450 + | 0.2216 | 0.02690 |
| | KP1_0306-25-752 + KP1_0306-470-752 + KP1_0306-25-469) | | |
| | Mixture of 6 peptides: SEQ ID NO: 25, residues 35-790 + SEQ ID NO: 25, | | |
| | residues 450-790 + SEQ ID NO: 25, residues 35-450 + SEQ ID NO: 23, | | |
| | residues 25-752 + SEQ ID NO: 23, residues 470-752 + SEQ ID NO: 23, | | |
| | residues 25-4690 | | |
| EXPERIMENT # 3 | | | |
| (IN model) | Protein ID | Log rank test | Fisher's exact test |
| Group 1 vs PBS | (KP1_5221-23-650 (NaCl) + KP1_5221-651-1159 (Urea)) | 0.0156 | 0.2759 |
| | Mixture of 2 peptides: SEQ ID NO. 29, residues 23-650 (in saline) + SEQ | | |
| | ID NO: 29, residues 651-1159 (in Urea) | | |
| Group 2 vs PBS | (KP1_1186-35-484 (NaCl)) | 0.3488 | 1.0000 |
| | 1 peptide: SEQ ID NO: 16, residues 25-484 | | |
| Group 3 vs PBS | (KP1_3995-22-119 (NaCl) + KP1_3995-22-119 (Urea)) | 0.8585 | 0.4839 |
| | Mixture of 2 peptides: SEQ ID NO: 3, residues 22-119 (in saline) + SEQ | | |
| | ID NO: 3, residues 22-119 (in Urea) | | |
| Group 4 vs PBS | (KP1_1032-21-809 (Urea) + KP1_1032-451-809 (Urea) + KP1_1032-21- | 0.0576 | 0.1129 |
| | 450 (NaCl)) | | |
| | Mixture of 3 peptides: SEQ ID NO: 26, residues 21-809 (in Urea) + SEQ | | |
| | ID NO: 26, residues 451-809 (in Urea) + SEQ ID NO: 26, residues 21- | | |
| | 450 (in saline) | | |
| EXPERIMENT # 4 | | | |
| (IN model) | Protein ID | Log rank test | Fisher's exact test |
| Group 1 vs PBS | (KP1_1709-1-121 (NaCl)) | 0.2473 | 1.0000 |
| | 1 peptide: SEQ ID NO: 5, residues 1-121 (in saline) | | |
| Group 2 vs PBS | (KP1_2999-22-94 (NaCl)) | 0.7392 | 1.0000 |
| | 1 peptide: SEQ ID NO: 1, residues 22-94 (in saline) | | |
| Group 3 vs PBS | (KP1_1274-36-624 (NaCl)) | 0.5190 | 1.0000 |
| | 1 peptide: SEQ ID NO: 19, residues 36-624 (in saline) | | |
| Group 4 vs PBS | (KP1_0026-1-168 (NaCl)) | 0.3482 | 1.0000 |
| | 1 peptide: SEQ ID NO: 9, residues 1-168 (in saline) | | |
| EXPERIMENT # 5 | | | |
| (IN model) | Protein ID | Log rank test | Fisher's exact test |
| Group 1 vs PBS | (KP1_4102-582-1099 + KP1_4102-1100-1649, i.e. two fragments of | 0.0012 | 0.0217 |
| | NCBI-ProteinID: BAH64645) | | |
| | Mixture of 2 peptides: SEQ ID NO: 94, residues 582-1099 + SEQ ID NO: | | |
| | 94, residues 1100-1649 | | |
| | Postive control | | |
| Group 2 vs PBS | (KP1_3820-26-657 + KP1_3820-392-657 + KP1_3820-26-391 + | <0.0001 | <0.0001 |
| | KP1_3820-26-153 + KP1_0306-61-177) | | |
| | Mixture of 5 peptides: SEQ ID NO: 20, residues 26-657 + SEQ ID NO: 20, | | |
| | residues 392-657 + SEQ ID NO: 20, residues 26-391 + SEQ ID NO: 20, | | |
| | residues 26-153 + SEQ ID NO: 23, residues 61-177 | | |
| Group 3 vs PBS | (KP1_2284-1-761 + KP1_2284-515-761 + KP1_2284-1-514 + | 0.0019 | 0.0217 |
| | KP1_2284-61-176) | | |
| | Mixture of 4 peptides: SEQ ID NO: 24, residues 1-761 + SEQ ID NO: 24, | | |
| | residues 515-761 + SEQ ID NO: 24, residues 1-514 + SEQ ID NO: 24, | | |
| | residues 61-176 | | |
| Group 4 vs PBS | (KP1_2972-25-701 + KP1_2972-450-696 + KP1_2972-25-449 + | <0.0001 | 0.0088 |
| | KP1_2972-20-154) | | |
| | Mixture of 4 peptides: SEQ ID NO: 22, residues 25-701 + SEQ ID NO: 22, | | |
| | residues 450-696 + SEQ ID NO: 22, residues 25-449 + SEQ ID NO: 22, | | |
| | residues 20-154 | | |
| EXPERIMENT # 6 | | | |
| (IN model) | Protein ID | Log rank test | Fisher's exact test |
| Group 1 vs PBS | (KP1_5089-121-428 + KP1_0947-1-535 + KP1_0947-21-535) | 0.0148 | 0.1129 |
| | Mixture of 3 peptides: SEQ D NO: 14, residues 121-428 + SEQ ID NO: | | |
| | 17, residues 1-535 + SEQ ID NO: 17, residues 21-535 | | |
| Group 2 vs PBS | (KP1_1620-1-382 + KP1_1620-21-382 + KP1_4374-27-376) | 0.0006 | 0.0506 |
| | Mixture of 3 peptides: SEQ ID NO: 13, residues 1-382 + SEQ ID NO: 13, | | |
| | residues 21-382 + SEQ ID NO: 12, residues 27-376 | | |
| Group 3 vs PBS | (KP1_0953-31-500 + KP1_0953-31-675 + KP1_0355-22-117) | <0.0001 | 0.0004 |
| | Mixture of 3 peptides: SEQ ID NO: 21, residues 31-500 + SEQ ID NO: 21, | | |
| | residues 31-675 + SEQ ID NO: 2, residues 22-117 | | |
| Group 4 vs PBS | (KP1_4356-35-790 + KP1_4356-450-790 + KP1_4356-35-450 + | 0.0012 | 0.0004 |
| | KP1_0306-25-752 + KP1_0306-470-752 + KP1_0306-25-469) | | |
| | Mixture of 6 peptides: SEQ ID NO: 25, residues 35-790 + SEQ ID NO: 25, | | |
| | residues 450-790 + SEQ ID NO: 25, residues 35-450 + SEQ ID NO: 23, | | |
| | residues 25-752 + SEQ ID NO: 23, residues 470-752 + SEQ ID NO: 23, | | |
| | residues 25-469 | | |

TABLE 1
survival in Experiment 1

| | Sur- | Day | Day | Day | Day | Day | Day | Day | Day |
|---|---|---|---|---|---|---|---|---|---|
| Group | vival | 0 | 1 | 2 | 3 | 4 | 5 | 6 | 7 |
| 1 | # | 16 | 16 | 16 | 16 | 7 | 0 | 0 | 0 |
| | Alive | | | | | | | | |
| | % | 100.0 | 100.0 | 100.0 | 100.0 | 43.8 | 0.0 | 0.0 | 0.0 |
| | Sur- | | | | | | | | |
| | vival | | | | | | | | |
| 2 | # | 15 | 15 | 15 | 15 | 8 | 3 | 1 | 1 |
| | Alive | | | | | | | | |
| | % | 100.0 | 100.0 | 100.0 | 100.0 | 53.3 | 20.0 | 6.7 | 6.7 |
| | Sur- | | | | | | | | |
| | vival | | | | | | | | |
| 3 | # | 16 | 16 | 16 | 16 | 14 | 12 | 11 | 9 |
| | Alive | | | | | | | | |
| | % | 100.0 | 100.0 | 100.0 | 100.0 | 87.5 | 75.0 | 68.8 | 56.3 |
| | Sur- | | | | | | | | |
| | vival | | | | | | | | |
| 4 | # | 16 | 16 | 16 | 16 | 16 | 16 | 15 | 14 |
| | Alive | | | | | | | | |
| | % | 100.0 | 100.0 | 100.0 | 100.0 | 100.0 | 100.0 | 93.8 | 87.5 |
| | Sur- | | | | | | | | |
| | vival | | | | | | | | |
| 5 | # | 16 | 16 | 16 | 16 | 7 | 4 | 1 | 1 |
| | Alive | | | | | | | | |
| | % | 100.0 | 100.0 | 100.0 | 100.0 | 43.8 | 25.0 | 6.3 | 6.3 |
| | Sur- | | | | | | | | |
| | vival | | | | | | | | |

TABLE 2
survival in Experiment 2

| | | Day | Day | Day | Day | Day | Day | Day | Day |
|---|---|---|---|---|---|---|---|---|---|
| Group | | 0 | 1 | 2 | 3 | 4 | 5 | 6 | 7 |
| 1 | # | 16 | 13 | 4 | 4 | 4 | 4 | 4 | 4 |
| | Alive | | | | | | | | |
| | % | 100 | 81.3 | 25.0 | 25.0 | 25.0 | 25.0 | 25.0 | 25.0 |
| | Sur- | | | | | | | | |
| | vival | | | | | | | | |
| 2 | # | 16 | 15 | 9 | 9 | 9 | 9 | 8 | 8 |
| | Alive | | | | | | | | |
| | % | 100 | 94 | 56 | 56 | 56 | 56 | 50 | 50 |
| | Sur- | | | | | | | | |
| | vival | | | | | | | | |
| 3 | # | 16 | 15 | 9 | 9 | 9 | 9 | 9 | 9 |
| | Alive | | | | | | | | |
| | % | 100 | 94 | 56 | 56 | 56 | 56 | 56 | 56 |
| | Sur- | | | | | | | | |
| | vival | | | | | | | | |
| 4 | # | 16 | 11 | 8 | 8 | 8 | 8 | 8 | 8 |
| | Alive | | | | | | | | |
| | % | 100 | 69 | 50 | 50 | 50 | 50 | 50 | 50 |
| | Sur- | | | | | | | | |
| | vival | | | | | | | | |
| 5 | # | 16 | 14 | 2 | 2 | 2 | 2 | 2 | 2 |
| | Alive | | | | | | | | |
| | % | 100 | 87.5 | 12.5 | 12.5 | 12.5 | 12.5 | 12.5 | 12.5 |
| | Sur- | | | | | | | | |
| | vival | | | | | | | | |

TABLE 3
survival in Experiment 3

| | | Day | Day | Day | Day | Day | Day | Day | Day |
|---|---|---|---|---|---|---|---|---|---|
| Group | | 0 | 1 | 2 | 3 | 4 | 5 | 6 | 7 |
| 1 | # | 16 | 16 | 16 | 16 | 10 | 5 | 3 | 1 |
| | Alive | | | | | | | | |
| | % | 100.0 | 100.0 | 100.0 | 100.0 | 62.5 | 31.3 | 18.8 | 6.3 |
| | Sur- | | | | | | | | |
| | vival | | | | | | | | |
| 2 | # | 15 | 15 | 15 | 15 | 7 | 1 | 0 | |
| | Alive | | | | | | | | |
| | % | 100.0 | 100.0 | 100.0 | 100.0 | 46.7 | 6.7 | 0.0 | |
| | Sur- | | | | | | | | |
| | vival | | | | | | | | |
| 3 | # | 15 | 15 | 15 | 15 | 3 | 1 | 1 | 1 |
| | Alive | | | | | | | | |
| | % | 100.0 | 100.0 | 100.0 | 100.0 | 20.0 | 6.7 | 6.7 | 6.7 |
| | Sur- | | | | | | | | |
| | vival | | | | | | | | |
| 4 | # | 16 | 16 | 16 | 16 | 7 | 4 | 3 | 3 |
| | Alive | | | | | | | | |
| | % | 100.0 | 100.0 | 100.0 | 100.0 | 43.8 | 25.0 | 18.8 | 18.8 |
| | Sur- | | | | | | | | |
| | vival | | | | | | | | |
| 5 | # | 16 | 16 | 16 | 16 | 4 | 1 | 0 | 0 |
| | Alive | | | | | | | | |
| | % | 100.0 | 100.0 | 100.0 | 100.0 | 25.0 | 6.3 | 0.0 | 0.0 |
| | Sur- | | | | | | | | |
| | vival | | | | | | | | |

TABLE 4
survival in Experiment 4

| | | Day | Day | Day | Day | Day | Day | Day | Day |
|---|---|---|---|---|---|---|---|---|---|
| Group | | 0 | 1 | 2 | 3 | 4 | 5 | 6 | 7 |
| 1 | # | 10 | 10 | 10 | 10 | 1 | 0 | 0 | 0 |
| | Alive | | | | | | | | |
| | % | 100.0 | 100.0 | 100.0 | 100.0 | 10.0 | 0.0 | 0.0 | 0.0 |
| | Sur- | | | | | | | | |
| | vival | | | | | | | | |
| 2 | # | 15 | 15 | 15 | 15 | 6 | 0 | 0 | 0 |
| | Alive | | | | | | | | |
| | % | 93.8 | 93.8 | 93.8 | 93.8 | 37.5 | 0.0 | 0.0 | 0.0 |
| | Sur- | | | | | | | | |
| | vival | | | | | | | | |
| 3 | # | 14 | 14 | 14 | 14 | 3 | 0 | 0 | 0 |
| | Alive | | | | | | | | |
| | % | 100.0 | 100.0 | 100.0 | 100.0 | 21.4 | 0.0 | 0.0 | 0.0 |
| | Sur- | | | | | | | | |
| | vival | | | | | | | | |
| 4 | # | 15 | 15 | 14 | 14 | 3 | 0 | 0 | 0 |
| | Alive | | | | | | | | |
| | % | 100.0 | 100.0 | 93.3 | 93.3 | 20.0 | 0.0 | 0.0 | 0.0 |
| | Sur- | | | | | | | | |
| | vival | | | | | | | | |
| 5 | # | 16 | 15 | 15 | 15 | 6 | 0 | 0 | 0 |
| | Alive | | | | | | | | |
| | % | 100.0 | 93.8 | 93.8 | 93.8 | 37.5 | 0.0 | 0.0 | 0.0 |
| | Sur- | | | | | | | | |
| | vival | | | | | | | | |

TABLE 5
survival in Experiment 5

| | | Day | Day | Day | Day | Day | Day | Day | Day |
|---|---|---|---|---|---|---|---|---|---|
| Group | | 0 | 1 | 2 | 3 | 4 | 5 | 6 | 7 |
| 1 | # | 16 | 16 | 16 | 16 | 14 | 8 | 7 | 5 |
| | Alive | | | | | | | | |
| | % | 100.0 | 100.0 | 100.0 | 100.0 | 87.5 | 50.0 | 43.8 | 31.3 |
| | Sur- | | | | | | | | |
| | vival | | | | | | | | |
| 2 | # | 16 | 16 | 16 | 16 | 15 | 14 | 13 | 12 |
| | Alive | | | | | | | | |
| | % | 100.0 | 100.0 | 100.0 | 100.0 | 93.8 | 87.5 | 81.3 | 75.0 |
| | Sur- | | | | | | | | |
| | vival | | | | | | | | |
| 3 | # | 16 | 16 | 16 | 16 | 14 | 8 | 6 | 5 |
| | Alive | | | | | | | | |
| | % | 100.0 | 100.0 | 100.0 | 100.0 | 87.5 | 50.0 | 37.5 | 31.3 |
| | Sur- | | | | | | | | |
| | vival | | | | | | | | |
| 4 | # | 16 | 16 | 16 | 16 | 16 | 11 | 9 | 6 |
| | Alive | | | | | | | | |
| | % | 100.0 | 100.0 | 100.0 | 100.0 | 100.0 | 68.8 | 56.3 | 37.5 |
| | Sur- | | | | | | | | |
| | vival | | | | | | | | |
| 5 | # | 16 | 16 | 16 | 16 | 8 | 2 | 0 | |
| | Alive | | | | | | | | |
| | % | 100.0 | 100.0 | 100.0 | 100.0 | 50.0 | 12.5 | 0.0 | |
| | Sur- | | | | | | | | |
| | vival | | | | | | | | |

TABLE 6
survival in Experiment 6

| | | Day | Day | Day | Day | Day | Day | Day | Day |
|---|---|---|---|---|---|---|---|---|---|
| Group | | 0 | 1 | 2 | 3 | 4 | 5 | 6 | 7 |
| 1 | # | 16 | 16 | 16 | 15 | 10 | 7 | 5 | 3 |
| | Alive | | | | | | | | |
| | % | 100.0 | 100.0 | 100.0 | 93.8 | 62.5 | 43.8 | 31.3 | 18.8 |
| | Sur- | | | | | | | | |
| | vival | | | | | | | | |
| 2 | # | 16 | 16 | 15 | 15 | 13 | 9 | 8 | 4 |
| | Alive | | | | | | | | |
| | % | 100.0 | 100.0 | 93.8 | 93.8 | 81.3 | 56.3 | 50.0 | 25.0 |
| | Sur- | | | | | | | | |
| | vival | | | | | | | | |
| 3 | # | 16 | 16 | 16 | 16 | 16 | 12 | 10 | 9 |
| | Alive | | | | | | | | |
| | % | 100.0 | 100.0 | 100.0 | 100.0 | 100.0 | 75.0 | 62.5 | 56.3 |
| | Sur- | | | | | | | | |
| | vival | | | | | | | | |
| 4 | # | 16 | 15 | 15 | 14 | 13 | 9 | 9 | 9 |
| | Alive | | | | | | | | |
| | % | 100.0 | 93.8 | 93.8 | 87.5 | 81.3 | 56.3 | 56.3 | 56.3 |
| | Sur- | | | | | | | | |
| | vival | | | | | | | | |
| 5 | # | 16 | 16 | 16 | 16 | 6 | 0 | | |
| | Alive | | | | | | | | |
| | % | 100.0 | 100.0 | 100.0 | 100.0 | 37.5 | 0.0 | | |
| | Sur- | | | | | | | | |
| | vival | | | | | | | | |

1. A method for treatment or amelioration of infection with K. pneumoniae, in particular infection with multi-resistant K. pneumoniae, comprising administering to an individual in need thereof a therapeutically effective amount of a monoclonal antibody, which specifically binds to a polypeptide consisting of an amino acid sequence selected from the group consisting of SEQ ID NO: 20, residues 26-657 of SEQ ID NO: 20, residues 392-657 of SEQ ID NO: 20, residues 26-391 of SEQ ID NO: 20, and resides 26-153 of SEQ ID NO: 20.

2. The method according to claim 1, wherein the monoclonal antibody specifically binds to a polypeptide consisting of residues 26-657 of SEQ ID NO: 20.

3. The method according to claim 1, wherein the monoclonal antibody specifically binds to a polypeptide consisting of residues 392-657 of SEQ ID NO: 20.

4. The method according to claim 1, wherein the monoclonal antibody specifically binds to a polypeptide consisting of residues 26-391 of SEQ ID NO: 20.

5. The method according to claim 1, wherein the monoclonal antibody specifically binds to a polypeptide consisting of residues 26-153 of SEQ ID NO: 20.

6. The method according to claim 1, wherein the monoclonal antibody is selected from a multi-domain antibody and a single-domain antibody.

7. The method according to claim 6, wherein the multi-domain antibody is selected from a murine antibody, a humanized antibody, and a fully human antibody.

8. A method for prophylaxis, treatment or amelioration of infection with K. pneumoniae, in particular infection with multi-resistant K. pneumoniae, comprising administering to an individual in need thereof a therapeutically effective amount of an antibody analogue, which specifically binds to a polypeptide consisting of an amino acid sequence selected from the group consisting of SEQ ID NO: 20, residues 26-657 of SEQ ID nO: 20, residues 392-657 of SEQ ID NO: 20, residues 26-391 of SEQ ID NO: 20, and residues 26-153 OF seq id no: 20, wherein the antibody analogue is selected from an Fab or an F(ab′)2, and an scFV.

9. The method according to claim 8, wherein the antibody analogue specifically binds to a polypeptide consisting of residues 26-657 of SEQ ID NO: 20.

10. The method according to claim 8, wherein the antibody analogue specifically binds to a polypeptide consisting of residues 392-657 of SEQ ID NO: 20.

11. The method according to claim 8, wherein the antibody analogue specifically binds to a polypeptide consisting of residues 26-391 of SEQ ID NO: 20.

12. The method according to claim 8, wherein the antibody analogue specifically binds to a polypeptide consisting of residues 26-153 of SEQ ID NO: 20.
